SEQUENCE LISTING (602 sequences)

SEQ ID NO: 1 - protein, 5 aa, synthetic construct
TYTMH

SEQ ID NO: 2 - protein, 5 aa, synthetic construct
TYPMH

SEQ ID NO: 3 - protein, 5 aa, synthetic construct
TYGMH

SEQ ID NO: 4 - protein, 10 aa, synthetic construct
GFTFRTYTMH

SEQ ID NO: 5 - protein, 10 aa, synthetic construct
GFTFRTYPMH

SEQ ID NO: 6 - protein, 10 aa, synthetic construct
GFTFRTYGMH

SEQ ID NO: 7 - protein, 8 aa, synthetic construct
GFTFRTYT

SEQ ID NO: 8 - protein, 8 aa, synthetic construct
GFTFRTYP

SEQ ID NO: 9 - protein, 8 aa, synthetic construct
GFTFRTYG

SEQ ID NO: 10 - protein, 17 aa, synthetic construct
VIWYDGSNKHYADSVKG

SEQ ID NO: 11 - protein, 6 aa, synthetic construct
WYDGSN

SEQ ID NO: 12 - protein, 8 aa, synthetic construct
IWYDGSNK

SEQ ID NO: 13 - protein, 13 aa, synthetic construct
APQWELVHEAFDI

SEQ ID NO: 14 - protein, 13 aa, synthetic construct
ALIWELFHEYFDI

SEQ ID NO: 15 - protein, 13 aa, synthetic construct
ALLWELFHEYFDI

SEQ ID NO: 16 - protein, 15 aa, synthetic construct
ARAPQWELVHEAFDI

SEQ ID NO: 17 - protein, 15 aa, synthetic construct
ARALIWELFHEYFDI

SEQ ID NO: 18 - protein, 15 aa, synthetic construct
ARALLWELFHEYFDI

SEQ ID NO: 19 - protein, 11 aa, synthetic construct
GGNNLGSKSVH

SEQ ID NO: 20 - protein, 11 aa, synthetic construct
GGNNLISKSVH

SEQ ID NO: 21 - protein, 11 aa, synthetic construct
GGNNLVSKSVH

SEQ ID NO: 22 - protein, 6 aa, synthetic construct
NLGSKS

SEQ ID NO: 23 - protein, 6 aa, synthetic construct
NLISKS

SEQ ID NO: 24 - protein, 6 aa, synthetic construct
NLVSKS

SEQ ID NO: 25 - protein, 7 aa, synthetic construct
DVSDRPS

SEQ ID NO: 26 - protein, 7 aa, synthetic construct
DDSDRPS

SEQ ID NO: 27 - protein, 11 aa, synthetic construct
QVWDSSSTFVV

SEQ ID NO: 28 - protein, 11 aa, synthetic construct
QVWDSSSTYVV

SEQ ID NO: 29 - protein, 11 aa, synthetic construct
QVWDSSSDHVV

SEQ ID NO: 30 - protein, 11 aa, synthetic construct
QVWDSSSTIVV

SEQ ID NO: 31 - protein, 11 aa, synthetic construct
QVWDKSSTYVV

SEQ ID NO: 32 - protein, 122 aa, synthetic construct
QMQLVESGGGVVQPGRSLRLSCAASGFTFRTYTMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 33 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 34 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 35 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARALLWELFHEYFDIWGQGTMVTVSS

SEQ ID NO: 36 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARALLWELFHEYFDIWGQGTMVTVSS

SEQ ID NO: 37 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTFVVFGGGTKLTVL

SEQ ID NO: 38 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTYVVFGGGTKLTVL

SEQ ID NO: 39 - protein, 108 aa, synthetic construct
SYVLTQPPSVSVAPGQTARITCGGNNLISKSVHWYQQKPGQAPVLVVYDDSDRPSWIPERFSGSNSGNTATLTISRGEAGDEADYYCQVWDSSSTYVVFGGGTKLTVL

SEQ ID NO: 40 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTYVVFGGGTKLTVL

SEQ ID NO: 41 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTFVVFGGGTKLTVL

SEQ ID NO: 42 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSDHVVFGGGTKLTVL

SEQ ID NO: 43 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTIVVFGGGTKLTVL

SEQ ID NO: 44 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDKSSTYVVFGGGTKLTVL

SEQ ID NO: 45 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTYVVFGGGTKLTVL

SEQ ID NO: 46 - protein, 107 aa, Homo sapiens [sequence omitted]

SEQ ID NO: 47 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 50 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 52 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 66 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 67 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 68 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 69 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 70 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 71 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 72 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 73 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 74 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 75 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 76 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 77 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 78 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 79 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 83 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 84 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 85 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 86 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 87 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 88 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 89 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPG

SEQ ID NO: 90 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPG

SEQ ID NO: 91 - protein, 326 aa, synthetic construct; identical to SEQ ID NO: 49

SEQ ID NO: 92 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 93 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 94 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 95 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 96 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 97 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 98 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 99 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 100 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 101 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 103 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 104 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 105 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 106 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 107 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 108 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 109 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 110 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 111 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 112 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 113 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 135 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 136 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 137 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 138 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 139 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 140 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 141 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 142 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 143 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 144 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 145 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 146 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 147 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 148 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 149 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 150 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 151 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 152 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPG

SEQ ID NO: 153 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 154 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 155 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 156 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 157 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 158 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 159 - protein, 330 aa, Homo sapiens [sequence omitted]

SEQ ID NO: 160 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 164 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 178 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 179 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 180 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 181 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 182 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 183 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 184 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 185 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 186 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 187 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 188 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 189 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 190 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 191 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 192 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 193 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 195 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 196 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 197 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 198 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 199 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 200 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 201 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 202 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 203 - protein, 327 aa, synthetic construct; identical to SEQ ID NO: 161

SEQ ID NO: 204 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 205 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 206 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 207 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 208 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 209 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 210 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 211 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 212 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 213 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 214 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 215 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 216 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 217 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 218 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 219 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 220 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 221 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 222 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 223 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 224 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 225 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 247 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 248 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 249 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 250 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 251 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 252 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 253 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 254 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 255 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 256 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 257 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 258 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 259 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 260 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 261 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 262 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 263 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 264 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 265 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 266 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 267 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 268 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 269 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 270 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 271 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 272 - protein, 159 aa, Macaca fascicularis
MKSLGQSKKEEVSFRKFFIFQLVGLVLTYDFTNCDFQKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 273 - protein, 5 aa, synthetic construct
NYAMT

SEQ ID NO: 274 - protein, 7 aa, synthetic construct
GFTFSNY

SEQ ID NO: 275 - protein, 8 aa, synthetic construct
GFTFSNYA

SEQ ID NO: 276 - protein, 5 aa, synthetic construct
NYDIN

SEQ ID NO: 277 - protein, 7 aa, synthetic construct
GYTFTNY

SEQ ID NO: 278 - protein, 8 aa, synthetic construct
GYTFTNYD

SEQ ID NO: 279 - protein, 7 aa, synthetic construct
SGGYYWS

SEQ ID NO: 280 - protein, 9 aa, synthetic construct
GGSISSGGY

SEQ ID NO: 281 - protein, 10 aa, synthetic construct
GGSISSGGYY

SEQ ID NO: 282 - protein, 5 aa, synthetic construct
SYAMT

SEQ ID NO: 283 - protein, 7 aa, synthetic construct
GFTFSSY

SEQ ID NO: 284 - protein, 8 aa, synthetic construct
GFTFSSYA

SEQ ID NO: 285 - protein, 17 aa, synthetic construct
LISGSGGLTKYADSVKG

SEQ ID NO: 286 - protein, 6 aa, synthetic construct
SGSGGL

SEQ ID NO: 287 - protein, 8 aa, synthetic construct
ISGSGGLT

SEQ ID NO: 288 - protein, 17 aa, synthetic construct
WIHPNTGKTGYAQKFQG

SEQ ID NO: 289 - protein, 6 aa, synthetic construct
HPNTGK

SEQ ID NO: 290 - protein, 8 aa, synthetic construct
IHPNTGKT

SEQ ID NO: 291 - protein, 16 aa, synthetic construct
HIYYSGSTKYNPSLKS

SEQ ID NO: 292 - protein, 5 aa, synthetic construct
YYSGS

SEQ ID NO: 293 - protein, 7 aa, synthetic construct
IYYSGST

SEQ ID NO: 294 - protein, 17 aa, synthetic construct
LISGSGGLTKYADSVKG

SEQ ID NO: 295 - protein, 6 aa, synthetic construct
SGSGGL

SEQ ID NO: 296 - protein, 8 aa, synthetic construct
ISGSGGLT

SEQ ID NO: 297 - protein, 9 aa, synthetic construct
DEGLTTGEY

SEQ ID NO: 298 - protein, 7 aa, synthetic construct
EGLTTGE

SEQ ID NO: 299 - protein, 11 aa, synthetic construct
VKDEGLTTGEY

SEQ ID NO: 300 - protein, 16 aa, synthetic construct
ESYDLWSGHSFNWFDP

SEQ ID NO: 301 - protein, 14 aa, synthetic construct
SYDLWSGHSFNWFD

SEQ ID NO: 302 - protein, 18 aa, synthetic construct
ARESYDLWSGHSFNWFDP

SEQ ID NO: 303 - protein, 12 aa, synthetic construct
DNWGSGWYYMDV

SEQ ID NO: 304 - protein, 10 aa, synthetic construct
NWGSGWYYMD

SEQ ID NO: 305 - protein, 14 aa, synthetic construct
ARDNWGSGWYYMDV

SEQ ID NO: 306 - protein, 9 aa, synthetic construct
DEGLTTGEY

SEQ ID NO: 307 - protein, 7 aa, synthetic construct
EGLTTGE

SEQ ID NO: 308 - protein, 11 aa, synthetic construct
AKDEGLTTGEY

SEQ ID NO: 309 - protein, 11 aa, synthetic construct
RASQDIRNDLA

SEQ ID NO: 310 - protein, 7 aa, synthetic construct
SQDIRND

SEQ ID NO: 311 - protein, 6 aa, synthetic construct
QDIRND

SEQ ID NO: 312 - protein, 11 aa, synthetic construct
RASQGITSYLA

SEQ ID NO: 313 - protein, 7 aa, synthetic construct
SQGITSY

SEQ ID NO: 314 - protein, 7 aa, synthetic construct
SQSVSNY

SEQ ID NO: 315 - protein, 6 aa, synthetic construct
QSVSNY

SEQ ID NO: 316 - protein, 11 aa, synthetic construct
RASQGIRNDLG

SEQ ID NO: 317 - protein, 7 aa, synthetic construct
SQGIRND

SEQ ID NO: 318 - protein, 6 aa, synthetic construct
QGIRND

SEQ ID NO: 319 - protein, 7 aa, synthetic construct
ASSSLQS

SEQ ID NO: 320 - skipped

SEQ ID NO: 321 - protein, 7 aa, synthetic construct
GATTLQS

SEQ ID NO: 322 - skipped

SEQ ID NO: 323 - protein, 7 aa, synthetic construct
DASNRAT

SEQ ID NO: 324 - skipped

SEQ ID NO: 325 - protein, 7 aa, synthetic construct
ATSSFQS

SEQ ID NO: 326 - skipped

SEQ ID NO: 327 - protein, 9 aa, synthetic construct
LQHNNYPFT

SEQ ID NO: 328 - protein, 6 aa, synthetic construct
HNNYPF

SEQ ID NO: 329 - protein, 9 aa, synthetic construct
QQLQSYPYT

SEQ ID NO: 330 - protein, 6 aa, synthetic construct
LQSYPY

SEQ ID NO: 331 - protein, 9 aa, synthetic construct
QHRSNWPLT

SEQ ID NO: 332 - protein, 6 aa, synthetic construct
RSNWPL

SEQ ID NO: 333 - protein, 9 aa, synthetic construct
LQHNSYPFT

SEQ ID NO: 334 - protein, 6 aa, synthetic construct
HNSYPF

SEQ ID NO: 335 - protein, 118 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 336 - protein, 125 aa, synthetic construct
QVQLVQSGTEVKKPGASVKVSCKASGYTFTNYDINWVRQATGQGLEWVGWIHPNTGKTGYAQKFQGRVTMTRDISIGTAYMELNSLRSEDTAVYYCARESYDLWSGHSFNWFDPWGQGTLVTVSS

SEQ ID NO: 337 - protein, 122 aa, synthetic construct
QVQLQESGPGLVKPSQTLSLTCTVSGGSISSGGYYWSWIRQYPGKGLEWIGHIYYSGSTKYNPSLKSRLTISVDTSKNQFSLKLSSVTAADTAVYYCARDNWGSGWYYMDVWGKGTTVTVSS

SEQ ID NO: 338 - protein, 118 aa, synthetic construct
EVRLVESGGGLVQPGGSLRLSCAASGFTFSSYAMTWVRQAPGKGLEWVSLISGSGGLTKYADSVKGRFTISRDNSKKTLYLQMNSLRAEDTAVYYCAKDEGLTTGEYWGQGTLVTVSS

SEQ ID NO: 339 - protein, 107 aa, synthetic construct
DIQMTQSPSSLSASVGDRVTITCRASQDIRNDLAWYQQKPGKAPKRLIYASSSLQSGVPSRFSGSGSGTEFTLTISSLQPEDFATYYCLQHNNYPFTFGPGTKVDIK

SEQ ID NO: 340 - protein, 107 aa, synthetic construct
DIQLTQSPSFLSASVGDRVIITCRASQGITSYLAWYQRKPGKAPKLLIYGATTLQSGVPSRFSGSGSGTEFTLTISSLQPEDFATYYCQQLQSYPYTFGQGTKLEIK

SEQ ID NO: 341 - protein, 107 aa, synthetic construct
EIVLTQSPATLSLSPGERATLSCRASQSVSNYFAWYRQKPGQAPRLLIYDASNRATGIPARFSGSGSGTDFTLTISSLEPEDFAVYYCQHRSNWPLTFGGGTKVEIK

SEQ ID NO: 342 - protein, 107 aa, synthetic construct
DIQMTQSPSSLSASVGDRVTITCRASQGIRNDLGWYQQKPGKAPKRLIYATSSFQSGVPSRFSGSGSGTEFTLTISSLQPEDFATYYCLQHNSYPFTFGPGTKVDVK

SEQ ID NO: 343 - protein, 6 aa, synthetic construct
QGITSY

SEQ ID NO: 344 - protein, 11 aa, synthetic construct
RASQSVSNYFA

SEQ ID NO: 345 - protein, 11 aa, synthetic construct
RTSQNIRYYLN

SEQ ID NO: 346 - protein, 11 aa, synthetic construct
GGDNIGSKSVH

SEQ ID NO: 347 - protein, 11 aa, synthetic construct
GGQNIGTKSVH

SEQ ID NO: 348 - protein, 11 aa, synthetic construct
GGNKIGRKSVH

SEQ ID NO: 349 - protein, 11 aa, synthetic construct
GGNNIGGKSVH

SEQ ID NO: 350 - protein, 11 aa, synthetic construct
GGNNIGSKSVH

SEQ ID NO: 351 - protein, 11 aa, synthetic construct
GGHNIGTKSVH

SEQ ID NO: 352 - protein, 11 aa, synthetic construct
GGDSIGSKYVH

SEQ ID NO: 353 - protein, 11 aa, synthetic construct
GGQNIGTRSVH

SEQ ID NO: 354 - protein, 11 aa, synthetic construct
GGNNIGSETVH

SEQ ID NO: 355 - protein, 11 aa, synthetic construct
RGDNIETYSVH

SEQ ID NO: 356 - protein, 11 aa, synthetic construct
GGNNIGRKSAH

SEQ ID NO: 357 - protein, 11 aa, synthetic construct
EGTYLGAKSVH

SEQ ID NO: 358 - protein, 11 aa, synthetic construct
GGNNIGTKSVH

SEQ ID NO: 359 - protein, 11 aa, synthetic construct
GGQSIGTKSVH

SEQ ID NO: 360 - protein, 11 aa, synthetic construct
GANSIEDKNVH

SEQ ID NO: 361 - protein, 11 aa, synthetic construct
GGDNIGSYSVH

SEQ ID NO: 362 - protein, 11 aa, synthetic construct
GGNNVEDYSVH

SEQ ID NO: 363 - protein, 6 aa, synthetic construct
QNIRYY

SEQ ID NO: 364 - protein, 6 aa, synthetic construct
NIGSKS

SEQ ID NO: 365 - protein, 6 aa, synthetic construct
NIGTKS

SEQ ID NO: 366 - protein, 6 aa, synthetic construct
KIGRKS

SEQ ID NO: 367 - protein, 6 aa, synthetic construct
NIGGKS

SEQ ID NO: 368 - protein, 6 aa, synthetic construct
SIGSKY

SEQ ID NO: 369 - protein, 6 aa, synthetic construct
NIGTRS

SEQ ID NO: 370 - protein, 6 aa, synthetic construct
NIGSET

SEQ ID NO: 371 - protein, 6 aa, synthetic construct
NIETYS

SEQ ID NO: 372 - protein, 6 aa, synthetic construct
NIGRKS

SEQ ID NO: 373 - protein, 6 aa, synthetic construct
SIGTKS

SEQ ID NO: 374 - protein, 6 aa, synthetic construct
SIEDKN

SEQ ID NO: 375 - protein, 6 aa, synthetic construct
NIGSYS

SEQ ID NO: 376 - protein, 6 aa, synthetic construct
NVEDYS

SEQ ID NO: 377 - protein, 7 aa, synthetic construct
DVSSLQS

SEQ ID NO: 378 - protein, 7 aa, synthetic construct
EDSDRPS

SEQ ID NO: 379 - protein, 7 aa, synthetic construct
DDTDRPS

SEQ ID NO: 380 - protein, 7 aa, synthetic construct
DDSDRPP

SEQ ID NO: 381 - protein, 7 aa, synthetic construct
DDKERPS

SEQ ID NO: 382 - protein, 7 aa, synthetic construct
DDSERPS

SEQ ID NO: 383 - protein, 7 aa, synthetic construct
DDLKRPS

SEQ ID NO: 384 - protein, 7 aa, synthetic construct
DDNDRPS

SEQ ID NO: 385 - protein, 9 aa, synthetic construct
QQGYSPPLT

SEQ ID NO: 386 - protein, 11 aa, synthetic construct
QVWDSDSDHVV

SEQ ID NO: 387 - protein, 11 aa, synthetic construct
QVWDSSSDQSV

SEQ ID NO: 388 - protein, 11 aa, synthetic construct
QVWDSSSDEGV

SEQ ID NO: 389 - protein, 11 aa, synthetic construct
QVWDGSSDQSV

SEQ ID NO: 390 - protein, 11 aa, synthetic construct
QVWDTTTDHVV

SEQ ID NO: 391 - protein, 11 aa, synthetic construct
QVWDSSSDPVV

SEQ ID NO: 392 - protein, 11 aa, synthetic construct
QVWDRSSDQSV

SEQ ID NO: 393 - protein, 11 aa, synthetic construct
QVWDSGSGQTV

SEQ ID NO: 394 - protein, 11 aa, synthetic construct
HVWDSSSDHVM

SEQ ID NO: 395 - protein, 11 aa, synthetic construct
QVWDSDSDHLE

SEQ ID NO: 396 - protein, 11 aa, synthetic construct
QVWDSYSNHVV

SEQ ID NO: 397 - protein, 11 aa, synthetic construct
QVWDSGGDHVV

SEQ ID NO: 398 - protein, 11 aa, synthetic construct
QVWDDISDHIV

SEQ ID NO: 399 - protein, 11 aa, synthetic construct
QVWDMASDRVV

SEQ ID NO: 400 - protein, 11 aa, synthetic construct
QVWDTSSDQGV

SEQ ID NO: 401 - protein, 11 aa, synthetic construct
QVWDSTSGVVF

SEQ ID NO: 402 - protein, 107 aa, synthetic construct
DIQLTQSPSSLSASVGDRVAITCRTSQNIRYYLNWYQRKPGKAPRLLIYDVSSLQSGVPSRFSGRGSGTDFTLTISSLQAEDFATYYCQQGYSPPLTFGGGTKVEIK

SEQ ID NO: 403 - protein, 108 aa, synthetic construct
NFMLTQPPSVSVAPGKTAKMTCGGDNIGSKSVHWYQQMPGQAPVLVIYEDSDRPSGTPERFSGSNSGHTATLTISRVEAGDEADYYCQVWDSDSDHVVFGGGTTLTVL

SEQ ID NO: 404 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 405 - protein, 108 aa, synthetic construct
SYELTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 406 - protein, 108 aa, synthetic construct
SYVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 407 - protein, 108 aa, synthetic construct
SYVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 408 - protein, 108 aa, synthetic construct
SYELTQPPSVSVAPGQAARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 409 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDGSSDQSVFGPGTRVTVL

SEQ ID NO: 410 - protein, 108 aa, synthetic construct
SYELTQPPSVSVAPGQTASITCGGNNIGGKSVHWYQLKPGQAPVLVVYDDTDRPSWIPERFSGSNSANTATLTISGVEAGDEADYQCQVWDTTTDHVVFGGGTKLTVL

SEQ ID NO: 411 - protein, 108 aa, synthetic construct
SYVLTQPPSVSVAPGQTARITCGGNNIGSKSVHWYQQKPGQAPLLVVYDDSDRPSWIPERFSGSNSGNAATLTVSRVEAGDEADYYCQVWDSSSDPVVFGGGTKLTVL

SEQ ID NO: 412 - protein, 108 aa, synthetic construct
FYVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 413 - protein, 108 aa, synthetic construct
QSVLTQPPSVSVAPGQTARITCGGQNIGTKSVHWYQQKSGQAPILVVYDDSDRPPWIPERFSGSNFGTTATLTISRVEAGDEADYFCQVWDSSSDQSVFGPGTRVTVL

SEQ ID NO: 414 - protein, 108 aa, synthetic construct
QAVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 415 - protein, 108 aa, synthetic construct
SYELTQPPSVSVAPGQTARITCGGHNIGTKSVHWYQQKSGQAPILVVYDDSDRPSWIPERFSGSNFGTTATLTISRVEAGDEADYFCQVWDRSSDQSVFGPGTRVTVL

SEQ ID NO: 416 - protein, 108 aa, synthetic construct
SYVLTQPPSVSVAPGQTARVTCGGDSIGSKYVHWYQQKPGQAPVLVVYDDSDRPSWIPERFSGSNFGTTATLTISRVEAGDEADYFCQVWDRSSDQSVFGPGTRVTVL

SEQ ID NO: 417 - protein, 108 aa, synthetic construct
SYELTQPPSVSVAPGQTARITCGGQNIGTKSVHWYQQKSGQAPILVVFDDSDRPSWVTERFSGSNFGTTATLTINGVEAGDEADYFCQVWDSGSGQTVFGPGTRVTVS

SEQ ID NO: 418 - protein, 108 aa, synthetic construct
SYELTQPPSVSVAPGQTARITCGGQNIGTRSVHWYQQKSGQAPILVVYDDSDRPSWIPERFSGSNFGTTATLTISRVEAGDEADYFCQVWDSSSDQSVFGPGTRVTVL

SEQ ID NO: 419 - protein, 108 aa, synthetic construct
SYELTQPPSVSVAPGQTARITCGGNNIGSETVHWYRQKAGQAPVLVVYDDSDRPSGIPERFSGSNSGNTAALTISRVEVGDEADYYCHVWDSSSDHVMFGGGTKLTVL

SEQ ID NO: 420 - protein, 108 aa, synthetic construct
SYELTQPPSVSVGPGKTATITCRGDNIETYSVHWYQQKPGQAPVLVVYDDSDRPSGIPERFSGSNSGNTATLTINRVGAGDEADYYCQVWDSDSDHLEFGGGTKLTVL

SEQ ID NO: 421 - protein, 108 aa, synthetic construct
SYELTQPPSVSVAPGQTARITCGGNNIGRKSAHWYQQRPGQAPVLVVYDDSDRPSWIPERFSGSNSGNTATLTISRVEAGDEADYYCQVWDSYSNHVVFGGGTKLTVL

SEQ ID NO: 422 - protein, 108 aa, synthetic construct
LPVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 423 - protein, 108 aa, synthetic construct
SYVLTQPPSVSVAPGQTARITCGGHNIGTKSVHWYQQKSGQAPILAVYDDSDRPSWIPERFSGSNFGTTATLTISRVEAGDEADYFCQVWDRSSDQSVFGPGTRVTVL

SEQ ID NO: 424 - protein, 108 aa, synthetic construct
SYELTQPPSVSVGPGKTATITCRGDNIETYSVHWYQQKPGQAPVLVVYDDSDRPSGIPERFSGSNSGNTATLTINRVEARDEADYFCQVWDSGGDHVVFGGGTKLTVL

SEQ ID NO: 425 - protein, 108 aa, synthetic construct
SYVLTQPPSVSVAPGQTARITCGGNNIGSKSVHWYQQKPGQAPVLVVYDDSDRPSWIPERFSGTNSGNTATLTISRVEAGDEADYYCQVWDSSSDHVVFGGGTKLTVL

SEQ ID NO: 426 - protein, 108 aa, synthetic construct
SYELTQPPSVSVAPGKRARITCEGTYLGAKSVHWYQQKPGQAPVLVIYDDKERPSGIPERFSASNSGNTATLTITRVEAGDEADYHCQVWDDISDHIVFGGGTKLSVL

SEQ ID NO: 427 - protein, 108 aa, synthetic construct
SYVLTQPPSVSVAPGQTARITCGGNNIGTKSVHWYQQKSGQAPILVVYDDSDRPSWIPERFSGSNFGTTATLTISRVEAGDEADYFCQVWDSSSDQSVFGPGTRVTVL

SEQ ID NO: 428 - protein, 108 aa, synthetic construct
SYVLTQPPSVSVAPGQTARITCGGQSIGTKSVHWYQQKSGQAPILVVYDDSDRPSWIPERFSGSNFGTTATLTISRVEAGDEADYFCQVWDSSSDQSVFGPGTRVTVL

SEQ ID NO: 429 - protein, 108 aa, synthetic construct
SYELTQPPSVSVAPGQAASLTCGANSIEDKNVHWYQQRPGQAPLLVIYDDSERPSGIPERFSGSNSGNTATLTITRVAAGDEADYYCQVWDMASDRVVFGPGTTVSVL

SEQ ID NO: 430 - protein, 108 aa, synthetic construct
SYELTQPPSVSVAPGQTARITCGGNKIGRKSVHWYQQRPGQAPVLVVFDDSDRPSWISERFSGSNSGNTATLTIGRVEAGDEADYYCQVWDSSSDEGVFGGGTKLAVL

SEQ ID NO: 431 - protein, 108 aa, synthetic construct
SYELTQPPSVSVAPGRTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 432 - protein, 108 aa, synthetic construct
QAGLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 433 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGTGTKVTVL

SEQ ID NO: 434 - protein, 108 aa, synthetic construct
SYVLTQPPSVSVGPGKTATITCRGDNIETYSVHWYQRKPGQAPVLVVYDDSDRPSGIPERFSGSNSGNTATLTISRVEAGDEADYYCQVWDSSSDHVVFGGGTKLTVL

SEQ ID NO: 435 - protein, 108 aa, synthetic construct
SYELTQPPSVSVAPGQTARITCGGNKIGRKSVHWYQQRPGQAPVLVVFDDSDRPSWISERFSGSNSGNTATLTISRVDAGDEADYYCQVWDSSSDEGVFGGGTKLTVL

SEQ ID NO: 436 - protein, 108 aa, synthetic construct
SYELTQPPSVSVAPGQTARITCGGNKIGRKSVHWYQQKPGQAPVLVVFDDSDRPSWISERFSGSNSGNTATLTISRVEAGDEADYHCQVWDTSSDQGVFGGGTKLTVL

SEQ ID NO: 437 - protein, 107 aa, synthetic construct
SYVLTQPPSVAVAPGQTAKITCGGDNIGSYSVHWYQQKPGQAPVLVVYDDLKRPSWIPERFFGSNSGNTATLTISRVEAGDEAVYYCQVWDSTSGVVFGGGTTVTVL

SEQ ID NO: 438 - protein, 108 aa, synthetic construct
SYELTQPPSVSVAPGKTARITCGGNNVEDYSVHWYHQKAGQAPVVVMYDDNDRPSGVPERFSGSNSGNTATLTIGRVEAGDEADYYCQVWDSSSDHVVFGGGTKLTVL

SEQ ID NO: 439 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 440 - protein, 216 aa, synthetic construct
QSALTQPPSASGSLGQSVTISCTGTSSDVGGYNYVSWYQQHAGKAPKVIIYEVNKRPSGVPDRFSGSKSGNTASLTVSGLQAEDEADYYCSSYEGSDNFVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 441 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 442 - protein, 120 aa, synthetic construct
QVQLVQSGAEVKKPGASVKVSCKASGYIFTDYAMHWVRQAPGQGLEWMGTFIPLLDTSDYNQNFKGRVTMTTDTSTSTAYMELRSLRSDDTAVYYCARMGVTHSYVMDAWGQGTLVTVSS

SEQ ID NO: 443 - protein, 108 aa, synthetic construct
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKR

SEQ ID NO: 444 - protein, 123 aa, synthetic construct
QMQLVESGGGAVQPGGSLRLSCAASGFTFRSYDMHWVRQAPGKGLEWVAVIWYDGSNENYADSVKGRFTTSRDNSKNTLFLQMNSLRAEDTAVYYCARSPLWYGEPDDAFDIWGQGTLVTVSS

SEQ ID NO: 445 - protein, 107 aa, synthetic construct
EIVMTQSPATLSVSPGERATLSCRASQSVSSNLAWYQQKPGQAPRLLISGASTRATGIPARFSGSGSGTDFTLTISSLQSEDFAVYYCQHYNNWPLTFGGGTKVEIK

SEQ ID NO: 446 - protein, 123 aa, synthetic construct
QMQLVESGGGAVQPGRSLRLSCAASGFTFRSYDMHWVRQAPGKGLEWVAVIWYDGSNENYADSVKGRFTTSRDNSKNTLFLQMNSLRAEDTAVYYCARSPLWYGEPDDAFDIWGQGTLVTVSS

SEQ ID NO: 447 - protein, 107 aa, synthetic construct
EIVMTQSPATLSVSPGERATLSCRASQSVSSNLAWYQQKPGQAPRLLISGASTRATGIPARFSGSGSGTDFTLTISSLRSEDFAVYYCQHYNNWPLTFGGGTKVEIK

SEQ ID NO: 448 - protein, 127 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 449 - protein, 107 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 450 - protein, 449 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 451 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 452 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARALIWELFHEYFDIWGQGTMVTVSS

SEQ ID NO: 453 - protein, 509 aa, unidentified
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 454 - protein, 474 aa, unidentified
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 455 - protein, 447 aa, synthetic construct [sequence omitted]

SEQ ID NO: 456 - protein, 446 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 457 - protein, 445 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 458 - protein, 444 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 459 - protein, 443 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 460 - protein, 442 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 461 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 462 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 463 - DNA, 1446 bp, synthetic construct
  source 1..1446: mol_type = other DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctac

SEQ ID NO: 464 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 465 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 466 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 467 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 468 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 469 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 470 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 471 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 472 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 473 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 474 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 475 - protein, 453 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 476 - protein, 430 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 477 - protein, 433 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 478 - protein, 436 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 479 - protein, 452 aa, synthetic construct
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 480 - protein, 452 aa, synthetic construct
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 481 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 482 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 483 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 484 - protein, 451 aa, synthetic construct
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 485 - protein, 449 aa, synthetic construct
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 486 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 487 - protein, 429 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 488 - protein, 425 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGPTLEDLEQFSEKFYCSCYSTLSCKEKADVKDTDAVDVCIAD

SEQ ID NO: 489 - protein, 423 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 490 - protein, 420 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 491 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 492 - protein, 435 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 493 - protein, 436 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 494 - protein, 437 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 495 - protein, 438 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 496 - protein, 439 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 497 - protein, 454 aa, synthetic construct
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 498 - protein, 452 aa, synthetic construct
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 499 - protein, 450 aa, synthetic construct
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 500 - protein, 428 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 501 - protein, 429 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 502 - protein, 430 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 503 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 504 - protein, 441 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 505 - protein, 443 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 506 - protein, 445 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 507 - protein, 447 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 508 - protein, 449 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 509 - protein, 451 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 510 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 511 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 512 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGCWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 513 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGDWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 514 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 515 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 516 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 517 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 518 - protein, 431 aa, synthetic construct
FRGPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 519 - protein, 431 aa, synthetic construct; identical to SEQ ID NO: 518

SEQ ID NO: 520 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 521 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 522 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 523 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 524 - protein, 452 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - protein, 214 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 526 - protein, 451 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 527 - protein, 452 aa, synthetic construct; identical to SEQ ID NO: 524

SEQ ID NO: 528 - protein, 214 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 529 - protein, 452 aa, synthetic construct; identical to SEQ ID NO: 524

SEQ ID NO: 530 - protein, 214 aa, synthetic construct
SYELTQPPSVSVAPGQTARITCGGHNIGTKSVHWYQQKSGQAPILVVYDDSDRPSWIPERFSGSNFGTTATLTISRVEAGDEADYFCQVWDRSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 531 - protein, 452 aa, synthetic construct; identical to SEQ ID NO: 524

SEQ ID NO: 532 - protein, 214 aa, synthetic construct
SYELTQPPSVSVAPGQTARITCGGNNIGRKSAHWYQQRPGQAPVLVVYDDSDRPSWIPERFSGSNSGNTATLTISRVEAGDEADYYCQVWDSYSNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 533 - protein, 452 aa, synthetic construct; identical to SEQ ID NO: 524

SEQ ID NO: 534 - protein, 214 aa, synthetic construct
SYVLTQPPSVSVAPGQTARITCGGHNIGTKSVHWYQQKSGQAPILAVYDDSDRPSWIPERFSGSNFGTTATLTISRVEAGDEADYFCQVWDRSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 535 - protein, 452 aa, synthetic construct; identical to SEQ ID NO: 524

SEQ ID NO: 536 - protein, 214 aa, synthetic construct
SYVLTQPPSVSVAPGQTARITCGGQSIGTKSVHWYQQKSGQAPILVVYDDSDRPSWIPERFSGSNFGTTATLTISRVEAGDEADYFCQVWDSSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 537 - protein, 452 aa, synthetic construct; identical to SEQ ID NO: 524

SEQ ID NO: 538 - protein, 214 aa, synthetic construct
SYELTQPPSVSVAPGQTARITCGGNKIGRKSVHWYQQRPGQAPVLVVFDDSDRPSWISERFSGSNSGNTATLTIGRVEAGDEADYYCQVWDSSSDEGVFGGGTKLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 539 - protein, 452 aa, synthetic construct; identical to SEQ ID NO: 524

SEQ ID NO: 540 - protein, 214 aa, synthetic construct
SYELTQPPSVSVAPGRTARITCGGQNIGTKSVHWYQQKSGQAPILVVYDDSDRPSWIPERFSGSNFGTTATLTISRVEAGDEADYFCQVWDSSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 541 - protein, 452 aa, synthetic construct; identical to SEQ ID NO: 524

SEQ ID NO: 542 - protein, 214 aa, synthetic construct
SYELTQPPSVSVAPGQTARITCGGNKIGRKSVHWYQQKPGQAPVLVVFDDSDRPSWISERFSGSNSGNTATLTISRVEAGDEADYHCQVWDTSSDQGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 543 - protein, 448 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 544 - protein, 447 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 545 - protein, 214 aa, synthetic construct
DIQMTQSPSSLSASVGDRVTITCRASQDIRNDLAWYQQKPGKAPKRLIYASSSLQSGVPSRFSGSGSGTEFTLTISSLQPEDFATYYCLQHNNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 546 - protein, 120 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 547 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 548 - protein, 454 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 549 - protein, 214 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 550 - protein, 448 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 551 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 552 - protein, 447 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 553 - protein, 214 aa, synthetic construct
DIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 554 - protein, 117 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSS

SEQ ID NO: 555 - protein, 107 aa, synthetic construct
DIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIK

SEQ ID NO: 556 - protein, 450 aa, synthetic construct
QVQLVQSGAEVKKPGASVKVSCKASGYIFTDYAMHWVRQAPGQGLEWMGTFIPLLDTSDYNQNFKGRVTMTTDTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 557 - protein, 214 aa, synthetic construct
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 558 - protein, 453 aa, synthetic construct
QMQLVESGGGAVQPGGSLRLSCAASGFTFRSYDMHWVRQAPGKGLEWVAVIWYDGSNENYADSVKGRFTTSRDNSKNTLFLQMNSLRAEDTAVYYCARSPLWYGEPDDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 559 - protein, 214 aa, synthetic construct
EIVMTQSPATLSVSPGERATLSCRASQSVSSNLAWYQQKPGQAPRLLISGASTRATGIPARFSGSGSGTDFTLTISSLQSEDFAVYYCQHYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 560 - protein, 450 aa, synthetic construct
QMQLVESGGGAVQPGRSLRLSCAASGFTFRSYDMHWVRQAPGKGLEWVAVIWYDGSNENYADSVKGRFTTSRDNSKNTLFLQMNSLRAEDTAVYYCARSPLWYGEPDDAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 561 - protein, 214 aa, synthetic construct
EIVMTQSPATLSVSPGERATLSCRASQSVSSNLAWYQQKPGQAPRLLISGASTRATGIPARFSGSGSGTDFTLTISSLRSEDFAVYYCQHYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 562 - protein, 120 aa, synthetic construct
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSS

SEQ ID NO: 563 - protein, 107 aa, synthetic construct
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIK

SEQ ID NO: 564 - protein, 450 aa, synthetic construct
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 214 aa, synthetic construct; identical to SEQ ID NO: 557

SEQ ID NO: 566 - protein, 11 aa, synthetic construct
QVWDSSSDFVV

SEQ ID NO: 567 - protein, 11 aa, synthetic construct
QVWDSSSTHVV

SEQ ID NO: 568 - protein, 11 aa, synthetic construct
QVWDSSSTFVV

SEQ ID NO: 569 - protein, 11 aa, synthetic construct
QVWDSSSTIVV

SEQ ID NO: 570 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSDHVVFGGGTKLTVL

SEQ ID NO: 571 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDFVVFGGGTKLTVL

SEQ ID NO: 572 - protein, 108 aa, synthetic construct; identical to SEQ ID NO: 42

SEQ ID NO: 573 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSDFVVFGGGTKLTVL

SEQ ID NO: 574 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTHVVFGGGTKLTVL

SEQ ID NO: 575 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSDFVVFGGGTKLTVL

SEQ ID NO: 576 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSTFVVFGGGTKLTVL

SEQ ID NO: 577 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTHVVFGGGTKLTVL

SEQ ID NO: 578 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSDFVVFGGGTKLTVL

SEQ ID NO: 579 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTIVVFGGGTKLTVL

SEQ ID NO: 580 - protein, 108 aa, synthetic construct; identical to SEQ ID NO: 37

SEQ ID NO: 581 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTFVVFGGGTKLTVL

SEQ ID NO: 582 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTIVVFGGGTKLTVL

SEQ ID NO: 583 - protein, 108 aa, synthetic construct; identical to SEQ ID NO: 441

SEQ ID NO: 584 - protein, 452 aa, synthetic construct; identical to SEQ ID NO: 524

SEQ ID NO: 585 - protein, 214 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 586 - protein, 452 aa, synthetic construct; identical to SEQ ID NO: 524

SEQ ID NO: 587 - protein, 214 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 588 - protein, 5 aa, Homo sapiens
SRDEL

SEQ ID NO: 589 - protein, 5 aa, Homo sapiens
SREEM

SEQ ID NO: 590 - protein, 6 aa, synthetic construct
YLGAKS

SEQ ID NO: 591 - protein, 5 aa, synthetic construct
NYAMN

SEQ ID NO: 592 - protein, 17 aa, synthetic construct
TISGSGGATRYADSVKG

SEQ ID NO: 593 - protein, 18 aa, synthetic construct
DRLIMATVRGPYYYGMDV

SEQ ID NO: 594 - protein, 7 aa, synthetic construct
GFTFSNY

SEQ ID NO: 595 - protein, 6 aa, synthetic construct
SGSGGA

SEQ ID NO: 596 - protein, 8 aa, synthetic construct
GFTFSNYA

SEQ ID NO: 597 - protein, 8 aa, synthetic construct
ISGSGGAT

SEQ ID NO: 598 - protein, 20 aa, synthetic construct
TKDRLIMATVRGPYYYGMDV

SEQ ID NO: 599 - protein, 11 aa, synthetic construct
RASQSISSYLN

SEQ ID NO: 600 - protein, 7 aa, synthetic construct
AASSLQS

SEQ ID NO: 601 - protein, 9 aa, synthetic construct
QQSHSVSFT

SEQ ID NO: 602 - protein, 6 aa, synthetic construct
QSISSY

(19) United States
(12) Patent Application Publication
(10) Pub. No.: US 2026/0109759 A1
(43) Pub. Date: Apr. 23, 2026

(54) ANTIBODIES THAT BIND OX40L AND ANTIBODIES THAT BIND TSLP IN COMBINATION WITH HYALURONIDASE OR A VARIANT THEREOF

(71) Applicant: Apogee Therapeutics, Inc., Waltham, MA (US)

(72) Inventors: Aaron NOYES, Apex, NC (US); Rebecca Lucille DABORA, Bethlehem, PA (US)

(21) Appl. No.: 19/361,402

(22) Filed: Oct. 17, 2025

Related U.S. Application Data
(60) Provisional application No. 63/708,883, filed on Oct. 18, 2024.
(60) Provisional application No. 63/827,209, filed on Jun. 20, 2025.

(51) Int. Cl. C07K 16/24; A61K 38/00; A61K 39/00; C07K 16/28; C12N 9/26

(52) U.S. Cl. CPC C07K 16/244; C07K 16/2875; C12N 9/2474; A61K 38/00; A61K 2039/54; C07K 2317/24; C07K 2317/52; C07K 2317/565; C07K 2317/92; C12Y 302/01035

(57) ABSTRACT

Described herein are compositions and combinations comprising an antibody or antigen binding fragment thereof that binds thymic stromal lymphopoietin (TSLP), an antibody or antigen binding fragment thereof that binds OX40L, and a hyaluronidase or variant thereof, as well as related methods of treating an inflammatory disorder or disease in a human patient by administering such compositions and combinations.

### CROSS REFERENCE TO RELATED APPLICATIONS

This application claims the benefit of U.S. Provisional Application No. 63/708,883 (filed Oct. 18, 2024) and U.S. Provisional Application No. 63/827,209 (filed Jun. 20, 2025), both of which are incorporated by reference herein in their entirety.

### SEQUENCE LISTING

The instant application contains a Sequence Listing which has been submitted electronically in XML file format and is hereby incorporated by reference in its entirety. Said XML copy, created on Oct. 16, 2025, is named AOJ-014_SL.xml and is 646, 390 bytes in size.

### BACKGROUND

Thymic stromal lymphopoietin (TSLP) is an epithelial cell-derived cytokine produced in response to pro-inflammatory stimuli. TSLP has been discovered to promote allergic inflammatory responses primarily through its activity on dendritic and mast cells. Human TSLP expression has been reported to be increased in asthmatic airways correlating to disease severity. In addition, TSLP protein levels are detectable in the concentrated bronchoalveolar lavage (BAL) fluid of asthma patients and other patients suffering from allergic disorders. In addition, TSLP has also been found to promote fibrosis.

TSLP binds to a heterodimeric receptor consisting of the TSLP receptor (TSLPR) and an IL-7 receptor a chain (IL-7Rα) in dendritic cells, thereby activating the dendritic cells. Upon activation, the dendritic cells express inflammatory chemokines such as thymus- and activation-regulated chemokine (TARC (CCL17)), macrophage-derived chemokine (MDC (CCL22)), and the like.

Activation of dendritic cells by TSLP through the TSLPR is associated with disease pathology, including allergic inflammatory diseases, such as asthma, and autoimmune disease, such as systemic sclerosis.

An emerging mechanism in treatments for atopic dermatitis (AD) is targeting OX40 or OX40L. OX40L is the ligand for OX40. OX40L is expressed on antigen presenting cells and its interaction with OX40 causes the accumulation of T cells by providing a survival signal. T cells are important types of white blood cells of the immune system that play a central role in the immune response. OX40L, by playing a role in activating T cells and reprogramming them into inflammatory subsets, contributes to immune overactivation in AD and other inflammatory conditions, such as Systemic Lupus Erythematosus. Additionally, OX40L activation of OX40 inhibits the expression of FOXP3 and the inhibitory function of regulatory T (Treg) cells. Treg cells can suppress the immune response that leads to worsening symptoms in inflammatory conditions. Therefore, OX40L blockade may lead to clinical benefit in AD and other inflammatory conditions by first suppressing inflammatory T cell activation, and next by increasing the proliferation of Treg cells, which can serve to further reduce inflammatory cells. Amlitelimab, which targets OX40L, and rocatinlimab, which targets OX40, have both demonstrated promising Phase 2 data in AD.

Accordingly, there is a need in the art for antagonists to TSLP and OX40L for preventing and treating diseases in which TSLP and OX40L are involved in the disease pathology, such as inflammatory and fibrotic disorders.

In addition, because targeting each of OX40L and TSLP may lead to various clinical benefits in inflammatory conditions, there is a need in the art for improved therapeutics that target both TSLP and OX40L, such as a combination of antibodies wherein, for example, one or more of the antibodies remains active for a longer term (i.e., have a longer half-life) as compared to another anti-TSLP antibody (e.g., tezepelumab) or anti-OX40L antibody (e.g., amlitelimab or oxelumab) when administered to a subject. There is also a need for delivering relatively large doses of these antibodies subcutaneously.

### SUMMARY

The present disclosure provides compositions and combinations comprising antibodies to TSLP, or antigen binding fragments thereof, such as recombinant human antibodies, antibodies that specifically bind human OX40L, or antigen binding fragments thereof, and a hyaluronidase or a variant thereof. Also featured are methods of treating diseases in which human TSLP and OX40L are involved in disease pathology by administering to a subject an antibody, or antigen fragment thereof, that binds TSLP, an antibody, or antigen binding fragment thereof, that binds OX40L, and a hyaluronidase or variant thereof. Diseases that can be treated according to the methods described herein include atopic dermatitis and asthma, among others. The antibodies of the disclosure can include Fc modifications and exhibit longer half-lives than an otherwise identical antibody that does not include the Fc modification, for example, certain anti-TSLP or anti-OX40L antibodies known in the art.

In a first aspect, the invention provides a composition comprising an isolated antibody, or antigen binding fragment thereof, that binds TSLP, an isolated antibody, or antigen binding fragment thereof, that binds OX40L, and a hyaluronidase or variant thereof.

In another aspect, the invention provides a composition comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; or (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396.

In another aspect, the invention provides a composition comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328; (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330; (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332; or (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In another aspect, the invention provides a combination comprising an isolated antibody, or antigen binding fragment thereof, that binds TSLP, an isolated antibody, or antigen binding fragment thereof, that binds OX40L, and a hyaluronidase or variant thereof.

In another aspect, the invention provides a combination comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; or (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396.

In another aspect, the invention provides a combination comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328; (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330; (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332; or (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In one aspect, provided herein is a method of treating an inflammatory disorder or disease in a human patient comprising administering to the patient an isolated antibody, or antigen binding fragment thereof, that binds TSLP, comprising: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; or (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396; an isolated antibody, or antigen binding fragment thereof, that binds OX40L; and a hyaluronidase or variant thereof.

In some embodiments, the isolated antibody, or antigen binding fragment thereof, that binds OX40L comprises a CDR-H1, CDR-H2, CDR-H3, CDR-L1, CDR-L2, and CDR-L3 of an antibody selected from amlitelimab and oxelumab.

In some embodiments, the isolated antibody, or antigen binding fragment thereof, that binds OX40L comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3, wherein the isolated antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328; (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330; (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332; or (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In another aspect, provided herein is a method of treating an inflammatory disorder or disease in a human patient comprising administering to the patient an isolated antibody, or antigen binding fragment thereof, that binds TSLP; a hyaluronidase or variant thereof; and an isolated antibody, or antigen binding fragment thereof, that binds OX40L, comprising: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328; (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330; (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332; or (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, the isolated antibody, or antigen binding fragment thereof, that binds TSLP comprises a CDR-H1, CDR-H2, CDR-H3, CDR-L1, CDR-L2, and CDR-L3 of an antibody selected from tezepelumab), hu23b12, 43B1-H2L2, 43B1-H6L1, GR2002 (described in U.S. Patent Application Publication No. US 2024/0132581, which is incorporated herein by reference), TQC2731 (also known as BSI-045B and bosakitug, described in U.S. Patent Application Publication No. US 2022/0289833, which is incorporated herein by reference), and solrikitug.

In some embodiments, the isolated antibody, or antigen binding fragment thereof, that binds TSLP comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3, wherein the isolated antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; or (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396.

In some embodiments, (a) the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; and (b) the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328.

In some embodiments, (a) the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; and (b) the antibody, or antigen binding fragment thereof, that binds OX40L comprises: (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any of SEQ ID NOs: 329 and 330.

In some embodiments, (a) the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; and (b) the antibody, or antigen binding fragment thereof, that binds OX40L comprises: (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332.

In some embodiments, (a) the antibody, or antigen binding fragment thereof, that binds TSLP comprises: (i) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27; and (b) the antibody, or antigen binding fragment thereof, that binds OX40L comprises: (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, (a) the antibody, or antigen binding fragment thereof, that binds TSLP comprises: (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; and (b) the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328.

In some embodiments, (a) the antibody, or antigen binding fragment thereof, that binds TSLP comprises: (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; and (b) the antibody, or antigen binding fragment thereof, that binds OX40L comprises: (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any of SEQ ID NOs: 329 and 330.

In some embodiments, (a) the antibody, or antigen binding fragment thereof, that binds TSLP comprises: (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; and (b) the antibody, or antigen binding fragment thereof, that binds OX40L comprises: (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332.

In some embodiments, (a) the antibody, or antigen binding fragment thereof, that binds TSLP comprises: (ii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30; and (b) the antibody, or antigen binding fragment thereof, that binds OX40L comprises: (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, (a) the antibody, or antigen binding fragment thereof, that binds TSLP comprises: (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; and (b) the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328.

In some embodiments, (a) the antibody, or antigen binding fragment thereof, that binds TSLP comprises: (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; and (b) the antibody, or antigen binding fragment thereof, that binds OX40L comprises: (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330.

In some embodiments, (a) the antibody, or antigen binding fragment thereof, that binds TSLP comprises: (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; and (b) the antibody, or antigen binding fragment thereof, that binds OX40L comprises: (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332.

In some embodiments, (a) the antibody, or antigen binding fragment thereof, that binds TSLP comprises: (iii) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392; and (b) the antibody, or antigen binding fragment thereof, that binds OX40L comprises: (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, (a) the antibody, or antigen binding fragment thereof, that binds TSLP comprises: (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396; and (b) the antibody, or antigen binding fragment thereof, that binds OX40L comprises: (i) a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328.

In some embodiments, (a) the antibody, or antigen binding fragment thereof, that binds TSLP comprises: (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396; and (b) the antibody, or antigen binding fragment thereof, that binds OX40L comprises: (ii) a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330.

In some embodiments, (a) the antibody, or antigen binding fragment thereof, that binds TSLP comprises: (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396; and (b) the antibody, or antigen binding fragment thereof, that binds OX40L comprises: (iii) a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332.

In some embodiments, (a) the antibody, or antigen binding fragment thereof, that binds TSLP comprises: (iv) a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396; and (b) the antibody, or antigen binding fragment thereof, that binds OX40L comprises: (iv) a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, the antibody, or antigen binding fragment thereof, that binds TSLP comprises a heavy chain variable domain (VH) sequence comprising the amino acid sequence set forth in any one of SEQ ID NOs: 32-36. In some embodiments, the antibody, or antigen binding fragment thereof, that binds TSLP comprises VH sequence comprising the amino acid sequence set forth in SEQ ID NO: 34.

In some embodiments, the antibody, or antigen binding fragment thereof, that binds OX40L comprises a VH sequence comprising the amino acid sequence set forth in any one of SEQ ID NOs: 335-338.

In some embodiments, the antibody, or antigen binding fragment thereof, that binds TSLP comprises a light chain variable domain (VL) sequence comprising the amino acid sequence set forth in any one of SEQ ID NOs: 37-45 and 402-438. In some embodiments, the antibody, or antigen binding fragment thereof, that binds TSLP comprises a VL sequence comprising the amino acid sequence set forth in any one of SEQ ID NOs: 41, 43, 415, and 421.

In some embodiments, the antibody, or antigen binding fragment thereof, that binds OX40L comprises a VL sequence comprising the amino acid sequence set forth in any one of SEQ ID NOs: 339-342.

In some embodiments, (a) the antibody, or antigen binding fragment thereof, that binds TSLP comprises: (i) a VH sequence comprising the amino acid sequence set forth in SEQ ID NO: 32; (ii) a VH sequence comprising the amino acid sequence set forth in SEQ ID NO: 33; or (iii) a VH sequence comprising the amino acid sequence set forth in SEQ ID NO: 34; and/or (iv) a VL sequence comprising the amino acid sequence set forth in SEQ ID NO: 41; (v) a VL sequence comprising the amino acid sequence set forth in SEQ ID NO: 43; (vi) a VL sequence comprising the amino acid sequence set forth in SEQ ID NO: 415; or (vii) a VL sequence comprising the amino acid sequence set forth in SEQ ID NO: 421; and/or (b) the antibody, or antigen binding fragment thereof, that binds OX40L comprises: (i) a VH sequence comprising the amino acid sequence set forth in SEQ ID NO: 335; (ii) a VH sequence comprising the amino acid sequence set forth in SEQ ID NO: 336; (iii) a VH sequence comprising the amino acid sequence set forth in SEQ ID NO: 337; or (iv) a VH sequence comprising the amino acid sequence set forth in SEQ ID NO: 338; and/or (v) a VL sequence comprising the amino acid sequence set forth in SEQ ID NO: 339; (vi) a VL sequence comprising the amino acid sequence set forth in SEQ ID NO: 400; (vii) a VL sequence comprising the amino acid sequence set forth in SEQ ID NO: 401; or (viii) a VL sequence comprising the amino acid sequence set forth in SEQ ID NO: 402.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421.

In some embodiments, the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

In some embodiments, the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

In some embodiments, the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

In some embodiments, the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth SEQ ID NO: 337; and a VL sequence comprising the amino acid sequence set forth in SEQ ID NO: 341.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth SEQ ID NO: 338; and a VL sequence comprising the amino acid sequence set forth in SEQ ID NO: 342.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

In some embodiments, the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

In some embodiments, antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

In some embodiments, the antibody, or antigen binding fragment thereof, that binds TSLP and/or the antibody, or antigen binding fragment thereof, that binds OX40L is a humanized, human, or chimeric antibody. In some embodiments, the antibody, or antigen binding fragment thereof, that binds TSLP and/or the antibody, or antigen binding fragment thereof, that binds OX40L is a humanized antibody.

In some embodiments, the antibody, or antigen binding fragment thereof, that binds TSLP and/or the antibody, or antigen binding fragment thereof, that binds OX40L comprises a heavy chain human constant region of a class selected from IgG, IgA, IgD, IgE, and IgM.

In some embodiments, the antibody, or antigen binding fragment thereof, that binds TSLP and/or the antibody, or antigen binding fragment thereof, that binds OX40L comprises a human Fc region, and wherein the human Fc region comprises a human heavy chain constant region of the class IgG and a subclass selected from IgG1, IgG2, IgG3, and IgG4.

In some embodiments, the human Fc region of the antibody, or antigen binding fragment thereof, that binds TSLP and/or the human Fc region of the antibody, or antigen binding fragment thereof, that binds OX40L comprises a human IgG1 Fc region.

In some embodiments, the human Fc region of the antibody, or antigen binding fragment thereof, that binds TSLP and/or the human Fc region of the antibody, or antigen binding fragment thereof, that binds OX40L comprises a human IgG4 Fc region.

In some embodiments, the human Fc region of the antibody, or antigen binding fragment thereof, that binds TSLP and/or the human Fc region of the antibody, or antigen binding fragment thereof, that binds OX40L comprises a human IgG2 Fc region.

In some embodiments, the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 47-270. In some embodiments, the heavy chain of the antibody, or antigen binding fragment thereof, that binds TSLP and/or the heavy chain of the antibody, or antigen binding fragment thereof, that binds OX40L comprises a constant heavy chain sequence selected from an amino acid sequence set forth in any one of SEQ ID NOs: 61 and 173.

In some embodiments, the antibody, or antigen binding fragment thereof, that binds TSLP and/or the antibody, or antigen binding fragment thereof, that binds OX40L comprises a constant light chain sequence set forth in SEQ ID NO: 46 or SEQ ID NO: 520.

In some embodiments, the antibody, or antigen binding fragment thereof, that binds TSLP and/or the antibody, or antigen binding fragment thereof, that binds OX40L comprises a human lambda light chain constant region. In some embodiments, the human lambda light chain constant region comprises a sequence set forth in any one of SEQ ID NOs: 520-523. In some embodiments, the human lambda light chain constant region comprises a sequence set forth in SEQ ID NO: 520.

In some embodiments, the antibody, or antigen binding fragment thereof, that binds TSLP comprises a light chain comprising a human lambda light chain constant region and/or the antibody, or antigen binding fragment thereof, that binds OX40L comprises a light chain comprising a human kappa light chain constant region.

In some embodiments, the light chain of the antibody, or antigen binding fragment thereof, that binds TSLP comprises a constant light chain sequence set forth in SEQ ID NO: 520.

In some embodiments, the light chain of the antibody, or antigen binding fragment thereof, that binds OX40L comprises a constant light chain sequence set forth in SEQ ID NO: 46.

In some embodiments, the antibody that binds OX40L comprises a heavy chain sequence set forth in SEQ ID NO: 543 or 544 and a light chain sequence set forth in SEQ ID NO: 545. In some embodiments, the antibody that binds OX40L comprises a heavy chain sequence set forth in SEQ ID NO: 543 and a light chain sequence set forth in SEQ ID NO: 545. The heavy chain sequence of SEQ ID NO: 543 contains a C-terminal lysine, which may be cleaved off during manufacture or after administration, resulting in the heavy chain sequence of SEQ ID NO: 544.

In some embodiments, the antibody that binds TSLP comprises a heavy chain sequence set forth in SEQ ID NO: 524 or 526 and a light chain sequence set forth in SEQ ID NO: 525. In some embodiments, the antibody that binds TSLP comprises a heavy chain sequence set forth in SEQ ID NO: 524 and a light chain sequence set forth in SEQ ID NO: 525. The heavy chain sequence of SEQ ID NO: 524 contains a C-terminal lysine, which may be cleaved off during manufacture or after administration, resulting in the heavy chain sequence of SEQ ID NO: 526.

In some embodiments, the antibody that binds TSLP is Antibody 5 and the antibody that binds OX40L is Construct 114.

In some embodiments, the antibody that binds TSLP is an antibody having a VH sequence and a VL sequence from the same row of TABLE 4 (i.e., a VH sequence and VL sequence from a single antibody in TABLE 4) and the antibody that binds OX40L is Construct 114.

In some embodiments, the antibody, or antigen binding fragment thereof, that binds TSLP is an antibody having a VH sequence and a VL sequence from the same row of TABLE 6 (i.e., a VH sequence and VL sequence from a single antibody in TABLE 6) and the antibody that binds OX40L is Construct 114.

In some embodiments, the antibody that binds TSLP is an antibody having a CDR-H1, a CDR-H2, a CDR-H3, a CDR-L1, a CDR-L2, and a CDR-L3 from the same row of TABLE 3 (i.e., having six CDRs from a single antibody in TABLE 3) and the antibody that binds OX40L is Construct 114.

In some embodiments, the antibody that binds TSLP is an antibody having a CDR-H1, a CDR-H2, a CDR-H3, a CDR-L1, a CDR-L2, and a CDR-L3 from the same row of TABLE 5 (i.e., having six CDRs from a single antibody in TABLE 5) and the antibody that binds OX40L is Construct 114.

In some embodiments, the antibody that binds OX40L is an antibody having a VH sequence and a VL sequence from the same row of TABLE 7 (i.e., a VH sequence and VL sequence from a single antibody in TABLE 7) and the antibody that binds TSLP is Antibody 5.

In some embodiments, the antibody, or antigen binding fragment thereof, that binds TSLP and/or the antibody, or antigen binding fragment thereof, that binds OX40L comprises a heavy chain constant domain having a means for increasing the half-life of the antibody.

In some embodiments, the antibody, or antigen binding fragment thereof, that binds TSLP and/or the antibody, or antigen binding fragment thereof, that binds OX40L comprises an Fc region comprising one or more amino acid substitutions, wherein the one or more amino acid substitutions result in an increase in one or more of antibody half-life, ADCC activity, ADCP activity, or CDC activity compared with the Fc without the one or more substitutions.

In some embodiments, the antibody, or antigen binding fragment thereof, that binds TSLP and/or the antibody, or antigen binding fragment thereof, that binds OX40L comprises an Fc region comprising one or more amino acid substitutions, wherein the one or more amino acid substitutions result in a decrease in one or more of, ADCC activity, ADCP activity or CDC activity compared to an antibody comprising a wild-type Fc region.

In some embodiments, the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W (or, e.g., L235A, L236A, L239A, L240A, M253Y, S255T, T257E, M257Y, S259T, or T261E using direct numbering); optionally, wherein the one or more amino acid substitutions comprises a plurality of amino acid substitutions selected from the group consisting of M428L/N434S (LS), M252Y/S254T/T256E (YTE) using EU numbering or M257Y/S259T/T261E (YTE) using direct numbering or M253Y/S255T/T257E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) using EU numbering or L239A/L240A (LALA) using direct numbering or L235A/L236A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV. In some embodiments, the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

In some embodiments, the Fc region of the antibody that binds TSLP and/or the antibody that binds OX40L comprises LALA/YTE substitutions.

Although the EU numbering system is typically used to identify the positions of the various Fc mutations described herein, direct numbering can also be used. For example, in certain embodiments, an antibody described herein comprises an Fc region with YTE mutations at positions 253/255/257 or 257/259/261, respectively. In certain embodiments, an antibody described herein comprises an Fc region with LALA mutations at positions 235/236 or 239/240, respectively. In certain embodiments, an antibody described herein comprises an Fc region with YTE mutations at positions 253/255/257 and with LALA mutations at positions 235/236. In certain embodiments, an antibody described herein comprises an Fc region with YTE mutations at positions 257/259/261 and with LALA mutations at positions 239/240. In certain embodiments, the OX40L antibody described herein comprises an Fc region comprising YTE mutations at positions 253/255/257, respectively and LALA mutations at positions 235/236, respectively. In certain embodiments, the OX40L antibody described herein comprises the VH and VL of Construct 114 and an Fc region comprising YTE mutations at positions 253/255/257, respectively and with LALA mutations at positions 235/236, respectively. In certain embodiments, the TSLP antibody described herein comprises an Fc region comprising YTE mutations at positions 257/259/261, respectively and LALA mutations at positions 239/240, respectively. In certain embodiments, the TSLP antibody described herein comprises the VH and VL of Antibody 5 and an Fc region comprising YTE mutations at positions 257/259/261, respectively and with LALA mutations at positions 239/240, respectively.

In some embodiments, the antibody, or antigen binding fragment thereof, that binds TSLP comprises a VH sequence comprising the amino acid sequence set forth in SEQ ID NO: 34, a VL sequence comprising the amino acid sequence set forth in SEQ ID NO: 41, and a human IgG1 Fc region comprising LALA and YTE mutations; and the antibody that binds OX40L comprises a VH sequence comprising the amino acid sequence set forth in SEQ ID NO: 335, a VL sequence comprising the amino acid sequence set forth in SEQ ID NO: 339, and a human IgG1 Fc region comprising LALA and YTE mutations. In some embodiments, the antibody that binds TSLP comprises a VH sequence comprising the amino acid sequence set forth in SEQ ID NO: 34, a constant heavy chain sequence comprising the amino acid sequence set forth SEQ ID NO: 173 and/or a constant heavy chain sequence comprising the amino acid sequence set forth SEQ ID NO: 61, a VL sequence comprising the amino acid sequence set forth in SEQ ID NO: 41, and a constant light chain sequence comprising the amino acid sequence set forth in SEQ ID NO: 520; and the antibody that binds OX40L comprises a VH sequence comprising the amino acid sequence set forth in SEQ ID NO: 335, a constant heavy chain sequence comprising the amino acid sequence set forth SEQ ID NO: 173 and/or a constant heavy chain sequence comprising the amino acid sequence set forth SEQ ID NO: 61, a VL sequence comprising the amino acid sequence set forth in SEQ ID NO: 339, and a constant light chain sequence comprising the amino acid sequence set forth in SEQ ID NO: 46. In some embodiments, the antibody that binds TSLP comprises a VH sequence comprising the amino acid sequence set forth in SEQ ID NO: 34, a constant heavy chain sequence comprising the amino acid sequence set forth SEQ ID NO: 173, a VL sequence comprising the amino acid sequence set forth in SEQ ID NO: 41, and a constant light chain sequence comprising the amino acid sequence set forth in SEQ ID NO: 520; and the antibody that binds OX40L comprises a VH sequence comprising the amino acid sequence set forth in SEQ ID NO: 335, a constant heavy chain sequence comprising the amino acid sequence set forth SEQ ID NO: 173, a VL sequence comprising the amino acid sequence set forth in SEQ ID NO: 339, and a constant light chain sequence comprising the amino acid sequence set forth in SEQ ID NO: 46. A C-terminal lysine is present in SEQ ID NO: 173, which may be cleaved off during manufacture or after administration, resulting in the sequence of SEQ ID NO: 61. Accordingly, a composition comprising an antibody that binds TSLP and/or an antibody that binds OX40L comprising the constant heavy chain sequence of SEQ ID NO: 173 that is administered to a subject may comprise antibodies having the constant heavy chain sequence set forth in SEQ ID NO: 173 or SEQ ID NO: 61, or a mixture thereof (e.g., a mixture of antibodies containing either a constant heavy chain sequence of SEQ ID NO: 173 or a constant heavy chain sequence of SEQ ID NO: 61 and/or antibodies containing both constant heavy chain sequences (e.g., in an antibody containing two constant heavy chain sequences)).

In some embodiments, the antibody that binds TSLP comprises a heavy chain sequence comprising the amino acid sequence set forth SEQ ID NO: 524 and/or 526 and a light chain sequence comprising the amino acid sequence set forth in SEQ ID NO: 525; and the antibody that binds OX40L comprises a heavy chain sequence comprising the amino acid sequence set forth SEQ ID NO: 543 and/or 544 and a light chain sequence comprising the amino acid sequence set forth in SEQ ID NO: 545. In some embodiments, the antibody that binds TSLP comprises a heavy chain sequence comprising the amino acid sequence set forth SEQ ID NO: 524 and a light chain sequence comprising the amino acid sequence set forth in SEQ ID NO: 525; and the antibody that binds OX40L comprises a heavy chain sequence comprising the amino acid sequence set forth SEQ ID NO: 543 and a light chain sequence comprising the amino acid sequence set forth in SEQ ID NO: 545. The C-terminal lysine in SEQ ID NO: 524 and/or SEQ ID NO: 543 may not be present if cleavage occurs during manufacture or after administration (which would result in the heavy chain sequence of SEQ ID NO: 526 and/or SEQ ID NO: 544, respectively).

In some embodiments, the antibody that binds TSLP is Antibody 5 and the antibody that binds OX40L is amlitelimab or oxelumab.

In some embodiments, the antibody that binds TSLP is tezepelumab, GR2002, TQC2731, solrikitug, hu23b12, 43B1-H2L2, or 43B1-H6L1 and the antibody that binds OX40L is Construct 114.

In some embodiments, the Fc region of the antibody, or antigen binding fragment thereof, that binds TSLP and/or the Fc region of the antibody, or antigen binding fragment thereof, that binds OX40L binds to Neonatal Fc receptor (FcRn).

In some embodiments, the Fc region of the antibody, or antigen binding fragment thereof, that binds TSLP and/or the Fc region of the antibody, or antigen binding fragment thereof, that binds OX40L binds an FcRn with higher affinity at pH 6.0 compared to an antibody comprising a wild-type Fc region.

In some embodiments, the Fc region of the antibody, or antigen binding fragment thereof, that binds TSLP and/or the Fc region of the antibody, or antigen binding fragment thereof, that binds OX40L binds to FcRn with a KD of <1×10-7 M at pH 6.0.

In some embodiments, the antibody, or antigen binding fragment thereof, that binds TSLP and/or the antibody, or antigen binding fragment thereof, that binds OX40L is a monoclonal antibody.

In some embodiments, the antibody, or antigen binding fragment thereof, that binds TSLP binds a TSLP sequence set forth in the amino acid sequence of any of SEQ ID NOs: 271-272.

In some embodiments, the antibody, or antigen binding fragment thereof, that binds OX40L binds an OX40L sequence set forth in the amino acid sequence of SEQ ID NO: 439.

In some embodiments, the isolated antibodies of any one disclosed here are for use in the treatment of an inflammatory disorder or disease.

In some embodiments, the isolated antibodies, or antigen binding fragments thereof, disclosed herein are for use in the treatment of atopic dermatitis (AD). In some embodiments, the treatment reduces disease severity in a patient and wherein disease severity is assessed by an atopic dermatitis disease severity outcome measure.

In some embodiments, the isolated antibodies, or antigen binding fragments thereof, disclosed herein are for use in the treatment of asthma, chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; idiopathic pulmonary fibrosis; alopecia areata; systemic sclerosis; allergic rhinitis; eosinophilic fasciitis; scleromyxedema; scleredema; or nephrogenic systemic fibrosis.

In another aspect, provided herein is an isolated polynucleotide or set of polynucleotides encoding any one of the isolated antibodies disclosed herein, a VH thereof, a VL thereof, a light chain thereof, a heavy chain thereof, or an antigen-binding portion thereof, and optionally, wherein the polynucleotide or set of polynucleotides comprises cDNA.

In another aspect, provided herein is a vector or set of vectors comprising the polynucleotide or set of polynucleotides disclosed herein.

In another aspect, provided herein is a host cell comprising the polynucleotide or set of polynucleotides disclosed herein or the vector or set of vectors disclosed herein.

In another aspect, provided herein is a method of producing one or more of the isolated antibodies, or antigen binding fragments thereof, disclosed herein, the method comprising expressing one or more of the antibodies within the host cell disclosed herein and isolating the one or more expressed antibodies.

In another aspect, provided herein is a pharmaceutical composition comprising any of the isolated antibodies, or antigen binding fragments thereof, disclosed herein and a pharmaceutically acceptable excipient (i.e., a pharmaceutical composition comprising an antibody, or antigen binding fragment thereof, that binds to TSLP and/or an antibody, or antigen binding fragment thereof, that binds to OX40L). In some embodiments, the pharmaceutical composition further comprises a hyaluronidase or a variant thereof.

In another aspect, provided herein is a kit comprising any of the isolated antibodies, or antigen binding fragments thereof, disclosed herein (e.g., a kit comprising an antibody, or antigen binding fragment thereof, that binds to TSLP and an antibody, or antigen binding fragment thereof, that binds to OX40L) or the pharmaceutical composition disclosed herein and instructions for use. In some embodiments, the kit further comprises a hyaluronidase or a variant thereof.

In another aspect, provided herein is a method for treating an inflammatory disorder or disease in a mammalian subject in need thereof, the method comprising administering to the mammalian subject a therapeutically effective amount of any of the isolated antibodies, or antigen binding fragments thereof, disclosed herein; a therapeutically effective amount of a pharmaceutical composition disclosed herein; or a therapeutically effective amount of a pharmaceutical composition comprising an isolated antibody, or antigen binding fragment thereof, that binds TSLP and a pharmaceutically acceptable excipient and a therapeutically effective amount of a pharmaceutical composition comprising an antibody, or antigen binding fragment thereof, that binds OX40L and a pharmaceutically acceptable excipient.

In some embodiments, the mammalian subject is a human.

In some embodiments, the inflammatory disorder or disease is atopic dermatitis.

In some embodiments, the inflammatory disorder or disease is asthma.

In some embodiments, the inflammatory disorder or disease is chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; systemic sclerosis; idiopathic pulmonary fibrosis; alopecia areata; celiac disease; allergic rhinitis; eosinophilic fasciitis; scleromyxedema; scleredema; or nephrogenic systemic fibrosis.

In another aspect, provided herein is a method for treating a pathology associated with elevated levels of TSLP and/or OX40L in a mammalian subject in need thereof, the method comprising administering to the mammalian subject a therapeutically effective amount of any of the isolated antibodies, or antigen binding fragments thereof, disclosed herein, a pharmaceutical composition disclosed herein, or a therapeutically effective amount of a pharmaceutical composition comprising an isolated antibody, or antigen binding fragment thereof, that binds TSLP and a pharmaceutically acceptable excipient and a therapeutically effective amount of a pharmaceutical composition comprising an antibody, or antigen binding fragment thereof, that binds OX40L and a pharmaceutically acceptable excipient.

In another aspect, provided herein is a method of reducing biological activity of TSLP and/or OX40L in a mammalian subject in need thereof, the method comprising administering to the mammalian subject a therapeutically effective amount of any of the isolated antibodies, or antigen binding fragments thereof, disclosed herein, a pharmaceutical composition disclosed herein, or a therapeutically effective amount of a pharmaceutical composition comprising an isolated antibody, or antigen binding fragment thereof, that binds TSLP and a pharmaceutically acceptable excipient and a therapeutically effective amount of a pharmaceutical composition comprising an antibody, or antigen binding fragment thereof, that binds OX40L and a pharmaceutically acceptable excipient.

In some embodiments, the mammalian subject is a human.

In some embodiments of any of the foregoing methods, the method further includes administering a hyaluronidase or a variant thereof. In some embodiments, the hyaluronidase or variant thereof is administered separately from the antibodies (e.g., in a separate composition, which may be administered before, after, or at about the same time as the antibodies). In some embodiments, the hyaluronidase or variant thereof is administered as part of a composition that contains the isolated antibody, or antigen binding fragment thereof, that binds TSLP and/or the isolated antibody, or antigen binding fragment thereof, that binds OX40L (e.g., the hyaluronidase or variant thereof can be administered in a composition containing the anti-TSLP antibody, a composition containing the anti-OX40L antibody, or a composition containing both of the antibodies).

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof. In certain aspects, described herein are compositions comprising any one of the antibodies, or antigen binding fragments thereof, described herein that binds TSLP, any one of the antibodies, or antigen binding fragments thereof, described herein that binds OX40L, and any one of the hyaluronidases or variants thereof described herein.

In certain aspects, described herein are compositions comprising hyaluronidase or a variant thereof and a multi-specific antibody comprising a first antigen binding region that binds TSLP and a second antigen binding region that binds OX40L.

In certain aspects, described herein are a combination of an antibody, or antigen binding fragment thereof, that binds TSLP, an antibody, or antigen binding fragment thereof, that binds OX40L, and a hyaluronidase or variant thereof. In certain aspects, described herein are a combination of any one of the antibodies, or antigen binding fragments thereof, described herein that binds TSLP, any one of the antibodies, or antigen binding fragments thereof, described herein that binds OX40L, and a hyaluronidase or a variant thereof.

In certain aspects, described herein are combinations comprising hyaluronidase or a variant thereof and a multi-specific antibody comprising a first antigen binding region that binds TSLP and a second antigen binding region that binds OX40L.

In certain aspects, described herein are methods of treating an inflammatory disorder or disease in a human patient comprising co-administering to the patient an isolated antibody, or antigen binding fragment thereof, that binds TSLP, an isolated antibody, or antigen binding fragment thereof, that binds OX40L, and a hyaluronidase or a variant thereof. In certain aspects, described herein are methods of treating an inflammatory disorder or disease in a human patient comprising co-administering to the patient any one of the antibodies, or antigen binding fragments thereof, described herein that binds TSLP, any one of the antibodies, or antigen binding fragments thereof, described herein that binds OX40L, and any one of the hyaluronidases or variants thereof described herein.

In certain aspects, described herein are methods of treating an inflammatory disorder or disease in a human patient comprising co-administering to the patient hyaluronidase or a variant thereof and a multi-specific antibody comprising a first antigen binding region that binds TSLP and a second antigen binding region that binds OX40L. In certain aspects, described herein are methods of treating an inflammatory disorder or disease in a human patient comprising co-administering to the patient any one of the hyaluronidases or variants thereof described herein and any one of the multi-specific antibodies described herein (e.g., comprising a first antigen binding region that binds TSLP and a second antigen binding region that binds OX40L).

In certain embodiments, the antibody, or antigen binding fragment thereof, that binds TSLP and/or the antibody, or antigen binding fragment thereof, that binds OX40L is a humanized, human, or chimeric antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

In certain embodiments, the human Fc region of the antibody, or antigen binding fragment thereof, that binds TSLP and/or the antibody, or antigen binding fragment thereof, that binds OX40L comprises a human IgG sequence selected from a sequence set forth in any one of SEQ ID NOs: 47-270. In certain embodiments, the heavy chain of the antibody, or antigen binding fragment thereof, that binds TSLP and/or the antibody, or antigen binding fragment thereof, that binds OX40L comprises a constant heavy chain sequence set forth in SEQ ID NO: 173 or 61. In certain embodiments, the heavy chain of the antibody, or antigen binding fragment thereof, that binds TSLP and/or the antibody, or antigen binding fragment thereof, that binds OX40L comprises a constant heavy chain sequence set forth in SEQ ID NO: 173. In certain embodiments, the light chain of the antibody, or antigen binding fragment thereof, that binds TSLP and/or the antibody, or antigen binding fragment thereof, that binds OX40L comprises a constant light chain sequence set forth in SEQ ID NO: 520 or SEQ ID NO: 46.

In some embodiments, the antibody, or antigen binding fragment thereof, that binds TSLP and/or the antibody, or antigen binding fragment thereof, that binds OX40L comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.

In some embodiments, the antibody, or antigen binding fragment thereof, that binds TSLP and/or the antibody, or antigen binding fragment thereof, that binds OX40L comprises an Fc region comprising one or more amino acid substitutions, wherein the one or more amino acid substitutions result in a decrease in one or more of, ADCC activity, ADCP activity or CDC activity compared to an antibody comprising a wild-type Fc region.

In certain embodiments, the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV. In certain embodiments, the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

In certain embodiments, the antibody, or antigen binding fragment thereof, that binds TSLP and/or the antibody, or antigen binding fragment thereof, that binds OX40L comprises a human IgG1 Fc region with LALA and YTE mutations.

Although the EU numbering system is typically used to identify the positions of the various Fc mutations described herein, direct numbering can also be used. For example, in certain embodiments, an antibody described herein comprises an Fc region with YTE mutations at positions 253/255/257 or 257/259/261, respectively. In certain embodiments, an antibody described herein comprises an Fc region with LALA mutations at positions 235/236 or 239/240, respectively. In certain embodiments, an antibody described herein comprises an Fc region (e.g., an IgG1 Fc region) with YTE mutations at positions 253/255/257, respectively and with LALA mutations at positions 235/236, respectively. In certain embodiments, an antibody described herein comprises an Fc region (e.g., an IgG1 Fc region) with YTE mutations at positions 257/259/261, respectively and with LALA mutations at positions 239/240, respectively. In certain embodiments, the TSLP antibody described herein comprises an Fc region (e.g., an IgG1 Fc region) comprising YTE mutations at positions 257/259/261, respectively and LALA mutations at positions 239/240, respectively. In certain embodiments, the TSLP antibody described herein comprises the VH and VL of Antibody 5 and an Fc region comprising YTE mutations at positions 257/259/261, respectively and with LALA mutations at positions 239/240, respectively. In certain embodiments, the OX40L antibody described herein comprises an Fc region (e.g., an IgG1 Fc region) comprising YTE mutations at positions 253/255/257, respectively and LALA mutations at positions 235/236, respectively. In certain embodiments, the OX40L antibody described herein comprises the VH and VL of Construct 114 and an Fc region comprising YTE mutations at positions 253/255/257, respectively and with LALA mutations at positions 235/236, respectively.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E (by direct numbering, e.g., for an OX40L antibody described herein). In another embodiment, the human Fc region comprises a human IgG1 Fc with LALA mutations at L239A/L240A and/or YTE mutations at M257Y/S259T/T261E (by direct numbering, e.g., for a TSLP antibody described herein).

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antibody, or antigen binding fragment thereof, that binds TSLP comprises a VH sequence set forth in SEQ ID NO: 34 and a VL sequence set forth in SEQ ID NO: 41. In one embodiment, the antibody, or antigen binding fragment thereof, that binds TSLP comprises a heavy chain comprising a constant heavy chain sequence set forth in SEQ ID NO: 61 or 173. In one embodiment, the antibody, or antigen binding fragment thereof, that binds TSLP comprises a light chain comprising a constant light chain sequence set forth in SEQ ID NO: 520. In certain embodiments, the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 520. In certain embodiments, the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 520. In certain embodiments, the antibody, or antigen binding fragment thereof, comprises a heavy chain sequence set forth in SEQ ID NO: 524 and a light chain sequence set forth in SEQ ID NO: 525 In certain embodiments, the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520. In certain embodiments, the antibody, or antigen binding fragment thereof, comprises a heavy chain sequence set forth in SEQ ID NO: 526 and a light chain sequence set forth in SEQ ID NO: 525.

In certain embodiments, the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antibody, or antigen binding fragment thereof, that binds OX40L comprises a VH sequence set forth in SEQ ID NO: 335 and a VL sequence set forth in SEQ ID NO: 339. In one embodiment, the antibody, or antigen binding fragment thereof, that binds OX40L comprises a heavy chain comprising a constant heavy chain sequence set forth in SEQ ID NO: 61 or 173. In one embodiment, the antibody, or antigen binding fragment thereof, that binds OX40L comprises a light chain comprising a constant light chain sequence set forth in SEQ ID NO: 46. In certain embodiments, the antibody, or antigen binding fragment thereof, comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173 and/or a constant heavy chain sequence set forth in SEQ ID NO: 61, and a constant light chain sequence set forth in SEQ ID NO: 46. In certain embodiments, the antibody, or antigen binding fragment thereof, comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173, and a constant light chain sequence set forth in SEQ ID NO: 46. In certain embodiments, the antibody, or antigen binding fragment thereof, comprises a heavy chain sequence set forth in SEQ ID NO: 543 and a light chain sequence set forth in SEQ ID NO: 545. In certain embodiments, the antibody, or antigen binding fragment thereof, comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61, and a constant light chain sequence set forth in SEQ ID NO: 46. In certain embodiments, the antibody, or antigen binding fragment thereof, comprises a heavy chain sequence set forth in SEQ ID NO: 544 and a light chain sequence set forth in SEQ ID NO: 545.

In certain embodiments, a multi-specific antibody comprising a first antigen binding region that binds TSLP and a second antigen binding region that binds OX40L is provided. In one embodiment, the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antigen binding region that binds TSLP comprises a VH sequence set forth in SEQ ID NO: 34 and a VL sequence set forth in SEQ ID NO: 41.

In another embodiment, the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antigen binding region that binds OX40L comprises a VH sequence set forth in SEQ ID NO: 335 and a VL sequence set forth in SEQ ID NO: 339.

In certain embodiments, the antibody or antigen binding region that binds TSLP and/or the antibody or antigen binding region that binds OX40L comprises a heavy chain human constant region of a class selected from IgG, IgA, IgD, IgE, and IgM. In certain embodiments, the antibody or antigen binding region that binds TSLP and/or the antibody or antigen binding region that binds OX40L comprises a human Fc region, and wherein the human Fc region comprises a human heavy chain constant region of the class IgG and a subclass selected from IgG1, IgG2, IgG3, and IgG4. In certain embodiments, the human Fc region of the antibody or antigen binding region that binds TSLP and/or the antibody or antigen binding region that binds OX40L comprises a human IgG1 Fc region. In certain embodiments, the human Fc region of the antibody or antigen binding region that binds TSLP and/or the antibody or antigen binding region that binds OX40L comprises a human IgG4 Fc region. In certain embodiments, the human Fc region of the antibody or antigen binding region that binds TSLP and/or the antibody or antigen binding region that binds OX40L comprises a human IgG2 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used, these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E (e.g., in an OX40L antibody described herein). In another embodiment, the human Fc region comprises a human IgG1 Fc region with LALA mutations at L239A/L240A and/or YTE mutations at M257Y/S259T/T261E (e.g., in a TSLP antibody described herein).

In certain embodiments, the Fc region of the antibody or antigen binding region that binds TSLP and/or the antibody or antigen binding region that binds OX40L binds to Neonatal Fc receptor (FcRn). In certain embodiments, the Fc region of the antibody or antigen binding region that binds TSLP and/or the antibody or antigen binding region that binds OX40L binds an FcRn with higher affinity at pH 6.0 compared to an antibody comprising a wild-type Fc region. In certain embodiments, the Fc region of the antibody or antigen binding region that binds TSLP and/or the antibody or antigen binding region that binds OX40L binds to FcRn with a KD of <1×10⁻⁷ M at pH 6.0.

In certain embodiments, the antibody or antigen binding region that binds TSLP and/or the antibody or antigen binding region that binds OX40L is a monoclonal antibody.

In certain embodiments, the antibody or antigen binding region that binds TSLP binds a TSLP sequence set forth in any one of the amino acid sequences of SEQ ID NOs: 271-272. In certain embodiments, the antibody or antigen binding region that binds OX40L binds an OX40L sequence set forth in the amino acid sequence of SEQ ID NO:439.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase comprises the amino acid sequence set forth in any one of SEQ ID NOs: 453-462 and 464-519, or a mixture thereof. In certain embodiments, the recombinant human hyaluronidase is rHuPH20 (i.e., a composition comprising one or more of SEQ ID NOs: 455-460). In certain embodiments, the rHuPH20 formulation is ENHANZE®.

In one embodiment, the recombinant human hyaluronidase includes a sequence of amino acids in any one of SEQ ID NOs: 453-462 and 464-519, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 453-462 and 464-519 and retains hyaluronidase activity. In one embodiment, the recombinant human hyaluronidase comprises a sequence having at least 95%, 96%, 97%, 98%, or 99% to the amino acid sequence of SEQ ID NO: 453. In one embodiment, the recombinant human hyaluronidase comprises a sequence having at least 95%, 96%, 97%, 98%, or 99% to the amino acid sequence of SEQ ID NO: 455.

In another embodiment, the recombinant human hyaluronidase comprises the amino acid sequence set forth in SEQ ID NO:453. In another embodiment, the recombinant human hyaluronidase consists of the amino acid sequence set forth in SEQ ID NO:453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO:454. In another embodiment, the recombinant human hyaluronidase comprises the amino acid sequence set forth in SEQ ID NO:455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO:456. In another embodiment, the recombinant human hyaluronidase consists of the amino acid sequence set forth in SEQ ID NO: 456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO:457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO:458. In another embodiment, the recombinant human hyaluronidase comprises the amino acid sequence set forth in SEQ ID NO:459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO:460. In another embodiment, the recombinant human hyaluronidase consists of the amino acid sequence set forth in SEQ ID NO: 460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO:461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO:462. In another embodiment, the recombinant human hyaluronidase comprises the amino acid sequence set forth in SEQ ID NO:464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO: 465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO:466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO:468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO: 469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO:470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO:472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO: 473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO:474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475. In another embodiment, the recombinant human hyaluronidase comprises the amino acid sequence set forth in SEQ ID NO:476. In another embodiment, the recombinant human hyaluronidase consists of the amino acid sequence set forth in SEQ ID NO:476. In another embodiment, the recombinant human hyaluronidase comprises the amino acid sequence set forth in SEQ ID NO:477. In another embodiment, the recombinant human hyaluronidase consists of the amino acid sequence set forth in SEQ ID NO: 477. In another embodiment, the recombinant human hyaluronidase comprises the amino acid sequence set forth in SEQ ID NO:478. In another embodiment, the recombinant human hyaluronidase consists of the amino acid sequence set forth in SEQ ID NO:478. In another embodiment, the recombinant human hyaluronidase comprises the amino acid sequence set forth in SEQ ID NO:479. In another embodiment, the recombinant human hyaluronidase consists of the amino acid sequence set forth in SEQ ID NO:479. In another embodiment, the recombinant human hyaluronidase comprises the amino acid sequence set forth in SEQ ID NO:480. In another embodiment, the recombinant human hyaluronidase consists of the amino acid sequence set forth in SEQ ID NO:480. In another embodiment, the recombinant human hyaluronidase comprises the amino acid sequence set forth in SEQ ID NO:481. In another embodiment, the recombinant human hyaluronidase consists of the amino acid sequence set forth in SEQ ID NO: 481. In another embodiment, the recombinant human hyaluronidase comprises the amino acid sequence set forth in SEQ ID NO:482. In another embodiment, the recombinant human hyaluronidase consists of the amino acid sequence set forth in SEQ ID NO:482. In another embodiment, the recombinant human hyaluronidase comprises the amino acid sequence set forth in SEQ ID NO:483. In another embodiment, the recombinant human hyaluronidase consists of the amino acid sequence set forth in SEQ ID NO:483. In another embodiment, the recombinant human hyaluronidase comprises the amino acid sequence set forth in SEQ ID NO:484. In another embodiment, the recombinant human hyaluronidase consists of the amino acid sequence set forth in SEQ ID NO:484. In another embodiment, the recombinant human hyaluronidase comprises the amino acid sequence set forth in SEQ ID NO:485. In another embodiment, the recombinant human hyaluronidase consists of the amino acid sequence set forth in SEQ ID NO: 485. In another embodiment, the recombinant human hyaluronidase comprises the amino acid sequence set forth in SEQ ID NO:486. In another embodiment, the recombinant human hyaluronidase consists of the amino acid sequence set forth in SEQ ID NO:486. In another embodiment, the recombinant human hyaluronidase comprises the amino acid sequence set forth in SEQ ID NO:487. In another embodiment, the recombinant human hyaluronidase consists of the amino acid sequence set forth in SEQ ID NO:487. In another embodiment, the recombinant human hyaluronidase comprises the amino acid sequence set forth in SEQ ID NO:488. In another embodiment, the recombinant human hyaluronidase consists of the amino acid sequence set forth in SEQ ID NO:488. In another embodiment, the recombinant human hyaluronidase comprises the amino acid sequence set forth in SEQ ID NO:489. In another embodiment, the recombinant human hyaluronidase consists of the amino acid sequence set forth in SEQ ID NO: 489. In another embodiment, the recombinant human hyaluronidase comprises the amino acid sequence set forth in SEQ ID NO:490. In another embodiment, the recombinant human hyaluronidase consists of the amino acid sequence set forth in SEQ ID NO:490. In another embodiment, the recombinant human hyaluronidase comprises the amino acid sequence set forth in SEQ ID NO:491. In another embodiment, the recombinant human hyaluronidase consists of the amino acid sequence set forth in SEQ ID NO:491. In another embodiment, the recombinant human hyaluronidase comprises the amino acid sequence set forth in SEQ ID NO:492. In another embodiment, the recombinant human hyaluronidase consists of the amino acid sequence set forth in SEQ ID NO:492. In another embodiment, the recombinant human hyaluronidase comprises the amino acid sequence set forth in SEQ ID NO:493. In another embodiment, the recombinant human hyaluronidase consists of the amino acid sequence set forth in SEQ ID NO: 493. In another embodiment, the recombinant human hyaluronidase comprises the amino acid sequence set forth in SEQ ID NO:494. In another embodiment, the recombinant human hyaluronidase consists of the amino acid sequence set forth in SEQ ID NO:494. In another embodiment, the recombinant human hyaluronidase comprises the amino acid sequence set forth in SEQ ID NO:495. In another embodiment, the recombinant human hyaluronidase consists of the amino acid sequence set forth in SEQ ID NO:495. In another embodiment, the recombinant human hyaluronidase comprises the amino acid sequence set forth in SEQ ID NO:496. In another embodiment, the recombinant human hyaluronidase consists of the amino acid sequence set forth in SEQ ID NO:496. In another embodiment, the recombinant human hyaluronidase comprises the amino acid sequence set forth in SEQ ID NO:497. In another embodiment, the recombinant human hyaluronidase consists of the amino acid sequence set forth in SEQ ID NO: 497. In another embodiment, the recombinant human hyaluronidase comprises the amino acid sequence set forth in SEQ ID NO:498. In another embodiment, the recombinant human hyaluronidase consists of the amino acid sequence set forth in SEQ ID NO:498. In another embodiment, the recombinant human hyaluronidase comprises the amino acid sequence set forth in SEQ ID NO:499. In another embodiment, the recombinant human hyaluronidase consists of the amino acid sequence set forth in SEQ ID NO:499. In another embodiment, the recombinant human hyaluronidase comprises the amino acid sequence set forth in SEQ ID NO:500. In another embodiment, the recombinant human hyaluronidase consists of the amino acid sequence set forth in SEQ ID NO:500. In another embodiment, the recombinant human hyaluronidase comprises the amino acid sequence set forth in SEQ ID NO:501. In another embodiment, the recombinant human hyaluronidase consists of the amino acid sequence set forth in SEQ ID NO: 501. In another embodiment, the recombinant human hyaluronidase comprises the amino acid sequence set forth in SEQ ID NO:502. In another embodiment, the recombinant human hyaluronidase consists of the amino acid sequence set forth in SEQ ID NO:502. In another embodiment, the recombinant human hyaluronidase comprises the amino acid sequence set forth in SEQ ID NO:503. In another embodiment, the recombinant human hyaluronidase consists of the amino acid sequence set forth in SEQ ID NO:503. In another embodiment, the recombinant human hyaluronidase comprises the amino acid sequence set forth in SEQ ID NO:504. In another embodiment, the recombinant human hyaluronidase consists of the amino acid sequence set forth in SEQ ID NO:504. In another embodiment, the recombinant human hyaluronidase comprises the amino acid sequence set forth in SEQ ID NO:505. In another embodiment, the recombinant human hyaluronidase consists of the amino acid sequence set forth in SEQ ID NO: 505. In another embodiment, the recombinant human hyaluronidase comprises the amino acid sequence set forth in SEQ ID NO:506. In another embodiment, the recombinant human hyaluronidase consists of the amino acid sequence set forth in SEQ ID NO:506. In another embodiment, the recombinant human hyaluronidase comprises the amino acid sequence set forth in SEQ ID NO:507. In another embodiment, the recombinant human hyaluronidase consists of the amino acid sequence set forth in SEQ ID NO:507. In another embodiment, the recombinant human hyaluronidase comprises the amino acid sequence set forth in SEQ ID NO:508. In another embodiment, the recombinant human hyaluronidase consists of the amino acid sequence set forth in SEQ ID NO:508. In another embodiment, the recombinant human hyaluronidase comprises the amino acid sequence set forth in SEQ ID NO:509. In another embodiment, the recombinant human hyaluronidase consists of the amino acid sequence set forth in SEQ ID NO: 509. In another embodiment, the recombinant human hyaluronidase comprises the amino acid sequence set forth in SEQ ID NO:510. In another embodiment, the recombinant human hyaluronidase consists of the amino acid sequence set forth in SEQ ID NO:510. In another embodiment, the recombinant human hyaluronidase comprises the amino acid sequence set forth in SEQ ID NO:511. In another embodiment, the recombinant human hyaluronidase consists of the amino acid sequence set forth in SEQ ID NO:511. In another embodiment, the recombinant human hyaluronidase comprises the amino acid sequence set forth in SEQ ID NO:512. In another embodiment, the recombinant human hyaluronidase consists of the amino acid sequence set forth in SEQ ID NO:512. In another embodiment, the recombinant human hyaluronidase comprises the amino acid sequence set forth in SEQ ID NO:513. In another embodiment, the recombinant human hyaluronidase consists of the amino acid sequence set forth in SEQ ID NO: 513. In another embodiment, the recombinant human hyaluronidase comprises the amino acid sequence set forth in SEQ ID NO:514. In another embodiment, the recombinant human hyaluronidase consists of the amino acid sequence set forth in SEQ ID NO:514. In another embodiment, the recombinant human hyaluronidase comprises the amino acid sequence set forth in SEQ ID NO:515. In another embodiment, the recombinant human hyaluronidase consists of the amino acid sequence set forth in SEQ ID NO:515. In another embodiment, the recombinant human hyaluronidase comprises the amino acid sequence set forth in SEQ ID NO:516. In another embodiment, the recombinant human hyaluronidase consists of the amino acid sequence set forth in SEQ ID NO:516. In another embodiment, the recombinant human hyaluronidase comprises the amino acid sequence set forth in SEQ ID NO:517. In another embodiment, the recombinant human hyaluronidase consists of the amino acid sequence set forth in SEQ ID NO: 517. In another embodiment, the recombinant human hyaluronidase comprises the amino acid sequence set forth in SEQ ID NO:518. In another embodiment, the recombinant human hyaluronidase consists of the amino acid sequence set forth in SEQ ID NO:518. In another embodiment, the recombinant human hyaluronidase comprises the amino acid sequence set forth in SEQ ID NO:519. In another embodiment, the recombinant human hyaluronidase consists of the amino acid sequence set forth in SEQ ID NO:519.

In certain embodiments, the hyaluronidase or variant thereof and antibodies (i.e., anti-OX40L antibody, or antigen binding fragment thereof, and anti-TSLP antibody, or antigen binding fragment thereof) or multi-specific antibody (i.e., comprising a first antigen binding region that binds TSLP and a second antigen binding region that binds OX40L) are administered in separate formulations. In certain embodiments, the hyaluronidase or variant thereof and antibodies or multi-specific antibody are administered simultaneously in separate formulations. In certain embodiments, the hyaluronidase or variant thereof is administered to the patient prior to administration of the antibodies or multi-specific antibody. In certain embodiments, the hyaluronidase or variant thereof is administered to the patient after administration of the antibodies or multi-specific antibody. In certain embodiments, the hyaluronidase or variant thereof is administered to the patient after administration of one of the antibodies (either the TSLP antibody or the OX40L antibody) but is administered before the administration of the second of the antibodies (either the TSLP antibody or the OX40L antibody).

In certain embodiments, the hyaluronidase or variant thereof and the antibodies or multi-specific antibody are mixed and administered in a single formulation. In certain embodiments, the hyaluronidase or variant thereof and the antibodies or multi-specific antibody are administered by subcutaneous injection. In certain embodiments, the hyaluronidase or variant thereof and the antibodies or multi-specific antibody are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind TSLP described herein and any one of the antibodies, or antigen binding fragments thereof, that bind OX40L described herein in combination with a hyaluronidase or variant thereof. In certain embodiments, methods of treating an inflammatory disorder or disease in a human patient are provided, wherein the method comprises co-administering to the patient any one of the multi-specific antibodies described herein in combination with a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; idiopathic pulmonary fibrosis; alopecia areata; systemic sclerosis; allergic rhinitis; eosinophilic fasciitis; scleromyxedema; scleredema; or nephrogenic systemic fibrosis.

In certain aspects, described herein are compositions comprising an isolated antibody, or antigen binding region thereof, that binds TSLP, an isolated antibody, or antigen binding region thereof, that binds OX40L, and a hyaluronidase or variant thereof.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds TSLP comprises a VH sequence set forth in SEQ ID NO: 34 and a VL sequence set forth in SEQ ID NO: 41.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds OX40L comprises a VH sequence set forth in SEQ ID NO: 335 and a VL sequence set forth in SEQ ID NO: 339.

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a VH sequence set forth in SEQ ID NO: 34 and a VL sequence set forth in SEQ ID NO: 41; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are compositions comprising hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain aspects, described herein are combinations comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are combinations comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a VH sequence set forth in SEQ ID NO: 34 and a VL sequence set forth in SEQ ID NO: 11; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are combinations comprising hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are combinations comprising hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain aspects, described herein are methods of treating an inflammatory disorder or disease in a human patient comprising administering to the patient an isolated antibody, or antigen binding fragment thereof, that binds TSLP, an isolated antibody, or antigen binding fragment thereof, that binds OX40L, and a hyaluronidase or a variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are methods of treating an inflammatory disorder or disease in a human patient comprising administering to the patient an isolated antibody, or antigen binding fragment thereof, that binds TSLP, an isolated antibody, or antigen binding fragment thereof, that binds OX40L, and a hyaluronidase or a variant thereof, wherein the antibody, or antigen binding fragment thereof, that binds TSLP comprises a VH sequence set forth in SEQ ID NO: 34 and a VL sequence set forth in SEQ ID NO: 41; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are methods of treating an inflammatory disorder or disease in a human patient comprising administering to the patient hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are methods of treating an inflammatory disorder or disease in a human patient comprising administering to the patient hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain embodiments of any of the foregoing aspects, the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 520. In certain embodiments, the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 520. In certain embodiments, the antibody, or antigen binding fragment thereof, comprises a heavy chain sequence set forth in SEQ ID NO: 524 and a light chain sequence set forth in SEQ ID NO: 525. In certain embodiments, the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520. In certain embodiments, the antibody, or antigen binding fragment thereof, comprises a heavy chain sequence set forth in SEQ ID NO: 526 and a light chain sequence set forth in SEQ ID NO: 525.

In certain embodiments of any of the foregoing aspects, the antibody, or antigen binding fragment thereof, that binds OX40L comprises a VH sequence set forth in SEQ ID NO: 335, a VL sequence set forth in SEQ ID NO: 339, a constant heavy chain sequence set forth in SEQ ID NO: 173 and/or a constant heavy chain sequence set forth in SEQ ID NO: 61, and a constant light chain sequence set forth in SEQ ID NO: 46. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 173, and a constant light chain sequence set forth in SEQ ID NO: 46. In certain embodiments, the antibody, or antigen binding fragment thereof, comprises a heavy chain sequence set forth in SEQ ID NO: 543 and a light chain sequence set forth in SEQ ID NO: 545. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 61, and a constant light chain sequence set forth in SEQ ID NO: 46. In certain embodiments, the antibody, or antigen binding fragment thereof, comprises a heavy chain sequence set forth in SEQ ID NO: 544 and a light chain sequence set forth in SEQ ID NO: 545.

In certain aspects, described herein is a kit comprising: (a) a dose of an antibody, or antigen binding fragment thereof, that binds TSLP, wherein the antibody comprises a VH sequence set forth in SEQ ID NO: 34 and a VL sequence set forth in SEQ ID NO: 41; (b) a dose of an antibody, or antigen binding fragment thereof, that binds OX40L, wherein the antibody comprises a VH sequence set forth in SEQ ID NO: 335 and a VL sequence set forth in SEQ ID NO: 339; (c) a dose of a hyaluronidase or variant thereof; and (d) instructions for use.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 453-462 and 464-519, or a mixture thereof. In certain embodiments, the recombinant human hyaluronidase is rHuPH20 (i.e., a composition comprising one or more of SEQ ID NOs: 455-460). In certain embodiments, the rHuPH20 formulation is ENHANZE®.

In certain embodiments, the kit is for use in treating an inflammatory disorder or disease. In certain embodiments, the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; idiopathic pulmonary fibrosis; alopecia areata; systemic sclerosis; allergic rhinitis; eosinophilic fasciitis; scleromyxedema; scleredema; or nephrogenic systemic fibrosis.

### DETAILED DESCRIPTION

Described herein are compositions and combinations containing an anti-OX40L antibody, or antigen binding fragment thereof, an anti-TSLP antibody, or antigen binding fragment thereof, and a hyaluronidase or variant thereof. The compositions and combinations can be used to treat a subject having an inflammatory disorder or disease, such as atopic dermatitis, asthma, or COPD. The antibodies, or antigen binding fragments thereof, can be administered to a subject separately from the hyaluronidase or variant thereof (e.g., in separate compositions) or may be co-formulated and administered in a single composition.

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably herein and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eukarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-TSLP antibody, or antigen binding fragment thereof, or an anti-OX40L antibody, or antigen binding fragment thereof, administered to an individual, either as a single dose or as part of a series of doses, which is effective to produce or contribute to a desired therapeutic effect, either alone or in combination with another therapeutic modality. Examples of a desired therapeutic effect are reducing an aberrant immune response, slowing or delaying disease development; stabilization of disease; and amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject,” “patient,” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody, or antigen binding fragment thereof, provided herein. In some aspects, the disease or condition is atopic dermatitis. In some aspects, the disease or condition is asthma.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

The terms “co-administration,” “co-administer,” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to, for example, the twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), Glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec-1), as used herein, refers to the dissociation rate constant of a particular antibody-antigen interaction. This value is also referred to as the koff value.

The term “ka” (M-1×sec-1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M-1), as used herein, refers to the association equilibrium constant of a particular antibody-antigen interaction. KA=ka/kd.

As used herein, “administration” refers to providing or giving a subject a therapeutic agent (e.g., an anti-OX40L antibody, or antigen binding fragment thereof, and/or an anti-TSLP antibody, or antigen binding fragment thereof, described herein) by any effective route. Exemplary routes of administration are described herein below.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in TABLE 1 below.

TABLE 1
Representative physicochemical properties of naturally occurring amino acids

| | | | | Electrostatic | |
|---|---|---|---|---|---|
| | | | | character at | |
| | 3 Letter | 1 Letter | Side-chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in cubic angstroms: 50-100 is small, 100-150 is intermediate, 150-200 is large, and >200 is bulky
From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

An “anti-TSLP antibody,” “TSLP antibody,” or “TSLP specific antibody” is an antibody, as provided herein, which specifically binds to the antigen TSLP.

An “anti-OX40L antibody,” “OX40L antibody,” or “OX40L specific antibody” is an antibody, as provided herein, which specifically binds to the antigen OX40L.

The term “epitope” means a portion of an antigen that specifically binds to an antibody.

The term “hypervariable region” or “HVR”, as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” or “fully human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises a polypeptide chain wherein the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains, respectively. The variable domains comprise the complementarity determining loops (CDR, also referred to as hypervariable region (HVR)) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv, see Pluckthun in The Pharmacology of Monoclonal Antibodies, vol. 113, Rosenburg and Moore eds., Springer-Verlag, New York, pp. 269-315 (1994).

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., (1998) FEBS Letters 414:521-526 and Muyldermans et al. (2001) Trends in Biochem. Sci. 26:230-245, each of which is incorporated by reference in its entirety. Single domain antibodies are also known as sdAbs or nanobodies. SdAbs are fairly stable and easy to express as fusion partner with the Fc chain of an antibody (Harmsen M M, De Haard H J (2007) “Properties, production, and applications of camelid single-domain antibody fragments” Appl. Microbiol Biotechnol. 77 (1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, (1981) Adv. Appl. Math. 2:482, by the homology alignment algorithm of Needleman & Wunsch, (1970) J. Mol. Biol. 48:443, by the search for similarity method of Pearson & Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of an algorithm that is suitable for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (www.ncbi.nlm.nih.gov/).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

The term “atopic dermatitis disease severity outcome measure” refers to a determination of certain signs, symptoms, features or parameters that have been associated with atopic dermatitis and that can be quantitatively or qualitatively assessed. Exemplary atopic dermatitis disease severity outcome measures include “Eczema Area and Severity Index” (EASI), “Severity Scoring of Atopic Dermatitis” (SCORAD), “Validated Investigator Global Assessment-Atopic Dermatitis” (vIGA-AD), “Investigator Global Assessment of Signs” (IGSA), Rajka/Langeland Atopic Dermatitis Severity Score, “Body Surface Area” (BSA), and Patient-Reported Outcomes including Pruritus Visual Analog Scale (an aspect of disease severity assessed as part of SCORAD), Sleep Loss Visual Analog Scale (an aspect of disease severity assessed as part of SCORAD), Atopic Dermatitis Symptom Diary (ADSD), Atopic Dermatitis Impact Questionnaire (ADIQ), Dermatology Life Quality Index (DLQI) (Finlay and Khan, Clin Exper Dermatol 1994; 19:210), 5-D Itch Scale (Elman et al., Br J Dermatol 2010; 162 (3): 587-593), Itch Numeric Rating Scale (I-NRS) (see Naegeli et al., International Journal of Dermatology. 2015; 54 (6): 715-722; and Newton L, et al., J. Patient Rep. Outcomes. 2019; 3 (1): 42), and the Patient-Oriented Eczema Measure (POEM) (www.nottingham.ac.uk/research/groups/cebd/resources/poem.aspx).

### Hyaluronidases

As used herein, “hyaluronidase” refers to an enzyme that degrades hyaluronic acid, which constitutes an essential part of the extracellular matrix. Included in this definition are naturally occurring hyaluronidases, recombinant hyaluronidases, both human and from other sources, as well as variants thereof. Such hyaluronidases include, but are not limited to, nonhuman hyaluronidases, including bacterial hyaluronidases, bovine hyaluronidases, ovine hyaluronidases, and variants thereof. Reference to hyaluronidase refers to all forms, including variants.

Hyaluronidases were initially discovered in bacteria, and are now known to be widely distributed in nature and have been found in many different species, including insects, snakes, and mammals. Human hyaluronidase is present both in organs (e.g., testis, spleen, skin, eyes, liver, kidneys, uterus, and placenta) and in body fluids (e.g., tears, blood, and semen). In the human, six different hyaluronidases, HYAL1-4, HYAL-P1 and PH-20, have been identified. PH-20 exerts the strongest biologic activity, is found in high concentrations in the testicles, and can be localized on the head and the acrosome of human spermatozoa (see, e.g., Weber G C, et al., Adv. Exp. Med. Biol. 2019; 1148:255-277).

Hyaluronidases are classified into three categories according to their mechanism of action (see, e.g., Jung H., Arch. Plast. Surg. 2020 July; 47 (4): 297-300). First, mammalian hyaluronidases are endo-β-N-acetylhexosaminidases that break down β-1,4 glycosidic linkages to form tetrasaccharides. Second, leech/hookworm hyaluronidases are endo-β-D-glucuronidases that break down β-1,3 glycosidic bonds to form pentasaccharides and hexasaccharides. Finally, microbial hyaluronidases are classified as hyaluronate lyases. Unlike other hyaluronidases, they do not catalyze hydrolysis reactions. Instead, they produce unsaturated disaccharides through a β-elimination reaction at β-1,4 glycosidic linkage.

Hyaluronidases can also be classified into two types according to the pH at which they are most active (see, e.g., Jung H., Arch. Plast. Surg. 2020 July; 47 (4): 297-300). Acid-active hyaluronidases are activated at a pH of 3 to 4. Neutral-active hyaluronidases, which include the hyaluronidase enzymes found in snake and bee venom, are activated at a pH of 5 to 8 (see, e.g., Cavallini M, et al., Aesthet. Surg. J. 2013; 33:1167-74).

Hyaluronidase use has become more diverse and widespread in clinical practice. Today, animal-derived bovine or ovine testicular hyaluronidases, as well as synthetic hyaluronidases, are clinically applied as adjuncts to increase the bioavailability of drugs, for the therapy of extravasations, or for the management of complications associated with the aesthetic injection of hyaluronic acid-based fillers.

Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase (e.g., see Sato et al. (1994) Appl. Microbiol. Biotechnol. 41 (1): 39-46)). Exemplary chondroitinase AC enzymes from the bacteria include, but are not limited to, those from Flavobacterium heparinum, Victivallis vadensis, and Arthrobacter aurescens (see, e.g., Tkalec et al. (2000) Applied and Environmental Microbiology 66 (1): 29-35; Ernst et al. (1995) Critical Reviews in Biochemistry and Molecular Biology 30 (5): 387-444). Exemplary chondroitinase C enzymes from the bacteria include, but are not limited to, those from Streptococcus and Flavobacterium (Hibi et al. (1989) FEMS-Microbiol-Lett. 48 (2): 121-4; Michelacci et al. (1976) J. Biol. Chem. 251:1154-8; Tsuda et al. (1999) Eur. J. Biochem. 262:127-133).

Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include hyaluronidases from cows (SEQ ID NOs: 10, 11, 64 of U.S. Pat. No. 8,568,713) and BH55 (U.S. Pat. Nos. 5,747,027 and 5,827,721), yellow jacket wasp (SEQ ID NOs: 12 and 13 of U.S. Pat. No. 8,568,713), honey bee (SEQ ID NO:14 of U.S. Pat. No. 8,568,713), white-face hornet (SEQ ID NO:15 of U.S. Pat. No. 8,568,713), paper wasp (SEQ ID NO: 16 of U.S. Pat. No. 8,568,713), mouse (SEQ ID NOs: 17-19, and 32 of U.S. Pat. No. 8,568,713), pig (SEQ ID NOs: 20-21 of U.S. Pat. No. 8,568,713), rat (SEQ ID NOs: 22-24, and 31 of U.S. Pat. No. 8,568,713), rabbit (SEQ ID NO:25 of U.S. Pat. No. 8,568,713), sheep (SEQ ID NOs: 26, 27, 63 and 65 of U.S. Pat. No. 8,568,713), orangutan (SEQ ID NO:28 of U.S. Pat. No. 8,568,713), cynomolgus monkey (SEQ ID NO:29 of U.S. Pat. No. 8,568,713), guinea pig (SEQ ID NO:30 of U.S. Pat. No. 8,568,713), Arthrobacter sp. (strain FB24) (SEQ ID NO: 67 of U.S. Pat. No. 8,568,713), Bdellovibrio bacteriovorus (SEQ ID NO:68 of U.S. Pat. No. 8,568,713), Propionibacterium acnes (SEQ ID NO:69 of U.S. Pat. No. 8,568,713), Streptococcus agalactiae ((SEQ ID NO:70 of U.S. Pat. No. 8,568,713); 18RS21 (SEQ ID NO: 71 of U.S. Pat. No. 8,568,713); serotype Ia (SEQ ID NO:72 of U.S. Pat. No. 8,568,713); serotype III (SEQ ID NO:73 of U.S. Pat. No. 8,568,713), Staphylococcus aureus (strain COL) (SEQ ID NO:74 of U.S. Pat. No. 8,568,713); strain MRSA252 (SEQ ID NOs: 75 and 76 of U.S. Pat. Nos. 8,568,713 8,568,713); strain MSSA476 (SEQ ID NO:77 of U.S. Pat. No. 8,568,713); strain NCTC 8325 (SEQ ID NO:78 of U.S. Pat. No. 8,568,713); strain bovine RF122 (SEQ ID NOs: 79 and 80 of U.S. Pat. No. 8,568,713); strain USA300 (SEQ ID NO: 81 of U.S. Pat. No. 8,568,713), Streptococcus pneumoniae (SEQ ID NO:82 of U.S. Pat. No. 8,568,713); strain ATCC BAA-255/R6 (SEQ ID NO:83 of U.S. Pat. No. 8,568,713); serotype 2, strain D39/NCTC 7466 (SEQ ID NO:84 of U.S. Pat. No. 8,568,713), Streptococcus pyogenes (serotype (SEQ ID NO:85 of U.S. Pat. No. 8,568,713); serotype M2, strain MGAS10270 (SEQ ID NO:86 of U.S. Pat. No. 8,568,713); serotype M4, strain MGAS10750 (SEQ ID NO:87 of U.S. Pat. No. 8,568,713); serotype M6 (SEQ ID NO:88 of U.S. Pat. No. 8,568,713); serotype M12, strain MGAS2096 (SEQ ID NOs: 89 and 90 of U.S. Pat. No. 8,568,713); serotype M12, strain MGAS9429 (SEQ ID NO:91 of U.S. Pat. No. 8,568,713); serotype M28 (SEQ ID NO:92 of U.S. Pat. No. 8,568,713); Streptococcus suis (SEQ ID NOs: 93-95 of U.S. Pat. No. 8,568,713); Vibrio fischeri (strain ATCC 700601/ES114 (SEQ ID NO: 96 of U.S. Pat. No. 8,568,713), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include PH20 (SEQ ID NO:1 of U.S. Pat. No. 8,568,713), HYAL1 (SEQ ID NO:36 of U.S. Pat. No. 8,568,713), HYAL2 (SEQ ID NO:37 of U.S. Pat. No. 8,568,713), HYAL3 (SEQ ID NO:38 of U.S. Pat. No. 8,568,713), and HYAL4 (SEQ ID NO:36 of U.S. Pat. No. 8,568,713). The sequences and contents of U.S. Pat. No. 8,568,713 are expressly incorporated herein by reference. Also included amongst hyaluronidases are soluble hyaluronidases, including, ovine and bovine PH20, soluble human PH20 and soluble rHuPH20. Examples of commercially available bovine or ovine soluble hyaluronidases include Vitrase® (ovine hyaluronidase) and Amphadase® (bovine hyaluronidase).

Hyaluronidases as described herein include precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 60%, 65%, 70%, 75%, 80%, 85%, 90%, 95%, 96%, 97%, 98%, 99% or more sequence identity to the polypeptides set forth in SEQ ID NOs: 453-462 and 464-519. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, a soluble hyaluronidase refers to a polypeptide characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as soluble rHuPH20, are known in the art. Exemplary assays include the microturbidity assay described below (see, e.g., Example 3 of U.S. Pat. No. 8,568,713) that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, “functionally equivalent amount” or grammatical variations thereof, with reference to a hyaluronan degrading enzyme, refers to the amount of hyaluronan degrading enzyme that achieves the same effect as an amount (such as a known number of Units of hyaluronidase activity) of a reference enzyme, such as a hyaluronidase. For example, the activity of any hyaluronan degrading enzyme can be compared to the activity of rHuPH20 to determine the functionally equivalent amount of a hyaluronan degrading enzyme that would achieve the same effect as a known amount of rHuPH20. For example, the ability of a hyaluronan degrading enzyme to act as a spreading or diffusing agent can be assessed by injecting it into the lateral skin of mice with trypan blue (see, e.g. U.S. Pat. Publication No. 20050260186), and the amount of hyaluronan degrading enzyme required to achieve the same amount of diffusion as, for example, 100 units of a Hyaluronidase Reference Standard, can be determined. The amount of hyaluronan degrading enzyme required is, therefore, functionally equivalent to 100 units.

Exemplary hyaluronan degrading enzymes are hyaluronidases, particularly soluble hyaluronidases, such as a PH20, or a truncated or variant form thereof. The PH20 can be, for example, an ovine, bovine or truncated human PH20. The human PH20 mRNA transcript is normally translated to generate a 509 amino acid precursor polypeptide (SEQ ID NO:453; and replicated below) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:454. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:453. Thus, a 474 amino acid GPI-anchored mature polypeptide with an amino acid sequence set forth in SEQ ID NO:454 is produced.

The amino acid sequence of the human PH20 precursor polypeptide (SEQ ID NO: 453; 509 amino acids) is as follows:

MGVLKFKHIFFRSFVKSSGVSQIVFTFLLIPCCLTLNFRAPPVIP
NVPFLWAWNAPSEFCLGKFDEPLDMSLFSFIGSPRINATGQGVTI
FYVDRLGYYPYIDSITGVTVNGGIPQKISLQDHLDKAKKDITFYM
PVDNLGMAVIDWEEWRPTWARNWKPKDVYKNRSIELVQQQNVQLS
LTEATEKAKQEFEKAGKDFLVETIKLGKLLRPNHLWGYYLFPDCY
NHHYKKPGYNGSCFNVEIKRNDDLSWLWNESTALYPSIYLNTQQS
PVAATLYVRNRVREAIRVSKIPDAKSPLPVFAYTRIVFTDQVLKF
LSQDELVYTFGETVALGASGIVIWGTLSIMRSMKSCLLLDNYMET
ILNPYIINVTLAAKMCSQVLCQEQGVCIRKNWNSSDYLHLNPDNF
AIQLEKGGKFTVRGKPTLEDLEQFSEKFYCSCYSTLSCKEKADVK
DTDAVDVCIADGVCIDAFLKPPMETEEPQIFYNASPSTLSATMFI
VSILFLIISSVASL.

The amino acid sequence of the mature PH20 polypeptide (SEQ ID NO: 454; 474 amino acids) is as follows:

LNFRAPPVIPNVPFLWAWNAPSEFCLGKFDEPLDMSLFSFIGSPR
INATGQGVTIFYVDRLGYYPYIDSITGVTVNGGIPQKISLQDHLD
KAKKDITFYMPVDNLGMAVIDWEEWRPTWARNWKPKDVYKNRSIE
LVQQQNVQLSLTEATEKAKQEFEKAGKDFLVETIKLGKLLRPNHL
WGYYLFPDCYNHHYKKPGYNGSCFNVEIKRNDDLSWLWNESTALY
PSIYLNTQQSPVAATLYVRNRVREAIRVSKIPDAKSPLPVFAYTR
IVFTDQVLKFLSQDELVYTFGETVALGASGIVIWGTLSIMRSMKS
CLLLDNYMETILNPYIINVTLAAKMCSQVLCQEQGVCIRKNWNSS
DYLHLNPDNFAIQLEKGGKFTVRGKPTLEDLEQFSEKFYCSCYST
LSCKEKADVKDTDAVDVCIADGVCIDAFLKPPMETEEPQIFYNAS
PSTLSATMFIVSILFLIISSVASL.

Human PH20 exhibits hyaluronidase activity at both neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at both neutral and acid pH. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:454 and positions 142-172 of the precursor polypeptide set forth in SEQ ID NO:453, is required for enzyme activity at neutral pH. Amino acids at positions 111 and 113 (corresponding to the mature PH20 polypeptide set forth in SEQ ID NO:454) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:454, and positions 277-297 of the precursor polypeptide set forth in SEQ ID NO:453, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247: 810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO: 453 and positions 170-200 of the mature polypeptide set forth in SEQ ID NO:454. This region is highly conserved among hyaluronidases and is similar to the heparin binding motif. Mutation of the arginine residue at position 176 (corresponding to the mature PH20 polypeptide set forth in SEQ ID NO: 454) to a glycine results in a polypeptide with only about 1% of the hyaluronidase activity of the wild type polypeptide (Arming et al., (1997) Eur. J. Biochem. 247: 810-814).

There are seven potential N-linked glycosylation sites in human PH20 at N82, N166, N235, N254, N368, N393, N490 of the polypeptide exemplified in SEQ ID NO:453. Because amino acids 36 to 464 of SEQ ID NO:453 appear to contain the minimally active human PH20 hyaluronidase domain, the N-linked glycosylation site N-490 is not required for proper hyaluronidase activity. There are six disulfide bonds in human PH20. Two disulfide bonds between the cysteine residues C60 and C351 and between C224 and C238 of the polypeptide exemplified in SEQ ID NO:453 (corresponding to residues C25 and C316, and C189 and C203 of the mature polypeptide set forth in SEQ ID NO:454, respectively). A further four disulfide bonds are formed between the cysteine residues C376 and C387; between C381 and C435; between C437 and C443; and between C458 and C464 of the polypeptide exemplified in SEQ ID NO: 453 (corresponding to residues C341 and C352; between C346 and C400; between C402 and C408; and between C423 and C429 of the mature polypeptide set forth in SEQ ID NO:454, respectively).

As used herein, soluble recombinant human PH20 (rHuPH20) refers to a soluble form of human PH20 that is recombinantly expressed in Chinese Hamster Ovary (CHO) cells. Soluble human PH20 or sHuPH20 includes mature polypeptides lacking all or a portion of the glycosylphospatidylinositol (GPI) attachment site at the C-terminus such that upon expression, the polypeptides are soluble.

Soluble rHuPH20 is encoded by a nucleic acid that includes the signal sequence and is set forth in SEQ ID NO:463. Also included are DNA molecules that are allelic variants thereof and other soluble variants. The nucleic acid encoding soluble rHuPH20 is expressed in CHO cells which secrete the mature polypeptide.

SEQ ID NO: 463 encodes amino acids 1-482 of the human PH20 precursor polypeptide (amino acids 1-482 of SEQ ID NO: 453). Post translational processing removes the 35 amino acid signal sequence, leaving a 447 amino acid soluble recombinant human PH20 (SEQ ID NO: 455). As produced in the culture medium, there is heterogeneity at the C-terminus such that the product, designated rHuPH20, includes a mixture of species that can include any one or more of SEQ ID NOs: 455-460 in various abundance. Typically, rHuPH20 is produced in cells that facilitate correct N-glycosylation to retain activity, such as CHO cells (e.g., DG44 CHO cells). rHuPH20 may be produced by expressing a polynucleotide encoding amino acids 36-482 of SEQ ID NO: 453 in a cell, such as a CHO cell, and the polynucleotide encoding amino acids 36-482 of SEQ ID NO: 453 may be linked to the native signal sequence (amino acids 1-35 of SEQ ID NO: 453, as in SEQ ID NO: 463) or a heterologous signal sequence. The production of rHuPH20 is described in U.S. Pat. Nos. 8,568,713 and 8,343,487, each of which is incorporated herein by reference.

Exemplary sHuPH20 polypeptides include mature polypeptides having an amino acid sequence set forth in any one of SEQ ID NOs: 455-462 and 464-519. Other variants can have 60%, 70%, 80%, 85%, 90%, 91%, 92%, 93%, 94%, 95%, 96%, 97%, 98%, 99% or more sequence identity to any of SEQ ID NOs: 455-462 and 464-519, as long they retain a hyaluronidase activity and are soluble. Corresponding allelic variants and other variants also are included, including those corresponding to the precursor human PH20 polypeptide set forth in SEQ ID NOs: 453 and the mature human PH20 polypeptide set forth in SEQ ID NO: 454.

rHuPH20 was approved by the US Food and Drug Administration in 2005 as an adjuvant to increase the dispersion and absorption of other injected drugs. Recombinant human hyaluronidase PH20 is a transiently and locally-acting permeation enhancer that increases the dispersion and absorption of other injected agents. Recombinant human hyaluronidase PH20 depolymerizes hyaluronic acid (HA) at the injection site causing rapid decrease in the viscosity of the extracellular matrix, allowing bulk fluid flow and facilitating dispersion and absorption of coadministered agents (rHuPH20 Investigator's Brochure, 2018).

rHuPH20 is a glycosylated single chain protein with up to 447 amino acids, synthesized in CHO cells. As described above, rHuPH20 may be produced from a polynucleotide encoding amino acids 1-482 of SEQ ID NO: 453 (set forth in SEQ ID NO: 463), and post translational processing removes the 35 amino acid signal sequence, resulting in a polypeptide containing amino acids 36-482 of SEQ ID NO: 453 (set forth in SEQ ID NO: 455). As produced in the culture medium there is heterogeneity at the C-terminus such that the resulting rHuPH20 includes a mixture of species that can include any one or more of SEQ ID NOs: 455-460 in various abundance. Recombinant human hyaluronidase PH20 degrades HA under physiologic conditions and acts as a spreading factor in vivo. Therefore, when combined (co-mixed) or co-formulated with certain injectable drugs, rHUPH20 facilitates the absorption and dispersion of these drugs by temporarily reducing resistance to bulk fluid flow in the subcutaneous space. The permeability barrier in these tissues is restored to pre-injection levels within 24 to 48 hours after injection of rHuPH20.

Any suitable hyaluronidase (e.g., a recombinant human hyaluronidase) can be used in the methods, compositions, and combinations described herein, including, but not limited to, those described in U.S. Pat. No. 7,767,429 (e.g., SEQ ID NO:1), U.S. Pat. No. 7,846,431 (e.g., SEQ ID NO:1), U.S. Pat. No. 7,871,607 (e.g., SEQ ID NO:1), U.S. Pat. No. 8,105,586 (e.g., SEQ ID NO:1), U.S. Pat. No. 8,202,517 (e.g., SEQ ID NO:1), U.S. Pat. No. 8,257,699 (e.g., SEQ ID NO:1), U.S. Pat. No. 8,450,470 (e.g., SEQ ID NO:1), U.S. Pat. No. 8,431,124 (e.g., SEQ ID NO:1), U.S. Pat. No. 8,431,380 (e.g., SEQ ID NO:1), U.S. Pat. No. 8,580,252 (e.g., SEQ ID NO:1), U.S. Pat. No. 8,765,685 (e.g., SEQ ID NO:1), U.S. Pat. No. 8,772,246 (e.g., SEQ ID NO:1), U.S. Pat. No. 9,211,315 (e.g., SEQ ID NO:1), U.S. Pat. No. 9,562,223 (e.g., SEQ ID NO:1), U.S. Pat. No. 9,677,061 (e.g., SEQ ID NO:1), U.S. Pat. No. 9,677,062 (e.g., SEQ ID NO:1), U.S. Pat. No. 5,721,348 (e.g., SEQ ID NO: 6), US20210155913, US20210363270, and US20220289864, the contents of each of which are expressly incorporated herein by reference. The generation of such recombinant human hyaluronidases is described in U.S. Pat. Nos. 7,767,429; 7,871,607; and US20060104968, the contents of each of which are expressly incorporated herein by reference.

An exemplary recombinant human hyaluronidase is a soluble hyaluronidase provided as a composition designated rHuPH20, i.e., the active ingredient in the commercial product Hylenex® recombinant (hyaluronidase human injection), which is supplied as ENHANZE® drug product. ENHANZE® is a formulation containing rHuPH20 that can be admixed with an antibody prior to subcutaneous injection.

Hyaluronidases that have altered properties, such as increased stability and/or activity, have been produced and can also be used, including, but not limited to those described in U.S. Pat. Nos. 9,447,401, 10,865,400, and 11,041,149, the contents of each of which are expressly incorporated herein by reference. These patents provide about 7000 examples in which the effects of replacing each amino acid with 15 other amino acids on activity and stability were identified and described.

Other variants are known to those of skill in the art including those described, for example, in WO2020/022791 and WO2020197230A, the contents of each of which are expressly incorporated herein by reference. These variant polypeptides include replacements, insertions, and deletions, including one or more amino acid residues S343E, M345T, K349E, L353A, L3541, N356E, and 136 IT. Variants that contain such modifications and others are set forth in SEQ ID NOs: 60-115 from WO2020/022791. WO2021/150079 also provides variant polypeptides described as having increased stability.

In one embodiment, the recombinant human hyaluronidase includes a sequence of amino acids in any one of SEQ ID NOs: 453-462 and 464-519, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 453-462 and 464-519 and retains hyaluronidase activity. In one embodiment, the recombinant human hyaluronidase comprises a sequence having at least 95%, 96%, 97%, 98%, or 99% to the amino acid sequence of SEQ ID NO: 453. In one embodiment, the recombinant human hyaluronidase comprises a sequence having at least 95%, 96%, 97%, 98%, or 99% to the amino acid sequence of SEQ ID NO: 455. In another embodiment, the recombinant human hyaluronidase comprises the amino acid sequence set forth in SEQ ID NO:453. In another embodiment, the recombinant human hyaluronidase consists of the amino acid sequence set forth in SEQ ID NO:453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO:454. In another embodiment, the recombinant human hyaluronidase comprises the amino acid sequence set forth in SEQ ID NO: 455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO:456. In another embodiment, the recombinant human hyaluronidase consists of the amino acid sequence set forth in SEQ ID NO:456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO:457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO:458. In another embodiment, the recombinant human hyaluronidase comprises the amino acid sequence set forth in SEQ ID NO: 459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO:460. In another embodiment, the recombinant human hyaluronidase consists of the amino acid sequence set forth in SEQ ID NO:460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO:461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO:462. In another embodiment, the recombinant human hyaluronidase comprises the amino acid sequence set forth in SEQ ID NO: 464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO:465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO:466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO: 468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO:469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO:470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO: 472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO:473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO:474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475. In another embodiment, the recombinant human hyaluronidase comprises the amino acid sequence set forth in SEQ ID NO: 476. In another embodiment, the recombinant human hyaluronidase consists of the amino acid sequence set forth in SEQ ID NO:476. In another embodiment, the recombinant human hyaluronidase comprises the amino acid sequence set forth in SEQ ID NO:477. In another embodiment, the recombinant human hyaluronidase consists of the amino acid sequence set forth in SEQ ID NO:477. In another embodiment, the recombinant human hyaluronidase comprises the amino acid sequence set forth in SEQ ID NO:478. In another embodiment, the recombinant human hyaluronidase consists of the amino acid sequence set forth in SEQ ID NO:478. In another embodiment, the recombinant human hyaluronidase comprises the amino acid sequence set forth in SEQ ID NO:479. In another embodiment, the recombinant human hyaluronidase consists of the amino acid sequence set forth in SEQ ID NO:479. In another embodiment, the recombinant human hyaluronidase comprises the amino acid sequence set forth in SEQ ID NO: 480. In another embodiment, the recombinant human hyaluronidase consists of the amino acid sequence set forth in SEQ ID NO:480. In another embodiment, the recombinant human hyaluronidase comprises the amino acid sequence set forth in SEQ ID NO:481. In another embodiment, the recombinant human hyaluronidase consists of the amino acid sequence set forth in SEQ ID NO:481. In another embodiment, the recombinant human hyaluronidase comprises the amino acid sequence set forth in SEQ ID NO:482. In another embodiment, the recombinant human hyaluronidase consists of the amino acid sequence set forth in SEQ ID NO:482. In another embodiment, the recombinant human hyaluronidase comprises the amino acid sequence set forth in SEQ ID NO:483. In another embodiment, the recombinant human hyaluronidase consists of the amino acid sequence set forth in SEQ ID NO:483. In another embodiment, the recombinant human hyaluronidase comprises the amino acid sequence set forth in SEQ ID NO: 484. In another embodiment, the recombinant human hyaluronidase consists of the amino acid sequence set forth in SEQ ID NO:484. In another embodiment, the recombinant human hyaluronidase comprises the amino acid sequence set forth in SEQ ID NO:485. In another embodiment, the recombinant human hyaluronidase consists of the amino acid sequence set forth in SEQ ID NO:485. In another embodiment, the recombinant human hyaluronidase comprises the amino acid sequence set forth in SEQ ID NO:486. In another embodiment, the recombinant human hyaluronidase consists of the amino acid sequence set forth in SEQ ID NO:486. In another embodiment, the recombinant human hyaluronidase comprises the amino acid sequence set forth in SEQ ID NO:487. In another embodiment, the recombinant human hyaluronidase consists of the amino acid sequence set forth in SEQ ID NO:487. In another embodiment, the recombinant human hyaluronidase comprises the amino acid sequence set forth in SEQ ID NO: 488. In another embodiment, the recombinant human hyaluronidase consists of the amino acid sequence set forth in SEQ ID NO:488. In another embodiment, the recombinant human hyaluronidase comprises the amino acid sequence set forth in SEQ ID NO:489. In another embodiment, the recombinant human hyaluronidase consists of the amino acid sequence set forth in SEQ ID NO:489. In another embodiment, the recombinant human hyaluronidase comprises the amino acid sequence set forth in SEQ ID NO:490. In another embodiment, the recombinant human hyaluronidase consists of the amino acid sequence set forth in SEQ ID NO:490. In another embodiment, the recombinant human hyaluronidase comprises the amino acid sequence set forth in SEQ ID NO:491. In another embodiment, the recombinant human hyaluronidase consists of the amino acid sequence set forth in SEQ ID NO:491. In another embodiment, the recombinant human hyaluronidase comprises the amino acid sequence set forth in SEQ ID NO: 492. In another embodiment, the recombinant human hyaluronidase consists of the amino acid sequence set forth in SEQ ID NO:492. In another embodiment, the recombinant human hyaluronidase comprises the amino acid sequence set forth in SEQ ID NO:493. In another embodiment, the recombinant human hyaluronidase consists of the amino acid sequence set forth in SEQ ID NO:493. In another embodiment, the recombinant human hyaluronidase comprises the amino acid sequence set forth in SEQ ID NO:494. In another embodiment, the recombinant human hyaluronidase consists of the amino acid sequence set forth in SEQ ID NO:494. In another embodiment, the recombinant human hyaluronidase comprises the amino acid sequence set forth in SEQ ID NO:495. In another embodiment, the recombinant human hyaluronidase consists of the amino acid sequence set forth in SEQ ID NO:495. In another embodiment, the recombinant human hyaluronidase comprises the amino acid sequence set forth in SEQ ID NO: 496. In another embodiment, the recombinant human hyaluronidase consists of the amino acid sequence set forth in SEQ ID NO:496. In another embodiment, the recombinant human hyaluronidase comprises the amino acid sequence set forth in SEQ ID NO:497. In another embodiment, the recombinant human hyaluronidase consists of the amino acid sequence set forth in SEQ ID NO:497. In another embodiment, the recombinant human hyaluronidase comprises the amino acid sequence set forth in SEQ ID NO:498. In another embodiment, the recombinant human hyaluronidase consists of the amino acid sequence set forth in SEQ ID NO:498. In another embodiment, the recombinant human hyaluronidase comprises the amino acid sequence set forth in SEQ ID NO:499. In another embodiment, the recombinant human hyaluronidase consists of the amino acid sequence set forth in SEQ ID NO:499. In another embodiment, the recombinant human hyaluronidase comprises the amino acid sequence set forth in SEQ ID NO: 500. In another embodiment, the recombinant human hyaluronidase consists of the amino acid sequence set forth in SEQ ID NO:500. In another embodiment, the recombinant human hyaluronidase comprises the amino acid sequence set forth in SEQ ID NO:501. In another embodiment, the recombinant human hyaluronidase consists of the amino acid sequence set forth in SEQ ID NO:501. In another embodiment, the recombinant human hyaluronidase comprises the amino acid sequence set forth in SEQ ID NO:502. In another embodiment, the recombinant human hyaluronidase consists of the amino acid sequence set forth in SEQ ID NO:502. In another embodiment, the recombinant human hyaluronidase comprises the amino acid sequence set forth in SEQ ID NO:503. In another embodiment, the recombinant human hyaluronidase consists of the amino acid sequence set forth in SEQ ID NO:503. In another embodiment, the recombinant human hyaluronidase comprises the amino acid sequence set forth in SEQ ID NO: 504. In another embodiment, the recombinant human hyaluronidase consists of the amino acid sequence set forth in SEQ ID NO:504. In another embodiment, the recombinant human hyaluronidase comprises the amino acid sequence set forth in SEQ ID NO:505. In another embodiment, the recombinant human hyaluronidase consists of the amino acid sequence set forth in SEQ ID NO:505. In another embodiment, the recombinant human hyaluronidase comprises the amino acid sequence set forth in SEQ ID NO:506. In another embodiment, the recombinant human hyaluronidase consists of the amino acid sequence set forth in SEQ ID NO:506. In another embodiment, the recombinant human hyaluronidase comprises the amino acid sequence set forth in SEQ ID NO:507. In another embodiment, the recombinant human hyaluronidase consists of the amino acid sequence set forth in SEQ ID NO:507. In another embodiment, the recombinant human hyaluronidase comprises the amino acid sequence set forth in SEQ ID NO: 508. In another embodiment, the recombinant human hyaluronidase consists of the amino acid sequence set forth in SEQ ID NO:508. In another embodiment, the recombinant human hyaluronidase comprises the amino acid sequence set forth in SEQ ID NO:509. In another embodiment, the recombinant human hyaluronidase consists of the amino acid sequence set forth in SEQ ID NO:509. In another embodiment, the recombinant human hyaluronidase comprises the amino acid sequence set forth in SEQ ID NO:510. In another embodiment, the recombinant human hyaluronidase consists of the amino acid sequence set forth in SEQ ID NO:510. In another embodiment, the recombinant human hyaluronidase comprises the amino acid sequence set forth in SEQ ID NO:511. In another embodiment, the recombinant human hyaluronidase consists of the amino acid sequence set forth in SEQ ID NO:511. In another embodiment, the recombinant human hyaluronidase comprises the amino acid sequence set forth in SEQ ID NO: 512. In another embodiment, the recombinant human hyaluronidase consists of the amino acid sequence set forth in SEQ ID NO:512. In another embodiment, the recombinant human hyaluronidase comprises the amino acid sequence set forth in SEQ ID NO:513. In another embodiment, the recombinant human hyaluronidase consists of the amino acid sequence set forth in SEQ ID NO:513. In another embodiment, the recombinant human hyaluronidase comprises the amino acid sequence set forth in SEQ ID NO:514. In another embodiment, the recombinant human hyaluronidase consists of the amino acid sequence set forth in SEQ ID NO:514. In another embodiment, the recombinant human hyaluronidase comprises the amino acid sequence set forth in SEQ ID NO:515. In another embodiment, the recombinant human hyaluronidase consists of the amino acid sequence set forth in SEQ ID NO:515. In another embodiment, the recombinant human hyaluronidase comprises the amino acid sequence set forth in SEQ ID NO: 516. In another embodiment, the recombinant human hyaluronidase consists of the amino acid sequence set forth in SEQ ID NO:516. In another embodiment, the recombinant human hyaluronidase comprises the amino acid sequence set forth in SEQ ID NO:517. In another embodiment, the recombinant human hyaluronidase consists of the amino acid sequence set forth in SEQ ID NO:517. In another embodiment, the recombinant human hyaluronidase comprises the amino acid sequence set forth in SEQ ID NO:518. In another embodiment, the recombinant human hyaluronidase consists of the amino acid sequence set forth in SEQ ID NO:518. In another embodiment, the recombinant human hyaluronidase comprises the amino acid sequence set forth in SEQ ID NO:519. In another embodiment, the recombinant human hyaluronidase consists of the amino acid sequence set forth in SEQ ID NO:519.

### Anti-TSLP and Anti-OX40L Antibodies

### Antibody Structure

The present application provides antibodies, or antigen binding fragments thereof, and compositions comprising an antibody, or antigen binding fragment thereof, which binds TSLP and an antibody, or antigen binding fragment thereof, which binds OX40L. In certain embodiments, the TSLP and/or OX40L antibody, or antigen binding fragment thereof, is an antibody comprising a modified Fc region.

The recognized immunoglobulin genes include the kappa, lambda, alpha, gamma, delta, epsilon and mu constant region genes, as well as the myriad immunoglobulin variable region genes. Light chains are classified as either kappa or lambda. There are four subtypes of human lambda light chain constant region (21, 22, 23, 27), which are encoded by the genes IGLC1, IGCL2, IGLC3, and IGLC7. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, ¿, Y, and u, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains, respectively. The IgG1 heavy chain comprises the VH, CH1, CH2, and CH3 domains, respectively from the N-to C-terminus. The light chain comprises the VL and CL domains from N- to C-terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or is derived from an immunoglobulin-based construct such as a diabody or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six hypervariable regions (HVRs); three in the VH (H1, H2, and H3), and three in the VL (L1, L2, and L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. HVRs are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al. (1987) J Mol Biol 196:901-917, where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al. (1997) J. Mol. Biol., 273:927-948 (“Chothia” numbering scheme); MacCallum et al. (1996) J. Mol. Biol. 262:732-745 (“Contact” numbering scheme); Lefranc et al. (2003) Dev. Comp. Immunol. 27:55-77 (“IMGT” numbering scheme); and Honegge and Plückthun (2001) J. Mol. Biol. 309:657-70 (“AHo” numbering scheme); each of which is incorporated by reference in its entirety.

TABLE 2 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 2
Residues in CDRs according to Kabat
and Chothia numbering schemes

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TSLP or OX40L variants with different point-mutations, or to chimeric TSLP or OX40L variants.

To screen for antibodies which bind to an epitope on a target antigen bound by an antibody of interest (e.g., TSLP and/or OX40L), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Anti-TSLP and anti-OX40L antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments, the antibodies are monoclonal antibodies.

In some embodiments, the antibodies are polyclonal antibodies.

In some embodiments, the antibodies are produced by hybridomas. In other embodiments, the antibodies are produced by recombinant cells engineered to express the desired variable and constant domains.

In some embodiments, the antibodies may be single chain antibodies or other antibody derivatives retaining the antigen specificity and the lower hinge region or a variant thereof.

In some embodiments, the antibodies may be polyfunctional antibodies, recombinant antibodies, fully human antibodies, humanized antibodies, or fragments or variants thereof. In particular embodiments, the antibody fragment or a derivative thereof is selected from a Fab fragment, a Fab′2 fragment, a CDR and scFv.

### Anti-TSLP Antibodies

The present application provides antibodies or antigen binding fragments thereof and compositions comprising an antibody, or an antigen binding fragment thereof, which binds TSLP. In some embodiments, an anti-TSLP antibody, or antigen binding fragment thereof, described herein is selected from tezepelumab, GR2002 (described in U.S. Patent Application Publication No. US 2024/0132581, which is incorporated herein by reference), TQC2731 (also known as BSI-045B and bosakitug, described in U.S. Patent Application Publication No. US 2022/0289833, which is incorporated herein by reference), solrikitug, hu23b12 (described in U.S. Pat. No. 8,232,372, which is incorporated herein by reference), 43B1-H2L2 (described in U.S. Patent Application Publication No. US 2022/0363781, which is incorporated herein by reference), and 43B1-H6L1 (also described in U.S. Patent Application Publication No. US 2022/0363781) or a variant thereof comprising an Fc region with one or more amino acid substitutions as described herein. In some embodiments, tezepelumab comprises a variable heavy (VH) chain sequence having the amino acid sequence of SEQ ID NO: 34 and a variable light (VL) chain sequence having the amino acid sequence of SEQ ID NO: 441. In some embodiments, TQC2731 comprises a VH chain sequence having the amino acid sequence of SEQ ID NO: 554 and a VL chain sequence having the amino acid sequence of SEQ ID NO: 555. In some embodiments, solrikitug comprises a VH chain sequence having the amino acid sequence of SEQ ID NO: 562 and a VL chain sequence having the amino acid sequence of SEQ ID NO: 563. In some embodiments, hu23b12 comprises a VH chain sequence having the amino acid sequence of SEQ ID NO: 442 and a VL chain sequence having the amino acid sequence of SEQ ID NO: 443. In some embodiments, 43B1-H2L2 comprises a VH chain sequence having the amino acid sequence of SEQ ID NO: 444 and a VL chain sequence having the amino acid sequence of SEQ ID NO: 445. In some embodiments, 43B1-H6L1 comprises a VH chain sequence having the amino acid sequence of SEQ ID NO: 446 and a VL chain sequence having the amino acid sequence of SEQ ID NO: 447. In some embodiments, tezepelumab comprises a heavy chain having the amino acid sequence of SEQ ID NO: 550 and a light chain sequence having the amino acid sequence of SEQ ID NO: 551. In some embodiments, TQC2731 comprises a heavy chain having the amino acid sequence of SEQ ID NO: 552 and a light chain sequence having the amino acid sequence of SEQ ID NO: 553. In some embodiments, solrikitug comprises a heavy chain having the amino acid sequence of SEQ ID NO: 564 and a light chain sequence having the amino acid sequence of SEQ ID NO: 565. In some embodiments, hu23b12 comprises a heavy chain having the amino acid sequence of SEQ ID NO: 556 and a light chain sequence having the amino acid sequence of SEQ ID NO: 557. In some embodiments, 43B1-H2L2 comprises a heavy chain having the amino acid sequence of SEQ ID NO: 558 and a light chain sequence having the amino acid sequence of SEQ ID NO: 559. In some embodiments, 43B1-H6L1 comprises a heavy chain having the amino acid sequence of SEQ ID NO: 560 and a light chain sequence having the amino acid sequence of SEQ ID NO: 561. Additional anti-TSLP antibodies are described in PCT Application No. PCT/US2025/034623, which is incorporated herein by reference in its entirety. In some embodiments, an anti-TSLP antibody, or antigen binding fragment thereof, described herein is selected from an anti-TSLP antibody, or antigen binding fragment thereof, described therein. Further TSLP antibodies are described in U.S. Pat. No. 8,232,372 and U.S. Patent Application Publication No. US 2022/0363781, each of which is incorporated herein by reference in its entirety.

Sequences of exemplary anti-TSLP antibodies, or antigen binding fragments thereof, are provided in TABLE 3, TABLE 4, TABLE 5, and TABLE 6.

### Sequences of Exemplary Anti-TSLP Antibodies

TABLE 3
Sequences of anti-TSLP Antibody Constructs - CDRs

| | | | | | | | | | | | | |
|---|---|---|---|---|---|---|---|---|---|---|---|---|
| | | SEQ | | SEQ | | SEQ | | SEQ | | SEQ | | SEQ |
| | | ID | | ID | | ID | | ID | | ID | | ID |
| Antibody | CDRH1 | NO: | CDRH2 | NO: | CDRH3 | NO: | CDRL1 | NO: | CDRL2 | NO: | CDRL3 | NO: |

Kabat Numbering

| | | | | | | | | | | | | |
|---|---|---|---|---|---|---|---|---|---|---|---|---|
| Antibody | TYTMH | 1 | VIWYD | 10 | APQWEL | 13 | GGNNLG | 19 | DVSD | 25 | QVWD | 27 |
| 1 | | | GSNKH | | VHEAFDI | | SKSVH | | RPS | | SSSTFV | |
| | | | YADSV | | | | | | | | V | |
| | | | KG | | | | | | | | | |
| Antibody | TYPMH | 2 | VIWYD | 10 | APQWEL | 13 | GGNNLG | 19 | DVSD | 25 | QVWD | 28 |
| 2 | | | GSNKH | | VHEAFDI | | SKSVH | | RPS | | SSSTY | |
| | | | YADSV | | | | | | | | VV | |
| | | | KG | | | | | | | | | |
| Antibody | TYPMH | 2 | VIWYD | 10 | ALIWELF | 14 | GGNNLI | 20 | DDSD | 26 | QVWD | 28 |
| 3 | | | GSNKH | | HEYFDI | | SKSVH | | RPS | | SSSTY | |
| | | | YADSV | | | | | | | | VV | |
| | | | KG | | | | | | | | | |
| Antibody | TYPMH | 2 | VIWYD | 10 | APQWEL | 13 | GGNNLV | 21 | DVSD | 25 | QVWD | 28 |
| 4 | | | GSNKH | | VHEAFDI | | SKSVH | | RPS | | SSSTY | |
| | | | YADSV | | | | | | | | VV | |
| | | | KG | | | | | | | | | |
| Antibody | TYGMH | 3 | VIWYD | 10 | APQWEL | 13 | GGNNLV | 21 | DDSD | 26 | QVWD | 27 |
| 5 | | | GSNKH | | VHEAFDI | | SKSVH | | RPS | | SSSTFV | |
| | | | YADSV | | | | | | | | V | |
| | | | KG | | | | | | | | | |
| Antibody | TYGMH | 3 | VIWYD | 10 | ALLWEL | 15 | GGNNLV | 21 | DVSD | 25 | QVWD | 29 |
| 6 | | | GSNKH | | FHEYFDI | | SKSVH | | RPS | | SSSDH | |
| | | | YADSV | | | | | | | | VV | |
| | | | KG | | | | | | | | | |
| Antibody | TYGMH | 3 | VIWYD | 10 | APQWEL | 13 | GGNNLG | 19 | DVSD | 25 | QVWD | 30 |
| 7 | | | GSNKH | | VHEAFDI | | SKSVH | | RPS | | SSSTIV | |
| | | | YADSV | | | | | | | | V | |
| | | | KG | | | | | | | | | |
| Antibody | TYTMH | 1 | VIWYD | 10 | APQWEL | 13 | GGNNLG | 19 | DDSD | 26 | QVWD | 31 |
| 8 | | | GSNKH | | VHEAFDI | | SKSVH | | RPS | | KSSTY | |
| | | | YADSV | | | | | | | | VV | |
| | | | KG | | | | | | | | | |
| Antibody | TYPMH | 2 | VIWYD | 10 | ALLWEL | 15 | GGNNLV | 21 | DDSD | 26 | QVWD | 28 |
| 9 | | | GSNKH | | FHEYFDI | | SKSVH | | RPS | | SSSTY | |
| | | | YADSV | | | | | | | | VV | |
| | | | KG | | | | | | | | | |
| Antibody | TYGMH | 3 | VIWYD | 10 | APQWEL | 13 | RTSQNI | 345 | DVSSL | 377 | QQGYS | 385 |
| 10 | | | GSNKH | | VHEAFDI | | RYYLN | | QS | | PPLT | |
| | | | YADSV | | | | | | | | | |
| | | | KG | | | | | | | | | |
| Antibody | TYGMH | 3 | VIWYD | 10 | APQWEL | 13 | GGDNIG | 346 | EDSD | 378 | QVWD | 386 |
| 11 | | | GSNKH | | VHEAFDI | | SKSVH | | RPS | | SDSDH | |
| | | | YADSV | | | | | | | | VV | |
| | | | KG | | | | | | | | | |
| Antibody | TYGMH | 3 | VIWYD | 10 | APQWEL | 13 | GGQNIG | 347 | DDSD | 26 | QVWD | 387 |
| 12 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGQNIG | 347 | DDSD | 26 | QVWD | 387 |
| 13 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGQNIG | 347 | DDSD | 26 | QVWD | 387 |
| 14 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGNKIG | 348 | DDSD | 26 | QVWD | 388 |
| 15 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGNKIG | 348 | DDSD | 26 | QVWD | 388 |
| 16 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGQNIG | 347 | DDSD | 26 | QVWD | 389 |
| 17 | | | GSNKH | | VHEAFDI | | TKSVH | | RPS | | GSSDQ | |
| | | | YADSV | | | | | | | | SV | |
| | | | KG | | | | | | | | | |
| Antibody | TYGMH | 3 | VIWYD | 10 | APQWEL | 13 | GGNNIG | 349 | DDTD | 379 | QVWD | 390 |
| 18 | | | GSNKH | | VHEAFDI | | GKSVH | | RPS | | TTTDH | |
| | | | YADSV | | | | | | | | VV | |
| | | | KG | | | | | | | | | |
| Antibody | TYGMH | 3 | VIWYD | 10 | APQWEL | 13 | GGNNIG | 350 | DDSD | 26 | QVWD | 391 |
| 19 | | | GSNKH | | VHEAFDI | | SKSVH | | RPS | | SSSDP | |
| | | | YADSV | | | | | | | | VV | |
| | | | KG | | | | | | | | | |
| Antibody | TYGMH | 3 | VIWYD | 10 | APQWEL | 13 | GGQNIG | 347 | DDSD | 26 | QVWD | 387 |
| 20 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGQNIG | 347 | DDSD | 380 | QVWD | 387 |
| 21 | | | GSNKH | | VHEAFDI | | TKSVH | | RPP | | SSSDQ | |
| | | | YADSV | | | | | | | | SV | |
| | | | KG | | | | | | | | | |
| Antibody | TYGMH | 3 | VIWYD | 10 | APQWEL | 13 | GGNKIG | 348 | DDSD | 26 | QVWD | 388 |
| 22 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGHNIG | 351 | DDSD | 26 | QVWD | 392 |
| 23 | | | GSNKH | | VHEAFDI | | TKSVH | | RPS | | RSSDQ | |
| | | | YADSV | | | | | | | | SV | |
| | | | KG | | | | | | | | | |
| Antibody | TYGMH | 3 | VIWYD | 10 | APQWEL | 13 | GGDSIG | 352 | DDSD | 26 | QVWD | 392 |
| 24 | | | GSNKH | | VHEAFDI | | SKYVH | | RPS | | RSSDQ | |
| | | | YADSV | | | | | | | | SV | |
| | | | KG | | | | | | | | | |
| Antibody | TYGMH | 3 | VIWYD | 10 | APQWEL | 13 | GGQNIG | 347 | DDSD | 26 | QVWD | 393 |
| 25 | | | GSNKH | | VHEAFDI | | TKSVH | | RPS | | SGSGQ | |
| | | | YADSV | | | | | | | | TV | |
| | | | KG | | | | | | | | | |
| Antibody | TYGMH | 3 | VIWYD | 10 | APQWEL | 13 | GGQNIG | 353 | DDSD | 26 | QVWD | 387 |
| 26 | | | GSNKH | | VHEAFDI | | TRSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGNNIG | 354 | DDSD | 26 | HVWD | 394 |
| 27 | | | GSNKH | | VHEAFDI | | SETVH | | RPS | | SSSDH | |
| | | | YADSV | | | | | | | | VM | |
| | | | KG | | | | | | | | | |
| Antibody | TYGMH | 3 | VIWYD | 10 | APQWEL | 13 | RGDNIE | 355 | DDSD | 26 | QVWD | 395 |
| 28 | | | GSNKH | | VHEAFDI | | TYSVH | | RPS | | SDSDH | |
| | | | YADSV | | | | | | | | LE | |
| | | | KG | | | | | | | | | |
| Antibody | TYGMH | 3 | VIWYD | 10 | APQWEL | 13 | GGNNIG | 356 | DDSD | 26 | QVWD | 396 |
| 29 | | | GSNKH | | VHEAFDI | | RKSAH | | RPS | | SYSNH | |
| | | | YADSV | | | | | | | | VV | |
| | | | KG | | | | | | | | | |
| Antibody | TYGMH | 3 | VIWYD | 10 | APQWEL | 13 | GGNKIG | 348 | DDSD | 26 | QVWD | 388 |
| 30 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGHNIG | 351 | DDSD | 26 | QVWD | 392 |
| 31 | | | GSNKH | | VHEAFDI | | TKSVH | | RPS | | RSSDQ | |
| | | | YADSV | | | | | | | | SV | |
| | | | KG | | | | | | | | | |
| Antibody | TYGMH | 3 | VIWYD | 10 | APQWEL | 13 | RGDNIE | 355 | DDSD | 26 | QVWD | 397 |
| 32 | | | GSNKH | | VHEAFDI | | TYSVH | | RPS | | SGGDH | |
| | | | YADSV | | | | | | | | VV | |
| | | | KG | | | | | | | | | |
| Antibody | TYGMH | 3 | VIWYD | 10 | APQWEL | 13 | GGNNIG | 350 | DDSD | 26 | QVWD | 29 |
| 33 | | | GSNKH | | VHEAFDI | | SKSVH | | RPS | | SSSDH | |
| | | | YADSV | | | | | | | | VV | |
| | | | KG | | | | | | | | | |
| Antibody | TYGMH | 3 | VIWYD | 10 | APQWEL | 13 | EGTYLG | 357 | DDKE | 381 | QVWD | 398 |
| 34 | | | GSNKH | | VHEAFDI | | AKSVH | | RPS | | DISDHI | |
| | | | YADSV | | | | | | | | V | |
| | | | KG | | | | | | | | | |
| Antibody | TYGMH | 3 | VIWYD | 10 | APQWEL | 13 | GGNNIG | 358 | DDSD | 26 | QVWD | 387 |
| 35 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGQSIG | 359 | DDSD | 26 | QVWD | 387 |
| 36 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GANSIE | 360 | DDSE | 382 | QVWD | 399 |
| 37 | | | GSNKH | | VHEAFDI | | DKNVH | | RPS | | MASDR | |
| | | | YADSV | | | | | | | | VV | |
| | | | KG | | | | | | | | | |
| Antibody | TYGMH | 3 | VIWYD | 10 | APQWEL | 13 | GGNKIG | 348 | DDSD | 26 | QVWD | 388 |
| 38 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGQNIG | 347 | DDSD | 26 | QVWD | 387 |
| 39 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | GGNKIG | 348 | DDSD | 26 | QVWD | 388 |
| 40 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGQNIG | 347 | DDSD | 26 | QVWD | 387 |
| 41 | | | GSNKH | | VHEAFDI | | TKSVH | | RPS | | SSSDQ | |
| | | | YADSV | | | | | | | | SV | |
| | | | KG | | | | | | | | | |
| Antibody | TYGMH | 3 | VIWYD | 10 | APQWEL | 13 | RGDNIE | 355 | DDSD | 26 | QVWD | 29 |
| 42 | | | GSNKH | | VHEAFDI | | TYSVH | | RPS | | SSSDH | |
| | | | YADSV | | | | | | | | VV | |
| | | | KG | | | | | | | | | |
| Antibody | TYGMH | 3 | VIWYD | 10 | APQWEL | 13 | GGNKIG | 348 | DDSD | 26 | QVWD | 388 |
| 43 | | | GSNKH | | VHEAFDI | | RKSVH | | RPS | | SSSDE | |
| | | | YADSV | | | | | | | | GV | |
| | | | KG | | | | | | | | | |
| Antibody | TYGMH | 3 | VIWYD | 10 | APQWEL | 13 | GGNKIG | 348 | DDSD | 26 | QVWD | 400 |
| 44 | | | GSNKH | | VHEAFDI | | RKSVH | | RPS | | TSSDQ | |
| | | | YADSV | | | | | | | | GV | |
| | | | KG | | | | | | | | | |
| Antibody | TYGMH | 3 | VIWYD | 10 | APQWEL | 13 | GGDNIG | 361 | DDLK | 383 | QVWD | 401 |
| 45 | | | GSNKH | | VHEAFDI | | SYSVH | | RPS | | STSGV | |
| | | | YADSV | | | | | | | | VF | |
| | | | KG | | | | | | | | | |
| Antibody | TYGMH | 3 | VIWYD | 10 | APQWEL | 13 | GGNNVE | 362 | DDND | 384 | QVWD | 29 |
| 46 | | | GSNKH | | VHEAFDI | | DYSVH | | RPS | | SSSDH | |
| | | | YADSV | | | | | | | | VV | |
| | | | KG | | | | | | | | | |
| Tezepelumab | TYGMH | 3 | VIWYD | 10 | APQWEL | 13 | GGNNLG | 19 | DDSD | 26 | QVWD | 29 |
| | | | GSNKH | | VHEAFDI | | SKSVH | | RPS | | SSSDH | |
| | | | YADSV | | | | | | | | VV | |
| | | | KG | | | | | | | | | |

Chothia Numbering

| | | | | | | | | | | | | |
|---|---|---|---|---|---|---|---|---|---|---|---|---|
| Antibody | GFTFRT | 4 | WYDG | 11 | APQWEL | 13 | GGNNLG | 19 | DVSD | 25 | QVWD | 27 |
| 1 | YTMH | | SN | | VHEAFDI | | SKSVH | | RPS | | SSSTFV | |
| | | | | | | | | | | | V | |
| Antibody | GFTFRT | 5 | WYDG | 11 | APQWEL | 13 | GGNNLG | 19 | DVSD | 25 | QVWD | 28 |
| 2 | YPMH | | SN | | VHEAFDI | | SKSVH | | RPS | | SSSTY | |
| | | | | | | | | | | | VV | |
| Antibody | GFTFRT | 5 | WYDG | 11 | ALIWELF | 14 | GGNNLI | 20 | DDSD | 26 | QVWD | 28 |
| 3 | YPMH | | SN | | HEYFDI | | SKSVH | | RPS | | SSSTY | |
| | | | | | | | | | | | VV | |
| Antibody | GFTFRT | 5 | WYDG | 11 | APQWEL | 13 | GGNNLV | 21 | DVSD | 25 | QVWD | 28 |
| 4 | YPMH | | SN | | VHEAFDI | | SKSVH | | RPS | | SSSTY | |
| | | | | | | | | | | | VV | |
| Antibody | GFTFRT | 6 | WYDG | 11 | APQWEL | 13 | GGNNLV | 21 | DDSD | 26 | QVWD | 27 |
| 5 | YGMH | | SN | | VHEAFDI | | SKSVH | | RPS | | SSSTFV | |
| | | | | | | | | | | | V | |
| Antibody | GFTFRT | 6 | WYDG | 11 | ALLWEL | 15 | GGNNLV | 21 | DVSD | 25 | QVWD | 29 |
| 6 | YGMH | | SN | | FHEYFDI | | SKSVH | | RPS | | SSSDH | |
| | | | | | | | | | | | VV | |
| Antibody | GFTFRT | 6 | WYDG | 11 | APQWEL | 13 | GGNNLG | 19 | DVSD | 25 | QVWD | 30 |
| 7 | YGMH | | SN | | VHEAFDI | | SKSVH | | RPS | | SSSTIV | |
| | | | | | | | | | | | V | |
| Antibody | GFTFRT | 4 | WYDG | 11 | APQWEL | 13 | GGNNLG | 19 | DDSD | 26 | QVWD | 31 |
| 8 | YTMH | | SN | | VHEAFDI | | SKSVH | | RPS | | KSSTY | |
| | | | | | | | | | | | VV | |
| Antibody | GFTFRT | 5 | WYDG | 11 | ALLWEL | 15 | GGNNLV | 21 | DDSD | 26 | QVWD | 28 |
| 9 | YPMH | | SN | | FHEYFDI | | SKSVH | | RPS | | SSSTY | |
| | | | | | | | | | | | VV | |
| Antibody | GFTFRT | 6 | WYDG | 11 | APQWEL | 13 | RTSQNI | 345 | DVSSL | 377 | QQGYS | 385 |
| 10 | YGMH | | SN | | VHEAFDI | | RYYLN | | QS | | PPLT | |
| Antibody | GFTFRT | 6 | WYDG | 11 | APQWEL | 13 | GGDNIG | 346 | EDSD | 378 | QVWD | 386 |
| 11 | YGMH | | SN | | VHEAFDI | | SKSVH | | RPS | | SDSDH | |
| | | | | | | | | | | | VV | |
| Antibody | GFTFRT | 6 | WYDG | 11 | APQWEL | 13 | GGQNIG | 347 | DDSD | 26 | QVWD | 387 |
| 12 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGQNIG | 347 | DDSD | 26 | QVWD | 387 |
| 13 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGQNIG | 347 | DDSD | 26 | QVWD | 387 |
| 14 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGNKIG | 348 | DDSD | 26 | QVWD | 388 |
| 15 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGNKIG | 348 | DDSD | 26 | QVWD | 388 |
| 16 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGQNIG | 347 | DDSD | 26 | QVWD | 389 |
| 17 | YGMH | | SN | | VHEAFDI | | TKSVH | | RPS | | GSSDQ | |
| | | | | | | | | | | | SV | |
| Antibody | GFTFRT | 6 | WYDG | 11 | APQWEL | 13 | GGNNIG | 349 | DDTD | 379 | QVWD | 390 |
| 18 | YGMH | | SN | | VHEAFDI | | GKSVH | | RPS | | TTTDH | |
| | | | | | | | | | | | VV | |
| Antibody | GFTFRT | 6 | WYDG | 11 | APQWEL | 13 | GGNNIG | 350 | DDSD | 26 | QVWD | 391 |
| 19 | YGMH | | SN | | VHEAFDI | | SKSVH | | RPS | | SSSDP | |
| | | | | | | | | | | | VV | |
| Antibody | GFTFRT | 6 | WYDG | 11 | APQWEL | 13 | GGQNIG | 347 | DDSD | 26 | QVWD | 387 |
| 20 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGQNIG | 347 | DDSD | 380 | QVWD | 387 |
| 21 | YGMH | | SN | | VHEAFDI | | TKSVH | | RPP | | SSSDQ | |
| | | | | | | | | | | | SV | |
| Antibody | GFTFRT | 6 | WYDG | 11 | APQWEL | 13 | GGNKIG | 348 | DDSD | 26 | QVWD | 388 |
| 22 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGHNIG | 351 | DDSD | 26 | QVWD | 392 |
| 23 | YGMH | | SN | | VHEAFDI | | TKSVH | | RPS | | RSSDQ | |
| | | | | | | | | | | | SV | |
| Antibody | GFTFRT | 6 | WYDG | 11 | APQWEL | 13 | GGDSIG | 352 | DDSD | 26 | QVWD | 392 |
| 24 | YGMH | | SN | | VHEAFDI | | SKYVH | | RPS | | RSSDQ | |
| | | | | | | | | | | | SV | |
| Antibody | GFTFRT | 6 | WYDG | 11 | APQWEL | 13 | GGQNIG | 347 | DDSD | 26 | QVWD | 393 |
| 25 | YGMH | | SN | | VHEAFDI | | TKSVH | | RPS | | SGSGQ | |
| | | | | | | | | | | | TV | |
| Antibody | GFTFRT | 6 | WYDG | 11 | APQWEL | 13 | GGQNIG | 353 | DDSD | 26 | QVWD | 387 |
| 26 | YGMH | | SN | | VHEAFDI | | TRSVH | | RPS | | SSSDQ | |
| | | | | | | | | | | | SV | |
| Antibody | GFTFRT | 6 | WYDG | 11 | APQWEL | 13 | GGNNIG | 354 | DDSD | 26 | HVWD | 394 |
| 27 | YGMH | | SN | | VHEAFDI | | SETVH | | RPS | | SSSDH | |
| | | | | | | | | | | | VM | |
| Antibody | GFTFRT | 6 | WYDG | 11 | APQWEL | 13 | RGDNIE | 355 | DDSD | 26 | QVWD | 395 |
| 28 | YGMH | | SN | | VHEAFDI | | TYSVH | | RPS | | SDSDH | |
| | | | | | | | | | | | LE | |
| Antibody | GFTFRT | 6 | WYDG | 11 | APQWEL | 13 | GGNNIG | 356 | DDSD | 26 | QVWD | 396 |
| 29 | YGMH | | SN | | VHEAFDI | | RKSAH | | RPS | | SYSNH | |
| | | | | | | | | | | | VV | |
| Antibody | GFTFRT | 6 | WYDG | 11 | APQWEL | 13 | GGNKIG | 348 | DDSD | 26 | QVWD | 388 |
| 30 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGHNIG | 351 | DDSD | 26 | QVWD | 392 |
| 31 | YGMH | | SN | | VHEAFDI | | TKSVH | | RPS | | RSSDQ | |
| | | | | | | | | | | | SV | |
| Antibody | GFTFRT | 6 | WYDG | 11 | APQWEL | 13 | RGDNIE | 355 | DDSD | 26 | QVWD | 397 |
| 32 | YGMH | | SN | | VHEAFDI | | TYSVH | | RPS | | SGGDH | |
| | | | | | | | | | | | VV | |
| Antibody | GFTFRT | 6 | WYDG | 11 | APQWEL | 13 | GGNNIG | 350 | DDSD | 26 | QVWD | 29 |
| 33 | YGMH | | SN | | VHEAFDI | | SKSVH | | RPS | | SSSDH | |
| | | | | | | | | | | | VV | |
| Antibody | GFTFRT | 6 | WYDG | 11 | APQWEL | 13 | EGTYLG | 357 | DDKE | 381 | QVWD | 398 |
| 34 | YGMH | | SN | | VHEAFDI | | AKSVH | | RPS | | DISDHI | |
| | | | | | | | | | | | V | |
| Antibody | GFTFRT | 6 | WYDG | 11 | APQWEL | 13 | GGNNIG | 358 | DDSD | 26 | QVWD | 387 |
| 35 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGQSIG | 359 | DDSD | 26 | QVWD | 387 |
| 36 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GANSIE | 360 | DDSE | 382 | QVWD | 399 |
| 37 | YGMH | | SN | | VHEAFDI | | DKNVH | | RPS | | MASDR | |
| | | | | | | | | | | | VV | |
| Antibody | GFTFRT | 6 | WYDG | 11 | APQWEL | 13 | GGNKIG | 348 | DDSD | 26 | QVWD | 388 |
| 38 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGQNIG | 347 | DDSD | 26 | QVWD | 387 |
| 39 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | GGNKIG | 348 | DDSD | 26 | QVWD | 388 |
| 40 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGQNIG | 347 | DDSD | 26 | QVWD | 387 |
| 41 | YGMH | | SN | | VHEAFDI | | TKSVH | | RPS | | SSSDQ | |
| | | | | | | | | | | | SV | |
| Antibody | GFTFRT | 6 | WYDG | 11 | APQWEL | 13 | RGDNIE | 355 | DDSD | 26 | QVWD | 29 |
| 42 | YGMH | | SN | | VHEAFDI | | TYSVH | | RPS | | SSSDH | |
| | | | | | | | | | | | VV | |
| Antibody | GFTFRT | 6 | WYDG | 11 | APQWEL | 13 | GGNKIG | 348 | DDSD | 26 | QVWD | 388 |
| 43 | YGMH | | SN | | VHEAFDI | | RKSVH | | RPS | | SSSDE | |
| | | | | | | | | | | | GV | |
| Antibody | GFTFRT | 6 | WYDG | 11 | APQWEL | 13 | GGNKIG | 348 | DDSD | 26 | QVWD | 400 |
| 44 | YGMH | | SN | | VHEAFDI | | RKSVH | | RPS | | TSSDQ | |
| | | | | | | | | | | | GV | |
| Antibody | GFTFRT | 6 | WYDG | 11 | APQWEL | 13 | GGDNIG | 361 | DDLK | 383 | QVWD | 401 |
| 45 | YGMH | | SN | | VHEAFDI | | SYSVH | | RPS | | STSGV | |
| | | | | | | | | | | | VF | |
| Antibody | GFTFRT | 6 | WYDG | 11 | APQWEL | 13 | GGNNVE | 362 | DDND | 384 | QVWD | 29 |
| 46 | YGMH | | SN | | VHEAFDI | | DYSVH | | RPS | | SSSDH | |
| | | | | | | | | | | | VV | |
| Tezepelumab | GFTFRT | 6 | WYDGS | 11 | APQWELV | 13 | GGNNLG | 19 | DDSDR | 26 | QVWDS | 29 |
| | YGMH | | N | | HEAFDI | | SKSVH | | PS | | SSDHV | |
| | | | | | | | | | | | V | |

IMGT Numbering

| | | | | | | | | | | | | |
|---|---|---|---|---|---|---|---|---|---|---|---|---|
| Antibody | GFTFRT | 7 | IWYDG | 12 | ARAPQW | 16 | NLGSKS | 22 | DVS | — | QVWD | 27 |
| | | | | | ELVHEA | | | | | | SSSTFV | |
| 1 | YT | | SNK | | FDI | | | | | | V | |
| Antibody | GFTFRT | 8 | IWYDG | 12 | ARAPQW | 16 | NLGSKS | 22 | DVS | — | QVWD | 28 |
| 2 | YP | | SNK | | ELVHEAF | | | | | | SSSTY | |
| | | | | | DI | | | | | | VV | |
| Antibody | GFTFRT | 8 | IWYDG | 12 | ARALIW | 17 | NLISKS | 23 | DDS | — | QVWD | 28 |
| 3 | YP | | SNK | | ELFHEYF | | | | | | SSSTY | |
| | | | | | DI | | | | | | VV | |
| Antibody | GFTFRT | 8 | IWYDG | 12 | ARAPQW | 16 | NLVSKS | 24 | DVS | — | QVWD | 28 |
| 4 | YP | | SNK | | ELVHEAF | | | | | | SSSTY | |
| | | | | | DI | | | | | | VV | |
| Antibody | GFTFRT | 9 | IWYDG | 12 | ARAPQW | 16 | NLVSKS | 24 | DDS | — | QVWD | 27 |
| 5 | YG | | SNK | | ELVHEAF | | | | | | SSSTFV | |
| | | | | | DI | | | | | | V | |
| Antibody | GFTFRT | 9 | IWYDG | 12 | ARALLW | 18 | NLVSKS | 24 | DVS | — | QVWD | 29 |
| 6 | YG | | SNK | | ELFHEYF | | | | | | SSSDH | |
| | | | | | DI | | | | | | VV | |
| Antibody | GFTFRT | 9 | IWYDG | 12 | ARAPQW | 16 | NLGSKS | 22 | DVS | — | QVWD | 30 |
| 7 | YG | | SNK | | ELVHEAF | | | | | | SSSTIV | |
| | | | | | DI | | | | | | V | |
| Antibody | GFTFRT | 7 | IWYDG | 12 | ARAPQW | 16 | NLGSKS | 22 | DDS | — | QVWD | 31 |
| 8 | YT | | SNK | | ELVHEAF | | | | | | KSSTY | |
| | | | | | DI | | | | | | VV | |
| Antibody | GFTFRT | 8 | IWYDG | 12 | ARALLW | 18 | NLVSKS | 24 | DDS | — | QVWD | 28 |
| 9 | YP | | SNK | | ELFHEYF | | | | | | SSSTY | |
| | | | | | DI | | | | | | VV | |
| Antibody | GFTFRT | 9 | IWYDG | 12 | ARAPQW | 16 | QNIRYY | 363 | DVS | — | QQGYS | 385 |
| 10 | YG | | SNK | | ELVHEAF | | | | | | PPLT | |
| | | | | | DI | | | | | | | |
| Antibody | GFTFRT | 9 | IWYDG | 12 | ARAPQW | 16 | NIGSKS | 364 | EDS | — | QVWD | 386 |
| 11 | YG | | SNK | | ELVHEAF | | | | | | SDSDH | |
| | | | | | DI | | | | | | VV | |
| Antibody | GFTFRT | 9 | IWYDG | 12 | ARAPQW | 16 | NIGTKS | 365 | DDS | — | QVWD | 387 |
| 12 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | NIGTKS | 365 | DDS | — | QVWD | 387 |
| 13 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | NIGTKS | 365 | DDS | — | QVWD | 387 |
| 14 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | KIGRKS | 366 | DDS | — | QVWD | 388 |
| 15 | YG | | SNK | | ELVHEAF | | | | | | SSSDE | |
| | | | | | DI | | | | | | GV | |
| Antibody | GFTFRT | 9 | IWYDG | 12 | ARAPQW | 16 | KIGRKS | 366 | DDS | — | QVWD | 388 |
| 16 | YG | | SNK | | ELVHEAF | | | | | | SSSDE | |
| | | | | | DI | | | | | | GV | |
| Antibody | GFTFRT | 9 | IWYDG | 12 | ARAPQW | 16 | NIGTKS | 365 | DDS | — | QVWD | 389 |
| 17 | YG | | SNK | | ELVHEAF | | | | | | GSSDQ | |
| | | | | | DI | | | | | | SV | |
| Antibody | GFTFRT | 9 | IWYDG | 12 | ARAPQW | 16 | NIGGKS | 367 | DDT | — | QVWD | 390 |
| 18 | YG | | SNK | | ELVHEAF | | | | | | TTTDH | |
| | | | | | DI | | | | | | VV | |
| Antibody | GFTFRT | 9 | IWYDG | 12 | ARAPQW | 16 | NIGSKS | 364 | DDS | — | QVWD | 391 |
| 19 | YG | | SNK | | ELVHEAF | | | | | | SSSDP | |
| | | | | | DI | | | | | | VV | |
| Antibody | GFTFRT | 9 | IWYDG | 12 | ARAPQW | 16 | NIGTKS | 365 | DDS | — | QVWD | 387 |
| 20 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | NIGTKS | 365 | DDS | — | QVWD | 387 |
| 21 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | KIGRKS | 366 | DDS | — | QVWD | 388 |
| 22 | YG | | SNK | | ELVHEAF | | | | | | SSSDE | |
| | | | | | DI | | | | | | GV | |
| Antibody | GFTFRT | 9 | IWYDG | 12 | ARAPQW | 16 | NIGTKS | 365 | DDS | — | QVWD | 392 |
| 23 | YG | | SNK | | ELVHEAF | | | | | | RSSDQ | |
| | | | | | DI | | | | | | SV | |
| Antibody | GFTFRT | 9 | IWYDG | 12 | ARAPQW | 16 | SIGSKY | 368 | DDS | — | QVWD | 392 |
| 24 | YG | | SNK | | ELVHEAF | | | | | | RSSDQ | |
| | | | | | DI | | | | | | SV | |
| Antibody | GFTFRT | 9 | IWYDG | 12 | ARAPQW | 16 | NIGTKS | 365 | DDS | — | QVWD | 393 |
| 25 | YG | | SNK | | ELVHEAF | | | | | | SGSGQ | |
| | | | | | DI | | | | | | TV | |
| Antibody | GFTFRT | 9 | IWYDG | 12 | ARAPQW | 16 | NIGTRS | 369 | DDS | | QVWD | 387 |
| 26 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | NIGSET | 370 | DDS | — | HVWD | 394 |
| 27 | YG | | SNK | | ELVHEAF | | | | | | SSSDH | |
| | | | | | DI | | | | | | VM | |
| Antibody | GFTFRT | 9 | IWYDG | 12 | ARAPQW | 16 | NIETYS | 371 | DDS | — | QVWD | 395 |
| 28 | YG | | SNK | | ELVHEAF | | | | | | SDSDH | |
| | | | | | DI | | | | | | LE | |
| Antibody | GFTFRT | 9 | IWYDG | 12 | ARAPQW | 16 | NIGRKS | 372 | DDS | — | QVWD | 396 |
| 29 | YG | | SNK | | ELVHEAF | | | | | | SYSNH | |
| | | | | | DI | | | | | | VV | |
| Antibody | GFTFRT | 9 | IWYDG | 12 | ARAPQW | 16 | KIGRKS | 366 | DDS | — | QVWD | 388 |
| 30 | YG | | SNK | | ELVHEAF | | | | | | SSSDE | |
| | | | | | DI | | | | | | GV | |
| Antibody | GFTFRT | 9 | IWYDG | 12 | ARAPQW | 16 | NIGTKS | 365 | DDS | — | QVWD | 392 |
| 31 | YG | | SNK | | ELVHEAF | | | | | | RSSDQ | |
| | | | | | DI | | | | | | SV | |
| Antibody | GFTFRT | 9 | IWYDG | 12 | ARAPQW | 16 | NIETYS | 371 | DDS | — | QVWD | 397 |
| 32 | YG | | SNK | | ELVHEAF | | | | | | SGGDH | |
| | | | | | DI | | | | | | VV | |
| Antibody | GFTFRT | 9 | IWYDG | 12 | ARAPQW | 16 | NIGSKS | 364 | DDS | — | QVWD | 29 |
| 33 | YG | | SNK | | ELVHEAF | | | | | | SSSDH | |
| | | | | | DI | | | | | | VV | |
| Antibody | GFTFRT | 9 | IWYDG | 12 | ARAPQW | 16 | YLGAKS | 590 | DDK | — | QVWD | 398 |
| 34 | YG | | SNK | | ELVHEAF | | | | | | DISDHI | |
| | | | | | DI | | | | | | V | |
| Antibody | GFTFRT | 9 | IWYDG | 12 | ARAPQW | 16 | NIGTKS | 365 | DDS | — | QVWD | 387 |
| 35 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | SIGTKS | 373 | DDS | — | QVWD | 387 |
| 36 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | SIEDKN | 374 | DDS | — | QVWD | 399 |
| 37 | YG | | SNK | | ELVHEAF | | | | | | MASDR | |
| | | | | | DI | | | | | | VV | |
| Antibody | GFTFRT | 9 | IWYDG | 12 | ARAPQW | 16 | KIGRKS | 366 | DDS | — | QVWD | 388 |
| 38 | YG | | SNK | | ELVHEAF | | | | | | SSSDE | |
| | | | | | DI | | | | | | GV | |
| Antibody | GFTFRT | 9 | IWYDG | 12 | ARAPQW | 16 | NIGTKS | 365 | DDS | — | QVWD | 387 |
| 39 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | KIGRKS | 366 | DDS | — | QVWD | 388 |
| 40 | YG | | SNK | | ELVHEAF | | | | | | SSSDE | |
| | | | | | DI | | | | | | GV | |
| Antibody | GFTFRT | 9 | IWYDG | 12 | ARAPQW | 16 | NIGTKS | 365 | DDS | — | QVWD | 387 |
| 41 | YG | | SNK | | ELVHEAF | | | | | | SSSDQ | |
| | | | | | DI | | | | | | SV | |
| Antibody | GFTFRT | 9 | IWYDG | 12 | ARAPQW | 16 | NIETYS | 371 | DDS | — | QVWD | 29 |
| 42 | YG | | SNK | | ELVHEAF | | | | | | SSSDH | |
| | | | | | DI | | | | | | VV | |
| Antibody | GFTFRT | 9 | IWYDG | 12 | ARAPQW | 16 | KIGRKS | 366 | DDS | — | QVWD | 388 |
| 43 | YG | | SNK | | ELVHEAF | | | | | | SSSDE | |
| | | | | | DI | | | | | | GV | |
| Antibody | GFTFRT | 9 | IWYDG | 12 | ARAPQW | 16 | KIGRKS | 366 | DDS | — | QVWD | 400 |
| 44 | YG | | SNK | | ELVHEAF | | | | | | TSSDQ | |
| | | | | | DI | | | | | | GV | |
| Antibody | GFTFRT | 9 | IWYDG | 12 | ARAPQW | 16 | NIGSYS | 375 | DDL | — | QVWD | 401 |
| 45 | YG | | SNK | | ELVHEAF | | | | | | STSGV | |
| | | | | | DI | | | | | | VF | |
| Antibody | GFTFRT | 9 | IWYDG | 12 | ARAPQW | 16 | NVEDYS | 376 | DDN | — | QVWD | 29 |
| 46 | YG | | SNK | | ELVHEAF | | | | | | SSSDH | |
| | | | | | DI | | | | | | VV | |
| Tezepelumab | GFTFRT | 9 | IWYDG | 12 | ARAPQW | 16 | NLGSKS | 22 | DDS | — | QVWD | 29 |
| | YG | | SNK | | ELVHEAF | | | | | | SSSDH | |
| | | | | | DI | | | | | | VV | |

TABLE 4
Sequences of anti-TSLP Antibody Constructs - VH and VL

| | SEQ | | SEQ | |
|---|---|---|---|---|
| | ID | | ID | |
| Antibody | NO | VH | NO | VL |
| Antibody 1 | 32 | QMQLVESGGGVVQPGRS | 37 | SYVLTQPPSVSVAPGQTA |
| | | LRLSCAASGFTFRTYTM | | RITCGGNNLGSKSVHWY |
| | | HWVRQAPGKGLEWVAV | | QQKPGQAPVLVVYDVSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSTFVVFGGGTK |
| | | QWELVHEAFDIWGQGT | | LTVL |
| | | MVTVSS | | |
| Antibody 2 | 33 | QMQLVESGGGVVQPGRS | 38 | SYVLTQPPSVSVAPGQTA |
| | | LRLSCAASGFTFRTYPM | | RITCGGNNLGSKSVHWY |
| | | HWVRQAPGKGLEWVAV | | QQKPGQAPVLVVYDVSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSTYVVFGGGTK |
| | | QWELVHEAFDIWGQGT | | LTVL |
| | | MVTVSS | | |
| Antibody 3 | 452 | QMQLVESGGGVVQPGRS | 39 | SYVLTQPPSVSVAPGQTA |
| | | LRLSCAASGFTFRTYPM | | RITCGGNNLISKSVHWY |
| | | HWVRQAPGKGLEWVAV | | QQKPGQAPVLVVYDDSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAL | | QVWDSSSTYVVFGGGTK |
| | | IWELFHEYFDIWGQGTM | | LTVL |
| | | VTVSS | | |
| Antibody 4 | 33 | QMQLVESGGGVVQPGRS | 40 | SYVLTQPPSVSVAPGQTA |
| | | LRLSCAASGFTFRTYPM | | RITCGGNNLVSKSVHWY |
| | | HWVRQAPGKGLEWVAV | | QQKPGQAPVLVVYDVSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSTYVVFGGGTK |
| | | QWELVHEAFDIWGQGT | | LTVL |
| | | MVTVSS | | |
| Antibody 5 | 34 | QMQLVESGGGVVQPGRS | 41 | SYVLTQPPSVSVAPGQTA |
| | | LRLSCAASGFTFRTYGM | | RITCGGNNLVSKSVHWY |
| | | HWVRQAPGKGLEWVAV | | QQKPGQAPVLVVYDDSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSTFVVFGGGTK |
| | | QWELVHEAFDIWGQGT | | LTVL |
| | | MVTVSS | | |
| Antibody 6 | 35 | QMQLVESGGGVVQPGRS | 42 | SYVLTQPPSVSVAPGQTA |
| | | LRLSCAASGFTFRTYGM | | RITCGGNNLVSKSVHWY |
| | | HWVRQAPGKGLEWVAV | | QQKPGQAPVLVVYDVSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAL | | QVWDSSSDHVVFGGGT |
| | | LWELFHEYFDIWGQGTM | | KLTVL |
| | | VTVSS | | |
| Antibody 7 | 34 | QMQLVESGGGVVQPGRS | 43 | SYVLTQPPSVSVAPGQTA |
| | | LRLSCAASGFTFRTYGM | | RITCGGNNLGSKSVHWY |
| | | HWVRQAPGKGLEWVAV | | QQKPGQAPVLVVYDVSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSTIVVFGGGTK |
| | | QWELVHEAFDIWGQGT | | LTVL |
| | | MVTVSS | | |
| Antibody 8 | 32 | QMQLVESGGGVVQPGRS | 44 | SYVLTQPPSVSVAPGQTA |
| | | LRLSCAASGFTFRTYTM | | RITCGGNNLGSKSVHWY |
| | | HWVRQAPGKGLEWVAV | | QQKPGQAPVLVVYDDSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDKSSTYVVFGGGT |
| | | QWELVHEAFDIWGQGT | | KLTVL |
| | | MVTVSS | | |
| Antibody 9 | 36 | QMQLVESGGGVVQPGRS | 45 | SYVLTQPPSVSVAPGQTA |
| | | LRLSCAASGFTFRTYPM | | RITCGGNNLVSKSVHWY |
| | | HWVRQAPGKGLEWVAV | | QQKPGQAPVLVVYDDSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAL | | QVWDSSSTYVVFGGGTK |
| | | LWELFHEYFDIWGQGTM | | LTVL |
| | | VTVSS | | |
| Antibody 10 | 34 | QMQLVESGGGVVQPGRS | 402 | DIQLTQSPSSLSASVGDR |
| | | LRLSCAASGFTFRTYGM | | VAITCRTSQNIRYYLNW |
| | | HWVRQAPGKGLEWVAV | | YQRKPGKAPRLLIYDVSS |
| | | IWYDGSNKHYADSVKG | | LQSGVPSRFSGRGSGTDF |
| | | RFTITRDNSKNTLNLQM | | TLTISSLQAEDFATYYCQ |
| | | NSLRAEDTAVYYCARAP | | QGYSPPLTFGGGTKVEIK |
| | | QWELVHEAFDIWGQGT | | |
| | | MVTVSS | | |
| Antibody 11 | 34 | QMQLVESGGGVVQPGRS | 403 | NFMLTQPPSVSVAPGKT |
| | | LRLSCAASGFTFRTYGM | | AKMTCGGDNIGSKSVH |
| | | HWVRQAPGKGLEWVAV | | WYQQMPGQAPVLVIYE |
| | | IWYDGSNKHYADSVKG | | DSDRPSGTPERFSGSNSG |
| | | RFTITRDNSKNTLNLQM | | HTATLTISRVEAGDEAD |
| | | NSLRAEDTAVYYCARAP | | YYCQVWDSDSDHVVFG |
| | | QWELVHEAFDIWGQGT | | GGTTLTVL |
| | | MVTVSS | | |
| Antibody 12 | 34 | QMQLVESGGGVVQPGRS | 404 | QSVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 13 | 34 | QMQLVESGGGVVQPGRS | 405 | SYELTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 14 | 34 | QMQLVESGGGVVQPGRS | 406 | SYVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 15 | 34 | QMQLVESGGGVVQPGRS | 407 | SYVLTQPPSVSVAPGQTA |
| | | LRLSCAASGFTFRTYGM | | RITCGGNKIGRKSVHWY |
| | | HWVRQAPGKGLEWVAV | | QQRPGQAPVLVVFDDSD |
| | | IWYDGSNKHYADSVKG | | RPSWISERFSGSNSGNTA |
| | | RFTITRDNSKNTLNLQM | | TLTISRVEAGDEADYYC |
| | | NSLRAEDTAVYYCARAP | | QVWDSSSDEGVFGGGTK |
| | | QWELVHEAFDIWGQGT | | LTVL |
| | | MVTVSS | | |
| Antibody 16 | 34 | QMQLVESGGGVVQPGRS | 408 | SYELTQPPSVSVAPGQAA |
| | | LRLSCAASGFTFRTYGM | | RITCGGNKIGRKSVHWY |
| | | HWVRQAPGKGLEWVAV | | QQRPGQAPVLVVFDDSD |
| | | IWYDGSNKHYADSVKG | | RPSWISERFSGSNSGNTA |
| | | RFTITRDNSKNTLNLQM | | TLTISRVEAGDEADYYC |
| | | NSLRAEDTAVYYCARAP | | QVWDSSSDEGVFGGGTK |
| | | QWELVHEAFDIWGQGT | | LTVL |
| | | MVTVSS | | |
| Antibody 17 | 34 | QMQLVESGGGVVQPGRS | 409 | QSVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDGSSDQSVFGPGTRV |
| | | QWELVHEAFDIWGQGT | | TVL |
| | | MVTVSS | | |
| Antibody 18 | 34 | QMQLVESGGGVVQPGRS | 410 | SYELTQPPSVSVAPGQTA |
| | | LRLSCAASGFTFRTYGM | | SITCGGNNIGGKSVHWY |
| | | HWVRQAPGKGLEWVAV | | QLKPGQAPVLVVYDDTD |
| | | IWYDGSNKHYADSVKG | | RPSWIPERFSGSNSANTA |
| | | RFTITRDNSKNTLNLQM | | TLTISGVEAGDEADYQC |
| | | NSLRAEDTAVYYCARAP | | QVWDTTTDHVVFGGGT |
| | | QWELVHEAFDIWGQGT | | KLTVL |
| | | MVTVSS | | |
| Antibody 19 | 34 | QMQLVESGGGVVQPGRS | 411 | SYVLTQPPSVSVAPGQTA |
| | | LRLSCAASGFTFRTYGM | | RITCGGNNIGSKSVHWY |
| | | HWVRQAPGKGLEWVAV | | QQKPGQAPLLVVYDDSD |
| | | IWYDGSNKHYADSVKG | | RPSWIPERFSGSNSGNAA |
| | | RFTITRDNSKNTLNLQM | | TLTVSRVEAGDEADYYC |
| | | NSLRAEDTAVYYCARAP | | QVWDSSSDPVVFGGGTK |
| | | QWELVHEAFDIWGQGT | | LTVL |
| | | MVTVSS | | |
| Antibody 20 | 34 | QMQLVESGGGVVQPGRS | 412 | FYVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 21 | 34 | QMQLVESGGGVVQPGRS | 413 | QSVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPPWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 22 | 34 | QMQLVESGGGVVQPGRS | 414 | QAVLTQPPSVSVAPGQT |
| | | LRLSCAASGFTFRTYGM | | ARITCGGNKIGRKSVHW |
| | | HWVRQAPGKGLEWVAV | | YQQRPGQAPVLVVFDDS |
| | | IWYDGSNKHYADSVKG | | DRPSWISERFSGSNSGNT |
| | | RFTITRDNSKNTLNLQM | | ATLTISRVEAGDEADYY |
| | | NSLRAEDTAVYYCARAP | | CQVWDSSSDEGVFGGGT |
| | | QWELVHEAFDIWGQGT | | KLTVL |
| | | MVTVSS | | |
| Antibody 23 | 34 | QMQLVESGGGVVQPGRS | 415 | SYELTQPPSVSVAPGQTA |
| | | LRLSCAASGFTFRTYGM | | RITCGGHNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDRSSDQSVFGPGTRV |
| | | QWELVHEAFDIWGQGT | | TVL |
| | | MVTVSS | | |
| Antibody 24 | 34 | QMQLVESGGGVVQPGRS | 416 | SYVLTQPPSVSVAPGQTA |
| | | LRLSCAASGFTFRTYGM | | RVTCGGDSIGSKYVHWY |
| | | HWVRQAPGKGLEWVAV | | QQKPGQAPVLVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDRSSDQSVFGPGTRV |
| | | QWELVHEAFDIWGQGT | | TVL |
| | | MVTVSS | | |
| Antibody 25 | 34 | QMQLVESGGGVVQPGRS | 417 | SYELTQPPSVSVAPGQTA |
| | | LRLSCAASGFTFRTYGM | | RITCGGQNIGTKSVHWY |
| | | HWVRQAPGKGLEWVAV | | QQKSGQAPILVVFDDSD |
| | | IWYDGSNKHYADSVKG | | RPSWVTERFSGSNFGTTA |
| | | RFTITRDNSKNTLNLQM | | TLTINGVEAGDEADYFC |
| | | NSLRAEDTAVYYCARAP | | QVWDSGSGQTVFGPGTR |
| | | QWELVHEAFDIWGQGT | | VTVS |
| | | MVTVSS | | |
| Antibody 26 | 34 | QMQLVESGGGVVQPGRS | 418 | SYELTQPPSVSVAPGQTA |
| | | LRLSCAASGFTFRTYGM | | RITCGGQNIGTRSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 27 | 34 | QMQLVESGGGVVQPGRS | 419 | SYELTQPPSVSVAPGQTA |
| | | LRLSCAASGFTFRTYGM | | RITCGGNNIGSETVHWY |
| | | HWVRQAPGKGLEWVAV | | RQKAGQAPVLVVYDDS |
| | | IWYDGSNKHYADSVKG | | DRPSGIPERFSGSNSGNT |
| | | RFTITRDNSKNTLNLQM | | AALTISRVEVGDEADYY |
| | | NSLRAEDTAVYYCARAP | | CHVWDSSSDHVMFGGG |
| | | QWELVHEAFDIWGQGT | | TKLTVL |
| | | MVTVSS | | |
| Antibody 28 | 34 | QMQLVESGGGVVQPGRS | 420 | SYELTQPPSVSVGPGKTA |
| | | LRLSCAASGFTFRTYGM | | TITCRGDNIETYSVHWY |
| | | HWVRQAPGKGLEWVAV | | QQKPGQAPVLVVYDDSD |
| | | IWYDGSNKHYADSVKG | | RPSGIPERFSGSNSGNTA |
| | | RFTITRDNSKNTLNLQM | | TLTINRVGAGDEADYYC |
| | | NSLRAEDTAVYYCARAP | | QVWDSDSDHLEFGGGTK |
| | | QWELVHEAFDIWGQGT | | LTVL |
| | | MVTVSS | | |
| Antibody 29 | 34 | QMQLVESGGGVVQPGRS | 421 | SYELTQPPSVSVAPGQTA |
| | | LRLSCAASGFTFRTYGM | | RITCGGNNIGRKSAHWY |
| | | HWVRQAPGKGLEWVAV | | QQRPGQAPVLVVYDDSD |
| | | IWYDGSNKHYADSVKG | | RPSWIPERFSGSNSGNTA |
| | | RFTITRDNSKNTLNLQM | | TLTISRVEAGDEADYYC |
| | | NSLRAEDTAVYYCARAP | | QVWDSYSNHVVFGGGT |
| | | QWELVHEAFDIWGQGT | | KLTVL |
| | | MVTVSS | | |
| Antibody 30 | 34 | QMQLVESGGGVVQPGRS | 422 | LPVLTQPPSVSVAPGQTA |
| | | LRLSCAASGFTFRTYGM | | RITCGGNKIGRKSVHWY |
| | | HWVRQAPGKGLEWVAV | | QQRPGQAPVLVVFDDSD |
| | | IWYDGSNKHYADSVKG | | RPSWISERFSGSNSGNTA |
| | | RFTITRDNSKNTLNLQM | | TLTISRVEAGDEADYYC |
| | | NSLRAEDTAVYYCARAP | | QVWDSSSDEGVFGGGTK |
| | | QWELVHEAFDIWGQGT | | LTVL |
| | | MVTVSS | | |
| Antibody 31 | 34 | QMQLVESGGGVVQPGRS | 423 | SYVLTQPPSVSVAPGQTA |
| | | LRLSCAASGFTFRTYGM | | RITCGGHNIGTKSVHWY |
| | | HWVRQAPGKGLEWVAV | | QQKSGQAPILAVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDRSSDQSVFGPGTRV |
| | | QWELVHEAFDIWGQGT | | TVL |
| | | MVTVSS | | |
| Antibody 32 | 34 | QMQLVESGGGVVQPGRS | 424 | SYELTQPPSVSVGPGKTA |
| | | LRLSCAASGFTFRTYGM | | TITCRGDNIETYSVHWY |
| | | HWVRQAPGKGLEWVAV | | QQKPGQAPVLVVYDDSD |
| | | IWYDGSNKHYADSVKG | | RPSGIPERFSGSNSGNTA |
| | | RFTITRDNSKNTLNLQM | | TLTINRVEARDEADYFC |
| | | NSLRAEDTAVYYCARAP | | QVWDSGGDHVVFGGGT |
| | | QWELVHEAFDIWGQGT | | KLTVL |
| | | MVTVSS | | |
| Antibody 33 | 34 | QMQLVESGGGVVQPGRS | 425 | SYVLTQPPSVSVAPGQTA |
| | | LRLSCAASGFTFRTYGM | | RITCGGNNIGSKSVHWY |
| | | HWVRQAPGKGLEWVAV | | QQKPGQAPVLVVYDDSD |
| | | IWYDGSNKHYADSVKG | | RPSWIPERFSGTNSGNTA |
| | | RFTITRDNSKNTLNLQM | | TLTISRVEAGDEADYYC |
| | | NSLRAEDTAVYYCARAP | | QVWDSSSDHVVFGGGT |
| | | QWELVHEAFDIWGQGT | | KLTVL |
| | | MVTVSS | | |
| Antibody 34 | 34 | QMQLVESGGGVVQPGRS | 426 | SYELTQPPSVSVAPGKRA |
| | | LRLSCAASGFTFRTYGM | | RITCEGTYLGAKSVHWY |
| | | HWVRQAPGKGLEWVAV | | QQKPGQAPVLVIYDDKE |
| | | IWYDGSNKHYADSVKG | | RPSGIPERFSASNSGNTA |
| | | RFTITRDNSKNTLNLQM | | TLTITRVEAGDEADYHC |
| | | NSLRAEDTAVYYCARAP | | QVWDDISDHIVFGGGTK |
| | | QWELVHEAFDIWGQGT | | LSVL |
| | | MVTVSS | | |
| Antibody 35 | 34 | QMQLVESGGGVVQPGRS | 427 | SYVLTQPPSVSVAPGQTA |
| | | LRLSCAASGFTFRTYGM | | RITCGGNNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 36 | 34 | QMQLVESGGGVVQPGRS | 428 | SYVLTQPPSVSVAPGQTA |
| | | LRLSCAASGFTFRTYGM | | RITCGGQSIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 37 | 34 | QMQLVESGGGVVQPGRS | 429 | SYELTQPPSVSVAPGQAA |
| | | LRLSCAASGFTFRTYGM | | SLTCGANSIEDKNVHWY |
| | | HWVRQAPGKGLEWVAV | | QQRPGQAPLLVIYDDSER |
| | | IWYDGSNKHYADSVKG | | PSGIPERFSGSNSGNTATL |
| | | RFTITRDNSKNTLNLQM | | TITRVAAGDEADYYCQV |
| | | NSLRAEDTAVYYCARAP | | WDMASDRVVFGPGTTV |
| | | QWELVHEAFDIWGQGT | | SVL |
| | | MVTVSS | | |
| Antibody 38 | 34 | QMQLVESGGGVVQPGRS | 430 | SYELTQPPSVSVAPGQTA |
| | | LRLSCAASGFTFRTYGM | | RITCGGNKIGRKSVHWY |
| | | HWVRQAPGKGLEWVAV | | QQRPGQAPVLVVFDDSD |
| | | IWYDGSNKHYADSVKG | | RPSWISERFSGSNSGNTA |
| | | RFTITRDNSKNTLNLQM | | TLTIGRVEAGDEADYYC |
| | | NSLRAEDTAVYYCARAP | | QVWDSSSDEGVFGGGTK |
| | | QWELVHEAFDIWGQGT | | LAVL |
| | | MVTVSS | | |
| Antibody 39 | 34 | QMQLVESGGGVVQPGRS | 431 | SYELTQPPSVSVAPGRTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGPGTRV |
| | | QWELVHEAFDIWGQGT | | TVL |
| | | MVTVSS | | |
| Antibody 40 | 34 | QMQLVESGGGVVQPGRS | 432 | QAGLTQPPSVSVAPGQT |
| | | LRLSCAASGFTFRTYGM | | ARITCGGNKIGRKSVHW |
| | | HWVRQAPGKGLEWVAV | | YQQRPGQAPVLVVFDDS |
| | | IWYDGSNKHYADSVKG | | DRPSWISERFSGSNSGNT |
| | | RFTITRDNSKNTLNLQM | | ATLTISRVEAGDEADYY |
| | | NSLRAEDTAVYYCARAP | | CQVWDSSSDEGVFGGGT |
| | | QWELVHEAFDIWGQGT | | KLTVL |
| | | MVTVSS | | |
| Antibody 41 | 34 | QMQLVESGGGVVQPGRS | 433 | QSVLTQPPSVSVAPGQTA |
| | | LRLSCAASGFTFRTYGM | | RITCGGQNIGTKSVHWY |
| | | HWVRQAPGKGLEWVAV | | QQKSGQAPILVVYDDSD |
| | | IWYDGSNKHYADSVKG | | RPSWIPERFSGSNFGTTA |
| | | RFTITRDNSKNTLNLQM | | TLTISRVEAGDEADYFCQ |
| | | NSLRAEDTAVYYCARAP | | VWDSSSDQSVFGTGTKV |
| | | QWELVHEAFDIWGQGT | | TVL |
| | | MVTVSS | | |
| Antibody 42 | 34 | QMQLVESGGGVVQPGRS | 434 | SYVLTQPPSVSVGPGKTA |
| | | LRLSCAASGFTFRTYGM | | TITCRGDNIETYSVHWY |
| | | HWVRQAPGKGLEWVAV | | QRKPGQAPVLVVYDDSD |
| | | IWYDGSNKHYADSVKG | | RPSGIPERFSGSNSGNTA |
| | | RFTITRDNSKNTLNLQM | | TLTISRVEAGDEADYYC |
| | | NSLRAEDTAVYYCARAP | | QVWDSSSDHVVFGGGT |
| | | QWELVHEAFDIWGQGT | | KLTVL |
| | | MVTVSS | | |
| Antibody 43 | 34 | QMQLVESGGGVVQPGRS | 435 | SYELTQPPSVSVAPGQTA |
| | | LRLSCAASGFTFRTYGM | | RITCGGNKIGRKSVHWY |
| | | HWVRQAPGKGLEWVAV | | QQRPGQAPVLVVFDDSD |
| | | IWYDGSNKHYADSVKG | | RPSWISERFSGSNSGNTA |
| | | RFTITRDNSKNTLNLQM | | TLTISRVDAGDEADYYC |
| | | NSLRAEDTAVYYCARAP | | QVWDSSSDEGVFGGGTK |
| | | QWELVHEAFDIWGQGT | | LTVL |
| | | MVTVSS | | |
| Antibody 44 | 34 | QMQLVESGGGVVQPGRS | 436 | SYELTQPPSVSVAPGQTA |
| | | LRLSCAASGFTFRTYGM | | RITCGGNKIGRKSVHWY |
| | | HWVRQAPGKGLEWVAV | | QQKPGQAPVLVVFDDSD |
| | | IWYDGSNKHYADSVKG | | RPSWISERFSGSNSGNTA |
| | | RFTITRDNSKNTLNLQM | | TLTISRVEAGDEADYHC |
| | | NSLRAEDTAVYYCARAP | | QVWDTSSDQGVFGGGT |
| | | QWELVHEAFDIWGQGT | | KLTVL |
| | | MVTVSS | | |
| Antibody 45 | 34 | QMQLVESGGGVVQPGRS | 437 | SYVLTQPPSVAVAPGQT |
| | | LRLSCAASGFTFRTYGM | | AKITCGGDNIGSYSVHW |
| | | HWVRQAPGKGLEWVAV | | YQQKPGQAPVLVVYDD |
| | | IWYDGSNKHYADSVKG | | LKRPSWIPERFFGSNSGN |
| | | RFTITRDNSKNTLNLQM | | TATLTISRVEAGDEAVY |
| | | NSLRAEDTAVYYCARAP | | YCQVWDSTSGVVFGGG |
| | | QWELVHEAFDIWGQGT | | TTVTVL |
| | | MVTVSS | | |
| Antibody 46 | 34 | QMQLVESGGGVVQPGRS | 438 | SYELTQPPSVSVAPGKTA |
| | | LRLSCAASGFTFRTYGM | | RITCGGNNVEDYSVHWY |
| | | HWVRQAPGKGLEWVAV | | HQKAGQAPVVVMYDDN |
| | | IWYDGSNKHYADSVKG | | DRPSGVPERFSGSNSGNT |
| | | RFTITRDNSKNTLNLQM | | ATLTIGRVEAGDEADYY |
| | | NSLRAEDTAVYYCARAP | | CQVWDSSSDHVVFGGGT |
| | | QWELVHEAFDIWGQGT | | KLTVL |
| | | MVTVSS | | |
| Tezepelumab | 34 | QMQLVESGGGVVQPGRS | 441 | SYVLTQPPSVSVAPGQTA |
| | | LRLSCAASGFTFRTYGM | | RITCGGNNLGSKSVHWY |
| | | HWVRQAPGKGLEWVAV | | QQKPGQAPVLVVYDDSD |
| | | IWYDGSNKHYADSVKG | | RPSWIPERFSGSNSGNTA |
| | | RFTITRDNSKNTLNLQM | | TLTISRGEAGDEADYYC |
| | | NSLRAEDTAVYYCARAP | | QVWDSSSDHVVFGGGT |
| | | QWELVHEAFDIWGQGT | | KLTVL |
| | | MVTVSS | | |
| hu23b12 | 442 | QVQLVQSGAEVKKPGAS | 443 | EIVLTQSPGTLSLSPGERA |
| | | VKVSCKASGYIFTDYAM | | TLSCRASQPISISVHWYQ |
| | | HWVRQAPGQGLEWMGT | | QKPGQAPRLLIYFASQSIS |
| | | FIPLLDTSDYNQNFKGRV | | GIPDRFSGSGSGTDFTLTI |
| | | TMTTDTSTSTAYMELRS | | SRLEPEDFAVYYCQQTFS |
| | | LRSDDTAVYYCARMGV | | LPYTFGQGTKVEIKR |
| | | THSYVMDAWGQGTLVT | | |
| | | VSS | | |
| 43B1-H2L2 | 444 | QMQLVESGGGAVQPGG | 445 | EIVMTQSPATLSVSPGER |
| | | SLRLSCAASGFTFRSYDM | | ATLSCRASQSVSSNLAW |
| | | HWVRQAPGKGLEWVAV | | YQQKPGQAPRLLISGAST |
| | | IWYDGSNENYADSVKGR | | RATGIPARFSGSGSGTDF |
| | | FTTSRDNSKNTLFLQMN | | TLTISSLQSEDFAVYYCQ |
| | | SLRAEDTAVYYCARSPL | | HYNNWPLTFGGGTKVEI |
| | | WYGEPDDAFDIWGQGTL | | K |
| | | VTVSS | | |
| 43B1-H6L1 | 446 | QMQLVESGGGAVQPGRS | 447 | EIVMTQSPATLSVSPGER |
| | | LRLSCAASGFTFRSYDM | | ATLSCRASQSVSSNLAW |
| | | HWVRQAPGKGLEWVAV | | YQQKPGQAPRLLISGAST |
| | | IWYDGSNENYADSVKGR | | RATGIPARFSGSGSGTDF |
| | | FTTSRDNSKNTLFLQMN | | TLTISSLRSEDFAVYYCQ |
| | | SLRAEDTAVYYCARSPL | | HYNNWPLTFGGGTKVEI |
| | | WYGEPDDAFDIWGQGTL | | K |
| | | VTVSS | | |
| TQC2731 (also | 554 | QVQLVQSGAEVKKPGAS | 555 | DIQMTQSPSSLSASVGDR |
| known as BSI- | | VKVSCKASGYTFTTYW | | VTITCRPTENIYSYLAWY |
| 045B and | | MHWVRQAPGQGLEWM | | QQKPGKAPKLLIYFARTL |
| bosakitug) | | GVIDPSDSDTTYNQKFK | | AEGVPSRFSGSGSGTDFT |
| | | GRVTMTRDTSTSTVYME | | LTISSLQPEDFATYYCQH |
| | | LSSLRSEDTAVYYCTRSL | | HYGTPWTFGGGTKVEIK |
| | | DGYYDYWGQGTLVTVS | | |
| | | S | | |
| Solrikitug | 562 | QVQLVQSGAEVKKPGAS | 563 | EIVLTQSPGTLSLSPGERA |
| | | VKVSCKASGYIFTDYAM | | TLSCRASQPISISVHWYQ |
| | | HWVRQAPGQGLEWMGT | | QKPGQAPRLLIYFASQSIS |
| | | FIPLLDTSDYAQKFQGRV | | GIPDRFSGSGSGTDFTLTI |
| | | TMTADTSTSTAYMELRS | | SRLEPEDFAVYYCQQTFS |
| | | LRSDDTAVYYCARMGV | | LPYTFGQGTKVEIK |
| | | THSYVMDAWGQGTLVT | | |
| | | VSS | | |

TABLE 5
Sequences of additional anti-TSLP antibody constructs - CDRs

| | | | | | | | | | | | | |
|---|---|---|---|---|---|---|---|---|---|---|---|---|
| | | SEQ | | SEQ | | SEQ | | SEQ | | SEQ | | SEQ |
| | | ID | | ID | | ID | | ID | | ID | | ID |
| Antibody | CDRH1 | NO: | CDRH2 | NO: | CDRH3 | NO: | CDRL1 | NO: | CDRL2 | NO: | CDRL3 | NO: |

Kabat Numbering

| | | | | | | | | | | | | |
|---|---|---|---|---|---|---|---|---|---|---|---|---|
| Antibody | TYGMH | 3 | VIWYD | 10 | APQWEL | 13 | GGNNLG | 19 | DVSD | 25 | QVWD | 29 |
| 47 | | | GSNKH | | VHEAFDI | | SKSVH | | RPS | | SSSDH | |
| | | | YADSV | | | | | | | | VV | |
| | | | KG | | | | | | | | | |
| Antibody | TYGMH | 3 | VIWYD | 10 | APQWEL | 13 | GGNNLG | 19 | DDSD | 26 | QVWD | 566 |
| 48 | | | GSNKH | | VHEAFDI | | SKSVH | | RPS | | SSSDF | |
| | | | YADSV | | | | | | | | VV | |
| | | | KG | | | | | | | | | |
| Antibody | TYGMH | 3 | VIWYD | 10 | APQWEL | 13 | GGNNLV | 21 | DVSD | 25 | QVWD | 29 |
| 49 | | | GSNKH | | VHEAFDI | | SKSVH | | RPS | | SSSDH | |
| | | | YADSV | | | | | | | | VV | |
| | | | KG | | | | | | | | | |
| Antibody | TYGMH | 3 | VIWYD | 10 | APQWEL | 13 | GGNNLV | 21 | DDSD | 26 | QVWD | 566 |
| 50 | | | GSNKH | | VHEAFDI | | SKSVH | | RPS | | SSSDF | |
| | | | YADSV | | | | | | | | VV | |
| | | | KG | | | | | | | | | |
| Antibody | TYGMH | 3 | VIWYD | 10 | APQWEL | 13 | GGNNLG | 19 | DVSD | 25 | QVWD | 567 |
| 51 | | | GSNKH | | VHEAFDI | | SKSVH | | RPS | | SSSTH | |
| | | | YADSV | | | | | | | | VV | |
| | | | KG | | | | | | | | | |
| Antibody | TYGMH | 3 | VIWYD | 10 | APQWEL | 13 | GGNNLG | 19 | DVSD | 25 | QVWD | 566 |
| 52 | | | GSNKH | | VHEAFDI | | SKSVH | | RPS | | SSSDF | |
| | | | YADSV | | | | | | | | VV | |
| | | | KG | | | | | | | | | |
| Antibody | TYGMH | 3 | VIWYD | 10 | APQWEL | 13 | GGNNLG | 19 | DDSD | 26 | QVWD | 568 |
| 53 | | | GSNKH | | VHEAFDI | | SKSVH | | RPS | | SSSTFV | |
| | | | YADSV | | | | | | | | V | |
| | | | KG | | | | | | | | | |
| Antibody | TYGMH | 3 | VIWYD | 10 | APQWEL | 13 | GGNNLV | 21 | DVSD | 25 | QVWD | 567 |
| 54 | | | GSNKH | | VHEAFDI | | SKSVH | | RPS | | SSSTH | |
| | | | YADSV | | | | | | | | VV | |
| | | | KG | | | | | | | | | |
| Antibody | TYGMH | 3 | VIWYD | 10 | APQWEL | 13 | GGNNLV | 21 | DVSD | 25 | QVWD | 566 |
| 55 | | | GSNKH | | VHEAFDI | | SKSVH | | RPS | | SSSDF | |
| | | | YADSV | | | | | | | | VV | |
| | | | KG | | | | | | | | | |
| Antibody | TYGMH | 3 | VIWYD | 10 | APQWEL | 13 | GGNNLV | 21 | DDSD | 26 | QVWD | 568 |
| 56 | | | GSNKH | | VHEAFDI | | SKSVH | | RPS | | SSSTFV | |
| | | | YADSV | | | | | | | | V | |
| | | | KG | | | | | | | | | |
| Antibody | TYGMH | 3 | VIWYD | 10 | APQWEL | 13 | GGNNLV | 21 | DDSD | 26 | QVWD | 569 |
| 57 | | | GSNKH | | VHEAFDI | | SKSVH | | RPS | | SSSTIV | |
| | | | YADSV | | | | | | | | V | |
| | | | KG | | | | | | | | | |
| Antibody | TYGMH | 3 | VIWYD | 10 | APQWEL | 13 | GGNNLG | 19 | DVSD | 25 | QVWD | 568 |
| 58 | | | GSNKH | | VHEAFDI | | SKSVH | | RPS | | SSSTFV | |
| | | | YADSV | | | | | | | | V | |
| | | | KG | | | | | | | | | |
| Antibody | TYGMH | 3 | VIWYD | 10 | APQWEL | 13 | GGNNLV | 21 | DVSD | 25 | QVWD | 568 |
| 59 | | | GSNKH | | VHEAFDI | | SKSVH | | RPS | | SSSTFV | |
| | | | YADSV | | | | | | | | V | |
| | | | KG | | | | | | | | | |
| Antibody | TYGMH | 3 | VIWYD | 10 | APQWEL | 13 | GGNNLV | 21 | DVSD | 25 | QVWD | 569 |
| 60 | | | GSNKH | | VHEAFDI | | SKSVH | | RPS | | SSSTIV | |
| | | | YADSV | | | | | | | | V | |
| | | | KG | | | | | | | | | |
| Antibody | TYGMH | 3 | VIWYD | 10 | ALLWEL | 15 | GGNNLG | 19 | DDSD | 26 | QVWD | 29 |
| 61 | | | GSNKH | | FHEYFDI | | SKSVH | | RPS | | SSSDH | |
| | | | YADSV | | | | | | | | VV | |
| | | | KG | | | | | | | | | |

Chothia Numbering

| | | | | | | | | | | | | |
|---|---|---|---|---|---|---|---|---|---|---|---|---|
| Antibody | GFTFRT | 6 | WYDG | 11 | APQWEL | 13 | GGNNLG | 19 | DVSD | 25 | QVWD | 29 |
| | | | | | | | | | | | SSSDH | |
| 47 | YGMH | | SN | | VHEAFDI | | SKSVH | | RPS | | VV | |
| Antibody | GFTFRT | 6 | WYDG | 11 | APQWEL | 13 | GGNNLG | 19 | DDSD | 26 | QVWD | 566 |
| 48 | YGMH | | SN | | VHEAFDI | | SKSVH | | RPS | | SSSDF | |
| | | | | | | | | | | | VV | |
| Antibody | GFTFRT | 6 | WYDG | 11 | APQWEL | 13 | GGNNLV | 21 | DVSD | 25 | QVWD | 29 |
| 49 | YGMH | | SN | | VHEAFDI | | SKSVH | | RPS | | SSSDH | |
| | | | | | | | | | | | VV | |
| Antibody | GFTFRT | 6 | WYDG | 11 | APQWEL | 13 | GGNNLV | 21 | DDSD | 26 | QVWD | 566 |
| 50 | YGMH | | SN | | VHEAFDI | | SKSVH | | RPS | | SSSDF | |
| | | | | | | | | | | | VV | |
| Antibody | GFTFRT | 6 | WYDG | 11 | APQWEL | 13 | GGNNLG | 19 | DVSD | 25 | QVWD | 567 |
| 51 | YGMH | | SN | | VHEAFDI | | SKSVH | | RPS | | SSSTH | |
| | | | | | | | | | | | VV | |
| Antibody | GFTFRT | 6 | WYDG | 11 | APQWEL | 13 | GGNNLG | 19 | DVSD | 25 | QVWD | 566 |
| 52 | YGMH | | SN | | VHEAFDI | | SKSVH | | RPS | | SSSDF | |
| | | | | | | | | | | | VV | |
| Antibody | GFTFRT | 6 | WYDG | 11 | APQWEL | 13 | GGNNLG | 19 | DDSD | 26 | QVWD | 568 |
| 53 | YGMH | | SN | | VHEAFDI | | SKSVH | | RPS | | SSSTFV | |
| | | | | | | | | | | | V | |
| Antibody | GFTFRT | 6 | WYDG | 11 | APQWEL | 13 | GGNNLV | 21 | DVSD | 25 | QVWD | 567 |
| 54 | YGMH | | SN | | VHEAFDI | | SKSVH | | RPS | | SSSTH | |
| | | | | | | | | | | | VV | |
| Antibody | GFTFRT | 6 | WYDG | 11 | APQWEL | 13 | GGNNLV | 21 | DVSD | 25 | QVWD | 566 |
| 55 | YGMH | | SN | | VHEAFDI | | SKSVH | | RPS | | SSSDF | |
| | | | | | | | | | | | VV | |
| Antibody | GFTFRT | 6 | WYDG | 11 | APQWEL | 13 | GGNNLV | 21 | DDSD | 26 | QVWD | 568 |
| | | | | | | | | | | | SSSTFV | |
| 56 | YGMH | | SN | | VHEAFDI | | SKSVH | | RPS | | V | |
| Antibody | GFTFRT | 6 | WYDG | 11 | APQWEL | 13 | GGNNLV | 21 | DDSD | 26 | QVWD | 569 |
| 57 | YGMH | | SN | | VHEAFDI | | SKSVH | | RPS | | SSSTIV | |
| | | | | | | | | | | | V | |
| Antibody | GFTFRT | 6 | WYDG | 11 | APQWEL | 13 | GGNNLG | 19 | DVSD | 25 | QVWD | 568 |
| 58 | YGMH | | SN | | VHEAFDI | | SKSVH | | RPS | | SSSTFV | |
| | | | | | | | | | | | V | |
| Antibody | GFTFRT | 6 | WYDG | 11 | APQWEL | 13 | GGNNLV | 21 | DVSD | 25 | QVWD | 568 |
| 59 | YGMH | | SN | | VHEAFDI | | SKSVH | | RPS | | SSSTFV | |
| | | | | | | | | | | | V | |
| Antibody | GFTFRT | 6 | WYDG | 11 | APQWEL | 13 | GGNNLV | 21 | DVSD | 25 | QVWD | 569 |
| 60 | YGMH | | SN | | VHEAFDI | | SKSVH | | RPS | | SSSTIV | |
| | | | | | | | | | | | V | |
| Antibody | GFTFRT | 6 | WYDG | 11 | ALLWEL | 15 | GGNNLG | 19 | DDSD | 26 | QVWD | 29 |
| 61 | YGMH | | SN | | FHEYFDI | | SKSVH | | RPS | | SSSDH | |
| | | | | | | | | | | | VV | |

IMGT Numbering

| | | | | | | | | | | | | |
|---|---|---|---|---|---|---|---|---|---|---|---|---|
| Antibody | GFTFRT | 9 | IWYDG | 12 | ARAPQW | 16 | NLGSKS | 22 | DVS | — | QVWD | 29 |
| 47 | YG | | SNK | | ELVHEAF | | | | | | SSSDH | |
| | | | | | DI | | | | | | VV | |
| Antibody | GFTFRT | 9 | IWYDG | 12 | ARAPQW | 16 | NLGSKS | 22 | DDS | — | QVWD | 566 |
| 48 | YG | | SNK | | ELVHEAF | | | | | | SSSDF | |
| | | | | | DI | | | | | | VV | |
| Antibody | GFTFRT | 9 | IWYDG | 12 | ARAPQW | 16 | NLVSKS | 24 | DVS | — | QVWD | 29 |
| 49 | YG | | SNK | | ELVHEAF | | | | | | SSSDH | |
| | | | | | DI | | | | | | VV | |
| Antibody | GFTFRT | 9 | IWYDG | 12 | ARAPQW | 16 | NLVSKS | 24 | DDS | — | QVWD | 566 |
| 50 | YG | | SNK | | ELVHEAF | | | | | | SSSDF | |
| | | | | | DI | | | | | | VV | |
| Antibody | GFTFRT | 9 | IWYDG | 12 | ARAPQW | 16 | NLGSKS | 22 | DVS | — | QVWD | 567 |
| 51 | YG | | SNK | | ELVHEAF | | | | | | SSSTH | |
| | | | | | DI | | | | | | VV | |
| Antibody | GFTFRT | 9 | IWYDG | 12 | ARAPQW | 16 | NLGSKS | 22 | DVS | — | QVWD | 566 |
| 52 | YG | | SNK | | ELVHEAF | | | | | | SSSDF | |
| | | | | | DI | | | | | | VV | |
| Antibody | GFTFRT | 9 | IWYDG | 12 | ARAPQW | 16 | NLGSKS | 22 | DDS | — | QVWD | 568 |
| 53 | YG | | SNK | | ELVHEAF | | | | | | SSSTFV | |
| | | | | | DI | | | | | | V | |
| Antibody | GFTFRT | 9 | IWYDG | 12 | ARAPQW | 16 | NLVSKS | 24 | DVS | — | QVWD | 567 |
| 54 | YG | | SNK | | ELVHEAF | | | | | | SSSTH | |
| | | | | | DI | | | | | | VV | |
| Antibody | GFTFRT | 9 | IWYDG | 12 | ARAPQW | 16 | NLVSKS | 24 | DVS | — | QVWD | 566 |
| 55 | YG | | SNK | | ELVHEAF | | | | | | SSSDF | |
| | | | | | DI | | | | | | VV | |
| Antibody | GFTFRT | 9 | IWYDG | 12 | ARAPQW | 16 | NLVSKS | 24 | DDS | — | QVWD | 568 |
| 56 | YG | | SNK | | ELVHEAF | | | | | | SSSTFV | |
| | | | | | DI | | | | | | V | |
| Antibody | GFTFRT | 9 | IWYDG | 12 | ARAPQW | 16 | NLVSKS | 24 | DDS | — | QVWD | 569 |
| 57 | YG | | SNK | | ELVHEAF | | | | | | SSSTIV | |
| | | | | | DI | | | | | | V | |
| Antibody | GFTFRT | 9 | IWYDG | 12 | ARAPQW | 16 | NLGSKS | 22 | DVS | — | QVWD | 568 |
| 58 | YG | | SNK | | ELVHEAF | | | | | | SSSTFV | |
| | | | | | DI | | | | | | V | |
| Antibody | GFTFRT | 9 | IWYDG | 12 | ARAPQW | 16 | NLVSKS | 24 | DVS | — | QVWD | 568 |
| 59 | YG | | SNK | | ELVHEAF | | | | | | SSSTFV | |
| | | | | | DI | | | | | | V | |
| Antibody | GFTFRT | 9 | IWYDG | 12 | ARAPQW | 16 | NLVSKS | 24 | DVS | — | QVWD | 569 |
| 60 | YG | | SNK | | ELVHEAF | | | | | | SSSTIV | |
| | | | | | DI | | | | | | V | |
| Antibody | GFTFRT | 9 | IWYDG | 12 | ARALLW | 18 | NLGSKS | 22 | DDS | — | QVWD | 29 |
| 61 | YG | | SNK | | ELFHEYF | | | | | | SSSDH | |
| | | | | | DI | | | | | | VV | |

TABLE 6
Sequences of additional anti-TSLP antibody constructs - VH/VL

| | SEQ | | SEQ | |
|---|---|---|---|---|
| | ID | | ID | |
| Antibody | NO | VH | NO | VL |
| Antibody | 34 | QMQLVESGGGVVQPGRSLRLSCAASGFTFRTYGMHWVR | 570 | SYVLTQPPSVSVAPGQTARITCGGNNLGSK |
| 47 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDHVVFGGGTKLTVL |
| Antibody | 34 | QMQLVESGGGVVQPGRSLRLSCAASGFTFRTYGMHWVR | 571 | SYVLTQPPSVSVAPGQTARITCGGNNLGSK |
| 48 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDFVVFGGGTKLTVL |
| Antibody | 34 | QMQLVESGGGVVQPGRSLRLSCAASGFTFRTYGMHWVR | 572 | SYVLTQPPSVSVAPGQTARITCGGNNLVSK |
| 49 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDHVVFGGGTKLTVL |
| Antibody | 34 | QMQLVESGGGVVQPGRSLRLSCAASGFTFRTYGMHWVR | 573 | SYVLTQPPSVSVAPGQTARITCGGNNLVSK |
| 50 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDFVVFGGGTKLTVL |
| Antibody | 34 | QMQLVESGGGVVQPGRSLRLSCAASGFTFRTYGMHWVR | 574 | SYVLTQPPSVSVAPGQTARITCGGNNLGSK |
| 51 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTHVVFGGGTKLTVL |
| Antibody | 34 | QMQLVESGGGVVQPGRSLRLSCAASGFTFRTYGMHWVR | 575 | SYVLTQPPSVSVAPGQTARITCGGNNLGSK |
| 52 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDFVVFGGGTKLTVL |
| Antibody | 34 | QMQLVESGGGVVQPGRSLRLSCAASGFTFRTYGMHWVR | 576 | SYVLTQPPSVSVAPGQTARITCGGNNLGSK |
| 53 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTFVVFGGGTKLTVL |
| Antibody | 34 | QMQLVESGGGVVQPGRSLRLSCAASGFTFRTYGMHWVR | 577 | SYVLTQPPSVSVAPGQTARITCGGNNLVSK |
| 54 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTHVVFGGGTKLTVL |
| Antibody | 34 | QMQLVESGGGVVQPGRSLRLSCAASGFTFRTYGMHWVR | 578 | SYVLTQPPSVSVAPGQTARITCGGNNLVSK |
| 55 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSDFVVFGGGTKLTVL |
| Antibody | 34 | QMQLVESGGGVVQPGRSLRLSCAASGFTFRTYGMHWVR | 41 | SYVLTQPPSVSVAPGQTARITCGGNNLVSK |
| 56 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTFVVFGGGTKLTVL |
| Antibody | 34 | QMQLVESGGGVVQPGRSLRLSCAASGFTFRTYGMHWVR | 579 | SYVLTQPPSVSVAPGQTARITCGGNNLVSK |
| 57 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTIVVFGGGTKLTVL |
| Antibody | 34 | QMQLVESGGGVVQPGRSLRLSCAASGFTFRTYGMHWVR | 580 | SYVLTQPPSVSVAPGQTARITCGGNNLGSK |
| 58 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTFVVFGGGTKLTVL |
| Antibody | 34 | QMQLVESGGGVVQPGRSLRLSCAASGFTFRTYGMHWVR | 581 | SYVLTQPPSVSVAPGQTARITCGGNNLVSK |
| 59 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTFVVFGGGTKLTVL |
| Antibody | 34 | QMQLVESGGGVVQPGRSLRLSCAASGFTFRTYGMHWVR | 582 | SYVLTQPPSVSVAPGQTARITCGGNNLVSK |
| 60 | | QAPGKGLEWVAVIWYDGSNKHYADSVKGRFTITRDNSK | | SVHWYQQKPGQAPVLVVYDVSDRPSWIP |
| | | NTLNLQMNSLRAEDTAVYYCARAPQWELVHEAFDIWGQ | | ERFSGSNSGNTATLTISRGEAGDEADYYCQ |
| | | GTMVTVSS | | VWDSSSTIVVFGGGTKLTVL |
| Antibody | 35 | QMQLVESGGGVVQPGRSLRLSCAASGFTFRTYGMHWVR | 583 | SYVLTQPPSVSVAPGQTARITCGGNNLGSK |
| 61 | | QAPGKGLEWVAVIWYDGSNKHYADSVKGRFTITRDNSK | | SVHWYQQKPGQAPVLVVYDDSDRPSWIP |
| | | NTLNLQMNSLRAEDTAVYYCARALLWELFHEYFDIWGQ | | ERFSGSNSGNTATLTISRGEAGDEADYYCQ |
| | | GTMVTVSS | | VWDSSSDHVVFGGGTKLTVL |

### Anti-TSLP VH Domains

In some embodiments, an antibody, or antigen binding fragment thereof, provided herein comprises a VH sequence selected from any one of SEQ ID NOs: 32-36 and 452.

In some embodiments, an antibody, or antigen binding fragment thereof, provided herein comprises a VH sequence having at least about 80%, 90%, 91%, 92%, 93%, 94%, 95%, 96%, 97%, 98%, or 99% identity to a VH sequence provided in any one of SEQ ID NOs: 32-36 and 452. In some embodiments, an antibody provided herein comprises a VH sequence provided in any one of SEQ ID NOs: 32-36 and 452, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### Anti-TSLP VL Domains

In some embodiments, an antibody, or antigen binding fragment thereof, provided herein comprises a VL sequence selected from any one of SEQ ID NOs: 37-45, 402-438, and 570-583.

In some embodiments, an antibody, or antigen binding fragment thereof, provided herein comprises a VL sequence having at least about 80%, 90%, 91%, 92%, 93%, 94%, 95%, 96%, 97%, 98%, or 99% identity to a VL sequence provided in any one of SEQ ID NOs: 37-45, 402-438, and 570-583. In some embodiments, an antibody, or antigen binding fragment thereof, provided herein comprises a VL sequence provided in any one of SEQ ID NOs: 37-45, 402-438, and 570-583, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### Anti-TSLP VH-VL Combinations

In some embodiments, an antibody, or antigen binding fragment thereof, provided herein comprises a VH sequence selected from any one of SEQ ID NOs: 32-36 and 452; and a VL sequence selected from any one of SEQ ID NOs: 37-45, 402-438, and 570-583.

In certain aspects, any one of SEQ ID NOs: 32-36 and 452 can be combined with any one of SEQ ID NOs: 37-45, 402-438, and 570-583.

In certain aspects, an antibody, or antigen binding fragment thereof, provided herein comprises a VH sequence and a VL sequence of a construct provided in TABLE 4 (e.g., a VH sequence and a VL sequence from the same row of TABLE 4). In certain aspects, an antibody, or an antigen binding fragment thereof, provided herein comprises a VH sequence and a VL sequence of a construct provided in TABLE 6 (e.g., a VH sequence and a VL sequence from the same row of TABLE 6).

In certain aspects, an antibody, or an antigen binding fragment thereof, provided herein comprises a VH sequence and a VL sequence of a construct provided in PCT Application No. PCT/US2025/034623, incorporated by reference herein in its entirety. In certain aspects, an antibody, or antigen binding fragment thereof, provided herein comprises a VH sequence and a VL sequence from Table 4 or Table 11 in PCT Application No. PCT/US2025/034623 (e.g., a VH sequence and a VL sequence from the same row of Table 4 or Table 11).

In some embodiments, an antibody, or antigen binding fragment thereof, provided herein comprises a VH sequence having at least about 80%, 90%, 91%, 92%, 93%, 94%, 95%, 96%, 97%, 98%, or 99% identity to a VH sequence provided in any one of SEQ ID NOs: 32-36 and 452; and a VL sequence having at least about 80%, 90%, 91%, 92%, 93%, 94%, 95%, 96%, 97%, 98%, or 99% identity to a VL sequence provided in any one of SEQ ID NOs: 37-45, 402-438, and 570-583. In some embodiments, an antibody, or antigen binding fragment thereof, provided herein comprises a VH sequence provided in any one SEQ ID NOs: 32-36 and 452, with up to 1, 2, 3, 4, 5, 6, 7, 8, 9, 10, 11, 12, 13, 14, 15, 16, 17, 18, 19, or 20 amino acid substitutions, and a VL sequence provided in any one of SEQ ID NOs: 37-45, 402-438, and 570-583, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody, or antigen binding fragment thereof, comprises a VH sequence set forth in SEQ ID NO: 32 and a VL sequence set forth in SEQ ID NO: 37.

In certain embodiments, the antibody, or antigen binding fragment thereof, comprises a VH sequence set forth in SEQ ID NO: 33 and a VL sequence set forth in SEQ ID NO: 38.

In certain embodiments, the antibody, or antigen binding fragment thereof, comprises a VH sequence set forth in SEQ ID NO: 452 and a VL sequence set forth in SEQ ID NO: 39.

In certain embodiments, the antibody, or antigen binding fragment thereof, comprises a VH sequence set forth in SEQ ID NO: 33 and a VL sequence set forth in SEQ ID NO: 40.

In certain embodiments, the antibody, or antigen binding fragment thereof, comprises a VH sequence set forth in SEQ ID NO: 34 and a VL sequence set forth in SEQ ID NO: 41.

In certain embodiments, the antibody, or antigen binding fragment thereof, comprises a VH sequence set forth in SEQ ID NO: 35 and a VL sequence set forth in SEQ ID NO: 42.

In certain embodiments, the antibody, or antigen binding fragment thereof, comprises a VH sequence set forth in SEQ ID NO: 34 and a VL sequence set forth in SEQ ID NO: 43.

In certain embodiments, the antibody, or antigen binding fragment thereof, comprises a VH sequence set forth in SEQ ID NO: 32 and a VL sequence set forth in SEQ ID NO: 44.

In certain embodiments, the antibody, or antigen binding fragment thereof, comprises a VH sequence set forth in SEQ ID NO: 36 and a VL sequence set forth in SEQ ID NO: 45.

In certain embodiments, the antibody, or antigen binding fragment thereof, comprises a VH sequence set forth in SEQ ID NO: 34 and a VL sequence set forth in SEQ ID NO: 415.

In certain embodiments, the antibody, or antigen binding fragment thereof, comprises a VH sequence set forth in SEQ ID NO: 34 and a VL sequence set forth in SEQ ID NO: 421.

In certain embodiments of any of the antibodies, or antigen binding fragments thereof, described above, the antibody further comprises a heavy chain comprising a human IgG sequence selected from a sequence set forth in any of SEQ ID NOs: 47-270. In certain embodiments, the heavy chain comprises a human IgG sequence selected from SEQ ID NO: 61 and SEQ ID NO: 173.

In certain embodiments, the antibody comprises a VH sequence set forth in SEQ ID NO: 34; a VL sequence set forth in SEQ ID NO: 41; and the antibody further comprises a heavy chain comprising a human IgG sequence selected from a sequence set forth in any of SEQ ID NOs: 47-270. In certain embodiments, the heavy chain comprises a human IgG sequence selected from SEQ ID NO: 61 and SEQ ID NO: 173. In certain embodiments of any of the antibodies described above, the antibody, or antigen binding fragment thereof, further comprises a constant light chain sequence comprising a sequence set forth in SEQ ID NO: 520.

Although the antibody may be produced using a polynucleotide sequence that encodes a constant heavy chain region containing a C-terminal lysine (e.g., a sequence encoding any one of SEQ ID NOs: 159-270), the C-terminal lysine may be cleaved off during manufacture or after administration (resulting in, e.g., an antibody containing a constant heavy chain sequence of any one of SEQ ID NOs: 47-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 47-158), or a mixture thereof (e.g., a mixture of the same heavy chain constant region sequence with and without a C-terminal lysine, such as a mixture of SEQ ID NO: 173 and SEQ ID NO: 61).

In certain embodiments, the antibody comprises a VH sequence set forth in SEQ ID NO: 34, a VL sequence set forth in SEQ ID NO: 41, a heavy chain constant region (e.g., an IgG1 constant region) comprising LALA/YTE substitutions, and a human lambda light chain constant region (e.g., a lambda light chain constant region of subtype 1, 2, 3, or 7, such as a lambda light chain constant region set forth in any one of SEQ ID NOs: 520-523). In certain embodiments, the antibody comprises a VH sequence set forth in SEQ ID NO: 34, a VL sequence set forth in SEQ ID NO: 41, a heavy chain constant region comprising a sequence set forth in SEQ ID NO: 61 or SEQ ID NO: 173, and a light chain constant region comprising a sequence set forth in SEQ ID NO: 520. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 34; and a VL sequence having at least about 80%, 90%, 91%, 92%, 93%, 94%, 95%, 96%, 97%, 98%, or 99% identity to a VL sequence provided in SEQ ID NO: 41, a heavy chain constant region (e.g., an IgG1 constant region) comprising LALA/YTE substitutions, and a human lambda light chain constant region (e.g., a lambda light chain constant region of subtype 1, 2, 3, or 7, such as a lambda light chain constant region set forth in any one of SEQ ID NOs: 520-523). In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 34; and a VL sequence having at least about 80%, 90%, 91%, 92%, 93%, 94%, 95%, 96%, 97%, 98%, or 99% identity to a VL sequence provided in SEQ ID NO: 41, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 520 or a sequence having at least about 80%, 90%, 91%, 92%, 93%, 94%, 95%, 96%, 97%, 98%, or 99% identity to SEQ ID NO: 520.

In certain embodiments, the antibody comprises a VH sequence set forth in SEQ ID NO: 34, a VL sequence set forth in SEQ ID NO: 41, a heavy chain constant region comprising a sequence set forth in SEQ ID NO: 61 or SEQ ID NO: 173, and a light chain constant region comprising a sequence set forth in SEQ ID NO: 520. In certain embodiments, the antibody comprises a VH sequence set forth in SEQ ID NO: 34, a VL sequence set forth in SEQ ID NO: 41, a heavy chain constant region comprising a sequence set forth in SEQ ID NO: 173, and a light chain constant region comprising a sequence set forth in SEQ ID NO: 520. In some embodiments, the antibody comprises a heavy chain sequence set forth in SEQ ID NO: 524 or 526 and a light chain sequence set forth in SEQ ID NO: 525. In some embodiments, the antibody comprises a heavy chain sequence set forth in SEQ ID NO: 524 and a light chain sequence set forth in SEQ ID NO: 525. The heavy chain sequence of SEQ ID NO: 524 contains a C-terminal lysine, which may be cleaved off during manufacture or after administration, resulting in an antibody containing the heavy chain sequence of SEQ ID NO: 526.

In n certain embodiments, the antibody comprises a VH sequence set forth in SEQ ID NO: 34, a VL sequence set forth in SEQ ID NO: 43, a heavy chain constant region (e.g., an IgG1 constant region) comprising LALA/YTE substitutions, and a human lambda light chain constant region (e.g., a lambda light chain constant region of subtype 1, 2, 3, or 7, such as a lambda light chain constant region set forth in any one of SEQ ID NOs: 520-523). In certain embodiments, the antibody comprises a VH sequence set forth in SEQ ID NO: 34, a VL sequence set forth in SEQ ID NO: 43, a heavy chain constant region comprising a sequence set forth in SEQ ID NO: 61 or SEQ ID NO: 173, and a light chain constant region comprising a sequence set forth in SEQ ID NO: 520. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 34; and a VL sequence having at least about 80%, 90%, 91%, 92%, 93%, 94%, 95%, 96%, 97%, 98%, or 99% identity to a VL sequence provided in SEQ ID NO: 43, a heavy chain constant region (e.g., an IgG1 constant region) comprising LALA/YTE substitutions, and a human lambda light chain constant region (e.g., a lambda light chain constant region of subtype 1, 2, 3, or 7, such as a lambda light chain constant region set forth in any one of SEQ ID NOs: 520-523). In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 34; and a VL sequence having at least about 80%, 90%, 91%, 92%, 93%, 94%, 95%, 96%, 97%, 98%, or 99% identity to a VL sequence provided in SEQ ID NO: 43, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 520 or a sequence having at least about 80%, 90%, 91%, 92%, 93%, 94%, 95%, 96%, 97%, 98%, or 99% identity to SEQ ID NO: 520.

In certain embodiments, the antibody comprises a VH sequence set forth in SEQ ID NO: 34, a VL sequence set forth in SEQ ID NO: 43, a heavy chain constant region comprising a sequence set forth in SEQ ID NO: 61 or SEQ ID NO: 173, and a light chain constant region comprising a sequence set forth in SEQ ID NO: 520. In certain embodiments, the antibody comprises a VH sequence set forth in SEQ ID NO: 34, a VL sequence set forth in SEQ ID NO: 43, a heavy chain constant region comprising a sequence set forth in SEQ ID NO: 173, and a light chain constant region comprising a sequence set forth in SEQ ID NO: 520. In some embodiments, the antibody comprises a heavy chain sequence set forth in SEQ ID NO: 527 and a light chain sequence set forth in SEQ ID NO: 528. The heavy chain sequence of SEQ ID NO: 527 contains a C-terminal lysine, which may be cleaved off during manufacture or after administration.

### Anti-TSLP CDRs

In some embodiments, disclosed herein is an antibody, or antigen binding fragment thereof, comprising 1, 2, 3, 4, 5, or 6 of the CDRs of TABLE 3. In some embodiments, disclosed herein is an antibody, or antigen binding fragment thereof, comprising 6 of the Kabat CDRs of TABLE 3, 6 of the Chothia CDRs of TABLE 3, or 6 of the IMGT CDRs of TABLE 3. In some embodiments, the antibody, or antigen binding fragment thereof, comprises 6 of the Kabat CDRs, 6 of the Chothia CDRs, or 6 of the IMGT CDRs from a single row of TABLE 3 (e.g., 6 CDRs from the same antibody).

In some embodiments, disclosed herein is an antibody, or an antigen binding fragment thereof, comprising 1, 2, 3, 4, 5, or 6 of the CDRs of TABLE 5. In some embodiments, disclosed herein is an antibody, or an antigen binding fragment thereof, comprising 6 of the Kabat CDRs of TABLE 5, 6 of the Chothia CDRs of TABLE 5, or 6 of the IMGT CDRs of TABLE 5. In some embodiments, the antibody, or an antigen binding fragment thereof, comprises 6 of the Kabat CDRs, 6 of the Chothia CDRs, or 6 of the IMGT CDRs from a single row of TABLE 5 (e.g., 6 CDRs from the same antibody).

In some embodiments, disclosed herein is an antibody, or an antigen binding fragment thereof, comprising 1, 2, 3, 4, 5, or 6 of the CDRs of an antibody provided in PCT Application No. PCT/US2025/034623, incorporated by reference herein in its entirety, such as 1, 2, 3, 4, 5, or 6 of the CDRs in Table 3 or Table 10 in PCT Application No. PCT/US2025/034623. In some embodiments, disclosed herein is an antibody, or an antigen binding fragment thereof, comprising 1, 2, 3, 4, 5, or 6 of the Kabat CDRs of an antibody, or an antigen binding fragment thereof, provided in PCT Application No. PCT/US2025/034623. In some embodiments, disclosed herein is an antibody, or an antigen binding fragment thereof, comprising 1, 2, 3, 4, 5, or 6 of the Chothia CDRs of an antibody, or an antigen binding fragment thereof, provided in PCT Application No. PCT/US2025/034623. In some embodiments, disclosed herein is an antibody, or an antigen binding fragment thereof, comprising 1, 2, 3, 4, 5, or 6 of the IMGT CDRs of an antibody provided in PCT Application No. PCT/US2025/034623. In some embodiments, the antibody, or an antigen binding fragment thereof, comprises 6 of the Kabat CDRs, 6 of the Chothia CDRs, or 6 of the IMGT CDRs from a single row of Table 3 or Table 10 in in PCT Application No. PCT/US2025/034623 (e.g., 6 CDRs from the same antibody).

In some embodiments, an antibody, or antigen binding fragment thereof, provided herein comprises one to three CDRs of a VH domain selected from any one of SEQ ID NOs: 32-36 and 452. In some embodiments, an antibody, or antigen binding fragment thereof, provided herein comprises two to three CDRs of a VH domain selected from any one of SEQ ID NOs: 32-36 and 452. In some embodiments, an antibody provided herein comprises three CDRs of a VH domain selected from any one of SEQ ID NOs: 32-36 and 452. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-H1, CDR-H2, or CDR-H3 selected from SEQ ID NOs: 1-18. In some embodiments, the CDR-H1 is a CDR-H1 of a VH domain selected from any one of SEQ ID NOs: 32-36 and 452, with up to 1, 2, 3, 4, or 5 amino acid substitutions. In some embodiments, the CDR-H2 is a CDR-H2 of a VH domain selected from any one of SEQ ID NOs: 32-36 and 452, with up to 1, 2, 3, 4, 5, 6, 7, or 8 amino acid substitutions. In some embodiments, the CDR-H3 is a CDR-H3 of a VH domain selected from any one of SEQ ID NOs: 32-36 and 452, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VL domain selected from any one of SEQ ID NOs: 37-45, 402-438, and 570-583. In some embodiments, an antibody, or antigen binding fragment thereof, provided herein comprises two to three CDRs of a VL domain selected from any one of SEQ ID NOs: 37-45, 402-438, and 570-583. In some embodiments, an antibody, or antigen binding fragment thereof, provided herein comprises three CDRs of a VL domain selected from any one of SEQ ID NOs: 37-45, 402-438, and 570-583. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-L1, CDR-L2, or CDR-L3 of any of SEQ ID NOs: 19-31, 345-401, 566-569, 590, and the amino acid sequences DVS, DDS, EDS, DDT, DDK, DDL, and DDN. In some embodiments, the CDR-L1 is a CDR-L1 of a VL domain of any one of SEQ ID NOs: 37-45, 402-438, and 570-583, with up to 1, 2, 3, 4, or 5 amino acid substitutions. In some embodiments, the CDR-L2 is a CDR-L2 of a VL domain of any one of SEQ ID NOs: 37-45, 402-438, and 570-583, with up to 1, 2, 3, 4, 5, 6, 7, or 8 amino acid substitutions. In some embodiments, the CDR-L3 is a CDR-L3 of a VL domain of any one of SEQ ID NOs: 37-45, 402-438, and 570-583, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VH domain selected from any one of SEQ ID NOs: 32-36 and 452 and one to three CDRs of a VL domain selected from any one of SEQ ID NOs: 37-45, 402-438, and 570-583. In some embodiments, an antibody, or antigen binding fragment thereof, provided herein comprises two to three CDRs of a VH domain selected from any one of SEQ ID NOs: 32-36 and 452 and two to three CDRs of a VL domain selected from any one of SEQ ID NOs: 37-45, 402-438, and 570-583. In some embodiments, an antibody, or antigen binding fragment thereof, provided herein comprises three CDRs of a VH domain selected from any one of SEQ ID NOs: 32-36 and 452 and three CDRs of a VL domain selected from any one of SEQ ID NOs: 37-45, 402-438, and 570-583. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody, or antigen binding fragment thereof, provided herein comprises a CDR-H3 selected from any one of SEQ ID NOs: 13-18. In some aspects, the CDR-H3 has at least about 80%, 90%, 91%, 92%, 93%, 94%, 95%, 96%, 97%, 98%, or 99% identity to a CDR-H3 selected from any one of SEQ ID NOs: 13-18. In some embodiments, the CDR-H3 is a CDR-H3 selected from any one of SEQ ID NOs: 13-18, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H1 selected from any one of SEQ ID NOs: 1-9. In some aspects, the CDR-H1 has at least about 80%, 90%, 91%, 92%, 93%, 94%, 95%, 96%, 97%, 98%, or 99% identity to a CDR-H1 selected from any one of SEQ ID NOs: 1-9. In some embodiments, the CDR-H1 is a CDR-H1 selected from any one of SEQ ID NO: 1-9, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H2 selected from any one of SEQ ID NOs: 10-12. In some aspects, the CDR-H2 has at least about 80%, 90%, 91%, 92%, 93%, 94%, 95%, 96%, 97%, 98%, or 99% identity to a CDR-H2 selected from any one of SEQ ID NOs: 10-12. In some embodiments, the CDR-H2 is a CDR-H2 selected from any one of SEQ ID NOs: 10-12, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L3 selected from any one of SEQ ID NOs: 27-31, 385-401, and 566-569. In some aspects, the CDR-L3 has at least about 80%, 90%, 91%, 92%, 93%, 94%, 95%, 96%, 97%, 98%, or 99% identity to a CDR-L3 selected from any one of SEQ ID NOs: 27-31, 385-401, and 566-569. In some embodiments, the CDR-L3 is a CDR-L3 selected from any one of SEQ ID NOs: 27-31, 385-401, and 566-569, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L2 selected from any one of SEQ ID NOs: 25-26, 377-384, and the amino acid sequences DDS, DVS, EDS, DDT, DDK, DDL, and DDN. In some aspects, the CDR-L2 has at least about 80%, 90%, 91%, 92%, 93%, 94%, 95%, 96%, 97%, 98%, or 99% identity to a CDR-L2 selected from any one of SEQ ID NOs: 25-26, 377-384, and the amino acid sequences DDS, DVS, EDS, DDT, DDK, DDL, and DDN. In some embodiments, the CDR-L2 is a CDR-L2 selected from any one of SEQ ID NOs: 25-26, 377-384, and the amino acid sequences DDS, DVS, EDS, DDT, DDK, DDL, and DDN, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L1 selected from any one of SEQ ID NOs: 19-24, 345-376, and 590. In some aspects, the CDR-L1 has at least about 80%, 90%, 91%, 92%, 93%, 94%, 95%, 96%, 97%, 98%, or 99% identity to a CDR-L1 selected from any one of SEQ ID NOs: 19-24, 345-376, and 590. In some embodiments, the CDR-L1 is a CDR-L1 selected from any one of SEQ ID NOs: 19-24, 345-376, and 590, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H3 selected from any one of SEQ ID NOs: 13-18, a CDR-H2 selected from any one of SEQ ID NOs: 10-12, a CDR-H1 selected from any one of SEQ ID NOs: 1-9, a CDR-L3 selected from any one of SEQ ID NOs: 27-31, 385-401, and 566-569, a CDR-L2 selected from any one of SEQ ID NOs: 25-26, 377-384, and the amino acid sequences DDS, DVS, EDS, DDT, DDK, DDL, and DDN, and a CDR-L1 selected from any one of SEQ ID NOs: 19-24, 345-376, and 590. In some embodiments, the CDR-H3 has at least about 80%, 90%, 91%, 92%, 93%, 94%, 95%, 96%, 97%, 98%, or 99% identity to a CDR-H3 selected from any one of SEQ ID NOs: 13-18, the CDR-H2 has at least about 80%, 90%, 91%, 92%, 93%, 94%, 95%, 96%, 97%, 98%, or 99% identity to a CDR-H2 selected from any one of SEQ ID NOs: 10-12, the CDR-H1 has at least about 80%, 90%, 91%, 92%, 93%, 94%, 95%, 96%, 97%, 98%, or 99% identity to a CDR-H1 selected from any one of SEQ ID NOs: 1-9, the CDR-L3 has at least about 80%, 90%, 91%, 92%, 93%, 94%, 95%, 96%, 97%, 98%, or 99% identity to a CDR-L3 selected from any one of SEQ ID NOs: 27-31, 385-401, and 566-569, the CDR-L2 has at least about 80%, 90%, 91%, 92%, 93%, 94%, 95%, 96%, 97%, 98%, or 99% identity to a CDR-L2 selected from any one of SEQ ID NOs: 25-26, 377-384, and the amino acid sequences DDS, DVS, EDS, DDT, DDK, DDL, and DDN, and the CDR-L1 has at least about 80%, 90%, 91%, 92%, 93%, 94%, 95%, 96%, 97%, 98%, or 99% identity to a CDR-L1 selected from any one of SEQ ID NOs: 19-24, 345-376, and 590. In some embodiments, the CDR-H3 is a CDR-H3 selected from any one of SEQ ID NOs: 13-18, with up to 1, 2, 3, 4, 5, 6, 7, or 8 amino acid substitutions; the CDR-H2 is a CDR-H2 selected from any one of SEQ ID NOs: 10-12, with up to 1, 2, 3, 4, 5, 6, 7, or 8 amino acid substitutions; the CDR-H1 is a CDR-H1 selected from any one of SEQ ID NOs: 1-9, with up to 1, 2, 3, 4, or 5 amino acid substitutions; the CDR-L3 is a CDR-L3 selected from any one of SEQ ID NOs: 27-31, 385-401, and 566-569, with up to 1, 2, 3, 4, or 5 amino acid substitutions; the CDR-L2 is a CDR-L2 selected from any one of SEQ ID NOs: 25-26, 377-384, and the amino acid sequences DDS, DVS, EDS, DDT, DDK, DDL, and DDN, with up to 1, 2, 3, or 4 amino acid substitutions; and the CDR-L1 is a CDR-L1 selected from any one of SEQ ID NOs: 19-24, 345-376, and 590, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27. In some embodiments, the CDR-H3 has at least about 80%, 90%, 91%, 92%, 93%, 94%, 95%, 96%, 97%, 98%, or 99% identity to a CDR-H3 selected from any one of SEQ ID NOs: 13 and 16, the CDR-H2 has at least about 80%, 90%, 91%, 92%, 93%, 94%, 95%, 96%, 97%, 98%, or 99% identity to a CDR-H2 selected from any one of SEQ ID NOs: 10, 11, and 12, the CDR-H1 has at least about 80%, 90%, 91%, 92%, 93%, 94%, 95%, 96%, 97%, 98%, or 99% identity to a CDR-H1 selected from any one of SEQ ID NOs: 3, 6, and 9, the CDR-L3 has at least about 80%, 90%, 91%, 92%, 93%, 94%, 95%, 96%, 97%, 98%, or 99% identity to a CDR-L3 of SEQ ID NO: 27, the CDR-L2 has at least about 80%, 90%, 91%, 92%, 93%, 94%, 95%, 96%, 97%, 98%, or 99% identity to a CDR-L2 selected from SEQ ID NO: 26 and the amino acid sequence DDS, and the CDR-L1 has at least about 80%, 90%, 91%, 92%, 93%, 94%, 95%, 96%, 97%, 98%, or 99% identity to a CDR-L1 selected from any one of SEQ ID NOs: 21 and 24. In some embodiments, the CDR-H3 is a CDR-H3 selected from any one of SEQ ID NOs: 13 and 16, with up to 1, 2, 3, 4, 5, 6, 7, or 8 amino acid substitutions; the CDR-H2 is a CDR-H2 selected from any one of SEQ ID NOs: 10, 11, and 12, with up to 1, 2, 3, 4, 5, 6, 7, or 8 amino acid substitutions; the CDR-H1 is a CDR-H1 selected from any one of SEQ ID NOs: 3, 6, and 9, with up to 1, 2, 3, 4, or 5 amino acid substitutions; the CDR-L3 is a CDR-L3 set forth in SEQ ID NO: 27 with up to 1, 2, 3, 4, or 5 amino acid substitutions; the CDR-L2 is a CDR-L2 selected from SEQ ID NO: 26 and the amino acid sequence DDS, with up to 1, 2, 3, or 4 amino acid substitutions; and the CDR-L1 is a CDR-L1 selected from any one of SEQ ID NOs: 21 and 24, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 13 and 16, the CDR-H2 has at least about 80%, 90%, 91%, 92%, 93%, 94%, 95%, 96%, 97%, 98%, or 99% identity to a CDR-H2 set forth in any one of SEQ ID NOs: 10, 11, and 12, the CDR-H1 has at least about 80%, 90%, 91%, 92%, 93%, 94%, 95%, 96%, 97%, 98%, or 99% identity to a CDR-H1 set forth in any one of SEQ ID NOs: 3, 6, and 9, the CDR-L3 has at least about 80%, 90%, 91%, 92%, 93%, 94%, 95%, 96%, 97%, 98%, or 99% identity to a CDR-L3 set forth in SEQ ID NO: 30, the CDR-L2 has at least about 80%, 90%, 91%, 92%, 93%, 94%, 95%, 96%, 97%, 98%, or 99% identity to a CDR-L2 set forth in SEQ ID NO: 25 or the amino acid sequence DVS, and the CDR-L1 has at least about 80%, 90%, 91%, 92%, 93%, 94%, 95%, 96%, 97%, 98%, or 99% identity to a CDR-L1 set forth in any one of SEQ ID NOs: 19 and 22. In some embodiments, the CDR-H3 is a CDR-H3 set forth in any one of SEQ ID NOs: 13 and 16, with up to 1, 2, 3, 4, 5, 6, 7, or 8 amino acid substitutions; the CDR-H2 is a CDR-H2 set forth in any one of SEQ ID NOs: 10, 11, and 12, with up to 1, 2, 3, 4, 5, 6, 7, or 8 amino acid substitutions; the CDR-H1 is a CDR-H1 set forth in any one of SEQ ID NOs: 3, 6, and 9, with up to 1, 2, 3, 4, or 5 amino acid substitutions; the CDR-L3 is a CDR-L3 set forth in SEQ ID NO: 30 with up to 1, 2, 3, 4, or 5 amino acid substitutions; the CDR-L2 is a CDR-L2 set forth in SEQ ID NO: 25 or the amino acid sequence DVS, with up to 1, 2, 3, or 4 amino acid substitutions; and the CDR-L1 is a CDR-L1 set forth in any one of SEQ ID NOs: 19 and 22, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 13 and 16, the CDR-H2 has at least about 80%, 90%, 91%, 92%, 93%, 94%, 95%, 96%, 97%, 98%, or 99% identity to a CDR-H2 set forth in any one of SEQ ID NOs: 10, 11, and 12, the CDR-H1 has at least about 80%, 90%, 91%, 92%, 93%, 94%, 95%, 96%, 97%, 98%, or 99% identity to a CDR-H1 set forth in any one of SEQ ID NOs: 3, 6, and 9, the CDR-L3 has at least about 80%, 90%, 91%, 92%, 93%, 94%, 95%, 96%, 97%, 98%, or 99% identity to a CDR-L3 set forth in SEQ ID NO: 392, the CDR-L2 has at least about 80%, 90%, 91%, 92%, 93%, 94%, 95%, 96%, 97%, 98%, or 99% identity to a CDR-L2 set forth in SEQ ID NO: 26 or the amino acid sequence DDS, and the CDR-L1 has at least about 80%, 90%, 91%, 92%, 93%, 94%, 95%, 96%, 97%, 98%, or 99% identity to a CDR-L1 set forth in any one of SEQ ID NOs: 351 and 365. In some embodiments, the CDR-H3 is a CDR-H3 set forth in any one of SEQ ID NOs: 13 and 16, with up to 1, 2, 3, 4, 5, 6, 7, or 8 amino acid substitutions; the CDR-H2 is a CDR-H2 set forth in any one of SEQ ID NOs: 10, 11, and 12, with up to 1, 2, 3, 4, 5, 6, 7, or 8 amino acid substitutions; the CDR-H1 is a CDR-H1 set forth in any one of SEQ ID NOs: 3, 6, and 9, with up to 1, 2, 3, 4, or 5 amino acid substitutions; the CDR-L3 is a CDR-L3 set forth in SEQ ID NO: 392 with up to 1, 2, 3, 4, or 5 amino acid substitutions; the CDR-L2 is a CDR-L2 set forth in SEQ ID NO: 26 or the amino acid sequence DDS, with up to 1, 2, 3, or 4 amino acid substitutions; and the CDR-L1 is a CDR-L1 set forth in any one of SEQ ID NOs: 351 and 365, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 351 and 365; a CDR-L2 comprising the sequence set forth in SEQ ID NO: 26 or the amino acid sequence DDS; and a CDR-L3 comprising the sequence set forth in SEQ ID NO: 392.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 13 and 16, the CDR-H2 has at least about 80%, 90%, 91%, 92%, 93%, 94%, 95%, 96%, 97%, 98%, or 99% identity to a CDR-H2 set forth in any one of SEQ ID NOs: 10, 11, and 12, the CDR-H1 has at least about 80%, 90%, 91%, 92%, 93%, 94%, 95%, 96%, 97%, 98%, or 99% identity to a CDR-H1 set forth in any one of SEQ ID NOs: 3, 6, and 9, the CDR-L3 has at least about 80%, 90%, 91%, 92%, 93%, 94%, 95%, 96%, 97%, 98%, or 99% identity to a CDR-L3 set forth in SEQ ID NO: 396, the CDR-L2 has at least about 80%, 90%, 91%, 92%, 93%, 94%, 95%, 96%, 97%, 98%, or 99% identity to a CDR-L2 set forth in SEQ ID NO: 26 or the amino acid sequence DDS, and the CDR-L1 has at least about 80%, 90%, 91%, 92%, 93%, 94%, 95%, 96%, 97%, 98%, or 99% identity to a CDR-L1 set forth in any one of SEQ ID NOs: 356 and 372. In some embodiments, the CDR-H3 is a CDR-H3 set forth in any one of SEQ ID NOs: 13 and 16, with up to 1, 2, 3, 4, 5, 6, 7, or 8 amino acid substitutions; the CDR-H2 is a CDR-H2 set forth in any one of SEQ ID NOs: 10, 11, and 12, with up to 1, 2, 3, 4, 5, 6, 7, or 8 amino acid substitutions; the CDR-H1 is a CDR-H1 set forth in any one of SEQ ID NOs: 3, 6, and 9, with up to 1, 2, 3, 4, or 5 amino acid substitutions; the CDR-L3 is a CDR-L3 set forth in SEQ ID NO: 396, with up to 1, 2, 3, 4, or 5 amino acid substitutions; the CDR-L2 is a CDR-L2 set forth in SEQ ID NO: 26 or the amino acid sequence DDS, with up to 1, 2, 3, or 4 amino acid substitutions; and the CDR-L1 is a CDR-L1 set forth in any one of SEQ ID NOs: 356 and 372, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 356 and 372; a CDR-L2 comprising the sequence set forth in SEQ ID NO: 26 or the amino acid sequence DDS; and a CDR-L3 comprising the sequence set forth in SEQ ID NO: 396.

In some embodiments, an antibody, or antigen binding fragment thereof, provided herein comprises a CDR-H1 comprising an amino acid sequence set forth in any one of SEQ ID NOs: 1, 4, and 7, a CDR-H2 comprising an amino acid sequence set forth in any one of SEQ ID NOs: 10, 11, and 12, a CDR-H3 comprising an amino acid sequence set forth in any one of SEQ ID NOs: 13 and 16, a CDR-L1 comprising an amino acid sequence set forth in any one of SEQ ID NOs: 19 and 22, a CDR-L2 comprising an amino acid sequence set forth in SEQ ID NO: 25 or the amino acid sequence DVS, and a CDR-L3 comprising an amino acid sequence set forth in SEQ ID NO: 27.

In some embodiments, an antibody, or antigen binding fragment thereof, provided herein comprises a CDR-H1 comprising an amino acid sequence set forth in any one of SEQ ID NOs: 2, 5, and 8, a CDR-H2 comprising an amino acid sequence set forth in any one of SEQ ID NOs: 10, 11, and 12, a CDR-H3 comprising an amino acid sequence set forth in any one of SEQ ID NOs: 13 and 16, a CDR-L1 comprising an amino acid sequence set forth in any one of SEQ ID NOs: 19 and 22, a CDR-L2 comprising an amino acid sequence set forth in SEQ ID NO: 25 or the amino acid sequence DVS, and a CDR-L3 comprising an amino acid sequence set forth in SEQ ID NO: 28.

In some embodiments, an antibody, or antigen binding fragment thereof, provided herein comprises a CDR-H1 comprising an amino acid sequence set forth in any one of SEQ ID NOs: 2, 5, and 8, a CDR-H2 comprising an amino acid sequence set forth in any one of SEQ ID NOs: 10, 11, and 12, a CDR-H3 comprising an amino acid sequence set forth in any one of SEQ ID NOs: 14 and 17, a CDR-L1 comprising an amino acid sequence set forth in any one of SEQ ID NOs: 20 and 23, a CDR-L2 comprising an amino acid sequence set forth in SEQ ID NO: 26 or the amino acid sequence DDS, and a CDR-L3 comprising an amino acid sequence set forth in SEQ ID NO: 28.

In some embodiments, an antibody, or antigen binding fragment thereof, provided herein comprises a CDR-H1 comprising an amino acid sequence set forth in any one of SEQ ID NOs: 2, 5, and 8, a CDR-H2 comprising an amino acid sequence set forth in any one of SEQ ID NOs: 10, 11, and 12, a CDR-H3 comprising an amino acid sequence set forth in any one of SEQ ID NOs: 13 and 16, a CDR-L1 comprising an amino acid sequence set forth in any one of SEQ ID NOs: 21 and 24, a CDR-L2 comprising an amino acid sequence set forth in SEQ ID NO: 25 or the amino acid sequence DVS, and a CDR-L3 comprising an amino acid sequence set forth in SEQ ID NO: 28.

In some embodiments, an antibody, or antigen binding fragment thereof, provided herein comprises a CDR-H1 comprising an amino acid sequence set forth in any one of SEQ ID NOs: 3, 6, and 9, a CDR-H2 comprising an amino acid sequence set forth in any one of SEQ ID NOs: 10, 11, and 12, a CDR-H3 comprising an amino acid sequence set forth in any one of SEQ ID NOs: 15 and 18, a CDR-L1 comprising an amino acid sequence set forth in any one of SEQ ID NOs: 21 and 24, a CDR-L2 comprising an amino acid sequence set forth in SEQ ID NO: 25 or the amino acid sequence DVS, and a CDR-L3 comprising an amino acid sequence set forth in SEQ ID NO: 29.

In some embodiments, an antibody, or antigen binding fragment thereof, provided herein comprises a CDR-H1 comprising an amino acid sequence set forth in any one of SEQ ID NOs: 1, 4, and 7, a CDR-H2 comprising an amino acid sequence set forth in any one of SEQ ID NOs: 10, 11, and 12, a CDR-H3 comprising an amino acid sequence set forth in any one of SEQ ID NOs: 13 and 16, a CDR-L1 comprising an amino acid sequence set forth in any one of SEQ ID NOs: 19 and 22, a CDR-L2 comprising an amino acid sequence set forth in SEQ ID NO: 26 or the amino acid sequence DDS, and a CDR-L3 comprising an amino acid sequence set forth in SEQ ID NO: 31.

In some embodiments, an antibody, or antigen binding fragment thereof, provided herein comprises a CDR-H1 comprising an amino acid sequence set forth in any one of SEQ ID NOs: 2, 5, and 8, a CDR-H2 comprising an amino acid sequence set forth in any one of SEQ ID NOs: 10, 11, and 12, a CDR-H3 comprising an amino acid sequence set forth in any one of SEQ ID NOs: 15 and 18, a CDR-L1 comprising an amino acid sequence set forth in any one of SEQ ID NOs: 21 and 24, a CDR-L2 comprising an amino acid sequence set forth in SEQ ID NO: 26 or the amino acid sequence DDS, and a CDR-L3 comprising an amino acid sequence set forth in SEQ ID NO: 28.

In some embodiments, the CDR-H3 has at least about 80%, 90%, 91%, 92%, 93%, 94%, 95%, 96%, 97%, 98%, or 99% identity to a CDR-H3 of TABLE 3; the CDR-H2 has at least about 80%, 90%, 91%, 92%, 93%, 94%, 95%, 96%, 97%, 98%, or 99% identity to a CDR-H2 of TABLE 3, the CDR-H1 has at least about 80%, 90%, 91%, 92%, 93%, 94%, 95%, 96%, 97%, 98%, or 99% identity to a CDR-H1 of TABLE 3, the CDR-L3 has at least about 80%, 90%, 91%, 92%, 93%, 94%, 95%, 96%, 97%, 98%, or 99% identity to a CDR-L3 of TABLE 3, the CDR-L2 has at least about 80%, 90%, 91%, 92%, 93%, 94%, 95%, 96%, 97%, 98%, or 99% identity to a CDR-L2 of TABLE 3, and the CDR-L1 has at least about 80%, 90%, 91%, 92%, 93%, 94%, 95%, 96%, 97%, 98%, or 99% identity to a CDR-L1 of TABLE 3. In some embodiments, the CDR-H3 is a CDR-H3 of TABLE 3, with up to 1, 2, 3, 4, 5, 6, 7, or 8 amino acid substitutions; the CDR-H2 is a CDR-H2 of TABLE 3, with up to 1, 2, 3, 4, 5, 6, 7, or 8 amino acid substitutions; the CDR-H1 is a CDR-H1 of TABLE 3, with up to 1, 2, 3, 4, or 5 amino acid substitutions; the CDR-L3 is a CDR-L3 of TABLE 3 with up to 1, 2, 3, 4, or 5 amino acid substitutions; the CDR-L2 is a CDR-L2 of TABLE 3, with up to 1, 2, 3, or 4 amino acid substitutions; and the CDR-L1 is a CDR-L1 of TABLE 3, with up to 1, 2, 3, 4, 5, or 6 amino acid substitutions.

In some embodiments, the CDR-H3 has at least about 80%, 90%, 91%, 92%, 93%, 94%, 95%, 96%, 97%, 98%, or 99% identity to a CDR-H3 of TABLE 5; the CDR-H2 has at least about 80%, 90%, 91%, 92%, 93%, 94%, 95%, 96%, 97%, 98%, or 99% identity to a CDR-H2 of TABLE 5, the CDR-H1 has at least about 80%, 90%, 91%, 92%, 93%, 94%, 95%, 96%, 97%, 98%, or 99% identity to a CDR-H1 of TABLE 5, the CDR-L3 has at least about 80%, 90%, 91%, 92%, 93%, 94%, 95%, 96%, 97%, 98%, or 99% identity to a CDR-L3 of TABLE 5, the CDR-L2 has at least about 80%, 90%, 91%, 92%, 93%, 94%, 95%, 96%, 97%, 98%, or 99% identity to a CDR-L2 of TABLE 5, and the CDR-L1 has at least about 80%, 90%, 91%, 92%, 93%, 94%, 95%, 96%, 97%, 98%, or 99% identity to a CDR-L1 of TABLE 5. In some embodiments, the CDR-H3 is a CDR-H3 of TABLE 5, with up to 1, 2, 3, 4, 5, 6, 7, or 8 amino acid substitutions; the CDR-H2 is a CDR-H2 of TABLE 5, with up to 1, 2, 3, 4, 5, 6, 7, or 8 amino acid substitutions; the CDR-H1 is a CDR-H1 of TABLE 5, with up to 1, 2, 3, 4, or 5 amino acid substitutions; the CDR-L3 is a CDR-L3 of TABLE 5, with up to 1, 2, 3, 4, or 5 amino acid substitutions; the CDR-L2 is a CDR-L2 of TABLE 5, with up to 1, 2, 3, or 4 amino acid substitutions; and the CDR-L1 is a CDR-L1 of TABLE 5, with up to 1, 2, 3, 4, 5, or 6 amino acid substitutions.

In certain embodiments of any of the antibodies, or antigen binding fragments thereof, described above, the antibody further comprises a heavy chain comprising a human IgG sequence selected from a sequence set forth in any one of SEQ ID NOs: 47-270. In certain embodiments of any of the antibodies, or antigen binding fragments thereof, described above, the antibody further comprises a constant light chain sequence comprising a sequence set forth in SEQ ID NO: 520. In certain embodiments of any of the antibodies, or antigen binding fragments thereof, described above, the antibody comprises a heavy chain comprising a human IgG sequence selected from SEQ ID NO: 61 or SEQ ID NO: 173 and a constant light chain sequence comprising a sequence set forth in SEQ ID NO: 520. In certain embodiments of any of the antibodies, or antigen binding fragments thereof, described above, the antibody comprises a heavy chain comprising a human IgG sequence set forth in SEQ ID NO: 173 and a constant light chain sequence comprising a sequence set forth in SEQ ID NO: 520.

In some embodiments, the antibody, or antigen binding fragment thereof, comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27. In certain embodiments, the antibody, or antigen binding fragment thereof, further comprises a heavy chain constant region (e.g., an IgG1 constant region) comprising LALA/YTE substitutions. In certain embodiments, the antibody, or antigen binding fragment thereof, comprises a heavy chain comprising a heavy chain constant region selected from SEQ ID NO: 61 or SEQ ID NO: 173. In certain embodiments, the antibody, or antigen binding fragment thereof, comprises a heavy chain comprising a heavy chain constant region set forth in SEQ ID NO: 173. In certain embodiments, the antibody, or antigen binding fragment thereof, comprises a human lambda light chain constant region (e.g., a lambda light chain constant region of subtype 1, 2, 3, or 7, such as a lambda light chain constant region set forth in any one of SEQ ID NOs: 520-523). In some embodiments, the antibody, or antigen binding fragment thereof, comprises a constant light chain sequence comprising a sequence set forth in SEQ ID NO: 520.

In some embodiments, an antibody, or antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NO: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30. In certain embodiments, the antibody, or antigen binding fragment thereof, further comprises a heavy chain constant region (e.g., an IgG1 constant region) comprising LALA/YTE substitutions. In certain embodiments, the antibody, or antigen binding fragment thereof, comprises a heavy chain comprising a heavy chain constant region selected from SEQ ID NO: 61 or SEQ ID NO: 173. In certain embodiments, the antibody, or antigen binding fragment thereof, comprises a heavy chain comprising a heavy chain constant region set forth in SEQ ID NO: 173. In certain embodiments, the antibody, or antigen binding fragment thereof, comprises a human lambda light chain constant region (e.g., a lambda light chain constant region of subtype 1, 2, 3, or 7, such as a lambda light chain constant region set forth in any one of SEQ ID NOs: 520-523). In some embodiments, the antibody, or antigen binding fragment thereof, comprises a constant light chain sequence comprising a sequence set forth in SEQ ID NO: 520.

Although the antibody may be produced using a polynucleotide sequence that encodes a constant heavy chain region containing a C-terminal lysine (e.g., a sequence encoding any one of SEQ ID NOs: 159-270), the C-terminal lysine may be cleaved off during manufacture or after administration (resulting in, e.g., an antibody containing a constant heavy chain sequence of any one of SEQ ID NOs: 47-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 40-158), or a mixture thereof (e.g., a mixture of the same heavy chain constant sequence with and without a C-terminal lysine, such as a mixture of SEQ ID NO: 173 and SEQ ID NO: 61).

In some aspects, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or clones are not derived from a sequence provided herein and may, for example, be isolated de novo according to the methods provided herein for obtaining antibodies.

### Anti-OX40L Antibodies

The present application provides antibodies or antigen binding fragments thereof and compositions comprising an antibody, or an antigen binding fragment thereof, which binds OX40L. In some embodiments, an anti-OX40L antibody, or antigen binding fragment thereof, described herein is selected from amlitelimab and oxelumab or a variant thereof comprising an Fc region with one or more amino acid substitutions as described herein. In some embodiments, amlitelimab comprises a variable heavy (VH) chain sequence having the amino acid sequence of SEQ ID NO: 448 and a variable light (VL) chain sequence having the amino acid sequence of SEQ ID NO: 449. In some embodiments, oxelumab comprises a VH chain sequence having the amino acid sequence of SEQ ID NO: 546 and a VL chain sequence having the amino acid sequence of SEQ ID NO: 547. In some embodiments, amlitelimab comprises a heavy chain having the amino acid sequence of SEQ ID NO: 548 and a light chain sequence having the amino acid sequence of SEQ ID NO: 549. In some embodiments, oxelumab comprises a heavy chain having the amino acid sequence of SEQ ID NO: 450 and a light chain sequence having the amino acid sequence of SEQ ID NO: 451, or a VH and/or VL therein. See also PCT Application No. PCT/US2024/026854 (corresponding to PCT Publication No. WO2024227174), which is incorporated herein by reference in its entirety. In some embodiments, an anti-OX40L antibody, or antigen binding fragment thereof, described herein is selected from an anti-OX40L antibody described therein.

Sequences of exemplary anti-OX40L antibodies, or antigen binding fragments thereof, are provided in TABLE 7.

TABLE 7
Sequences of OX40L Antibody Constructs - VH, VL, and Associated CDRs

| Construct | | |
|---|---|---|
| Identifier | VH and Heavy Chain CDRs | VL and Light Chain CDRs |
| 114 | VH (SEQ ID NO: 335) | VL (SEQ ID NO: 339) |
| | CDR1: Kabat (SEQ ID NO: 273) | CDR1: Kabat (SEQ ID NO: 309) |
| | Chothia (SEQ ID NO: 274) | Chothia (SEQ ID NO: 310) |
| | IMGT (SEQ ID NO: 275) | IMGT (SEQ ID NO: 311) |
| | CDR2: Kabat (SEQ ID NO: 285) | CDR2: Kabat (SEQ ID NO: 319) |
| | Chothia (SEQ ID NO: 286) | Chothia (amino acid sequence ASS) |
| | IMGT (SEQ ID NO: 287) | IMGT (amino acid sequence ASS) |
| | CDR3: Kabat (SEQ ID NO: 297) | CDR3: Kabat (SEQ ID NO: 327) |
| | Chothia (SEQ ID NO: 298) | Chothia (SEQ ID NO: 328) |
| | IMGT (SEQ ID NO: 299) | IMGT (SEQ ID NO: 327) |
| 231 | VH (SEQ ID NO: 336) | VL (SEQ ID NO: 340) |
| | CDR1: Kabat (SEQ ID NO: 276) | CDR1: Kabat (SEQ ID NO: 312) |
| | Chothia (SEQ ID NO: 277) | Chothia (SEQ ID NO: 313) |
| | IMGT (SEQ ID NO: 278) | IMGT (SEQ ID NO: 343) |
| | CDR2: Kabat (SEQ ID NO: 288) | CDR2: Kabat (SEQ ID NO: 321) |
| | Chothia (SEQ ID NO: 289) | Chothia (amino acid sequence GAT) |
| | IMGT (SEQ ID NO: 290) | IMGT (amino acid sequence GAT) |
| | CDR3: Kabat (SEQ ID NO: 300) | CDR3: Kabat (SEQ ID NO: 329) |
| | Chothia (SEQ ID NO: 301) | Chothia (SEQ ID NO: 330) |
| | IMGT (SEQ ID NO: 302) | IMGT (SEQ ID NO: 329) |
| 328 | VH (SEQ ID NO: 337) | VL (SEQ ID NO: 341) |
| | CDR1: Kabat (SEQ ID NO: 279) | CDR1: Kabat (SEQ ID NO: 344) |
| | Chothia (SEQ ID NO: 280) | Chothia (SEQ ID NO: 314) |
| | IMGT (SEQ ID NO: 281) | IMGT (SEQ ID NO: 315) |
| | CDR2: Kabat (SEQ ID NO: 291) | CDR2: Kabat (SEQ ID NO: 323) |
| | Chothia (SEQ ID NO: 292) | Chothia (amino acid sequence DAS) |
| | IMGT (SEQ ID NO: 293) | IMGT (amino acid sequence DAS) |
| | CDR3: Kabat (SEQ ID NO: 303) | CDR3: Kabat (SEQ ID NO: 331) |
| | Chothia (SEQ ID NO: 304) | Chothia (SEQ ID NO: 332) |
| | IMGT (SEQ ID NO: 305) | IMGT (SEQ ID NO: 331) |
| 105 | VH (SEQ ID NO: 338) | VL (SEQ ID NO: 342) |
| | CDR1: Kabat (SEQ ID NO: 282) | CDR1: Kabat (SEQ ID NO: 316) |
| | Chothia (SEQ ID NO: 283) | Chothia (SEQ ID NO: 317) |
| | IMGT (SEQ ID NO: 284) | IMGT (SEQ ID NO: 318) |
| | CDR2: Kabat (SEQ ID NO: 294) | CDR2: Kabat (SEQ ID NO: 325) |
| | Chothia (SEQ ID NO: 295) | Chothia (amino acid sequence ATS) |
| | IMGT (SEQ ID NO: 296) | IMGT (amino acid sequence ATS) |
| | CDR3: Kabat (SEQ ID NO: 306) | CDR3: Kabat (SEQ ID NO: 333) |
| | Chothia (SEQ ID NO: 307) | Chothia (SEQ ID NO: 334) |
| | IMGT (SEQ ID NO: 308) | IMGT (SEQ ID NO: 333) |
| Amlitelimab | VH (SEQ ID NO: 448) | VL (SEQ ID NO: 449) |
| | CDR1: Kabat (SEQ ID NO: 591) | CDR1: Kabat (SEQ ID NO: 599) |
| | Chothia (SEQ ID NO: 594) | Chothia (SEQ ID NO: 599) |
| | IMGT (SEQ ID NO: 596) | IMGT (SEQ ID NO: 602) |
| | CDR2: Kabat (SEQ ID NO: 592) | CDR2: Kabat (SEQ ID NO: 600) |
| | Chothia (SEQ ID NO: 595) | Chothia (SEQ ID NO: 600) |
| | IMGT (SEQ ID NO: 597) | IMGT (amino acid sequence: AAS) |
| | CDR3: Kabat (SEQ ID NO: 593) | CDR3: Kabat (SEQ ID NO: 601) |
| | Chothia (SEQ ID NO: 593) | Chothia (SEQ ID NO: 601) |
| | IMGT (SEQ ID NO: 598) | IMGT (SEQ ID NO: 601) |
| Oxelumab | VH (SEQ ID NO: 546) | VL (SEQ ID NO: 547) |

### Anti-OX40L VH Domains

In some embodiments, an antibody, or an antigen binding fragment thereof, provided herein comprises a VH sequence selected from any one of SEQ ID NOs: 335-338.

In some embodiments, an antibody, or an antigen binding fragment thereof, provided herein comprises a VH sequence having at least about 80%, 90%, 91%, 92%, 93%, 94%, 95%, 96%, 97%, 98%, or 99% identity to a VH sequence provided in any one of SEQ ID NOs: 335-338. In some embodiments, an antibody, or an antigen binding fragment thereof, provided herein comprises a VH sequence provided in any one of SEQ ID NOs: 335-338, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### Anti-OX40L VL Domains

In some embodiments, an antibody, or an antigen binding fragment thereof, provided herein comprises a VL sequence selected from any one of SEQ ID NOs: 339-342.

In some embodiments, an antibody, or an antigen binding fragment thereof, provided herein comprises a VL sequence having at least about 80%, 90%, 91%, 92%, 93%, 94%, 95%, 96%, 97%, 98%, or 99% identity to a VL sequence provided in any one of SEQ ID NOs: 339-342. In some embodiments, an antibody, or an antigen binding fragment thereof, provided herein comprises a VL sequence provided in any one of SEQ ID NOs: 339-342, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### Anti-OX40L VH-VL Combinations

In some embodiments, an antibody, or an antigen binding fragment thereof, provided herein comprises a VH sequence selected from any one of SEQ ID NOs: 335-338; and a VL sequence selected from any one of SEQ ID NO: 339-342.

In certain aspects, any one of SEQ ID NOs: 335-338 can be combined with any one of SEQ ID NOs: 339-342.

In certain aspects, an antibody, or an antigen binding fragment thereof, provided herein comprises a VH sequence and a VL sequence of a construct provided in TABLE 7 (e.g., a VH sequence and a VL sequence from the same row of TABLE 7) or in PCT Application No. PCT/US2024/026854 (corresponding to PCT Publication No. WO2024227174), incorporated by reference herein in its entirety. In certain aspects, an antibody, or antigen binding fragment thereof, provided herein comprises a VH sequence and a VL sequence from Table 2 in PCT Application No. PCT/US2024/026854 (e.g., a VH sequence and a VL sequence from the same row of Table 2).

In some embodiments, an antibody, or an antigen binding fragment thereof, provided herein comprises a VH sequence having at least about 80%, 90%, 91%, 92%, 93%, 94%, 95%, 96%, 97%, 98%, or 99% identity to a VH sequence provided in any one of SEQ ID NOs: 335-338; and a VL sequence having at least about 80%, 90%, 91%, 92%, 93%, 94%, 95%, 96%, 97%, 98%, or 99% identity to a VL sequence provided in any one of SEQ ID NOs: 339-342. In some embodiments, an antibody, or an antigen binding fragment thereof, provided herein comprises a VH sequence provided in any one of SEQ ID NOs: 335-338, with up to 1, 2, 3, 4, 5, 6, 7, 8, 9, 10, 11, 12, 13, 14, 15, 16, 17, 18, 19, or 20 amino acid substitutions, and a VL sequence provided in any one of SEQ ID NOs: 339-342, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody, or an antigen binding fragment thereof, comprises a VH sequence set forth in SEQ ID NO: 335 and a VL sequence set forth in SEQ ID NO: 339.

In certain embodiments, the antibody, or an antigen binding fragment thereof, comprises a VH sequence set forth in SEQ ID NO: 336 and a VL sequence set forth in SEQ ID NO: 340.

In certain embodiments, the antibody, or an antigen binding fragment thereof, comprises a VH sequence set forth in SEQ ID NO: 337 and a VL sequence set forth in SEQ ID NO: 341.

In certain embodiments, the antibody, or an antigen binding fragment thereof, comprises a VH sequence set forth in SEQ ID NO: 338 and a VL sequence set forth in SEQ ID NO: 342.

In certain embodiments of any of the antibodies, or antigen binding fragments thereof, described above, the antibody further comprises a heavy chain comprising a human IgG sequence selected from a sequence set forth in any of SEQ ID NOs: 47-270. In certain embodiments, the antibody, or antigen binding fragment thereof, comprises a heavy chain comprising a human IgG sequence set forth in SEQ ID NO: 61 or SEQ ID NO: 173. In certain embodiments, the antibody, or antigen binding fragment thereof, comprises a heavy chain comprising a human IgG sequence set forth in SEQ ID NO: 173.

In certain embodiments, the antibody, or antigen binding fragment thereof, comprises a VH sequence set forth in SEQ ID NO: 335; and a VL sequence set forth in SEQ ID NO: 339; and the antibody further comprises a heavy chain comprising a human IgG sequence selected from a sequence set forth in any of SEQ ID NOs: 47-270. In certain embodiments, the antibody, or antigen binding fragment thereof, comprises a heavy chain comprising a human IgG sequence set forth in SEQ ID NO: 61 or SEQ ID NO: 173. In certain embodiments, the antibody, or antigen binding fragment thereof, comprises a heavy chain comprising a human IgG sequence set forth in SEQ ID NO: 173. In certain embodiments of any of the antibodies, or antigen binding fragments thereof, described above, the antibody further comprises a constant light chain sequence comprising a sequence set forth in SEQ ID NO: 46.

In certain embodiments, the antibody comprises a VH sequence set forth in SEQ ID NO: 335, a VL sequence set forth in SEQ ID NO: 339, a heavy chain constant region (e.g., an IgG1 constant region) comprising LALA/YTE substitutions, and a human kappa light chain constant region. In certain embodiments, the antibody comprises a VH sequence set forth in SEQ ID NO: 335, a VL sequence set forth in SEQ ID NO: 339, a heavy chain constant region comprising a sequence set forth in SEQ ID NO: 61 or SEQ ID NO: 173, and a light chain constant region comprising a sequence set forth in SEQ ID NO: 46. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 335; and a VL sequence having at least about 80%, 90%, 91%, 92%, 93%, 94%, 95%, 96%, 97%, 98%, or 99% identity to a VL sequence provided in SEQ ID NO: 339, a heavy chain constant region (e.g., an IgG1 constant region) comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 335; and a VL sequence having at least about 80%, 90%, 91%, 92%, 93%, 94%, 95%, 96%, 97%, 98%, or 99% identity to a VL sequence provided in SEQ ID NO: 339, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 46 or a sequence having at least about 80%, 90%, 91%, 92%, 93%, 94%, 95%, 96%, 97%, 98%, or 99% identity to SEQ ID NO: 46.

In certain embodiments, the antibody comprises a VH sequence set forth in SEQ ID NO: 335, a VL sequence set forth in SEQ ID NO: 339, a heavy chain constant region comprising a sequence set forth in SEQ ID NO: 173, and a light chain constant region comprising a sequence set forth in SEQ ID NO: 46. In some embodiments, the antibody comprises a heavy chain sequence set forth in SEQ ID NO: 543 or 544 and a light chain sequence set forth in SEQ ID NO: 545. In some embodiments, the antibody comprises a heavy chain sequence set forth in SEQ ID NO: 543 and a light chain sequence set forth in SEQ ID NO: 545. The heavy chain sequence of SEQ ID NO: 543 contains a C-terminal lysine, which may be cleaved off during manufacture or after administration, resulting in an antibody containing the heavy chain sequence of SEQ ID NO: 544.

In certain embodiments, the antibody, or antigen binding fragment thereof, comprises a VH sequence set forth in SEQ ID NO: 336; and a VL sequence set forth in SEQ ID NO: 340; and the antibody further comprises a heavy chain comprising a human IgG sequence selected from a sequence set forth in any of SEQ ID NOs: 47-270. In certain embodiments, the antibody, or antigen binding fragment thereof, comprises a heavy chain comprising a human IgG sequence set forth in SEQ ID NO: 61 or SEQ ID NO: 173. In certain embodiments, the antibody, or antigen binding fragment thereof, comprises a heavy chain comprising a human IgG sequence set forth in SEQ ID NO: 173. In certain embodiments of any of the antibodies, or antigen binding fragments thereof, described above, the antibody further comprises a constant light chain sequence comprising a sequence set forth in SEQ ID NO: 46.

In certain embodiments, the antibody comprises a VH sequence set forth in SEQ ID NO: 336, a VL sequence set forth in SEQ ID NO: 340, a heavy chain constant region (e.g., an IgG1 constant region) comprising LALA/YTE substitutions, and a human kappa light chain constant region. In certain embodiments, the antibody comprises a VH sequence set forth in SEQ ID NO: 336, a VL sequence set forth in SEQ ID NO: 340, a heavy chain constant region comprising a sequence set forth in SEQ ID NO: 61 or SEQ ID NO: 173, and a light chain constant region comprising a sequence set forth in SEQ ID NO: 46. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 336; and a VL sequence having at least about 80%, 90%, 91%, 92%, 93%, 94%, 95%, 96%, 97%, 98%, or 99% identity to a VL sequence provided in SEQ ID NO: 340, a heavy chain constant region (e.g., an IgG1 constant region) comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 336; and a VL sequence having at least about 80%, 90%, 91%, 92%, 93%, 94%, 95%, 96%, 97%, 98%, or 99% identity to a VL sequence provided in SEQ ID NO: 340, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 46 or a sequence having at least about 80%, 90%, 91%, 92%, 93%, 94%, 95%, 96%, 97%, 98%, or 99% identity to SEQ ID NO: 46.

In certain embodiments, the antibody comprises a VH sequence set forth in SEQ ID NO: 336, a VL sequence set forth in SEQ ID NO: 340, a heavy chain constant region comprising a sequence set forth in SEQ ID NO: 173, and a light chain constant region comprising a sequence set forth in SEQ ID NO: 46.

In certain embodiments, the antibody, or antigen binding fragment thereof, comprises a VH sequence set forth in SEQ ID NO: 337; and a VL sequence set forth in SEQ ID NO: 341; and the antibody further comprises a heavy chain comprising a human IgG sequence selected from a sequence set forth in any of SEQ ID NOs: 47-270. In certain embodiments, the antibody, or antigen binding fragment thereof, comprises a heavy chain comprising a human IgG sequence set forth in SEQ ID NO: 61 or SEQ ID NO: 173. In certain embodiments, the antibody, or antigen binding fragment thereof, comprises a heavy chain comprising a human IgG sequence set forth in SEQ ID NO: 173. In certain embodiments of any of the antibodies, or antigen binding fragments thereof, described above, the antibody further comprises a constant light chain sequence comprising a sequence set forth in SEQ ID NO: 46.

In certain embodiments, the antibody comprises a VH sequence set forth in SEQ ID NO: 337, a VL sequence set forth in SEQ ID NO: 341, a heavy chain constant region (e.g., an IgG1 constant region) comprising LALA/YTE substitutions, and a human kappa light chain constant region. In certain embodiments, the antibody comprises a VH sequence set forth in SEQ ID NO: 337, a VL sequence set forth in SEQ ID NO: 341, a heavy chain constant region comprising a sequence set forth in SEQ ID NO: 61 or SEQ ID NO: 173, and a light chain constant region comprising a sequence set forth in SEQ ID NO: 46. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 337; and a VL sequence having at least about 80%, 90%, 91%, 92%, 93%, 94%, 95%, 96%, 97%, 98%, or 99% identity to a VL sequence provided in SEQ ID NO: 341, a heavy chain constant region (e.g., an IgG1 constant region) comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 337; and a VL sequence having at least about 80%, 90%, 91%, 92%, 93%, 94%, 95%, 96%, 97%, 98%, or 99% identity to a VL sequence provided in SEQ ID NO: 341, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 46 or a sequence having at least about 80%, 90%, 91%, 92%, 93%, 94%, 95%, 96%, 97%, 98%, or 99% identity to SEQ ID NO: 46.

In certain embodiments, the antibody comprises a VH sequence set forth in SEQ ID NO: 337, a VL sequence set forth in SEQ ID NO: 341, a heavy chain constant region comprising a sequence set forth in SEQ ID NO: 173, and a light chain constant region comprising a sequence set forth in SEQ ID NO: 46.

In certain embodiments, the antibody, or antigen binding fragment thereof, comprises a VH sequence set forth in SEQ ID NO: 338; and a VL sequence set forth in SEQ ID NO: 342; and the antibody further comprises a heavy chain comprising a human IgG sequence selected from a sequence set forth in any of SEQ ID NOs: 47-270. In certain embodiments, the antibody, or antigen binding fragment thereof, comprises a heavy chain comprising a human IgG sequence set forth in SEQ ID NO: 61 or SEQ ID NO: 173. In certain embodiments, the antibody, or antigen binding fragment thereof, comprises a heavy chain comprising a human IgG sequence set forth in SEQ ID NO: 173. In certain embodiments of any of the antibodies, or antigen binding fragments thereof, described above, the antibody further comprises a constant light chain sequence comprising a sequence set forth in SEQ ID NO: 46.

In certain embodiments, the antibody comprises a VH sequence set forth in SEQ ID NO: 338, a VL sequence set forth in SEQ ID NO: 342, a heavy chain constant region (e.g., an IgG1 constant region) comprising LALA/YTE substitutions, and a human kappa light chain constant region. In certain embodiments, the antibody comprises a VH sequence set forth in SEQ ID NO: 338, a VL sequence set forth in SEQ ID NO: 342, a heavy chain constant region comprising a sequence set forth in SEQ ID NO: 61 or SEQ ID NO: 173, and a light chain constant region comprising a sequence set forth in SEQ ID NO: 46. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 338; and a VL sequence having at least about 80%, 90%, 91%, 92%, 93%, 94%, 95%, 96%, 97%, 98%, or 99% identity to a VL sequence provided in SEQ ID NO: 342, a heavy chain constant region (e.g., an IgG1 constant region) comprising LALA/YTE substitutions, and a human kappa light chain constant region. In certain embodiments, the antibody comprises a VH sequence having at least about 80%, 90%, 91%, 92%, 93%, 94%, 95%, 96%, 97%, 98%, or 99% identity to a VH sequence provided in SEQ ID NO: 338; and a VL sequence having at least about 80%, 90%, 91%, 92%, 93%, 94%, 95%, 96%, 97%, 98%, or 99% identity to a VL sequence provided in SEQ ID NO: 342, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98%, or 99% identity to SEQ ID NO: 61 or SEQ ID NO: 173, and a light chain constant region comprising a sequence set forth in SEQ ID NO: 46 or a sequence having at least about 80%, 90%, 91%, 92%, 93%, 94%, 95%, 96%, 97%, 98%, or 99% identity to SEQ ID NO: 46.

In certain embodiments, the antibody comprises a VH sequence set forth in SEQ ID NO: 338, a VL sequence set forth in SEQ ID NO: 342, a heavy chain constant region comprising a sequence set forth in SEQ ID NO: 173, and a light chain constant region comprising a sequence set forth in SEQ ID NO: 46.

Although the antibody may be produced using a polynucleotide sequence that encodes a constant heavy chain region containing a C-terminal lysine (e.g., a sequence encoding any one of SEQ ID NOs: 159-270), the C-terminal lysine may be cleaved off during manufacture or after administration (resulting in, e.g., an antibody containing a constant heavy chain sequence of any one of SEQ ID NOs: 47-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 40-158), or a mixture thereof (e.g., a mixture of the same heavy chain constant region sequence with and without a C-terminal lysine, such as a mixture of SEQ ID NO: 173 and SEQ ID NO: 61).

### Anti-OX40L CDRs

In some embodiments, disclosed herein is an antibody, or antigen binding fragment thereof, comprising 1, 2, 3, 4, 5, or 6 of the CDRs of TABLE 7. In some embodiments, disclosed herein is an antibody, or antigen binding fragment thereof, comprising 6 of the Kabat CDRs of TABLE 7, 6 of the Chothia CDRs of TABLE 7, or 6 of the IMGT CDRs of TABLE 7. In some embodiments, the antibody, or an antigen binding fragment thereof, comprises 6 of the Kabat CDRs, 6 of the Chothia CDRs, or 6 of the IMGT CDRs from a single row of TABLE 7 (e.g., 6 CDRs from the same antibody).

In some embodiments, disclosed herein is an antibody, or an antigen binding fragment thereof, comprising 1, 2, 3, 4, 5, or 6 of the CDRs of an antibody provided in PCT Application No. PCT/US2024/026854 (corresponding to PCT Publication No. WO2024227174, incorporated by reference herein in its entirety, such as 1, 2, 3, 4, 5, or 6 of the CDRs in Table 2 in PCT Application No. PCT/US2024/026854. In some embodiments, disclosed herein is an antibody, or an antigen binding fragment thereof, comprising 1, 2, 3, 4, 5, or 6 of the Kabat CDRs of an antibody, or an antigen binding fragment thereof, provided in PCT Application No. PCT/US2024/026854. In some embodiments, disclosed herein is an antibody, or an antigen binding fragment thereof, comprising 1, 2, 3, 4, 5, or 6 of the Chothia CDRs of an antibody, or an antigen binding fragment thereof, provided in PCT Application No. PCT/US2024/026854. In some embodiments, disclosed herein is an antibody, or an antigen binding fragment thereof, comprising 1, 2, 3, 4, 5, or 6 of the IMGT CDRs of an antibody provided in PCT Application No. PCT/US2024/026854. In some embodiments, the antibody, or an antigen binding fragment thereof, comprises 6 of the Kabat CDRs, 6 of the Chothia CDRs, or 6 of the IMGT CDRs from a single row of Table 2 in PCT Application No. PCT/US2024/026854 (e.g., 6 CDRs from the same antibody).

In some embodiments, an antibody, or antigen binding fragment thereof, provided herein comprises one to three CDRs of a VH domain selected from any one of SEQ ID NOs: 335-338. In some embodiments, an antibody, or antigen binding fragment thereof, provided herein comprises two to three CDRs of a VH domain selected from any one of SEQ ID NOs: 335-338. In some embodiments, an antibody, or antigen binding fragment thereof, provided herein comprises three CDRs of a VH domain selected from any one of SEQ ID NOs: 335-338. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-H1, CDR-H2, or CDR-H3 selected from SEQ ID NOs: 273-308. In some embodiments, the CDR-H1 is a CDR-H1 of a VH domain selected from any one of SEQ ID NOs: 335-338, with up to 1, 2, 3, 4, or 5 amino acid substitutions. In some embodiments, the CDR-H2 is a CDR-H2 of a VH domain selected from any one of SEQ ID NOs: 335-338, with up to 1, 2, 3, 4, 5, 6, 7, or 8 amino acid substitutions. In some embodiments, the CDR-H3 is a CDR-H3 of a VH domain selected from any one of SEQ ID NOs: 335-338, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VL domain selected from any one of SEQ ID NOs: 339-342. In some embodiments, an antibody, or antigen binding fragment thereof, provided herein comprises two to three CDRs of a VL domain selected from any one of SEQ ID NOs: 339-342. In some embodiments, an antibody, or antigen binding fragment thereof, provided herein comprises three CDRs of a VL domain selected from any one of SEQ ID NOs: 339-342. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-L1, CDR-L2, or CDR-L3 selected from SEQ ID NOs: 309-319, 321, 323, 325, 327-334, 343, and 344 and any one of the amino acid sequences ASS, GAT, DAS, and ATS. In some embodiments, the CDR-L1 is a CDR-L1 of a VL domain selected from any one of SEQ ID NOs: 339-342, with up to 1, 2, 3, 4, or 5 amino acid substitutions. In some embodiments, the CDR-L2 is a CDR-L2 of a VL domain selected from any one of SEQ ID NOs: 339-342, with up to 1, 2, 3, 4, 5, 6, 7, or 8 amino acid substitutions. In some embodiments, the CDR-L3 is a CDR-L3 of a VL domain selected from any one of SEQ ID NOs: 339-342, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VH domain selected from any one of SEQ ID NOs: 335-338 and one to three CDRs of a VL domain selected from any one of SEQ ID NO: 339-342. In some embodiments, an antibody, or antigen binding fragment thereof, provided herein comprises two to three CDRs of a VH domain selected from any one of SEQ ID NOs: 335-338 and two to three CDRs of a VL domain selected from any one of SEQ ID NOs: 339-342. In some embodiments, an antibody, or antigen binding fragment thereof, provided herein comprises three CDRs of a VH domain selected from any one of SEQ ID NOs: 335-338 and three CDRs of a VL domain selected from any one of SEQ ID NOs: 339-342. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody, or antigen binding fragment thereof, provided herein comprises a CDR-H3 selected from any one of SEQ ID NOs: 297-308. In some aspects, the CDR-H3 has at least about 80%, 90%, 91%, 92%, 93%, 94%, 95%, 96%, 97%, 98%, or 99% identity to a CDR-H3 selected from any one of SEQ ID NOs: 297-308. In some embodiments, the CDR-H3 is a CDR-H3 selected from any one of SEQ ID NOs: 297-308, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H1 selected from any one of SEQ ID NOs: 273-284. In some aspects, the CDR-H1 has at least about 80%, 90%, 91%, 92%, 93%, 94%, 95%, 96%, 97%, 98%, or 99% identity to a CDR-H1 selected from any one of SEQ ID NOs: 273-284. In some embodiments, the CDR-H1 is a CDR-H1 selected from any one of SEQ ID NOs: 273-284, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H2 selected from any one of SEQ ID NOs: 285-296. In some aspects, the CDR-H2 has at least about 80%, 90%, 91%, 92%, 93%, 94%, 95%, 96%, 97%, 98%, or 99% identity to a CDR-H2 selected from any one of SEQ ID NOs: 285-296. In some embodiments, the CDR-H2 is a CDR-H2 selected from any one of SEQ ID NOs: 285-296, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L3 selected from any one of SEQ ID NOs: 327-334. In some aspects, the CDR-L3 has at least about 80%, 90%, 91%, 92%, 93%, 94%, 95%, 96%, 97%, 98% or 99% identity to a CDR-L3 of any one of SEQ ID NOs: 327-334. In some embodiments, the CDR-L3 selected from any one of SEQ ID NOs: 327-334, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L2 selected from any one of SEQ ID NOs: 319, 321, 323, and 325, and the amino acid sequences ASS, GAT, DAS, and ATS. In some aspects, the CDR-L2 has at least about 80%, 90%, 91%, 92%, 93%, 94%, 95%, 96%, 97%, 98%, or 99% identity to a CDR-L2 selected from any one of SEQ ID NOs: 319, 321, 323, and 325, and the amino acid sequences ASS, GAT, DAS, and ATS. In some embodiments, the CDR-L2 is a CDR-L2 selected from any one of SEQ ID NOs: 319, 321, 323, and 325, and the amino acid sequences ASS, GAT, DAS, and ATS, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L1 selected from any one of SEQ ID NOs: 309-318, 343, and 344. In some aspects, the CDR-L1 has at least about 80%, 90%, 91%, 92%, 93%, 94%, 95%, 96%, 97%, 98%, or 99% identity to a CDR-L1 selected from any one of SEQ ID NOs: 309-318, 343, and 344. In some embodiments, the CDR-L1 is a CDR-L1 selected from any one of SEQ ID NOs: 309-318, 343, and 344, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H3 selected from any one of SEQ ID NOs: 297-308, a CDR-H2 selected from any one of SEQ ID NOs: 285-296, a CDR-H1 selected from any one of SEQ ID NOs: 273-284, a CDR-L3 selected from any one of SEQ ID NOs: 327-334, a CDR-L2 selected from any one of SEQ ID NOs: 319, 321, 323, and 325, and the amino acid sequences ASS, GAT, DAS, and ATS, and a CDR-L1 selected from any one of SEQ ID NOs: 309-318, 343, and 344. In some embodiments, the CDR-H3 has at least about 80%, 90%, 91%, 92%, 93%, 94%, 95%, 96%, 97%, 98%, or 99% identity to a CDR-H3 selected from any one of SEQ ID NOs: 297-308, the CDR-H2 has at least about 80%, 90%, 91%, 92%, 93%, 94%, 95%, 96%, 97%, 98%, or 99% identity to a CDR-H2 selected from any one of SEQ ID NOs: 285-296, the CDR-H1 has at least about 80%, 90%, 91%, 92%, 93%, 94%, 95%, 96%, 97%, 98%, or 99% identity to a CDR-H1 selected from any one of SEQ ID NOs: 273-284, the CDR-L3 has at least about 80%, 90%, 91%, 92%, 93%, 94%, 95%, 96%, 97%, 98%, or 99% identity to a CDR-L3 selected from any one of SEQ ID NOs: 327-334, the CDR-L2 has at least about 80%, 90%, 91%, 92%, 93%, 94%, 95%, 96%, 97%, 98%, or 99% identity to a CDR-L2 selected from any one of SEQ ID NOs: 319, 321, 323, and 325, and the amino acid sequences ASS, GAT, DAS, and ATS, and the CDR-L1 has at least about 80%, 90%, 91%, 92%, 93%, 94%, 95%, 96%, 97%, 98%, or 99% identity to a CDR-L1 selected from any one of SEQ ID NOs: 309-318, 343, and 344. In some embodiments, the CDR-H3 is a CDR-H3 selected from any one of SEQ ID NOs: 297-308, with up to 1, 2, 3, 4, 5, 6, 7, or 8 amino acid substitutions; the CDR-H2 is a CDR-H2 selected from any one of SEQ ID NOs: 285-296, with up to 1, 2, 3, 4, 5, 6, 7, or 8 amino acid substitutions; the CDR-H1 is a CDR-H1 selected from any one of SEQ ID NOs: 273-284, with up to 1, 2, 3, 4, or 5 amino acid substitutions; the CDR-L3 is a CDR-L3 selected from any one of SEQ ID NOs: 327-334, with up to 1, 2, 3, 4, or 5 amino acid substitutions; the CDR-L2 is a CDR-L2 selected from any one of SEQ ID NOs: 319, 321, 323, and 325, and the amino acid sequence ASS, GAT, DAS, and ATS, with up to 1, 2, 3, or 4 amino acid substitutions; and the CDR-L1 is a CDR-L1 selected from any one of SEQ ID NOs: 309-318, 343, and 344, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 297, 298, and 299, the CDR-H2 has at least about 80%, 90%, 91%, 92%, 93%, 94%, 95%, 96%, 97%, 98%, or 99% identity to a CDR-H2 set forth in any one of SEQ ID NOs: 285, 286, and 287, the CDR-H1 has at least about 80%, 90%, 91%, 92%, 93%, 94%, 95%, 96%, 97%, 98%, or 99% identity to a CDR-H1 set forth in any one of SEQ ID NOs: 273, 274, and 275, the CDR-L3 has at least about 80%, 90%, 91%, 92%, 93%, 94%, 95%, 96%, 97%, 98%, or 99% identity to a CDR-L3 set forth in any one of SEQ ID NOs: 327 and 328, the CDR-L2 has at least about 80%, 90%, 91%, 92%, 93%, 94%, 95%, 96%, 97%, 98%, or 99% identity to a CDR-L2 set forth in SEQ ID NO: 319 or the amino acid sequence ASS, and the CDR-L1 has at least about 80%, 90%, 91%, 92%, 93%, 94%, 95%, 96%, 97%, 98%, or 99% identity to a CDR-L1 set forth in any one of SEQ ID NOs: 309, 310, and 311. In some embodiments, the CDR-H3 is a CDR-H3 set forth in any one of SEQ ID NOs: 297, 298, and 299, with up to 1, 2, 3, 4, 5, 6, 7, or 8 amino acid substitutions; the CDR-H2 is a CDR-H2 set forth in any one of SEQ ID NOs: 285, 286, and 287, with up to 1, 2, 3, 4, 5, 6, 7, or 8 amino acid substitutions; the CDR-H1 is a CDR-H1 set forth in any one of SEQ ID NOs: 273, 274, and 275, with up to 1, 2, 3, 4, or 5 amino acid substitutions; the CDR-L3 is a CDR-L3 set forth in any one of SEQ ID NO: 327 or 328, with up to 1, 2, 3, 4, or 5 amino acid substitutions; the CDR-L2 is a CDR-L2 set forth in SEQ ID NO: 319 or the amino acid sequence ASS, with up to 1, 2, 3, or 4 amino acid substitutions; and the CDR-L1 is a CDR-L1 set forth in any one of SEQ ID NOs: 309, 310, and 311, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328.

In some embodiments, an antibody, or antigen binding fragment thereof, provided herein comprises a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 300, 301, and 302, the CDR-H2 has at least about 80%, 90%, 91%, 92%, 93%, 94%, 95%, 96%, 97%, 98%, or 99% identity to a CDR-H2 set forth in any one of SEQ ID NOs: 288, 289, and 290, the CDR-H1 has at least about 80%, 90%, 91%, 92%, 93%, 94%, 95%, 96%, 97%, 98%, or 99% identity to a CDR-H1 set forth in any one of SEQ ID NOs: 276, 277, and 278, the CDR-L3 has at least about 80%, 90%, 91%, 92%, 93%, 94%, 95%, 96%, 97%, 98%, or 99% identity to a CDR-L3 set forth in any one of SEQ ID NOs: 329 and 330, the CDR-L2 has at least about 80%, 90%, 91%, 92%, 93%, 94%, 95%, 96%, 97%, 98%, or 99% identity to a CDR-L2 set forth in SEQ ID NO: 321 or the amino acid sequence GAT, and the CDR-L1 has at least about 80%, 90%, 91%, 92%, 93%, 94%, 95%, 96%, 97%, 98%, or 99% identity to a CDR-L1 set forth in any one of SEQ ID NOs: 312, 313, and 343. In some embodiments, the CDR-H3 is a CDR-H3 set forth in any one of SEQ ID NOs: 300, 301, and 302, with up to 1, 2, 3, 4, 5, 6, 7, or 8 amino acid substitutions; the CDR-H2 is a CDR-H2 set forth in any one of SEQ ID NOs: 288, 289, and 290, with up to 1, 2, 3, 4, 5, 6, 7, or 8 amino acid substitutions; the CDR-H1 is a CDR-H1 set forth in any one of SEQ ID NOs: 276, 277, and 278, with up to 1, 2, 3, 4, or 5 amino acid substitutions; the CDR-L3 is a CDR-L3 set forth in any one of SEQ ID NOs: 329 and 330 with up to 1, 2, 3, 4, or 5 amino acid substitutions; the CDR-L2 is a CDR-L2 set forth in SEQ ID NO: 321 or the amino acid sequence GAT, with up to 1, 2, 3, or 4 amino acid substitutions; and the CDR-L1 is a CDR-L1 set forth in any one of SEQ ID NOs: 312, 313, and 343, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NO: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330.

In some embodiments, an antibody, or antigen binding fragment thereof, provided herein comprises a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332. In some embodiments, the CDR-H3 has at least about 80%, 90%, 91%, 92%, 93%, 94%, 95%, 96%, 97%, 98%, or 99% identity to a CDR-H3 set forth in any one of SEQ ID NOs: 303, 304, and 305, the CDR-H2 has at least about 80%, 90%, 91%, 92%, 93%, 94%, 95%, 96%, 97%, 98%, or 99% identity to a CDR-H2 set forth in any one of SEQ ID NOs: 291, 292, and 293, the CDR-H1 has at least about 80%, 90%, 91%, 92%, 93%, 94%, 95%, 96%, 97%, 98%, or 99% identity to a CDR-H1 set forth in any one of SEQ ID NOs: 279, 280, and 281, the CDR-L3 has at least about 80%, 90%, 91%, 92%, 93%, 94%, 95%, 96%, 97%, 98%, or 99% identity to a CDR-L3 set forth in any one of SEQ ID NOs: 331 and 332, the CDR-L2 has at least about 80%, 90%, 91%, 92%, 93%, 94%, 95%, 96%, 97%, 98%, or 99% identity to a CDR-L2 set forth in SEQ ID NO: 323 or the amino acid sequence DAS, and the CDR-L1 has at least about 80%, 90%, 91%, 92%, 93%, 94%, 95%, 96%, 97%, 98%, or 99% identity to a CDR-L1 set forth in any one of SEQ ID NOs: 344, 314, and 315. In some embodiments, the CDR-H3 is a CDR-H3 set forth in any one of SEQ ID NOs: 303, 304, and 305, with up to 1, 2, 3, 4, 5, 6, 7, or 8 amino acid substitutions; the CDR-H2 is a CDR-H2 set forth in any one of SEQ ID NOs: 291, 292, and 293, with up to 1, 2, 3, 4, 5, 6, 7, or 8 amino acid substitutions; the CDR-H1 is a CDR-H1 set forth in any one of SEQ ID NOs: 279, 280, and 281, with up to 1, 2, 3, 4, or 5 amino acid substitutions; the CDR-L3 is a CDR-L3 set forth in any one of SEQ ID NOs: 331 and 332 with up to 1, 2, 3, 4, or 5 amino acid substitutions; the CDR-L2 is a CDR-L2 set forth in SEQ ID NO: 323 or the amino acid sequence DAS, with up to 1, 2, 3, or 4 amino acid substitutions; and the CDR-L1 is a CDR-L1 set forth in any one of SEQ ID NOs: 344, 314, and 315, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NO: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332.

In some embodiments, an antibody, or antigen binding fragment thereof, provided herein comprises a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334. In some embodiments, the CDR-H3 has at least about 80%, 90%, 91%, 92%, 93%, 94%, 95%, 96%, 97%, 98%, or 99% identity to a CDR-H3 set forth in any of SEQ ID NOs: 306, 307, and 308, the CDR-H2 has at least about 80%, 90%, 91%, 92%, 93%, 94%, 95%, 96%, 97%, 98%, or 99% identity to a CDR-H2 set forth in any one of SEQ ID NOs: 294, 295, and 296, the CDR-H1 has at least about 80%, 90%, 91%, 92%, 93%, 94%, 95%, 96%, 97%, 98%, or 99% identity to a CDR-H1 set forth in any one of SEQ ID NOs: 282, 283, and 284, the CDR-L3 has at least about 80%, 90%, 91%, 92%, 93%, 94%, 95%, 96%, 97%, 98%, or 99% identity to a CDR-L3 set forth in any one of SEQ ID NOs: 333 and 334, the CDR-L2 has at least about 80%, 90%, 91%, 92%, 93%, 94%, 95%, 96%, 97%, 98%, or 99% identity to a CDR-L2 set forth in SEQ ID NO: 325 or the amino acid sequence ATS, and the CDR-L1 has at least about 80%, 90%, 91%, 92%, 93%, 94%, 95%, 96%, 97%, 98%, or 99% identity to a CDR-L1 set forth in any one of SEQ ID NOs: 316, 317, and 318. In some embodiments, the CDR-H3 is a CDR-H3 set forth in any one of SEQ ID NOs: 306, 307, and 308, with up to 1, 2, 3, 4, 5, 6, 7, or 8 amino acid substitutions; the CDR-H2 is a CDR-H2 set forth in any one of SEQ ID NOs: 294, 295, and 296, with up to 1, 2, 3, 4, 5, 6, 7, or 8 amino acid substitutions; the CDR-H1 is a CDR-H1 set forth in any one of SEQ ID NOs: 282, 283, and 284, with up to 1, 2, 3, 4, or 5 amino acid substitutions; the CDR-L3 is a CDR-L3 set forth in any one of SEQ ID NOs: 333 and 334 with up to 1, 2, 3, 4, or 5 amino acid substitutions; the CDR-L2 is a CDR-L2 set forth in SEQ ID NO: 325 or the amino acid sequence ATS, with up to 1, 2, 3, or 4 amino acid substitutions; and the CDR-L1 is a CDR-L1 set forth in any one of SEQ ID NOs: 316, 317, and 318, with up to 1, 2, 3, 4, 5, or 6 amino acid substitutions.

In some embodiments, an antibody, or antigen binding fragment thereof, provided herein comprises a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NO: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334.

In some embodiments, the CDR-H3 has at least about 80%, 90%, 91%, 92%, 93%, 94%, 95%, 96%, 97%, 98%, or 99% identity to a CDR-H3 set forth in TABLE 7; the CDR-H2 has at least about 80%, 90%, 91%, 92%, 93%, 94%, 95%, 96%, 97%, 98%, or 99% identity to a CDR-H2 set forth in TABLE 7, the CDR-H1 has at least about 80%, 90%, 91%, 92%, 93%, 94%, 95%, 96%, 97%, 98%, or 99% identity to a CDR-H1 set forth in TABLE 7, the CDR-L3 has at least about 80%, 90%, 91%, 92%, 93%, 94%, 95%, 96%, 97%, 98%, or 99% identity to a CDR-L3 set forth in TABLE 7, the CDR-L2 has at least about 80%, 90%, 91%, 92%, 93%, 94%, 95%, 96%, 97%, 98%, or 99% identity to a CDR-L2 set forth in TABLE 7, and the CDR-L1 has at least about 80%, 90%, 91%, 92%, 93%, 94%, 95%, 96%, 97%, 98%, or 99% identity to a CDR-L1 set forth in TABLE 7. In some embodiments, the CDR-H3 is a CDR-H3 set forth in TABLE 7, with up to 1, 2, 3, 4, 5, 6, 7, or 8 amino acid substitutions; the CDR-H2 is a CDR-H2 set forth in TABLE 7, with up to 1, 2, 3, 4, 5, 6, 7, or 8 amino acid substitutions; the CDR-H1 is a CDR-H1 set forth in TABLE 7, with up to 1, 2, 3, 4, or 5 amino acid substitutions; the CDR-L3 is a CDR-L3 set forth in TABLE 7 with up to 1, 2, 3, 4, or 5 amino acid substitutions; the CDR-L2 is a CDR-L2 set forth in TABLE 7, with up to 1, 2, 3, or 4 amino acid substitutions; and the CDR-L1 is a CDR-L1 set forth in TABLE 7, with up to 1, 2, 3, 4, 5, or 6 amino acid substitutions.

In certain embodiments of any of the antibodies, or antigen binding fragments thereof, described above, the antibody further comprises a heavy chain comprising a human IgG sequence selected from a sequence set forth in any one of SEQ ID NOs: 47-270. In certain embodiments of any of the antibodies, or antigen binding fragments thereof, described above, the antibody further comprises a constant light chain sequence comprising a sequence set forth in SEQ ID NO: 46. In certain embodiments of any of the antibodies, or antigen binding fragments thereof, described above, the antibody further comprises a heavy chain comprising a human IgG sequence selected from SEQ ID NO: 61 or SEQ ID NO: 173 and a constant light chain sequence comprising a sequence set forth in SEQ ID NO: 46.

In some embodiments, the antibody, or antigen binding fragment thereof, comprises a CDR-H1 comprising the sequence set forth in any one of SEQ ID NOs: 273, 274, and 275; a CDR-H2 comprising the sequence set forth in any one of SEQ ID NOs: 285, 286, and 287; a CDR-H3 comprising the sequence set forth in any one of SEQ ID NOs: 297, 298, and 299; a CDR-L1 comprising the sequence set forth in any one of SEQ ID NOs: 309, 310, and 311; a CDR-L2 comprising the sequence set forth in SEQ ID NO: 319 or the amino acid sequence ASS; and a CDR-L3 comprising the sequence set forth in any one of SEQ ID NOs: 327 and 328. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 and SEQ ID NO: 173. In certain embodiments, the antibody comprises a heavy chain comprising a heavy chain constant region set forth in SEQ ID NO: 173. In certain embodiments, the antibody comprises a human kappa light chain constant region. In some embodiments, the antibody comprises a constant light chain sequence comprising a sequence set forth in SEQ ID NO: 46.

In some embodiments, an antibody, or antigen binding fragment thereof, provided herein comprises a CDR-H1 comprising the sequence set forth in any one of SEQ ID NOs: 276, 277, and 278; a CDR-H2 comprising the sequence set forth in any one of SEQ ID NOs: 288, 289, and 290; a CDR-H3 comprising the sequence set forth in any one of SEQ ID NOs: 300, 301, and 302; a CDR-L1 comprising the sequence set forth in any one of SEQ ID NOs: 312, 313, and 343; a CDR-L2 comprising the sequence set forth in SEQ ID NO: 321 or the amino acid sequence GAT; and a CDR-L3 comprising the sequence set forth in any one of SEQ ID NOs: 329 and 330. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 and SEQ ID NO: 173. In certain embodiments, the antibody comprises a heavy chain comprising a heavy chain constant region set forth in SEQ ID NO: 173. In certain embodiments, the antibody comprises a human kappa light chain constant region. In some embodiments, the antibody comprises a constant light chain sequence comprising a sequence set forth in SEQ ID NO: 46.

In some embodiments, an antibody, or antigen binding fragment thereof, provided herein comprises a CDR-H1 comprising the sequence set forth in any one of SEQ ID NOs: 279, 280, and 281; a CDR-H2 comprising the sequence set forth in any one of SEQ ID NOs: 291, 292, and 293; a CDR-H3 comprising the sequence set forth in any one of SEQ ID NOs: 303, 304, and 305; a CDR-L1 comprising the sequence set forth in any one of SEQ ID NOs: 344, 314, and 315; a CDR-L2 comprising the sequence set forth in SEQ ID NO: 323 or the amino acid sequence DAS; and a CDR-L3 comprising the sequence set forth in any one of SEQ ID NOs: 331 and 332. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 and SEQ ID NO: 173. In certain embodiments, the antibody comprises a heavy chain comprising a heavy chain constant region set forth in SEQ ID NO: 173. In certain embodiments, the antibody comprises a human kappa light chain constant region. In some embodiments, the antibody comprises a constant light chain sequence comprising a sequence set forth in SEQ ID NO: 46.

In some embodiments, an antibody, or antigen binding fragment thereof, provided herein comprises a CDR-H1 comprising the sequence set forth in any one of SEQ ID NOs: 282, 283, and 284; a CDR-H2 comprising the sequence set forth in any one of SEQ ID NOs: 294, 295, and 296; a CDR-H3 comprising the sequence set forth in any one of SEQ ID NOs: 306, 307, and 308; a CDR-L1 comprising the sequence set forth in any one of SEQ ID NOs: 316, 317, and 318; a CDR-L2 comprising the sequence set forth in SEQ ID NO: 325 or the amino acid sequence ATS; and a CDR-L3 comprising the sequence set forth in any one of SEQ ID NOs: 333 and 334. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 and SEQ ID NO: 173. In certain embodiments, the antibody comprises a heavy chain comprising a heavy chain constant region set forth in SEQ ID NO: 173. In certain embodiments, the antibody comprises a human kappa light chain constant region. In some embodiments, the antibody comprises a constant light chain sequence comprising a sequence set forth in SEQ ID NO: 46.

Although the antibody may be produced using a polynucleotide sequence that encodes a constant heavy chain region containing a C-terminal lysine (e.g., a sequence encoding any one of SEQ ID NOs: 159-270), the C-terminal lysine may be cleaved off during manufacture or after administration (resulting in, e.g., an antibody containing a constant heavy chain sequence of any one of SEQ ID NOs: 47-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 40-158), or a mixture thereof (e.g., a mixture of the same heavy chain constant region sequence with and without a C-terminal lysine, such as a mixture of SEQ ID NO: 173 and SEQ ID NO: 61).

In some aspects, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or clones are not derived from a sequence provided herein and may, for example, be isolated de novo according to the methods provided herein for obtaining antibodies

### Fc Region

The structures of the Fc regions of various immunoglobulins, and the glycosylation sites contained therein, are known in the art. See Schroeder and Cavacini, J. (2010) Allergy Clin. Immunol. 125: S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

In certain embodiments, provided human IgG1 Fc regions include an SRDEL (SEQ ID NO: 588) allotype or an SREEM (SEQ ID NO: 589) allotype.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known that can selectively alter the affinity of the Fc for different Fcgamma receptors. In some aspects, the Fc comprises one or more modifications designed to promote selective binding of Fc-gamma receptors.

Exemplary mutations that may alter the binding of FcRs to the Fc are listed below (in EU numbering format):
S298A/E333A/K334A, S298A/E333A/K334A/K326A (Lu Y, Vernes J M, Chiang N, et al., J Immunol Methods. 2011 Feb. 28; 365 (1-2): 132-41);
F243L/R292P/Y300L/V305I/P396L, F243L/R292P/Y300L/L235V/P396L (Stavenhagen J B, Gorlatov S, Tuaillon N, et al., Cancer Res. 2007 Sep. 15; 67 (18): 8882-90; Nordstrom J L, Gorlatov S, Zhang W, et al., Breast Cancer Res. 2011 Nov. 30; 13 (6): R123);
F243L (Stewart R, Thom G, Levens M, et al., Protein Eng Des Sel. 2011 September; 24 (9): 671-8), S298A/E333A/K334A (Shields R L, Namenuk A K, Hong K, et al., J Biol Chem. 2001 Mar. 2; 276 (9): 6591-604);
S239D/I332E/A330L, S239D/I332E (Lazar G A, Dang W, Karki S, et al., Proc Natl Acad Sci USA. 2006 Mar. 14; 103 (11): 4005-10);
S239D/S267E, S267E/L328F (Chu S Y, Vostiar I, Karki S, et al., Mol Immunol. 2008 September; 45 (15): 3926-33);
S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I332 E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, an antibody, or antigen binding fragment thereof, described herein includes modifications intended to improve its ability to mediate effector function. “Effector functions” refer to those biological activities mediated by the Fc region of an antibody, which activities may vary depending on the antibody isotype. Examples of antibody effector functions include receptor ligand blocking, agonism or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the anti-TSLP antibody and/or anti-OX40L antibody described herein includes modifications designed to improve its ability to mediate effector function. Such modifications that can have this effect are known in the art and include afucosylation, or engineering of the affinity of the Fc towards an activating receptor, mainly FCGR3a for ADCC, and towards C1q for CDC. The following TABLE 8 summarizes various designs reported in the literature for effector function engineering.

Methods of producing antibodies with little or no fucose on the Fc glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells (von Horsten et al. (2010) Glycobiology. 2010 December; 20 (12): 1607-18). Examples of cell lines capable of producing defucosylated antibody include CHO-DG44 with stable overexpression of the bacterial oxidoreductase GDP-6-deoxy-D-lyxo-4-hexylose reductase (RMD) (see von Horsten et al. (2010) supra) or Lec13 CHO cells, which are deficient in protein fucosylation (see Ripka et al. (1986) Arch. Biochem. Biophys. 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al. (2004) Biotech. Bioeng. 87:614-622; Kanda et al. (2006) Biotechnol. Bioeng. 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, the anti-OX40L and/or anti-TSLP antibody provided herein comprises an Fc domain (e.g., an IgG1 domain) with reduced fucose content at position Asn 297 (EU numbering) compared to a naturally occurring Fc domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In some embodiments, the anti-OX40L and/or anti-TSLP antibody described herein comprises an Fc region with one or more amino acid substitutions which improve ADCC, such as a substitution at one or more of positions 298, 333, and 334 of the Fc region. In some embodiments, the anti-OX40L and/or anti-TSLP antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87:614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

In some embodiments, the effector function of the anti-OX40L antibody, or antigen binding fragment thereof, described herein is antagonism and blocks OX40L binding to OX40.

In some embodiments, the effector function of the anti-TSLP antibody, or antigen binding fragment thereof, described herein is antagonism and blocks TSLP from binding to a TSLP receptor.

Thus, in one embodiment, an antibody described herein can include a dimeric Fc that comprises one or more amino acid modifications as noted in TABLE 8 that may confer improved effector function. In another embodiment, the antibody can be afucosylated to improve effector function.

TABLE 8
CH2 domains and effector function engineering

| Reference | Mutations | Effect |
|---|---|---|
| Lu (2011), supra, Ferrara | Afucosylated | Increased ADCC |
| (2011), supra, Mizushima | | |
| et al. (2011) Genes Cells | | |
| 16(11): 1071-1080. | | |
| Lu (2011), supra | S298A/E333A/K334A | Increased ADCC |
| Lu (2011), supra | S298A/E333A/K334A/K326A | Increased ADCC |
| Stavenhagen (2007), | F243L/R292P/Y300L/V305I/P396L | Increased ADCC |
| supra | | |
| Nordstrom (2011), supra | F243L/R292P/Y300L/L235V/P396L | Increased ADCC |
| Stewart (2011), supra | F243L | Increased ADCC |
| Shields (2001), supra | S298A/E333A/K334A | Increased ADCC |
| Lazar (2006), supra | S239D/1332E/A330L | Increased ADCC |
| Lazar (2006), supra | S239D/1332E | Increased ADCC |
| Bowles et al. (2006) | AME-D, not specified mutations | Increased ADCC |
| Blood 108(8):2648-54 | | |
| Heider et al. (2011) | 37.1, mutations not disclosed | Increased ADCC |
| Blood 118(15):4159-68 | | |
| Moore et al. (2010) MAbs | S267E/H268F/S324T | Increased CDC |
| 2(2): 181-9 | | |

Fc modifications intended to reduce FcgR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al. ((2012) J. Mol. Biol. 420:204-219), each of which is incorporated by reference in its entirety, describe specific modifications designed to reduce FcgR or complement binding to the Fc.

Specific, non-limiting examples of amino acid modifications intended to reduce FcgR or complement binding to the Fc include those identified in the following TABLE 9:

TABLE 9
Modifications designed to reduce FcgR
or complement binding to the Fc
Mutations

N297A
L234A/L235A
IgG2 V234A/G237A
IgG4 L235A/G237A/E318A
IgG4 S228P/L236E
IgG2/IgG4
IgG2 H268Q/V309L/A330S/A331S
C220S/C226S/C229S/P238S
C226S/C229S/E3233P/L235V/L235A
E. coli production, non glyco
L234F/L235E/P331S
Hinge mutant, e.g., C226S/P230S
D265A
L235A/G237A
L234A/L235A/G237A
L234A/L235A/P329G

In some embodiments, an antibody, or an antigen binding fragment thereof, provided herein comprises one or more alterations that is designed to improve or diminish C1q binding and/or CDC. See U.S. Pat. No. 6,194,551; WO 99/51642; and Idusogie et al., J. Immunol., 2000, 164:4178-4184; each of which is incorporated by reference in its entirety.

In certain embodiments, the heavy chain comprises a constant heavy chain sequence selected from a sequence set forth in any one of SEQ ID NOs: 47-270. In certain embodiments, the constant heavy chain sequence, (e.g., a constant heavy chain sequence selected from any one of SEQ ID NOs: 47-158) further comprises a C-terminal lysine (e.g., any one of SEQ ID NOs: 159-270). Although the antibody may be produced using a polynucleotide sequence that encodes a constant heavy chain region containing a C-terminal lysine, the C-terminal lysine may be cleaved off during manufacture or after administration. Accordingly, sequences of heavy chain constant regions with and without the C-terminal lysine are provided herein. Consequently, a composition resulting from the manufacture of an antibody using a polynucleotide sequence that encodes a constant heavy chain region containing a C-terminal lysine (e.g., an antibody with a coding sequence that encodes any one of SEQ ID NOs: 159-270) may comprise antibodies having a constant heavy chain sequence containing a C-terminal lysine (e.g., selected from SEQ ID NOs: 159-270), antibodies having a constant heavy chain sequence lacking a C-terminal lysine (e.g., the corresponding sequence of any one of SEQ ID NOs: 47-158), or a mixture thereof. For example, a composition comprising an antibody comprising the constant heavy chain sequence of SEQ ID NO: 173 that is administered to a subject may comprise antibodies having the constant heavy chain sequence set forth in SEQ ID NO: 173 or SEQ ID NO: 61, or a mixture thereof (e.g., a mixture of antibodies having either a constant heavy chain sequence of SEQ ID NO: 173 or a constant heavy chain sequence of SEQ ID NO: 61 and/or antibodies containing both constant heavy chain sequences (e.g., in a single antibody containing two constant heavy chain sequences)).

In certain embodiments, an anti-TSLP antibody, or antigen binding fragment thereof, provided herein comprises a VH sequence and a VL sequence provided in TABLE 4 (e.g., a VH sequence and a VL sequence from the same row of TABLE 4), together with a heavy chain constant region selected from a sequence set forth in any one of SEQ ID NOs: 47-270. In certain embodiments, an anti-TSLP antibody, or an antigen binding fragment thereof, provided herein comprises a VH sequence and a VL sequence provided in TABLE 4, together with a heavy chain constant region selected from a sequence set forth in any one of SEQ ID NOs: 47-270 and a light chain constant region selected from any one of SEQ ID NOs: 520-523.

In certain embodiments, an anti-TSLP antibody, or an antigen binding fragment thereof, provided herein comprises a VH sequence and a VL sequence provided in TABLE 6 (e.g., a VH sequence and a VL sequence from the same row of TABLE 6), together with a heavy chain constant region selected from a sequence set forth in any one of SEQ ID NOs: 47-270. In certain embodiments, an anti-TSLP antibody, or an antigen binding fragment thereof, provided herein comprises a VH sequence and a VL sequence provided in TABLE 6, together with a heavy chain constant region selected from a sequence set forth in any one of SEQ ID NOs: 47-270 and a light chain constant region selected from any one of SEQ ID NOs: 520-523.

In certain embodiments, the anti-TSLP antibody, or antigen binding fragment thereof, comprises a VH sequence set forth in SEQ ID NO: 34 and a VL sequence set forth in SEQ ID NO: 41; and the constant heavy chain comprises a human IgG sequence selected from a sequence set forth in any one of SEQ ID NOs: 47-270 (e.g., SEQ ID NO: 61 or SEQ ID NO: 173). In certain embodiments, the anti-TSLP antibody, or antigen binding fragment thereof, comprises a VH sequence set forth in SEQ ID NO: 34 and a VL sequence set forth in SEQ ID NO: 43; and the constant heavy chain comprises a human IgG sequence selected from a sequence set forth in any one of SEQ ID NOs: 47-270 (e.g., SEQ ID NO: 61 or SEQ ID NO: 173). In certain embodiments, the antibody, or antigen binding fragment thereof, comprises a human lambda light chain constant region sequence (e.g., any one of SEQ ID NOs: 520-523).

In certain embodiments, the anti-TSLP antibody comprises a heavy chain comprising an amino acid sequence set forth in SEQ ID NO: 524 and/or SEQ ID NO: 526. In certain embodiments, the anti-TSLP antibody comprises a light chain comprising an amino acid sequence set forth in SEQ ID NO: 525. In certain embodiments, the anti-TSLP antibody comprises a heavy chain comprising an amino acid sequence set forth in SEQ ID NO: 524 and a light chain comprising an amino acid sequence set forth in SEQ ID NO: 525. The heavy chain sequence of SEQ ID NO: 524 contains a C-terminal lysine, which may be cleaved off during manufacture or after administration, resulting in an antibody containing the heavy chain sequence of SEQ ID NO: 526. In certain embodiments, the anti-TSLP antibody comprises a heavy chain comprising an amino acid sequence set forth in SEQ ID NO: 526 and a light chain comprising an amino acid sequence set forth in SEQ ID NO: 525.

In certain embodiments, the anti-TSLP antibody comprises a heavy chain comprising an amino acid sequence set forth in SEQ ID NO: 527. In certain embodiments, the anti-TSLP antibody comprises a light chain comprising an amino acid sequence set forth in SEQ ID NO: 528. In certain embodiments, the anti-TSLP antibody comprises a heavy chain comprising an amino acid sequence set forth in SEQ ID NO: 527 and a light chain comprising an amino acid sequence set forth in SEQ ID NO: 528.

In certain embodiments, the anti-TSLP antibody comprises a heavy chain comprising an amino acid sequence set forth in SEQ ID NO: 529. In certain embodiments, the anti-TSLP antibody comprises a light chain comprising an amino acid sequence set forth in SEQ ID NO: 530. In certain embodiments, the anti-TSLP antibody comprises a heavy chain comprising an amino acid sequence set forth in SEQ ID NO: 529 and a light chain comprising an amino acid sequence set forth in SEQ ID NO: 530.

In certain embodiments, the anti-TSLP antibody comprises a heavy chain comprising an amino acid sequence set forth in SEQ ID NO: 531. In certain embodiments, the anti-TSLP antibody comprises a light chain comprising an amino acid sequence set forth in SEQ ID NO: 532. In certain embodiments, the anti-TSLP antibody comprises a heavy chain comprising an amino acid sequence set forth in SEQ ID NO: 531 and a light chain comprising an amino acid sequence set forth in SEQ ID NO: 532.

In certain embodiments, the anti-TSLP antibody comprises a heavy chain comprising an amino acid sequence set forth in SEQ ID NO: 533. In certain embodiments, the anti-TSLP antibody comprises a light chain comprising an amino acid sequence set forth in SEQ ID NO: 534. In certain embodiments, the anti-TSLP antibody comprises a heavy chain comprising an amino acid sequence set forth in SEQ ID NO: 533 and a light chain comprising an amino acid sequence set forth in SEQ ID NO: 534.

an amino acid sequence set forth in SEQ ID NO: 535. In certain embodiments, the anti-TSLP antibody comprises a light chain comprising an amino acid sequence set forth in SEQ ID NO: 536. In certain embodiments, the anti-TSLP antibody comprises a heavy chain comprising an amino acid sequence set forth in SEQ ID NO: 535 and a light chain comprising an amino acid sequence set forth in SEQ ID NO: 536.

In certain embodiments, the anti-TSLP antibody comprises a heavy chain comprising an amino acid sequence set forth in SEQ ID NO: 537. In certain embodiments, the anti-TSLP antibody comprises a light chain comprising an amino acid sequence set forth in SEQ ID NO: 538. In certain embodiments, the anti-TSLP antibody comprises a heavy chain comprising an amino acid sequence set forth in SEQ ID NO: 537 and a light chain comprising an amino acid sequence set forth in SEQ ID NO: 538.

In certain embodiments, the anti-TSLP antibody comprises a heavy chain comprising an amino acid sequence set forth in SEQ ID NO: 539. In certain embodiments, the anti-TSLP antibody comprises a light chain comprising an amino acid sequence set forth in SEQ ID NO: 540. In certain embodiments, the anti-TSLP antibody comprises a heavy chain comprising an amino acid sequence set forth in SEQ ID NO: 539 and a light chain comprising an amino acid sequence set forth in SEQ ID NO: 540.

In certain embodiments, the anti-TSLP antibody comprises a heavy chain comprising an amino acid sequence set forth in SEQ ID NO: 541. In certain embodiments, the anti-TSLP antibody comprises a light chain comprising an amino acid sequence set forth in SEQ ID NO: 542. In certain embodiments, the anti-TSLP antibody comprises a heavy chain comprising an amino acid sequence set forth in SEQ ID NO: 541 and a light chain comprising an amino acid sequence set forth in SEQ ID NO: 542.

In certain embodiments, the anti-TSLP antibody comprises a heavy chain comprising an amino acid sequence set forth in SEQ ID NO: 586. In certain embodiments, the anti-TSLP antibody comprises a light chain comprising an amino acid sequence set forth in SEQ ID NO: 587. In certain embodiments, the anti-TSLP antibody comprises a heavy chain comprising an amino acid sequence set forth in SEQ ID NO: 586 and a light chain comprising an amino acid sequence set forth in SEQ ID NO: 587.

an amino acid sequence set forth in SEQ ID NO: 584. In certain embodiments, the anti-TSLP antibody comprises a light chain comprising an amino acid sequence set forth in SEQ ID NO: 585. In certain embodiments, the anti-TSLP antibody comprises a heavy chain comprising an amino acid sequence set forth in SEQ ID NO: 584 and a light chain comprising an amino acid sequence set forth in SEQ ID NO: 585.

In certain embodiments, the Fc region of an anti-TSLP antibody, or antigen binding fragment thereof, comprises one or more amino acid substitutions, wherein the one or more substitutions result in an increase in one or more of antibody half-life, ADCC activity, ADCP activity, or CDC activity compared with the Fc region without the one or more amino acid substitutions. In certain embodiments, the one or more amino acid substitutions results in increased antibody half-life at pH 6.0 compared to an antibody comprising a wild-type Fc region. In certain embodiments, the anti-TSLP antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the anti-TSLP antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to tezepelumab. In certain embodiments, the anti-TSLP antibody has an increased half-life that is about 2.5-fold, 2.4-fold, 2.3-fold, 2.2-fold, 2.1-fold, 2.0-fold, 1.9-fold, or 1.8-fold longer compared to tezepelumab.

In certain embodiments, an anti-OX40L antibody, or antigen binding fragment thereof, provided herein comprises a VH sequence and a VL sequence provided in TABLE 7 (e.g., a VH sequence and a VL sequence from the same row of TABLE 7), together with a heavy chain constant region set forth in any one of SEQ ID NOs: 47-270 and a light chain constant region. In certain embodiments, an anti-OX40L antibody, or antigen binding fragment thereof, provided herein comprises a VH sequence and a VL sequence provided in TABLE 7, together with a heavy chain constant region selected from a sequence set forth in any one of SEQ ID NOs: 47-270 and a light chain constant region set forth in SEQ ID NO: 46.

In certain embodiments, the anti-OX40L antibody, or antigen binding fragment thereof, comprises a VH sequence set forth in SEQ ID NO: 335 and a VL sequence set forth in SEQ ID NO: 339; and the constant heavy chain comprises a human IgG sequence selected from a sequence set forth in any one of SEQ ID NOs: 47-270 (e.g., SEQ ID NO: 61 or SEQ ID NO: 173). In certain embodiments, the anti-OX40L antibody, or antigen binding fragment thereof, comprises a VH sequence set forth in SEQ ID NO: 336 and a VL sequence set forth in SEQ ID NO: 340; and the constant heavy chain comprises a human IgG sequence selected from a sequence set forth in any one of SEQ ID NOs: 47-270 (e.g., SEQ ID NO: 61 or SEQ ID NO: 173). In certain embodiments, the anti-OX40L antibody, or antigen binding fragment thereof, comprises a VH sequence set forth in SEQ ID NO: 337 and a VL sequence set forth in SEQ ID NO: 341; and the constant heavy chain comprises a human IgG sequence selected from a sequence set forth in any of SEQ ID NOs: 47-270 (e.g., SEQ ID NO: 61 or SEQ ID NO: 173). In certain embodiments, the anti-OX40L antibody, or antigen binding fragment thereof, comprises a VH sequence set forth in SEQ ID NO: 338 and a VL sequence set forth in SEQ ID NO: 342; and the constant heavy chain comprises a human IgG sequence selected from a sequence set forth in any of SEQ ID NOs: 47-270 (e.g., SEQ ID NO: 61 or SEQ ID NO: 173). In certain embodiments, the antibody, or antigen binding fragment thereof, comprises a light chain constant region set forth in SEQ ID NO: 46.

In certain embodiments, the anti-OX40L antibody comprises a heavy chain comprising an amino acid sequence set forth in SEQ ID NO: 543 and/or SEQ ID NO: 544. In certain embodiments, the anti-OX40L antibody comprises a light chain comprising an amino acid sequence set forth in SEQ ID NO: 545. In certain embodiments, the anti-OX40L antibody comprises a heavy chain comprising an amino acid sequence set forth in SEQ ID NO: 543 and a light chain comprising an amino acid sequence set forth in SEQ ID NO: 545. The heavy chain sequence of SEQ ID NO: 543 contains a C-terminal lysine, which may be cleaved off during manufacture or after administration, resulting in an antibody containing the heavy chain sequence of SEQ ID NO: 544. In certain embodiments, the anti-OX40L antibody comprises a heavy chain comprising an amino acid sequence set forth in SEQ ID NO: 544 and a light chain comprising an amino acid sequence set forth in SEQ ID NO: 545.

In certain embodiments, the Fc region of anti-OX40L antibody, or antigen binding fragment thereof, comprises one or more amino acid substitutions, wherein the one or more substitutions result in an increase in one or more of antibody half-life, ADCC activity, ADCP activity, or CDC activity compared with the Fc region without the one or more amino acid substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an OX40L antibody comprising a wild-type Fc region. In certain embodiments, the anti-OX40L antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to amlitelimab and oxelumab. In certain embodiments, the anti-OX40L antibody has an increased half-life that is about 2.5-fold, 2.4-fold, 2.3-fold, 2.2-fold, 2.1-fold, 2.0-fold, 1.9-fold, or 1.8-fold longer compared to amlitelimab and oxelumab.

In certain embodiments, an antibody described herein comprises a heavy chain constant domain having a means for increasing the half-life of the antibody.

In certain embodiments, the Fc region comprises one or more amino acid substitutions, wherein the one or more substitutions result in a decrease in one or more of ADCC activity, ADCP activity, or CDC activity compared with the Fc region without the one or more substitutions.

In certain embodiments, the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, the one or more amino acid substitutions comprises a specific combination of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

Although the EU numbering system is typically used to identify the positions of the various Fc mutations described herein, direct numbering can also be used. For example, in certain embodiments, an antibody described herein comprises an Fc region with YTE mutations at positions 257, 259, and 261 or 253, 255 and 257, respectively. In certain embodiments, an antibody described herein comprises an Fc region with LALA mutations at positions 239 and 240 or 235 and 236, respectively. In certain embodiments, an antibody (e.g., a TSLP antibody) described herein comprises an Fc region (e.g., a human IgG1 Fc region) with YTE mutations at positions 257, 259, and 261, respectively, and with LALA mutations at positions 239 and 240, respectively. In certain embodiments, an antibody (e.g., an OX40L antibody) described herein comprises an Fc region (e.g., a human IgG1 Fc region) with YTE mutations at positions 253, 255 and 257 and with LALA mutations at positions 235 and 236, respectively. In certain embodiments, a TSLP antibody described herein comprises the VH and VL of Antibody 5 and an Fc region (e.g., a human IgG1 Fc region) comprising YTE mutations at positions 257, 259, and 261 (M257Y/S259T/T261E), respectively, and with LALA mutations at positions 239 and 240 (L239A/L240A), respectively (e.g., as shown in the heavy chain sequences of SEQ ID NOs: 457 and 459) . . . . In certain embodiments, an OX40L antibody described herein comprises the VH and VL of Construct 114 and an Fc region comprising YTE mutations at positions 253, 255 and 257 (M253Y/S255T/T257E), respectively and with LALA mutations at positions 235 and 236 (L235A/L236A), respectively (e.g., as shown in the heavy chain sequences of SEQ ID NOs: 478 and 479).

In certain embodiments the human Fc region comprises a human IgG1 Fc region with LALA mutations. In certain embodiments, the human Fc region comprises a human IgG1 Fc region with YTE mutations. In certain embodiments, the human Fc region comprises a human IgG1 Fc region with LALA and YTE mutations. In certain embodiments, when direct numbering is used, “YTE” and “LALA” mutations can be located at different amino acid position numbers. For example, a human Fc region can comprise a human IgG1 Fc region with LALA mutations at L239A/L240A and/or YTE mutations at M257Y/S259T/T261E (e.g., in a TSLP antibody described herein). As another example, a human Fc region can comprise a human IgG1 Fc region with LALA mutations at L235A/L236A and/or YTE mutations at M253Y/S255T/T257E (e.g., in an OX40L antibody described herein).

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

### TSLP Binding

The affinity of an anti-TSLP antibody, or antigen binding fragment thereof, for TSLP can be represented by the dissociation equilibrium constant (K_{D}). Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., TSLP) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-TSLP antibody, or antigen binding fragment thereof, for a non-target molecule is less than about 50% of the affinity for TSLP. In some embodiments, the affinity of an anti-TSLP antibody, or antigen binding fragment thereof, for a non-target molecule is less than about 40% of the affinity for TSLP. In some embodiments, the affinity of an anti-TSLP antibody, or antigen binding fragment thereof, for a non-target molecule is less than about 30% of the affinity for TSLP. In some embodiments, the affinity of an anti-TSLP antibody, or antigen binding fragment thereof, for a non-target molecule is less than about 20% of the affinity for TSLP. In some embodiments, the affinity of an anti-TSLP antibody, or antigen binding fragment thereof, for a non-target molecule is less than about 10% of the affinity for TSLP. In some embodiments, the affinity of an anti-TSLP antibody, or antigen binding fragment thereof, for a non-target molecule is less than about 1% of the affinity for TSLP. In some embodiments, the affinity of an anti-TSLP antibody, or antigen binding fragment thereof, for a non-target molecule is less than about 0.1% of the affinity for TSLP.

In certain embodiments, the antibody, or antigen binding fragment thereof, binds a TSLP sequence set forth in SEQ ID NO: 271 or 272.

In certain embodiments, the antibody, or antigen binding fragment thereof, binds to a TSLP sequence set forth in SEQ ID NO: 271 or 272 with a K_{D} of less than or equal to about 1, 2, 3, 4, 5, 6, 7, 8, or 9×10⁻⁹ M, as measured by surface plasmon resonance (SPR). In certain embodiments, the antibody, or antigen binding fragment thereof, binds to a TSLP sequence set forth in SEQ ID NO: 271 or 272 with a K_{D} of less than or equal to about 1×10⁻¹⁰ M, as measured by surface plasmon resonance (SPR). In certain embodiments, the antibody, or antigen binding fragment thereof, binds to human TSLP with a K_{D} of less than or equal to about 1×10⁻⁹ M, as measured by surface plasmon resonance (SPR).

In some embodiments, an antibody, or antigen binding fragment thereof, provided herein binds TSLP with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody, or antigen binding fragment thereof, provided herein binds TSLP with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody, or antigen binding fragment thereof, provided herein for the binding of TSLP is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody, or antigen binding fragment thereof, provided herein binds TSLP with a K_{D} of less than or equal to about 1×10⁻⁸ M, or less than or equal to about 1×10⁻⁹ M as measured by ELISA or any other suitable method known in the art.

In some embodiments, the antibody, or antigen binding fragment thereof, provided herein binds TSLP with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody, or antigen binding fragment thereof, provided herein binds TSLP with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

In some embodiments, the antibody, or antigen binding fragment thereof, provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody, or antigen binding fragment thereof, provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### OX40L Binding

The affinity of an anti-OX40L antibody, or antigen binding fragment thereof, for OX40L can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., OX40L) and comparing it to binding to a non-target molecule. In some embodiments, the affinity of an anti-OX40L antibody, or antigen binding fragment thereof, for a non-target molecule is less than about 50% of the affinity for OX40L. In some embodiments, the affinity of an anti-OX40L antibody, or antigen binding fragment thereof, for a non-target molecule is less than about 40% of the affinity for OX40L. In some embodiments, the affinity of an anti-OX40L antibody, or antigen binding fragment thereof, for a non-target molecule is less than about 30% of the affinity for OX40L. In some embodiments, the affinity of an anti-OX40L antibody, or antigen binding fragment thereof, for a non-target molecule is less than about 20% of the affinity for OX40L. In some embodiments, the affinity of an anti-OX40L antibody, or antigen binding fragment thereof, for a non-target molecule is less than about 10% of the affinity for OX40L. In some embodiments, the affinity of an anti-OX40L antibody, or antigen binding fragment thereof, for a non-target molecule is less than about 1% of the affinity for OX40L. In some embodiments, the affinity of an anti-OX40L antibody, or antigen binding fragment thereof, for a non-target molecule is less than about 0.1% of the affinity for OX40L.

In certain embodiments, the antibody, or antigen binding fragment thereof, binds an OX40L sequence set forth in SEQ ID NO: 739.

In certain embodiments, the antibody, or antigen binding fragment thereof, binds to an OX40L sequence set forth in SEQ ID NO: 739 with a K_{D} of less than or equal to about 1, 2, 3, 4, 5, 6, 7, 8, or 9×10⁻⁹ M, as measured by surface plasmon resonance (SPR). In certain embodiments, the antibody, or antigen binding fragment thereof, binds to an OX40L sequence set forth in SEQ ID NO: 739 with a K_{D} of less than or equal to about 1×10⁻¹⁰ M, as measured by surface plasmon resonance (SPR). In certain embodiments, the antibody, or antigen binding fragment thereof, binds to human OX40L with a K_{D} of less than or equal to about 1×10⁻⁹ M, as measured by surface plasmon resonance (SPR).

In some embodiments, an antibody, or antigen binding fragment thereof, provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody, or antigen binding fragment thereof, provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody, or antigen binding fragment thereof, provided herein for the binding of OX40L is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody, or antigen binding fragment thereof, provided herein binds OX40L with a K_{D} of less than or equal to about 1×10⁻⁸ M, or less than or equal to about 1×10⁻⁹ M as measured by ELISA or any other suitable method known in the art.

In some embodiments, the antibody, or antigen binding fragment thereof, provided herein binds OX40L with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody, or antigen binding fragment thereof, provided herein binds OX40L with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

In some embodiments, the antibody, or antigen binding fragment thereof, provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody, or antigen binding fragment thereof, provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Combinations of Anti-TSLP Antibodies and Anti-OX40L Antibodies

Any of the anti-TSLP antibodies, or antigen binding fragments thereof, described herein can be combined with any of the anti-OX40L antibodies, or antigen binding fragments thereof, described herein (e.g., combined in a single composition or used in combination for treatment of a subject). In some embodiments, the anti-TSLP antibody, or antigen binding fragment thereof, described herein, is selected from TABLE 3, TABLE 4, TABLE 5, or TABLE 6 and the anti-OX40L antibody, or antigen fragment thereof, described herein is selected from TABLE 7.

In some embodiments, the anti-TSLP antibody, or antigen binding fragment thereof, is Antibody 5 and the anti-OX40L antibody, or antigen fragment thereof, described herein is selected from TABLE 7. In some embodiments, the anti-TSLP antibody, or antigen binding fragment thereof, is Antibody 7 and the anti-OX40L antibody, or antigen fragment thereof, described herein is selected from TABLE 7. In some embodiments, the anti-TSLP antibody, or antigen binding fragment thereof, is Antibody 23 and the anti-OX40L antibody, or antigen fragment thereof, described herein is selected from TABLE 7. In some embodiments, the anti-TSLP antibody, or antigen binding fragment thereof, is Antibody 29 and the anti-OX40L antibody, or antigen fragment thereof, described herein is selected from TABLE 7. In some embodiments, the anti-TSLP antibody, or antigen binding fragment thereof, is Antibody 38 and the anti-OX40L antibody, or antigen fragment thereof, described herein is selected from TABLE 7. In some embodiments, the anti-TSLP antibody, or antigen binding fragment thereof, is Antibody 39 and the anti-OX40L antibody, or antigen fragment thereof, described herein is selected from TABLE 7. In some embodiments, the anti-TSLP antibody, or antigen binding fragment thereof, is Antibody 31 and the anti-OX40L antibody, or antigen fragment thereof, described herein is selected from TABLE 7. In some embodiments, the anti-TSLP antibody, or antigen binding fragment thereof, is Antibody 36 and the anti-OX40L antibody, or antigen fragment thereof, described herein is selected from TABLE 7. In some embodiments, the anti-TSLP antibody, or antigen binding fragment thereof, is Antibody 44 and the anti-OX40L antibody, or antigen fragment thereof, described herein is selected from TABLE 7. In some embodiments, the anti-TSLP antibody, or antigen binding fragment thereof, is Antibody 51 and the anti-OX40L antibody, or antigen fragment thereof, described herein is selected from TABLE 7. In some embodiments, the anti-TSLP antibody, or antigen binding fragment thereof, is Antibody 59 and the anti-OX40L antibody, or antigen fragment thereof, described herein is selected from TABLE 7. In some embodiments, the anti-TSLP antibody, or antigen binding fragment thereof, is tezepelumab and the anti-OX40L antibody, or antigen fragment thereof, described herein is selected from TABLE 7.

In some embodiments, the anti-OX40L antibody, or antigen binding fragment thereof, is Construct 114 and the anti-TSLP antibody, or antigen fragment thereof, described herein is selected from TABLE 4. In some embodiments, the anti-OX40L antibody, or antigen binding fragment thereof, is Construct 114 and the anti-TSLP antibody, or antigen fragment thereof, described herein is selected from TABLE 6. In some embodiments, the anti-OX40L antibody, or antigen binding fragment thereof, is Construct 231 and the anti-TSLP antibody, or antigen fragment thereof, described herein is selected from TABLE 4. In some embodiments, the anti-OX40L antibody, or antigen binding fragment thereof, is Construct 231 and the anti-TSLP antibody, or antigen fragment thereof, described herein is selected from TABLE 6. In some embodiments, the anti-OX40L antibody, or antigen binding fragment thereof, is Construct 328 and the anti-TSLP antibody, or antigen fragment thereof, described herein is selected from TABLE 4. In some embodiments, the anti-OX40L antibody, or antigen binding fragment thereof, is Construct 328 and the anti-TSLP antibody, or antigen fragment thereof, described herein is selected from TABLE 6. In some embodiments, the anti-OX40L antibody, or antigen binding fragment thereof, is Construct 105 and the anti-TSLP antibody, or antigen fragment thereof, described herein is selected from TABLE 4. In some embodiments, the anti-OX40L antibody, or antigen binding fragment thereof, is Construct 105 and the anti-TSLP antibody, or antigen fragment thereof, described herein is selected from TABLE 6. In some embodiments, the anti-OX40L antibody, or antigen binding fragment thereof, is amlitelimab and the anti-TSLP antibody, or antigen fragment thereof, described herein is selected from TABLE 4. In some embodiments, the anti-OX40L antibody, or antigen binding fragment thereof, is amlitelimab and the anti-TSLP antibody, or antigen fragment thereof, described herein is selected from TABLE 6. In some embodiments, the anti-OX40L antibody, or antigen binding fragment thereof, is oxelumab and the anti-TSLP antibody, or antigen fragment thereof, described herein is selected from TABLE 4. In some embodiments, the anti-OX40L antibody, or antigen binding fragment thereof, is oxelumab and the anti-TSLP antibody, or antigen fragment thereof, described herein is selected from TABLE 6.

Exemplary combinations of anti-TSLP antibodies and anti-OX40L antibodies are set forth in TABLE 10, below.

TABLE 10
Combinations of anti-TSLP antibodies and anti-OX40L antibodies

| Combination | Anti-OX40L | Anti-TSLP |
|---|---|---|
| Number | Antibody | Antibody |
| 1 | Construct 114 | Antibody 1 |
| 2 | Construct 114 | Antibody 2 |
| 3 | Construct 114 | Antibody 3 |
| 4 | Construct 114 | Antibody 4 |
| 5 | Construct 114 | Antibody 5 |
| 6 | Construct 114 | Antibody 6 |
| 7 | Construct 114 | Antibody 7 |
| 8 | Construct 114 | Antibody 8 |
| 9 | Construct 114 | Antibody 9 |
| 10 | Construct 114 | Antibody 10 |
| 11 | Construct 114 | Antibody 11 |
| 12 | Construct 114 | Antibody 12 |
| 13 | Construct 114 | Antibody 13 |
| 14 | Construct 114 | Antibody 14 |
| 15 | Construct 114 | Antibody 15 |
| 16 | Construct 114 | Antibody 16 |
| 17 | Construct 114 | Antibody 17 |
| 18 | Construct 114 | Antibody 18 |
| 19 | Construct 114 | Antibody 19 |
| 20 | Construct 114 | Antibody 20 |
| 21 | Construct 114 | Antibody 21 |
| 22 | Construct 114 | Antibody 22 |
| 23 | Construct 114 | Antibody 23 |
| 24 | Construct 114 | Antibody 24 |
| 25 | Construct 114 | Antibody 25 |
| 26 | Construct 114 | Antibody 26 |
| 27 | Construct 114 | Antibody 27 |
| 28 | Construct 114 | Antibody 28 |
| 29 | Construct 114 | Antibody 29 |
| 30 | Construct 114 | Antibody 30 |
| 31 | Construct 114 | Antibody 31 |
| 32 | Construct 114 | Antibody 32 |
| 33 | Construct 114 | Antibody 33 |
| 34 | Construct 114 | Antibody 34 |
| 35 | Construct 114 | Antibody 35 |
| 36 | Construct 114 | Antibody 36 |
| 37 | Construct 114 | Antibody 37 |
| 38 | Construct 114 | Antibody 38 |
| 39 | Construct 114 | Antibody 39 |
| 40 | Construct 114 | Antibody 40 |
| 41 | Construct 114 | Antibody 41 |
| 42 | Construct 114 | Antibody 42 |
| 43 | Construct 114 | Antibody 43 |
| 44 | Construct 114 | Antibody 44 |
| 45 | Construct 114 | Antibody 45 |
| 46 | Construct 114 | Antibody 46 |
| 47 | Construct 114 | Antibody 47 |
| 48 | Construct 114 | Antibody 48 |
| 49 | Construct 114 | Antibody 49 |
| 50 | Construct 114 | Antibody 50 |
| 51 | Construct 114 | Antibody 51 |
| 52 | Construct 114 | Antibody 52 |
| 53 | Construct 114 | Antibody 53 |
| 54 | Construct 114 | Antibody 54 |
| 55 | Construct 114 | Antibody 55 |
| 56 | Construct 114 | Antibody 56 |
| 57 | Construct 114 | Antibody 57 |
| 58 | Construct 114 | Antibody 58 |
| 59 | Construct 114 | Antibody 59 |
| 60 | Construct 114 | Antibody 60 |
| 61 | Construct 114 | Antibody 61 |
| 62 | Construct 114 | Tezepelumab |
| 63 | Construct 231 | Antibody 1 |
| 64 | Construct 231 | Antibody 2 |
| 65 | Construct 231 | Antibody 3 |
| 66 | Construct 231 | Antibody 4 |
| 67 | Construct 231 | Antibody 5 |
| 68 | Construct 231 | Antibody 6 |
| 69 | Construct 231 | Antibody 7 |
| 70 | Construct 231 | Antibody 8 |
| 71 | Construct 231 | Antibody 9 |
| 72 | Construct 231 | Antibody 10 |
| 73 | Construct 231 | Antibody 11 |
| 74 | Construct 231 | Antibody 12 |
| 75 | Construct 231 | Antibody 13 |
| 76 | Construct 231 | Antibody 14 |
| 77 | Construct 231 | Antibody 15 |
| 78 | Construct 231 | Antibody 16 |
| 79 | Construct 231 | Antibody 17 |
| 80 | Construct 231 | Antibody 18 |
| 81 | Construct 231 | Antibody 19 |
| 82 | Construct 231 | Antibody 20 |
| 83 | Construct 231 | Antibody 21 |
| 84 | Construct 231 | Antibody 22 |
| 85 | Construct 231 | Antibody 23 |
| 86 | Construct 231 | Antibody 24 |
| 87 | Construct 231 | Antibody 25 |
| 88 | Construct 231 | Antibody 26 |
| 89 | Construct 231 | Antibody 27 |
| 90 | Construct 231 | Antibody 28 |
| 91 | Construct 231 | Antibody 29 |
| 92 | Construct 231 | Antibody 30 |
| 93 | Construct 231 | Antibody 31 |
| 94 | Construct 231 | Antibody 32 |
| 95 | Construct 231 | Antibody 33 |
| 96 | Construct 231 | Antibody 34 |
| 97 | Construct 231 | Antibody 35 |
| 98 | Construct 231 | Antibody 36 |
| 99 | Construct 231 | Antibody 37 |
| 100 | Construct 231 | Antibody 38 |
| 101 | Construct 231 | Antibody 39 |
| 102 | Construct 231 | Antibody 40 |
| 103 | Construct 231 | Antibody 41 |
| 104 | Construct 231 | Antibody 42 |
| 105 | Construct 231 | Antibody 43 |
| 106 | Construct 231 | Antibody 44 |
| 107 | Construct 231 | Antibody 45 |
| 108 | Construct 231 | Antibody 46 |
| 109 | Construct 231 | Antibody 47 |
| 110 | Construct 231 | Antibody 48 |
| 111 | Construct 231 | Antibody 49 |
| 112 | Construct 231 | Antibody 50 |
| 113 | Construct 231 | Antibody 51 |
| 114 | Construct 231 | Antibody 52 |
| 115 | Construct 231 | Antibody 53 |
| 116 | Construct 231 | Antibody 54 |
| 117 | Construct 231 | Antibody 55 |
| 118 | Construct 231 | Antibody 56 |
| 119 | Construct 231 | Antibody 57 |
| 120 | Construct 231 | Antibody 58 |
| 121 | Construct 231 | Antibody 59 |
| 122 | Construct 231 | Antibody 60 |
| 123 | Construct 231 | Antibody 61 |
| 124 | Construct 231 | Tezepelumab |
| 125 | Construct 328 | Antibody 1 |
| 126 | Construct 328 | Antibody 2 |
| 127 | Construct 328 | Antibody 3 |
| 128 | Construct 328 | Antibody 4 |
| 129 | Construct 328 | Antibody 5 |
| 130 | Construct 328 | Antibody 6 |
| 131 | Construct 328 | Antibody 7 |
| 132 | Construct 328 | Antibody 8 |
| 133 | Construct 328 | Antibody 9 |
| 134 | Construct 328 | Antibody 10 |
| 135 | Construct 328 | Antibody 11 |
| 136 | Construct 328 | Antibody 12 |
| 137 | Construct 328 | Antibody 13 |
| 138 | Construct 328 | Antibody 14 |
| 139 | Construct 328 | Antibody 15 |
| 140 | Construct 328 | Antibody 16 |
| 141 | Construct 328 | Antibody 17 |
| 142 | Construct 328 | Antibody 18 |
| 143 | Construct 328 | Antibody 19 |
| 144 | Construct 328 | Antibody 20 |
| 145 | Construct 328 | Antibody 21 |
| 146 | Construct 328 | Antibody 22 |
| 147 | Construct 328 | Antibody 23 |
| 148 | Construct 328 | Antibody 24 |
| 149 | Construct 328 | Antibody 25 |
| 150 | Construct 328 | Antibody 26 |
| 151 | Construct 328 | Antibody 27 |
| 152 | Construct 328 | Antibody 28 |
| 153 | Construct 328 | Antibody 29 |
| 154 | Construct 328 | Antibody 30 |
| 155 | Construct 328 | Antibody 31 |
| 156 | Construct 328 | Antibody 32 |
| 157 | Construct 328 | Antibody 33 |
| 158 | Construct 328 | Antibody 34 |
| 159 | Construct 328 | Antibody 35 |
| 160 | Construct 328 | Antibody 36 |
| 161 | Construct 328 | Antibody 37 |
| 162 | Construct 328 | Antibody 38 |
| 163 | Construct 328 | Antibody 39 |
| 164 | Construct 328 | Antibody 40 |
| 165 | Construct 328 | Antibody 41 |
| 166 | Construct 328 | Antibody 42 |
| 167 | Construct 328 | Antibody 43 |
| 168 | Construct 328 | Antibody 44 |
| 169 | Construct 328 | Antibody 45 |
| 170 | Construct 328 | Antibody 46 |
| 171 | Construct 328 | Antibody 47 |
| 172 | Construct 328 | Antibody 48 |
| 173 | Construct 328 | Antibody 49 |
| 174 | Construct 328 | Antibody 50 |
| 175 | Construct 328 | Antibody 51 |
| 176 | Construct 328 | Antibody 52 |
| 177 | Construct 328 | Antibody 53 |
| 178 | Construct 328 | Antibody 54 |
| 179 | Construct 328 | Antibody 55 |
| 180 | Construct 328 | Antibody 56 |
| 181 | Construct 328 | Antibody 57 |
| 182 | Construct 328 | Antibody 58 |
| 183 | Construct 328 | Antibody 59 |
| 184 | Construct 328 | Antibody 60 |
| 185 | Construct 328 | Antibody 61 |
| 186 | Construct 328 | Tezepelumab |
| 187 | Construct 105 | Antibody 1 |
| 188 | Construct 105 | Antibody 2 |
| 189 | Construct 105 | Antibody 3 |
| 190 | Construct 105 | Antibody 4 |
| 191 | Construct 105 | Antibody 5 |
| 192 | Construct 105 | Antibody 6 |
| 193 | Construct 105 | Antibody 7 |
| 194 | Construct 105 | Antibody 8 |
| 195 | Construct 105 | Antibody 9 |
| 196 | Construct 105 | Antibody 10 |
| 197 | Construct 105 | Antibody 11 |
| 198 | Construct 105 | Antibody 12 |
| 199 | Construct 105 | Antibody 13 |
| 200 | Construct 105 | Antibody 14 |
| 201 | Construct 105 | Antibody 15 |
| 202 | Construct 105 | Antibody 16 |
| 203 | Construct 105 | Antibody 17 |
| 204 | Construct 105 | Antibody 18 |
| 205 | Construct 105 | Antibody 19 |
| 206 | Construct 105 | Antibody 20 |
| 207 | Construct 105 | Antibody 21 |
| 208 | Construct 105 | Antibody 22 |
| 209 | Construct 105 | Antibody 23 |
| 210 | Construct 105 | Antibody 24 |
| 211 | Construct 105 | Antibody 25 |
| 212 | Construct 105 | Antibody 26 |
| 213 | Construct 105 | Antibody 27 |
| 214 | Construct 105 | Antibody 28 |
| 215 | Construct 105 | Antibody 29 |
| 216 | Construct 105 | Antibody 30 |
| 217 | Construct 105 | Antibody 31 |
| 218 | Construct 105 | Antibody 32 |
| 219 | Construct 105 | Antibody 33 |
| 220 | Construct 105 | Antibody 34 |
| 221 | Construct 105 | Antibody 35 |
| 222 | Construct 105 | Antibody 36 |
| 223 | Construct 105 | Antibody 37 |
| 224 | Construct 105 | Antibody 38 |
| 225 | Construct 105 | Antibody 39 |
| 226 | Construct 105 | Antibody 40 |
| 227 | Construct 105 | Antibody 41 |
| 228 | Construct 105 | Antibody 42 |
| 229 | Construct 105 | Antibody 43 |
| 230 | Construct 105 | Antibody 44 |
| 231 | Construct 105 | Antibody 45 |
| 232 | Construct 105 | Antibody 46 |
| 233 | Construct 105 | Antibody 47 |
| 234 | Construct 105 | Antibody 48 |
| 235 | Construct 105 | Antibody 49 |
| 236 | Construct 105 | Antibody 50 |
| 237 | Construct 105 | Antibody 51 |
| 238 | Construct 105 | Antibody 52 |
| 239 | Construct 105 | Antibody 53 |
| 240 | Construct 105 | Antibody 54 |
| 241 | Construct 105 | Antibody 55 |
| 242 | Construct 105 | Antibody 56 |
| 243 | Construct 105 | Antibody 57 |
| 244 | Construct 105 | Antibody 58 |
| 245 | Construct 105 | Antibody 59 |
| 246 | Construct 105 | Antibody 60 |
| 247 | Construct 105 | Antibody 61 |
| 248 | Construct 105 | Tezepelumab |
| 249 | Amlitelimab | Antibody 1 |
| 250 | Amlitelimab | Antibody 2 |
| 251 | Amlitelimab | Antibody 3 |
| 252 | Amlitelimab | Antibody 4 |
| 253 | Amlitelimab | Antibody 5 |
| 254 | Amlitelimab | Antibody 6 |
| 255 | Amlitelimab | Antibody 7 |
| 256 | Amlitelimab | Antibody 8 |
| 257 | Amlitelimab | Antibody 9 |
| 258 | Amlitelimab | Antibody 10 |
| 259 | Amlitelimab | Antibody 11 |
| 260 | Amlitelimab | Antibody 12 |
| 261 | Amlitelimab | Antibody 13 |
| 262 | Amlitelimab | Antibody 14 |
| 263 | Amlitelimab | Antibody 15 |
| 264 | Amlitelimab | Antibody 16 |
| 265 | Amlitelimab | Antibody 17 |
| 266 | Amlitelimab | Antibody 18 |
| 267 | Amlitelimab | Antibody 19 |
| 268 | Amlitelimab | Antibody 20 |
| 269 | Amlitelimab | Antibody 21 |
| 270 | Amlitelimab | Antibody 22 |
| 271 | Amlitelimab | Antibody 23 |
| 272 | Amlitelimab | Antibody 24 |
| 273 | Amlitelimab | Antibody 25 |
| 274 | Amlitelimab | Antibody 26 |
| 275 | Amlitelimab | Antibody 27 |
| 276 | Amlitelimab | Antibody 28 |
| 277 | Amlitelimab | Antibody 29 |
| 278 | Amlitelimab | Antibody 30 |
| 279 | Amlitelimab | Antibody 31 |
| 280 | Amlitelimab | Antibody 32 |
| 281 | Amlitelimab | Antibody 33 |
| 282 | Amlitelimab | Antibody 34 |
| 283 | Amlitelimab | Antibody 35 |
| 284 | Amlitelimab | Antibody 36 |
| 285 | Amlitelimab | Antibody 37 |
| 286 | Amlitelimab | Antibody 38 |
| 287 | Amlitelimab | Antibody 39 |
| 288 | Amlitelimab | Antibody 40 |
| 289 | Amlitelimab | Antibody 41 |
| 290 | Amlitelimab | Antibody 42 |
| 291 | Amlitelimab | Antibody 43 |
| 292 | Amlitelimab | Antibody 44 |
| 293 | Amlitelimab | Antibody 45 |
| 294 | Amlitelimab | Antibody 46 |
| 295 | Amlitelimab | Antibody 47 |
| 296 | Amlitelimab | Antibody 48 |
| 297 | Amlitelimab | Antibody 49 |
| 298 | Amlitelimab | Antibody 50 |
| 299 | Amlitelimab | Antibody 51 |
| 300 | Amlitelimab | Antibody 52 |
| 301 | Amlitelimab | Antibody 53 |
| 302 | Amlitelimab | Antibody 54 |
| 303 | Amlitelimab | Antibody 55 |
| 30 | Amlitelimab | Antibody 56 |
| 305 | Amlitelimab | Antibody 57 |
| 306 | Amlitelimab | Antibody 58 |
| 307 | Amlitelimab | Antibody 59 |
| 308 | Amlitelimab | Antibody 60 |
| 309 | Amlitelimab | Antibody 61 |
| 310 | Oxelumab | Antibody 1 |
| 311 | Oxelumab | Antibody 2 |
| 312 | Oxelumab | Antibody 3 |
| 313 | Oxelumab | Antibody 4 |
| 314 | Oxelumab | Antibody 5 |
| 315 | Oxelumab | Antibody 6 |
| 316 | Oxelumab | Antibody 7 |
| 317 | Oxelumab | Antibody 8 |
| 318 | Oxelumab | Antibody 9 |
| 319 | Oxelumab | Antibody 10 |
| 320 | Oxelumab | Antibody 11 |
| 321 | Oxelumab | Antibody 12 |
| 322 | Oxelumab | Antibody 13 |
| 323 | Oxelumab | Antibody 14 |
| 324 | Oxelumab | Antibody 15 |
| 325 | Oxelumab | Antibody 16 |
| 326 | Oxelumab | Antibody 17 |
| 327 | Oxelumab | Antibody 18 |
| 328 | Oxelumab | Antibody 19 |
| 329 | Oxelumab | Antibody 20 |
| 330 | Oxelumab | Antibody 21 |
| 331 | Oxelumab | Antibody 22 |
| 332 | Oxelumab | Antibody 23 |
| 333 | Oxelumab | Antibody 24 |
| 334 | Oxelumab | Antibody 25 |
| 335 | Oxelumab | Antibody 26 |
| 336 | Oxelumab | Antibody 27 |
| 337 | Oxelumab | Antibody 28 |
| 338 | Oxelumab | Antibody 29 |
| 339 | Oxelumab | Antibody 30 |
| 340 | Oxelumab | Antibody 31 |
| 341 | Oxelumab | Antibody 32 |
| 342 | Oxelumab | Antibody 33 |
| 343 | Oxelumab | Antibody 34 |
| 344 | Oxelumab | Antibody 35 |
| 345 | Oxelumab | Antibody 36 |
| 346 | Oxelumab | Antibody 37 |
| 347 | Oxelumab | Antibody 38 |
| 348 | Oxelumab | Antibody 39 |
| 349 | Oxelumab | Antibody 40 |
| 350 | Oxelumab | Antibody 41 |
| 351 | Oxelumab | Antibody 42 |
| 352 | Oxelumab | Antibody 43 |
| 353 | Oxelumab | Antibody 44 |
| 354 | Oxelumab | Antibody 45 |
| 355 | Oxelumab | Antibody 46 |
| 356 | Oxelumab | Antibody 47 |
| 357 | Oxelumab | Antibody 48 |
| 358 | Oxelumab | Antibody 49 |
| 359 | Oxelumab | Antibody 50 |
| 360 | Oxelumab | Antibody 51 |
| 361 | Oxelumab | Antibody 52 |
| 362 | Oxelumab | Antibody 53 |
| 363 | Oxelumab | Antibody 54 |
| 364 | Oxelumab | Antibody 55 |
| 365 | Oxelumab | Antibody 56 |
| 366 | Oxelumab | Antibody 57 |
| 367 | Oxelumab | Antibody 58 |
| 368 | Oxelumab | Antibody 59 |
| 369 | Oxelumab | Antibody 60 |
| 370 | Oxelumab | Antibody 61 |

In some embodiments, the anti-TSLP antibody, or antigen binding fragment thereof, and the anti-OX40L antibody, or antigen fragment thereof, are combined in a single composition (e.g., both antibodies, or antigen binding fragments thereof, are co-formulated in a single pharmaceutical composition with one or more pharmaceutically acceptable excipients). The composition can be administered to a subject (e.g., a mammalian subject, such as a human subject) for treatment of a disease or disorder in the subject, such as an inflammatory disorder or disease (e.g., atopic dermatitis, asthma, or COPD). In some embodiments, the composition further includes a hyaluronidase or variant thereof (e.g., any one of SEQ ID NOs: 453-462 and 464-519). In other embodiments, the composition comprising the anti-TSLP antibody, or antigen binding fragment thereof, and the anti-OX40L antibody, or antigen fragment thereof, is administered in combination with a hyaluronidase or variant thereof (i.e., the hyaluronidase or variant thereof is administered in a separate composition, which may be administered before, after, or at about the same time as the composition containing the TSLP and OX40L antibodies or antigen binding fragments thereof). A hyaluronidase or variant thereof may be co-administered or co-formulated with any combination of anti-TSLP antibody, or antigen binding fragment thereof, and anti-OX40L antibody, or antigen fragment thereof described herein.

In some embodiments, the anti-TSLP antibody, or antigen binding fragment thereof, and the anti-OX40L antibody, or antigen fragment thereof, are in separate compositions (e.g., a first pharmaceutical composition containing an anti-TSLP antibody, or an antigen binding fragment thereof, and a pharmaceutically acceptable excipient and a second pharmaceutical composition containing an anti-OX40L antibody, or an antigen binding fragment thereof, and a pharmaceutically acceptable excipient). These separate compositions can be co-administered to a subject (e.g., a mammalian subject, such as a human subject) for treatment of a disease or disorder in the subject, such as an inflammatory disorder or disease (e.g., atopic dermatitis, asthma, or COPD). In some embodiments, the composition containing the anti-TSLP antibody, or an antigen binding fragment thereof, is administered at about the same time as the composition containing the anti-OX40L antibody, or an antigen binding fragment thereof. In other embodiments, the composition containing the anti-TSLP antibody, or an antigen binding fragment thereof, and the composition containing the anti-OX40L antibody, or an antigen binding fragment thereof are administered sequentially, such that the action of the two overlaps and their combined effect is such that the reduction in a symptom, or other parameter related to the disease or disorder is greater than what would be observed with one antibody, or antigen binding fragment thereof, delivered alone or in the absence of the other. In some embodiments, the anti-TSLP antibody, or antigen binding fragment thereof, is administered before the anti-OX40L antibody, or antigen binding fragment thereof, by intervals ranging from minutes, hours, days to weeks. In some embodiments, the anti-OX40L antibody, or antigen binding fragment thereof, is administered before the anti-TSLP antibody, or antigen binding fragment thereof, by intervals ranging from minutes, hours, days to weeks. In embodiments in which the anti-TSLP antibody, or antigen binding fragment thereof, and the anti-OX40L antibody, or antigen fragment thereof, are in separate compositions, the hyaluronidase or a variant thereof (e.g., any one of SEQ ID NOs: 453-462 and 464-519) can be administered before the administration of both antibodies, after the administration of both antibodies, or at about the same time as the administration of both antibodies. In other embodiments, the hyaluronidase or a variant thereof can be administered after the first antibody is administered and before the second antibody is administered.

### Pharmaceutical Compositions

The present application provides compositions comprising the antibodies or antigen binding fragments thereof described herein, including pharmaceutical compositions comprising any one or more of the antibodies, or antigen binding fragments thereof, described herein with one or more pharmaceutically acceptable excipients. In some embodiments the composition is sterile. The pharmaceutical compositions generally comprise an effective amount of a TSLP antibody, or an antigen binding fragment thereof, and/or an OX40L antibody, or an antigen binding fragment thereof. In some embodiments, the pharmaceutical compositions further comprise a hyaluronidase or a variant thereof.

These compositions can comprise, in addition to one or more of the antibodies, or antigen binding fragments thereof, disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

For the anti-TSLP antibody, or antigen binding fragment thereof, and/or the anti-OX40L antibody, or antigen binding fragment thereof, that are to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on a number of factors, e.g., the nature and severity of the disease being treated. Prescription of treatment, e.g. decisions on dosage etc., can be within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies, or antigen binding fragments thereof, described herein can be produced using recombinant methods and compositions, e.g., as described in U.S. Pat. No. 4,816,567. In one embodiment, an isolated nucleic acid encoding an antibody, or antigen binding fragment thereof, described herein is provided. Such a nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such a nucleic acid are provided. In one embodiment, the nucleic acid is provided in a multicistronic vector. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, human embryonic kidney (HEK) cell, or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of the antibody, a nucleic acid encoding an antibody, e.g., as described above, is isolated and inserted into one or more vectors for further cloning and/or expression in a host cell. Such a nucleic acid may be readily isolated and sequenced using conventional procedures (e.g., by using oligonucleotide probes that are capable of binding specifically to genes encoding the heavy and light chains of the antibody).

When an antibody or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, a “substantially purified” antibody produced by the methods described herein has a purity level of at least about 30%, at least about 35%, at least about 40%, at least about 45%, at least about 50%, at least about 55%, at least about 60%, at least about 65%, at least about 70%, specifically, a purity level of at least about 75%, at least about 80%, at least about 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per. C6, or BHK.

For example, an antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, the method of producing an antibody in stable mammalian cells as described herein results in an expression product of the at least one stable mammalian cell comprising a larger percentage of the desired glycosylated antibody as compared to the monomeric heavy or light chain polypeptides, or other antibodies.

In some embodiments, the method of producing a glycosylated antibody in stable mammalian cells described herein comprises identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques.

Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni 2 affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting TSLP with an anti-TSLP antibody, or antigen binding fragment thereof, such as a human or humanized antibody, which results in inhibition of TSLP binding to TSLPR.

In an aspect, the present application provides methods of using the isolated anti-TSLP antibodies, or antigen binding fragments thereof, described herein for treatment of a disorder or disease in a subject. In certain aspects, described herein is a method for treating a subject in need thereof with an anti-TSLP antibody, or antigen binding fragment thereof, the method comprising administering to the subject (e.g., a mammalian subject) a therapeutically effective amount of an anti-TSLP antibody, or antigen binding fragment thereof, or pharmaceutical composition comprising an anti-TSLP antibody, or antigen binding fragment thereof, described herein. In certain embodiments, the present application provides methods of treating a disorder or disease associated with elevated levels of TSLP and/or IgE in a subject.

In certain aspects, described herein are methods for treating a pathology associated with TSLP activity, the method comprising administering to a mammalian subject a therapeutically effective amount of an isolated anti-TSLP antibody, or antigen binding fragment thereof, or a pharmaceutical composition comprising an isolated anti-TSLP antibody, or antigen binding fragment thereof, described herein.

In an aspect, the present application provides methods of contacting OX40L with an anti-OX40L antibody, or antigen binding fragment thereof, such as a human or humanized antibody, which results in inhibition of OX40L binding to an OX40 receptor expressed on a cell.

In an aspect, the present application provides methods of using the isolated anti-OX40L antibodies, or antigen binding fragments thereof, described herein for treatment of a disorder or disease in a subject. In certain aspects, described herein is a method for treating a subject in need thereof with an anti-OX40L antibody, or antigen binding fragment thereof, the method comprising administering to the subject (e.g., a mammalian subject) a therapeutically effective amount of an anti-OX40L antibody, or antigen binding fragment thereof, or pharmaceutical composition comprising an anti-OX40L antibody, or antigen binding fragment thereof, described herein. In certain embodiments, the present application provides methods of treating a disorder or disease associated with elevated levels of OX40L and/or IgE in a subject.

In certain aspects, described herein are methods for treating a pathology associated with OX40 or OX40L activity, the method comprising administering to a mammalian subject a therapeutically effective amount of an isolated anti-OX40L antibody, or antigen binding fragment thereof, or a pharmaceutical composition comprising an isolated anti-OX40L antibody, or antigen binding fragment thereof, described herein.

In an aspect, the present application provides methods of contacting TSLP and OX40L with an anti-TSLP antibody, or antigen binding fragment thereof, and an anti-OX40L antibody, or antigen binding fragment thereof, respectively (e.g., via administration of a single composition containing both antibodies or antigen binding fragments thereof or by administration of two different compositions, one containing the anti-TSLP antibody, or an antigen binding fragment thereof, and the other containing an anti-OX40L antibody or an antigen binding fragment thereof), such as a human or humanized antibody, which results in inhibition of TSLP binding to TSLPR and/or OX40L binding to OX40.

In an aspect, the present application provides methods of using the isolated anti-OX40L antibodies, or antigen binding fragments thereof, and isolated anti-TSLP antibodies, or antigen binding fragments thereof, described herein in combination for treatment of a disorder or disease in a subject. In certain aspects, described herein is a method for treating a subject in need thereof with an anti-TSLP antibody, or antigen binding fragment thereof, and an anti-OX40L antibody, or antigen binding fragments thereof, the method comprising administering to the subject (e.g., a mammalian subject) a therapeutically effective amount of an anti-OX40L antibody, or antigen binding fragment thereof, and a therapeutically effective amount of an anti-TSLP antibody, or antigen binding fragment thereof (e.g., administration two different pharmaceutical compositions, one comprising the anti-OX40L antibody, or an antigen binding fragment thereof, and the other comprising the anti-TSLP antibody, or an antigen binding fragment thereof), or pharmaceutical composition comprising such antibodies, or antigen binding fragments thereof, described herein (e.g., administration of a single pharmaceutical composition comprising both the anti-OX40L antibody, or an antigen binding fragment thereof, and the anti-TSLP antibody, or an antigen binding fragment thereof). In certain embodiments, the present application provides methods of treating a disorder or disease associated with elevated levels of TSLP, TSLPR, OX40L, OX40, and/or IgE in a subject. In certain cases, a therapeutically effective amount of each antibody may be different when the antibody is administered in combination with the other antibody or another therapeutic agent (e.g., as compared to the therapeutically effective amount of each antibody when administered alone).

In certain aspects, described herein are methods for treating a pathology associated with TSLP and/or OX40L activity, the method comprising administering to a mammalian subject a therapeutically effective amount of an isolated anti-OX40L antibody, or antigen binding fragment thereof, and a therapeutically effective amount of an isolated anti-TSLP antibody, or antigen binding fragment thereof, or a pharmaceutical composition comprising such antibodies, or antigen binding fragments thereof, described herein (e.g., via administration of a single composition containing both antibodies or antigen binding fragments thereof or by administration of two different compositions, one containing the anti-TSLP antibody, or an antigen binding fragment thereof, and the other containing an anti-OX40L antibody or an antigen binding fragment thereof). In certain cases, a therapeutically effective amount of each antibody may be different when the antibody is administered in combination with the other antibody or another therapeutic agent.

In certain embodiments, the isolated antibody, or antigen binding fragment thereof, or combination of anti-OX40L and anti-TSLP antibodies, or antigen binding fragments thereof, is used in the treatment of an inflammatory disorder or disease. In certain embodiments, the isolated antibody, or antigen binding fragment thereof, or combination of antibodies, or antigen binding fragments thereof, is used in the treatment of atopic dermatitis. In certain embodiments, the treatment reduces disease severity in a patient and wherein disease severity is assessed by an atopic dermatitis disease severity outcome measure. In certain embodiments, the isolated antibody, or antigen binding fragment thereof, or combination of antibodies, or antigen binding fragments thereof, is used in the treatment of asthma. In certain embodiments, the isolated antibody, or antigen binding fragment thereof, or combination of antibodies, or antigen binding fragments thereof, is used in the treatment of idiopathic pulmonary fibrosis. In certain embodiments, the isolated antibody, or antigen binding fragment thereof, or combination of antibodies, or antigen binding fragments thereof, is used in the treatment of alopecia areata. In certain embodiments, the isolated antibody, or antigen binding fragment thereof, or combination of antibodies, or antigen binding fragments thereof, is used in the treatment of chronic sinusitis with nasal polyps. In certain embodiments, the isolated antibody, or antigen binding fragment thereof, or combination of antibodies, or antigen binding fragments thereof, is used in the treatment of Chronic Rhinosinusitis without Nasal Polyps (CRSsNP). In certain embodiments, the isolated antibody, or antigen binding fragment thereof, or combination of antibodies, or antigen binding fragments thereof, is used in the treatment of eosinophilic esophagitis (EoE). In certain embodiments, the isolated antibody, or antigen binding fragment thereof, or combination of antibodies, or antigen binding fragments thereof, is used in the treatment of an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody, or antigen binding fragment thereof, or combination of antibodies, or antigen binding fragments thereof, is used in the treatment of Churg-Strauss syndrome/Eosinophilic granulomatosis with polyangiitis (EGPA). In certain embodiments, the isolated antibody, or antigen binding fragment thereof, or combination of antibodies, or antigen binding fragments thereof, is used in the treatment of Prurigo Nodularis (PN). In certain embodiments, the isolated antibody, or antigen binding fragment thereof, or combination of antibodies, or antigen binding fragments thereof, is used in the treatment of Chronic Spontaneous Urticaria (CSU). In certain embodiments, the isolated antibody, or antigen binding fragment thereof, or combination of antibodies, or antigen binding fragments thereof, is used in the treatment of Chronic Pruritis of Unknown Origin (CPUO). In certain embodiments, the isolated antibody, or antigen binding fragment thereof, or combination of antibodies, or antigen binding fragments thereof, is used in the treatment of Bullous Pemphigoid (BP). In certain embodiments, the isolated antibody, or antigen binding fragment thereof, or combination of antibodies, or antigen binding fragments thereof, is used in the treatment of Cold Inducible Urticaria (ColdU). In certain embodiments, the isolated antibody, or antigen binding fragment thereof, or combination of antibodies, or antigen binding fragments thereof, is used in the treatment of Allergic Fungal Rhinosinusitis (AFRS). In certain embodiments, the isolated antibody, or antigen binding fragment thereof, or combination of antibodies, or antigen binding fragments thereof, is used in the treatment of Allergic Bronchopulmonary Aspergillosis (ABPA). In certain embodiments, the isolated antibody, or antigen binding fragment thereof, or combination of antibodies, or antigen binding fragments thereof, is used in the treatment of Chronic Obstructive Pulmonary Disease (COPD). In certain embodiments, the isolated antibody, or antigen binding fragment thereof, or combination of antibodies, or antigen binding fragments thereof, is used in the treatment of inflammatory bowel disease, such as Crohn's disease or ulcerative colitis. In certain embodiments, the isolated antibody, or antigen binding fragment thereof, or combination of antibodies, or antigen binding fragments thereof, is used in the treatment of psoriasis. In certain embodiments, the isolated antibody, or antigen binding fragment thereof, or combination of antibodies, or antigen binding fragments thereof, is used in the treatment of lupus. In certain embodiments, the isolated antibody, or antigen binding fragment thereof, or combination of antibodies, or antigen binding fragments thereof, is used in the treatment of rheumatoid arthritis. In certain embodiments, the isolated antibody, or antigen binding fragment thereof, or combination of antibodies, or antigen binding fragments thereof, is used in the treatment of hidradenitis suppurativa. In certain embodiments, the isolated antibody, or antigen binding fragment thereof, or combination of antibodies, or antigen binding fragments thereof, is used in the treatment of celiac disease. In certain embodiments, the isolated antibody, or antigen binding fragment thereof, or combination of antibodies, or antigen binding fragments thereof, is used in the treatment of systemic sclerosis. In certain embodiments, the isolated antibody, or antigen binding fragment thereof, or combination of antibodies, or antigen binding fragments thereof, is used in the treatment of allergic rhinitis. In certain embodiments, the isolated antibody, or antigen binding fragment thereof, or combination of antibodies, or antigen binding fragments thereof, is used in the treatment of eosinophilic fasciitis. In certain embodiments, the isolated antibody, or antigen binding fragment thereof, or combination of antibodies, or antigen binding fragments thereof, is used in the treatment of scleromyxedema. In certain embodiments, the isolated antibody, or antigen binding fragment thereof, or combination of antibodies, or antigen binding fragments thereof, is used in the treatment of scleredema. In certain embodiments, the isolated antibody, or antigen binding fragment thereof, or combination of antibodies, or antigen binding fragments thereof, is used in the treatment of nephrogenic systemic fibrosis.

In certain aspects, described herein is a method for treating an inflammatory disorder or disease in a mammalian subject in need thereof, the method comprising administering to the mammalian subject a therapeutically effective amount of an antibody, or antigen binding fragment thereof, or combination of anti-OX40L and anti-TSLP antibodies, or antigen binding fragments thereof, described herein or a pharmaceutical composition of such antibody or antibodies or antigen binding fragments thereof described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn's disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease. In certain embodiments, the inflammatory disorder or disease is hidradenitis suppurativa. In certain embodiments, the inflammatory disorder or disease is systemic sclerosis. In certain embodiments, the inflammatory disorder or disease is allergic rhinitis. In certain embodiments, the inflammatory disorder or disease is eosinophilic fasciitis. In certain embodiments, the inflammatory disorder or disease is scleromyxedema. In certain embodiments, the inflammatory disorder or disease is scleredema. In certain embodiments, the inflammatory disorder or disease is nephrogenic systemic fibrosis.

In certain aspects, described herein are methods for treating a pathology associated with elevated levels of TSLP in a mammalian subject in need thereof, the method comprising administering to the mammalian subject a therapeutically effective amount of an antibody or antigen binding fragment thereof, combination of antibodies or antigen binding fragments thereof, or a pharmaceutical composition described herein.

In certain aspects, described herein are methods of reducing biological activity of TSLP in a mammalian subject in need thereof, the method comprising administering to the mammalian subject a therapeutically effective amount of an antibody or antigen binding fragment thereof, combination of antibodies or antigen binding fragments thereof, or a pharmaceutical composition described herein.

In certain aspects, described herein are methods for treating a pathology associated with elevated levels of OX40L in a mammalian subject in need thereof, the method comprising administering to the mammalian subject a therapeutically effective amount of an antibody or antigen binding fragment thereof, combination of antibodies or antigen binding fragments thereof, or a pharmaceutical composition described herein.

In certain aspects, described herein are methods of reducing biological activity of OX40L in a mammalian subject in need thereof, the method comprising administering to the mammalian subject a therapeutically effective amount of an antibody or antigen binding fragment thereof, combination of antibodies or antigen binding fragments thereof, or a pharmaceutical composition described herein.

In certain aspects, described herein are methods for inhibiting the TH2 type (Type 2) allergic response in a mammalian subject in need thereof, the method comprising administering to the mammalian subject a therapeutically effective amount of an antibody, combination of antibodies, or a pharmaceutical composition described herein.

In certain aspects, described herein are methods for inhibiting phosphorylation of STAT6 in a cell, the method comprising contacting the cell with an antibody or antigen binding fragment thereof, combination of antibodies or antigen binding fragments thereof, or a pharmaceutical composition described herein.

In certain aspects, described herein are methods for inhibiting CD23 expression in a cell, the method comprising contacting the cell with an antibody or antigen binding fragment thereof, combination of antibodies or antigen binding fragments thereof, or a pharmaceutical composition described herein.

In certain aspects, described herein are methods for inhibiting secretion of CCL2 and CCL26 from a cell, the method comprising contacting the cell with an antibody or antigen binding fragment thereof, combination of antibodies or antigen binding fragments thereof, or a pharmaceutical composition described herein.

In certain aspects, described herein are methods for inhibiting NTRK1 expression in a cell, the method comprising contacting the cell with an antibody or antigen binding fragment thereof, combination of antibodies or antigen binding fragments thereof, or a pharmaceutical composition described herein.

In certain aspects, described herein are methods for reducing a level of thymus- and activation-regulated chemokine (TARC)/CCL17 in a mammalian subject in need thereof, the method comprising administering to the mammalian subject a therapeutically effective amount an antibody or antigen binding fragment thereof, combination of antibodies or antigen binding fragments thereof, or a pharmaceutical composition described herein. In certain embodiments, the method reduces the level of TARC by at least about 50%, 60%, 70%, 80%, 85%, 90%, or 95%.

In certain aspects, described herein are methods of preventing an inflammatory disorder or disease in a mammalian subject in need thereof, the method comprising administering to the mammalian subject a therapeutically effective amount of an antibody or antigen binding fragment thereof, a combination of antibodies or antigen binding fragments thereof, or a pharmaceutical composition described herein.

In certain aspects, described herein are methods for reducing a level of a cytokine and/or a chemokine in a mammalian subject in need thereof, the method comprising administering to the mammalian subject a therapeutically effective amount of a combination of an anti-OX40L antibody, or antigen binding fragment thereof, and an anti-TSLP antibody, or antigen binding fragment thereof (e.g., in a single composition or in two separate compositions), or a pharmaceutical composition comprising an anti-OX40L antibody, or antigen binding fragment thereof, and an anti-TSLP antibody, or antigen binding fragment thereof, described herein. In certain embodiments, the chemokine is TARC and/or MDC. In certain embodiments, the chemokine is a type 2 chemokine. In certain embodiments, the type 2 chemokine is TARC or MDC. In certain embodiments, the cytokine is IL-13, IL-5, IL-31, IFN-γ, TNFα, or granulocyte-macrophage colony-stimulating factor (GM-CSF).

In certain aspects, described herein are methods for a reducing level of a type 2 chemokine in a mammalian subject in need thereof, the method comprising administering to the mammalian subject a therapeutically effective amount of a combination of an anti-OX40L antibody, or antigen binding fragment thereof, and an anti-TSLP antibody, or antigen binding fragment thereof (e.g., in a single composition or in two separate compositions), or a pharmaceutical composition comprising an anti-OX40L antibody, or antigen binding fragment thereof, and an anti-TSLP antibody, or antigen binding fragment thereof, described herein. In certain embodiments, the method reduces the level of the type 2 chemokine by at least about 50%, 60%, 70%, 80%, 85%, 90%, or 95%.

In certain aspects, described herein are methods for reducing a level of a type 2 cytokine and/or chemokine and a level of a type 1 cytokine and/or chemokine in a mammalian subject in need thereof, the method comprising administering to the mammalian subject a therapeutically effective amount of a combination of an anti-OX40L antibody, or antigen binding fragment thereof, and an anti-TSLP antibody, or antigen binding fragment thereof (e.g., in a single composition or in two separate compositions), or a pharmaceutical composition comprising an anti-OX40L antibody, or antigen binding fragment thereof, and an anti-TSLP antibody, or antigen binding fragment thereof, described herein. In certain embodiments, the method reduces the level of the type 2 cytokine and/or chemokine and the level of the type 1 cytokine and/or chemokine by at least about 50%, 60%, 70%, 80%, 85%, 90%, or 95%.

In certain aspects, described herein are methods for reducing a level of MDC in a mammalian subject in need thereof, the method comprising administering to the mammalian subject a therapeutically effective amount a combination of an anti-OX40L antibody, or antigen binding fragment thereof, and an anti-TSLP antibody, or antigen binding fragment thereof (e.g., in a single composition or in two separate compositions), or a pharmaceutical composition comprising an anti-OX40L antibody, or antigen binding fragment thereof, and an anti-TSLP antibody, or antigen binding fragment thereof, described herein. In certain embodiments, the method reduces the level of MDC by at least about 50%, 60%, 70%, 80%, 85%, 90%, or 95%.

In certain aspects, described herein are methods for reducing a level of IL-13, IL-5, IL-31, IFN-γ, TNFα, or GM-CSF in a mammalian subject in need thereof, the method comprising administering to the mammalian subject a therapeutically effective amount a combination of an anti-OX40L antibody, or antigen binding fragment thereof, and an anti-TSLP antibody, or antigen binding fragment thereof (e.g., in a single composition or in two separate compositions), or a pharmaceutical composition comprising an anti-OX40L antibody, or antigen binding fragment thereof, and an anti-TSLP antibody, or antigen binding fragment thereof, described herein. In certain embodiments, the method reduces the level of IL-13, IL-5, IL-31, IFN-γ, TNFα, or GM-CSF by at least about 20%, 30%, 40%, 50%, 60%, 70%, 80%, 85%, 90%, or 95%.

Any of the above-described methods may further include administration of a hyaluronidase or a variant thereof (e.g., rHuPH20). In some embodiments, the hyaluronidase or variant thereof is included in the same composition as the anti-OX40L antibody, or antigen binding fragment thereof and/or the anti-TSLP antibody, or antigen binding fragment thereof (i.e., the hyaluronidase or variant thereof may be included in a composition with one or both of the antibodies). In other embodiments, the hyaluronidase or variant thereof is administered separately from the anti-OX40L antibody, or antigen binding fragment thereof and the anti-TSLP antibody, or antigen binding fragment thereof. The hyaluronidase or variant thereof may be administered before or after both of the anti-OX40L antibody, or antigen binding fragment thereof, and the anti-TSLP antibody, or antigen binding fragment thereof, or may be administered at about the same time as the anti-OX40L antibody, or antigen binding fragment thereof, and the anti-TSLP antibody, or antigen binding fragment thereof (e.g., in a separate composition or via co-formulation). In certain embodiments, the hyaluronidase or variant thereof may be administered in between administration of the first antibody (e.g., an anti-OX40L antibody, or antigen binding fragment thereof) and the second antibody (e.g., an anti-TSLP antibody, or antigen binding fragment thereof).

### Methods of Administration

In some embodiments, the methods provided herein are useful for the treatment of a disease or disorder in an individual, e.g., a human. In an embodiment, the individual has received an anti-TSLP antibody, or antigen binding fragment thereof, and the method includes administering an anti-OX40L antibody, or antigen binding fragment thereof, described herein.

In an embodiment, the individual has received an anti-OX40L antibody, or antigen binding fragment thereof, and the method includes administering an anti-TSLP antibody, or antigen binding fragment thereof, described herein.

In an embodiment, the individual is administered an anti-TSLP antibody, or antigen binding fragment thereof, and an anti-OX40L antibody, or antigen binding fragment thereof. In certain embodiments, the treatment regimen of the present invention may involve administering the anti-TSLP antibody, or antigen binding fragment thereof, and the anti-OX40L antibody, or antigen binding fragment thereof, at about the same time. This may be achieved by administering a single composition or pharmacological formulation that includes both agents, or by administering two distinct compositions or formulations, at about the same time, wherein one composition includes the anti-TSLP antibody, or antigen binding fragment thereof, and the other includes the anti-OX40L antibody, or antigen binding fragment thereof. Alternatively, the anti-TSLP antibody, or antigen binding fragment thereof, may be administered before the anti-OX40L antibody, or antigen binding fragment thereof, by intervals ranging from minutes, hours, days to weeks, or the anti-OX40L antibody, or antigen binding fragment thereof, may be administered before the anti-TSLP antibody, or antigen binding fragment thereof, by intervals ranging from minutes, hours days to weeks.

In some embodiments, an antibody, or antigen binding fragment thereof, or combination of antibodies, or antigen binding fragments thereof, is administered intravenously, intramuscularly, subcutaneously, topically, orally, transdermally, intraperitoneally, intraorbitally, by implantation, by inhalation, intrathecally, intraventricularly, or intranasally. An effective amount of an anti-TSLP antibody, or antigen binding fragment thereof, and an anti-OX40L antibody, or antigen binding fragment thereof, may be administered for the treatment of a disease or disorder. In some embodiments, administration of the anti-TSLP antibody, or antigen binding fragment thereof, and the anti-OX40L antibody, or antigen binding fragment thereof, is intravenous or subcutaneous. In some embodiments, administration of the anti-TSLP antibody, or antigen binding fragment thereof, and the anti-OX40L antibody, or antigen binding fragment thereof, is intravenous. In some embodiments, administration of the anti-TSLP antibody, or antigen binding fragment thereof, and the anti-OX40L antibody, or antigen binding fragment thereof, is subcutaneous. The appropriate dosage of the anti-TSLP antibody, or antigen binding fragment thereof, and the anti-OX40L antibody, or antigen binding fragment thereof, may be determined based on the type of disease or disorder to be treated, the type of the anti-TSLP antibody and the anti-OX40L antibody, the severity and course of the disease or disorder, the clinical condition of the individual, the individual's clinical history and response to the treatment, and the discretion of the attending physician.

In some embodiments, the antibody, or antigen binding fragment thereof, or combination of antibodies, or antigen binding fragments thereof, provided herein is administered with at least one additional therapeutic agent. Any suitable additional therapeutic or immunotherapeutic agent may be administered with an antibody or combination of antibodies provided herein. Additional therapeutic agents include agents that are used to treat or prevent a disease or disorder such as, but not limited to, an inflammatory disease or disorder.

The additional therapeutic agent can be administered by any suitable means. In some embodiments, an antibody, or antigen binding fragment thereof, or combination of antibodies, or antigen binding fragments thereof, provided herein and the additional therapeutic agent are included in the same pharmaceutical composition. In some embodiments, an antibody, or antigen binding fragment thereof, or combination of antibodies, or antigen binding fragments thereof, provided herein and the additional therapeutic agent are included in different pharmaceutical compositions.

In embodiments where an antibody, or antigen binding fragment thereof, or combination of antibodies, or antigen binding fragments thereof, provided herein and the additional therapeutic agent are included in different pharmaceutical compositions, administration of the antibody, or antigen binding fragment thereof, or combination of antibodies, or antigen binding fragments thereof, can occur prior to, simultaneously, and/or following, administration of the additional therapeutic agent. In some embodiments, administration of an antibody, or antigen binding fragment thereof, or combination of antibodies, or antigen binding fragments thereof, provided herein and the additional therapeutic agent occur within about one month of each other. In some embodiments, administration of an antibody, or antigen binding fragment thereof, or combination of antibodies, or antigen binding fragments thereof, provided herein and the additional therapeutic agent occur within about one week of each other. In some embodiments, administration of an antibody, or antigen binding fragment thereof, or combination of antibodies, or antigen binding fragments thereof, provided herein and the additional therapeutic agent occur within about one day of each other. In some embodiments, administration of an antibody, or antigen binding fragment thereof, or combination of antibodies, or antigen binding fragments thereof, provided herein and the additional therapeutic agent occur within about twelve hours of each other. In some embodiments, administration of an antibody, or antigen binding fragment thereof, or combination of antibodies, or antigen binding fragments thereof, provided herein and the additional therapeutic agent occur within about one hour of each other.

In some embodiments, the method further comprises administration of a hyaluronidase or variant thereof. In certain embodiments, the hyaluronidase or variant thereof is included in the same composition with one or more of the antibodies (e.g., an anti-OX40L antibody, or antigen binding fragment thereof, and an anti-TSLP antibody, or antigen binding fragment thereof). In certain embodiments, the hyaluronidase or variant there if is administered in a separate formulation. In certain embodiments, the hyaluronidase or variant thereof and the antibodies or antigen binding fragments thereof are administered simultaneously in separate formulations (e.g., via two formulations: one containing the hyaluronidase or variant thereof and the other containing the combination of the TSLP antibody, or an antigen binding fragment thereof, and OX40L antibody, or an antigen binding fragment thereof, or via three formulations: one containing the hyaluronidase or variant thereof, another containing the TSLP antibody, or an antigen binding fragment thereof, and another containing the OX40L antibody, or an antigen binding fragment thereof). In certain embodiments, the hyaluronidase or variant thereof is administered to the patient prior to administration of the antibodies or antigen binding fragments thereof. In certain embodiments, the hyaluronidase or variant thereof is administered to the patient after administration of the antibodies or antigen binding fragments thereof. In certain embodiments, the hyaluronidase or variant thereof is administered to the patient after administration of one of the antibodies (either the TSLP antibody, or an antigen binding fragment thereof, or the OX40L antibody, or an antigen binding fragment thereof) but is administered before the administration of the second of the antibodies (either the TSLP antibody, or an antigen binding fragment thereof, or the OX40L antibody, or an antigen binding fragment thereof). In certain embodiments, the hyaluronidase or variant thereof and the antibodies or antigen binding fragments thereof are mixed and administered in a single formulation.

### Compositions, Combinations, and Methods Involving Co-Administration

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof. In certain aspects, described herein are compositions comprising any one of the antibodies, or antigen binding fragments thereof, described herein that binds TSLP, any one of the antibodies, or antigen binding fragments thereof, described herein that binds OX40L, and any one of the hyaluronidases or variants thereof described herein.

In certain aspects, described herein are compositions comprising hyaluronidase or a variant thereof and a multi-specific antibody comprising a first antigen binding region that binds TSLP and a second antigen binding region that binds OX40L.

In certain aspects, described herein are a combination of an antibody, or antigen binding fragment thereof, that binds TSLP, an antibody, or antigen binding fragment thereof, that binds OX40L, and a hyaluronidase or variant thereof. In certain aspects, described herein are a combination of any one of the antibodies, or antigen binding fragments thereof, described herein that binds TSLP, any one of the antibodies, or antigen binding fragments thereof, described herein that binds OX40L, and a hyaluronidase or a variant thereof.

In certain aspects, described herein are combinations comprising hyaluronidase or a variant thereof and a multi-specific antibody comprising a first antigen binding region that binds TSLP and a second antigen binding region that binds OX40L.

In certain aspects, described herein are methods of treating an inflammatory disorder or disease in a human patient comprising co-administering to the patient an isolated antibody, or antigen binding fragment thereof, that binds TSLP, an isolated antibody, or antigen binding fragment thereof, that binds OX40L, and a hyaluronidase or a variant thereof. In certain aspects, described herein are methods of treating an inflammatory disorder or disease in a human patient comprising co-administering to the patient any one of the antibodies, or antigen binding fragments thereof, described herein that binds TSLP, any one of the antibodies, or antigen binding fragments thereof, described herein that binds OX40L, and any one of the hyaluronidases or variants thereof described herein.

In certain aspects, described herein are methods of treating an inflammatory disorder or disease in a human patient comprising co-administering to the patient hyaluronidase or a variant thereof and a multi-specific antibody comprising a first antigen binding region that binds TSLP and a second antigen binding region that binds OX40L. In certain aspects, described herein are methods of treating an inflammatory disorder or disease in a human patient comprising co-administering to the patient any one of the hyaluronidases or variants thereof described herein and any one of the multi-specific antibodies described herein (e.g., comprising a first antigen binding region that binds TSLP and a second antigen binding region that binds OX40L).

In certain embodiments, the antibody, or antigen binding fragment thereof, that binds TSLP and/or the antibody, or antigen binding fragment thereof, that binds OX40L is a humanized, human, or chimeric antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E (e.g., for an OX40L antibody described herein). In another embodiment, the human Fc region comprises a human IgG1 Fc with LALA mutations at L239A/L240A and/or YTE mutations at M257Y/S259T/T261E (e.g., for a TSLP antibody described herein).

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antibody, or antigen binding fragment thereof, that binds TSLP comprises a VH sequence set forth in SEQ ID NO: 34 and a VL sequence set forth in SEQ ID NO: 41. In one embodiment, the antibody, or antigen binding fragment thereof, that binds TSLP comprises a heavy chain comprising a constant heavy chain sequence set forth in any one of SEQ ID NOs: 47-270. In one embodiment, the antibody, or antigen binding fragment thereof, that binds TSLP comprises a heavy chain comprising a constant heavy chain sequence set forth in SEQ ID NO: 61 or 173. In one embodiment, the antibody, or antigen binding fragment thereof, that binds TSLP comprises a light chain comprising a constant light chain sequence set forth in SEQ ID NO: 520. In certain embodiments, the antibody, or antigen binding fragment thereof, comprises a VH sequence set forth in SEQ ID NO: 34, a VL sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 520. In certain embodiments, the antibody, or antigen binding fragment thereof, comprises a VH sequence set forth in SEQ ID NO: 34, a VL sequence set forth in SEQ ID NO: 41, a constant heavy chain sequence set forth in SEQ ID NO: 173, and a constant light chain sequence set forth in SEQ ID NO: 520. In certain embodiments, the antibody, or antigen binding fragment thereof, comprises a heavy chain sequence set forth in SEQ ID NO: 524 and a light chain sequence set forth in SEQ ID NO: 525. In certain embodiments, the antibody, or antigen binding fragment thereof, comprises a VH sequence set forth in SEQ ID NO: 34, a VL sequence set forth in SEQ ID NO: 41, a constant heavy chain sequence set forth in SEQ ID NO: 61, and a constant light chain sequence set forth in SEQ ID NO: 520. In certain embodiments, the antibody, or antigen binding fragment thereof, comprises a heavy chain sequence set forth in SEQ ID NO: 526 and a light chain sequence set forth in SEQ ID NO: 525.

In certain embodiments, the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antibody, or antigen binding fragment thereof, that binds OX40L comprises a VH sequence set forth in SEQ ID NO: 335 and a VL sequence set forth in SEQ ID NO: 339. In one embodiment, the antibody, or antigen binding fragment thereof, that binds TSLP comprises a heavy chain comprising a constant heavy chain sequence set forth in any one of SEQ ID NOs: 47-270. In one embodiment, the antibody, or antigen binding fragment thereof, that binds OX40L comprises a heavy chain comprising a constant heavy chain sequence set forth in SEQ ID NO: 61 or 173. In one embodiment, the antibody, or antigen binding fragment thereof, that binds OX40L comprises a light chain comprising a constant light chain sequence set forth in SEQ ID NO: 46. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 173 and/or a constant heavy chain sequence set forth in SEQ ID NO: 61, and a constant light chain sequence set forth in SEQ ID NO: 46. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 173, and a constant light chain sequence set forth in SEQ ID NO: 46. In certain embodiments, the antibody, or antigen binding fragment thereof, comprises a heavy chain sequence set forth in SEQ ID NO: 543 and a light chain sequence set forth in SEQ ID NO: 545. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 61, and a constant light chain sequence set forth in SEQ ID NO: 46. In certain embodiments, the antibody, or antigen binding fragment thereof, comprises a heavy chain sequence set forth in SEQ ID NO: 544 and a light chain sequence set forth in SEQ ID NO: 545.

In certain embodiments, a multi-specific antibody comprising a first antigen binding region that binds TSLP and a second antigen binding region that binds OX40L is provided. In one embodiment, the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antigen binding region that binds TSLP comprises a VH sequence set forth in SEQ ID NO: 34 and a VL sequence set forth in SEQ ID NO: 41.

In another embodiment, the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antigen binding region that binds OX40L comprises a VH sequence set forth in SEQ ID NO: 335 and a VL sequence set forth in SEQ ID NO: 339.

In certain embodiments, the antibody or antigen binding region that binds TSLP and/or the antibody or antigen binding region that binds OX40L comprises a heavy chain human constant region of a class selected from IgG, IgA, IgD, IgE, and IgM. In certain embodiments, the antibody or antigen binding region that binds TSLP and/or the antibody or antigen binding region that binds OX40L comprises a human Fc region, and wherein the human Fc region comprises a human heavy chain constant region of the class IgG and a subclass selected from IgG1, IgG2, IgG3, and IgG4. In certain embodiments, the human Fc region of the antibody or antigen binding region that binds TSLP and/or the antibody or antigen binding region that binds OX40L comprises a human IgG1 Fc region. In certain embodiments, the human Fc region of the antibody or antigen binding region that binds TSLP and/or the antibody or antigen binding region that binds OX40L comprises a human IgG4 Fc region. In certain embodiments, the human Fc region of the antibody or antigen binding region that binds TSLP and/or the antibody or antigen binding region that binds OX40L comprises a human IgG2 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used, these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E (e.g., in an OX40L antibody described herein). In another embodiment, the human Fc region comprises a human IgG1 Fc region with LALA mutations at L239A/L240A and/or YTE mutations at M257Y/S259T/T261E (e.g., in a TSLP antibody described herein).

In certain embodiments, the Fc region of the antibody or antigen binding region that binds TSLP and/or the antibody or antigen binding region that binds OX40L binds to Neonatal Fc receptor (FcRn). In certain embodiments, the Fc region of the antibody or antigen binding region that binds TSLP and/or the antibody or antigen binding region that binds OX40L binds an FcRn with higher affinity at pH 6.0 compared to an antibody comprising a wild-type Fc region. In certain embodiments, the Fc region of the antibody or antigen binding region that binds TSLP and/or the antibody or antigen binding region that binds OX40L binds to FcRn with a KD of <1×10⁻⁷ M at pH 6.0.

In certain embodiments, the antibody or antigen binding region that binds TSLP and/or the antibody or antigen binding region that binds OX40L is a monoclonal antibody.

In certain embodiments, the antibody or antigen binding region that binds TSLP binds a TSLP sequence set forth in any one of the amino acid sequences of SEQ ID NOs: 271-272. In certain embodiments, the antibody or antigen binding region that binds OX40L binds an OX40L sequence set forth in the amino acid sequence of SEQ ID NO:439.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase comprises the amino acid sequence set forth in any one of SEQ ID NOs: 453-462 and 464-519, or a mixture thereof (e.g., the recombinant human hyaluronidase may comprise one or more of SEQ ID NOs: 453-462 and 464-519). In certain embodiments, the recombinant human hyaluronidase is rHuPH20 (i.e., a composition comprising one or more of SEQ ID NOs: 455-460). In certain embodiments, the rHuPH20 formulation is ENHANZE®.

In one embodiment, the recombinant human hyaluronidase includes a sequence of amino acids in any one of SEQ ID NOs: 453-462 and 464-519, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 453-462 and 464-519 and retains hyaluronidase activity. In one embodiment, the recombinant human hyaluronidase comprises a sequence having at least 95%, 96%, 97%, 98%, or 99% to the amino acid sequence of SEQ ID NO: 453. In one embodiment, the recombinant human hyaluronidase comprises a sequence having at least 95%, 96%, 97%, 98%, or 99% to the amino acid sequence of SEQ ID NO: 455.

In another embodiment, the recombinant human hyaluronidase comprises the amino acid sequence set forth in SEQ ID NO:453. In another embodiment, the recombinant human hyaluronidase consists of the amino acid sequence set forth in SEQ ID NO:453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO:454. In another embodiment, the recombinant human hyaluronidase comprises the amino acid sequence set forth in SEQ ID NO:455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO:456. In another embodiment, the recombinant human hyaluronidase consists of the amino acid sequence set forth in SEQ ID NO: 456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO:457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO:458. In another embodiment, the recombinant human hyaluronidase comprises the amino acid sequence set forth in SEQ ID NO:459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO:460. In another embodiment, the recombinant human hyaluronidase consists of the amino acid sequence set forth in SEQ ID NO: 460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO:461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO:462. In another embodiment, the recombinant human hyaluronidase comprises the amino acid sequence set forth in SEQ ID NO:464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO: 465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO:466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO:468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO: 469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO:470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO:472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO: 473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO:474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475. In another embodiment, the recombinant human hyaluronidase comprises the amino acid sequence set forth in SEQ ID NO:476. In another embodiment, the recombinant human hyaluronidase consists of the amino acid sequence set forth in SEQ ID NO:476. In another embodiment, the recombinant human hyaluronidase comprises the amino acid sequence set forth in SEQ ID NO:477. In another embodiment, the recombinant human hyaluronidase consists of the amino acid sequence set forth in SEQ ID NO: 477. In another embodiment, the recombinant human hyaluronidase comprises the amino acid sequence set forth in SEQ ID NO:478. In another embodiment, the recombinant human hyaluronidase consists of the amino acid sequence set forth in SEQ ID NO:478. In another embodiment, the recombinant human hyaluronidase comprises the amino acid sequence set forth in SEQ ID NO:479. In another embodiment, the recombinant human hyaluronidase consists of the amino acid sequence set forth in SEQ ID NO:479. In another embodiment, the recombinant human hyaluronidase comprises the amino acid sequence set forth in SEQ ID NO:480. In another embodiment, the recombinant human hyaluronidase consists of the amino acid sequence set forth in SEQ ID NO:480. In another embodiment, the recombinant human hyaluronidase comprises the amino acid sequence set forth in SEQ ID NO:481. In another embodiment, the recombinant human hyaluronidase consists of the amino acid sequence set forth in SEQ ID NO: 481. In another embodiment, the recombinant human hyaluronidase comprises the amino acid sequence set forth in SEQ ID NO:482. In another embodiment, the recombinant human hyaluronidase consists of the amino acid sequence set forth in SEQ ID NO:482. In another embodiment, the recombinant human hyaluronidase comprises the amino acid sequence set forth in SEQ ID NO:483. In another embodiment, the recombinant human hyaluronidase consists of the amino acid sequence set forth in SEQ ID NO:483. In another embodiment, the recombinant human hyaluronidase comprises the amino acid sequence set forth in SEQ ID NO:484. In another embodiment, the recombinant human hyaluronidase consists of the amino acid sequence set forth in SEQ ID NO:484. In another embodiment, the recombinant human hyaluronidase comprises the amino acid sequence set forth in SEQ ID NO:485. In another embodiment, the recombinant human hyaluronidase consists of the amino acid sequence set forth in SEQ ID NO: 485. In another embodiment, the recombinant human hyaluronidase comprises the amino acid sequence set forth in SEQ ID NO:486. In another embodiment, the recombinant human hyaluronidase consists of the amino acid sequence set forth in SEQ ID NO:486. In another embodiment, the recombinant human hyaluronidase comprises the amino acid sequence set forth in SEQ ID NO:487. In another embodiment, the recombinant human hyaluronidase consists of the amino acid sequence set forth in SEQ ID NO:487. In another embodiment, the recombinant human hyaluronidase comprises the amino acid sequence set forth in SEQ ID NO:488. In another embodiment, the recombinant human hyaluronidase consists of the amino acid sequence set forth in SEQ ID NO:488. In another embodiment, the recombinant human hyaluronidase comprises the amino acid sequence set forth in SEQ ID NO:489. In another embodiment, the recombinant human hyaluronidase consists of the amino acid sequence set forth in SEQ ID NO: 489. In another embodiment, the recombinant human hyaluronidase comprises the amino acid sequence set forth in SEQ ID NO:490. In another embodiment, the recombinant human hyaluronidase consists of the amino acid sequence set forth in SEQ ID NO:490. In another embodiment, the recombinant human hyaluronidase comprises the amino acid sequence set forth in SEQ ID NO:491. In another embodiment, the recombinant human hyaluronidase consists of the amino acid sequence set forth in SEQ ID NO:491. In another embodiment, the recombinant human hyaluronidase comprises the amino acid sequence set forth in SEQ ID NO:492. In another embodiment, the recombinant human hyaluronidase consists of the amino acid sequence set forth in SEQ ID NO:492. In another embodiment, the recombinant human hyaluronidase comprises the amino acid sequence set forth in SEQ ID NO:493. In another embodiment, the recombinant human hyaluronidase consists of the amino acid sequence set forth in SEQ ID NO: 493. In another embodiment, the recombinant human hyaluronidase comprises the amino acid sequence set forth in SEQ ID NO:494. In another embodiment, the recombinant human hyaluronidase consists of the amino acid sequence set forth in SEQ ID NO:494. In another embodiment, the recombinant human hyaluronidase comprises the amino acid sequence set forth in SEQ ID NO:495. In another embodiment, the recombinant human hyaluronidase consists of the amino acid sequence set forth in SEQ ID NO:495. In another embodiment, the recombinant human hyaluronidase comprises the amino acid sequence set forth in SEQ ID NO:496. In another embodiment, the recombinant human hyaluronidase consists of the amino acid sequence set forth in SEQ ID NO:496. In another embodiment, the recombinant human hyaluronidase comprises the amino acid sequence set forth in SEQ ID NO:497. In another embodiment, the recombinant human hyaluronidase consists of the amino acid sequence set forth in SEQ ID NO: 497. In another embodiment, the recombinant human hyaluronidase comprises the amino acid sequence set forth in SEQ ID NO:498. In another embodiment, the recombinant human hyaluronidase consists of the amino acid sequence set forth in SEQ ID NO:498. In another embodiment, the recombinant human hyaluronidase comprises the amino acid sequence set forth in SEQ ID NO:499. In another embodiment, the recombinant human hyaluronidase consists of the amino acid sequence set forth in SEQ ID NO:499. In another embodiment, the recombinant human hyaluronidase comprises the amino acid sequence set forth in SEQ ID NO:500. In another embodiment, the recombinant human hyaluronidase consists of the amino acid sequence set forth in SEQ ID NO:500. In another embodiment, the recombinant human hyaluronidase comprises the amino acid sequence set forth in SEQ ID NO:501. In another embodiment, the recombinant human hyaluronidase consists of the amino acid sequence set forth in SEQ ID NO: 501. In another embodiment, the recombinant human hyaluronidase comprises the amino acid sequence set forth in SEQ ID NO:502. In another embodiment, the recombinant human hyaluronidase consists of the amino acid sequence set forth in SEQ ID NO:502. In another embodiment, the recombinant human hyaluronidase comprises the amino acid sequence set forth in SEQ ID NO:503. In another embodiment, the recombinant human hyaluronidase consists of the amino acid sequence set forth in SEQ ID NO:503. In another embodiment, the recombinant human hyaluronidase comprises the amino acid sequence set forth in SEQ ID NO:504. In another embodiment, the recombinant human hyaluronidase consists of the amino acid sequence set forth in SEQ ID NO:504. In another embodiment, the recombinant human hyaluronidase comprises the amino acid sequence set forth in SEQ ID NO:505. In another embodiment, the recombinant human hyaluronidase consists of the amino acid sequence set forth in SEQ ID NO: 505. In another embodiment, the recombinant human hyaluronidase comprises the amino acid sequence set forth in SEQ ID NO:506. In another embodiment, the recombinant human hyaluronidase consists of the amino acid sequence set forth in SEQ ID NO:506. In another embodiment, the recombinant human hyaluronidase comprises the amino acid sequence set forth in SEQ ID NO:507. In another embodiment, the recombinant human hyaluronidase consists of the amino acid sequence set forth in SEQ ID NO:507. In another embodiment, the recombinant human hyaluronidase comprises the amino acid sequence set forth in SEQ ID NO:508. In another embodiment, the recombinant human hyaluronidase consists of the amino acid sequence set forth in SEQ ID NO:508. In another embodiment, the recombinant human hyaluronidase comprises the amino acid sequence set forth in SEQ ID NO:509. In another embodiment, the recombinant human hyaluronidase consists of the amino acid sequence set forth in SEQ ID NO: 509. In another embodiment, the recombinant human hyaluronidase comprises the amino acid sequence set forth in SEQ ID NO:510. In another embodiment, the recombinant human hyaluronidase consists of the amino acid sequence set forth in SEQ ID NO:510. In another embodiment, the recombinant human hyaluronidase comprises the amino acid sequence set forth in SEQ ID NO:511. In another embodiment, the recombinant human hyaluronidase consists of the amino acid sequence set forth in SEQ ID NO:511. In another embodiment, the recombinant human hyaluronidase comprises the amino acid sequence set forth in SEQ ID NO:512. In another embodiment, the recombinant human hyaluronidase consists of the amino acid sequence set forth in SEQ ID NO:512. In another embodiment, the recombinant human hyaluronidase comprises the amino acid sequence set forth in SEQ ID NO:513. In another embodiment, the recombinant human hyaluronidase consists of the amino acid sequence set forth in SEQ ID NO: 513. In another embodiment, the recombinant human hyaluronidase comprises the amino acid sequence set forth in SEQ ID NO:514. In another embodiment, the recombinant human hyaluronidase consists of the amino acid sequence set forth in SEQ ID NO:514. In another embodiment, the recombinant human hyaluronidase comprises the amino acid sequence set forth in SEQ ID NO:515. In another embodiment, the recombinant human hyaluronidase consists of the amino acid sequence set forth in SEQ ID NO:515. In another embodiment, the recombinant human hyaluronidase comprises the amino acid sequence set forth in SEQ ID NO:516. In another embodiment, the recombinant human hyaluronidase consists of the amino acid sequence set forth in SEQ ID NO:516. In another embodiment, the recombinant human hyaluronidase comprises the amino acid sequence set forth in SEQ ID NO:517. In another embodiment, the recombinant human hyaluronidase consists of the amino acid sequence set forth in SEQ ID NO: 517. In another embodiment, the recombinant human hyaluronidase comprises the amino acid sequence set forth in SEQ ID NO:518. In another embodiment, the recombinant human hyaluronidase consists of the amino acid sequence set forth in SEQ ID NO:518. In another embodiment, the recombinant human hyaluronidase comprises the amino acid sequence set forth in SEQ ID NO:519. In another embodiment, the recombinant human hyaluronidase consists of the amino acid sequence set forth in SEQ ID NO:519.

In certain embodiments, the hyaluronidase or variant thereof and antibodies (i.e., anti-OX40L antibody, or antigen binding fragment thereof, and anti-TSLP antibody, or antigen binding fragment thereof) or multi-specific antibody (i.e., comprising a first antigen binding region that binds TSLP and a second antigen binding region that binds OX40L) are administered in separate formulations (e.g., via two formulations: one containing the hyaluronidase or variant thereof and the other containing the combination of the TLSP antibody, or antigen binding fragment thereof, and OX40L antibody, or antigen binding fragment thereof, or via three formulations: one containing the hyaluronidase or variant thereof, another containing the TSLP antibody, or antigen binding fragment thereof, and another containing the OX40L antibody, or antigen binding fragment thereof). In certain embodiments, the hyaluronidase or variant thereof and antibodies or multi-specific antibody are administered simultaneously in separate formulations. In certain embodiments, the hyaluronidase or variant thereof is administered to the patient prior to administration of the antibodies or multi-specific antibody. In certain embodiments, the hyaluronidase or variant thereof is administered to the patient after administration of the antibodies or multi-specific antibody. In certain embodiments, the hyaluronidase or variant thereof is administered to the patient after administration of one of the antibodies (either the TSLP antibody, or antigen binding fragment thereof, or the OX40L antibody, or antigen binding fragment thereof) but is administered before the administration of the second of the antibodies (either the TSLP antibody, or antigen binding fragment thereof, or the OX40L antibody, or antigen binding fragment thereof).

In certain embodiments, the hyaluronidase or variant thereof and the antibodies or multi-specific antibody are mixed and administered in a single formulation. In certain embodiments, the hyaluronidase or variant thereof and the antibodies or multi-specific antibody are administered by subcutaneous injection. In certain embodiments, the hyaluronidase or variant thereof and the antibodies or multi-specific antibody are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind TSLP described herein and any one of the antibodies, or antigen binding fragments thereof, that bind OX40L described herein in combination with a hyaluronidase or variant thereof. In certain embodiments, methods of treating an inflammatory disorder or disease in a human patient are provided, wherein the method comprises co-administering to the patient any one of the multi-specific antibodies described herein in combination with a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is asthma, chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; idiopathic pulmonary fibrosis; alopecia areata; systemic sclerosis allergic rhinitis; eosinophilic fasciitis; scleromyxedema; scleredema; or nephrogenic systemic fibrosis.

In certain aspects, described herein are compositions comprising an isolated antibody, or antigen binding region thereof, that binds TSLP, an isolated antibody, or antigen binding region thereof, that binds OX40L, and a hyaluronidase or variant thereof.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds TSLP comprises a VH sequence set forth in SEQ ID NO: 34 and a VL sequence set forth in SEQ ID NO: 41.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising an isolated antibody, or antigen binding region thereof, that binds TSLP, an isolated antibody, or antigen binding region thereof, that binds OX40L, and a hyaluronidase or variant thereof, wherein the antibody or antigen binding region thereof that binds OX40L comprises a VH sequence set forth in SEQ ID NO: 335 and a VL sequence set forth in SEQ ID NO: 339.

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a VH sequence set forth in SEQ ID NO: 34 and a VL sequence set forth in SEQ ID NO: 41; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are compositions comprising hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are compositions comprising hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain aspects, described herein are combinations comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are combinations comprising an isolated antibody, or antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises a VH sequence set forth in SEQ ID NO: 34 and a VL sequence set forth in SEQ ID NO: 41; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are combinations comprising hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are combinations comprising hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain aspects, described herein are methods of treating an inflammatory disorder or disease in a human patient comprising administering to the patient an isolated antibody, or antigen binding fragment thereof, that binds TSLP, an isolated antibody, or antigen binding fragment thereof, that binds OX40L, and a hyaluronidase or a variant thereof, wherein the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are methods of treating an inflammatory disorder or disease in a human patient comprising administering to the patient an isolated antibody, or antigen binding fragment thereof, that binds TSLP, an isolated antibody, or antigen binding fragment thereof, that binds OX40L, and a hyaluronidase or a variant thereof, wherein the antibody, or antigen binding fragment thereof, that binds TSLP comprises a VH sequence set forth in SEQ ID NO: 34 and a VL sequence set forth in SEQ ID NO: 41; and wherein the antibody, or antigen binding fragment thereof, that binds OX40L comprises a VH sequence set forth in SEQ ID NO: 335 and a VL sequence set forth in SEQ ID NO: 339.

In certain aspects, described herein are methods of treating an inflammatory disorder or disease in a human patient comprising administering to the patient hyaluronidase or a variant thereof and a multi-specific antibody comprising a first antigen binding region that binds TSLP and a second antigen binding region that binds OX40L, wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

In certain aspects, described herein are methods of treating an inflammatory disorder or disease in a human patient comprising administering to the patient hyaluronidase or a variant thereof and a multi-specific antibody comprising a first antigen binding region that binds TSLP and a second antigen binding region that binds OX40L, wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339.

In certain embodiments of any of the foregoing aspects, the isolated antibody, or antigen binding fragment thereof, that binds TSLP comprises a VH sequence set forth in SEQ ID NO: 34, a VL sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 520. In certain embodiments, the antibody, or antigen binding fragment thereof, comprises a VH sequence set forth in SEQ ID NO: 34, a VL sequence set forth in SEQ ID NO: 41, a constant heavy chain sequence set forth in SEQ ID NO: 173, and a constant light chain sequence set forth in SEQ ID NO: 520. In certain embodiments, the antibody, or antigen binding fragment thereof, comprises a heavy chain sequence set forth in SEQ ID NO: 524 and a light chain sequence set forth in SEQ ID NO: 525. In certain embodiments, the antibody, or antigen binding fragment thereof, comprises a VH sequence set forth in SEQ ID NO: 34, a VL sequence set forth in SEQ ID NO: 41, a constant heavy chain sequence set forth in SEQ ID NO: 61, and a constant light chain sequence set forth in SEQ ID NO: 520. In certain embodiments, the antibody, or antigen binding fragment thereof, comprises a heavy chain sequence set forth in SEQ ID NO: 526 and a light chain sequence set forth in SEQ ID NO: 525.

In certain embodiments of any of the foregoing aspects, the isolated antibody, or antigen binding fragment thereof, that binds OX40L comprises a VH sequence set forth in SEQ ID NO: 335, a VL sequence set forth in SEQ ID NO: 339, a constant heavy chain sequence set forth in SEQ ID NO: 173 and/or a constant heavy chain sequence set forth in SEQ ID NO: 61, and a constant light chain sequence set forth in SEQ ID NO: 46. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 173, and a constant light chain sequence set forth in SEQ ID NO: 46. In certain embodiments, the antibody, or antigen binding fragment thereof, comprises a heavy chain sequence set forth in SEQ ID NO: 543 and a light chain sequence set forth in SEQ ID NO: 545. In certain embodiments, the antibody, or antigen binding fragment thereof, comprises a VH sequence set forth in SEQ ID NO: 335, a VL sequence set forth in SEQ ID NO: 339, a constant heavy chain sequence set forth in SEQ ID NO: 61, and a constant light chain sequence set forth in SEQ ID NO: 46. In certain embodiments, the antibody, or antigen binding fragment thereof, comprises a heavy chain sequence set forth in SEQ ID NO: 544 and a light chain sequence set forth in SEQ ID NO: 545.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 453-462 and 464-519, or a mixture thereof. In certain embodiments, the recombinant human hyaluronidase is rHuPH20 (i.e., a composition comprising one or more of SEQ ID NOs: 455-460). In certain embodiments, the rHuPH20 formulation is ENHANZE®.

### Kits and Articles of Manufacture

The present application provides kits comprising any one or more of the antibody compositions or compositions of or combinations of antibodies, or antigen binding fragments thereof, described herein and instructions for use. In some embodiments, the kits further contain a component selected from any of secondary antibodies, additional therapeutic agents, reagents for immunohistochemistry analysis, a pharmaceutically acceptable excipient, a package insert, and an instruction manual and any combination thereof. In one specific embodiment, the kit comprises a pharmaceutical composition comprising any one or more of the antibody compositions or compositions of or combinations of the antibodies, or antigen binding fragments thereof, described herein, with one or more pharmaceutically acceptable excipients.

The present application also provides articles of manufacture comprising any one of the antibody compositions or kits described herein. Examples of an article of manufacture include vials (including sealed vials).

In certain aspects, described herein are kits for treating an inflammatory disorder or disease in a human patient, the kit comprising:
(a) a dose of an antibody, or antigen binding fragment thereof, that binds TSLP (e.g., an antibody, or an antigen binding fragment thereof, that binds TSLP in unit dosage form);
(b) a dose of an antibody, or antigen binding fragment thereof, that binds OX40L (e.g., an antibody, or an antigen binding fragment thereof, that binds OX40L in unit dosage form);
(c) a dose of a hyaluronidase or variant thereof (e.g., a hyaluronidase or a variant thereof in unit dosage form); and
(d) instructions.

In certain aspects, described herein are kits for treating an inflammatory disorder or disease in a human patient, the kit comprising:
(a) a dose of an antibody, or antigen binding fragment thereof, that binds TSLP (e.g., an antibody, or an antigen binding fragment thereof, that binds TSLP in unit dosage form), wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27;
(b) a dose of an antibody, or antigen binding fragment thereof, that binds OX40L (e.g., an antibody, or an antigen binding fragment thereof, that binds OX40L in unit dosage form), wherein the antibody, or antigen binding fragment thereof, comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327;
(c) a dose of a hyaluronidase or variant thereof (e.g., a hyaluronidase or a variant thereof in unit dosage form); and
(d) instructions.

In certain aspects, described herein are kits for treating an inflammatory disorder or disease in a human patient, the kit comprising:
(a) a dose of an antibody, or antigen binding fragment thereof, that binds TSLP (e.g., an antibody, or an antigen binding fragment thereof, that binds TSLP in unit dosage form), wherein the antibody, or antigen binding fragment thereof, comprises a VH sequence set forth in SEQ ID NO: 34 and a VL sequence set forth in SEQ ID NO: 41;
(b) a dose of an antibody, or antigen binding fragment thereof, that binds OX40L (e.g., an antibody, or an antigen binding fragment thereof, that binds OX40L in unit dosage form), wherein the antibody, or antigen binding fragment thereof, comprises a VH sequence set forth in SEQ ID NO: 335 and a VL sequence set forth in SEQ ID NO: 339;
(c) a dose of a hyaluronidase or variant thereof (e.g., a hyaluronidase or a variant thereof in unit dosage form); and
(d) instructions.

In certain aspects, described herein are kits for treating an inflammatory disorder or disease in a human patient, the kit comprising:
(a) a dose of a multi-specific antibody comprising a first antigen binding region that binds TSLP and a second antigen binding region that binds OX40L (e.g., a multi-specific antibody in unit dosage form);
(b) a dose of a hyaluronidase or variant thereof (e.g., a hyaluronidase or a variant thereof in unit dosage form); and
(c) instructions.

In certain aspects, described herein are kits for treating an inflammatory disorder or disease in a human patient, the kit comprising:
(a) a dose of a multi-specific antibody comprising a first antigen binding region that binds TSLP and a second antigen binding region that binds OX40L (e.g., a multi-specific antibody in unit dosage form), wherein the antigen binding region that binds TSLP comprises a VH sequence set forth in SEQ ID NO: 34 and a VL sequence set forth in SEQ ID NO: 41; and wherein the antigen binding region that binds OX40L comprises a VH sequence set forth in SEQ ID NO: 335 and a VL sequence set forth in SEQ ID NO: 339;
(b) a dose of a hyaluronidase or variant thereof (e.g., a hyaluronidase or a variant thereof in unit dosage form); and
(c) instructions.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 453-462 and 464-519, or a mixture thereof. In certain embodiments, the recombinant human hyaluronidase is rHuPH20 (i.e., a composition comprising one or more of SEQ ID NOs: 455-460). In certain embodiments, the rHuPH20 formulation is ENHANZE®.

In certain embodiments, the kit is for use in treating an inflammatory disorder or disease. In certain embodiments, the inflammatory disorder or disease is atopic dermatitis. In certain embodiments, the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); inflammatory bowel disease, such as Crohn's disease or ulcerative colitis; lupus; rheumatoid arthritis (RA); psoriasis; hidradenitis suppurativa; celiac disease; idiopathic pulmonary fibrosis; alopecia areata; systemic sclerosis allergic rhinitis; eosinophilic fasciitis; scleromyxedema; scleredema; or nephrogenic systemic fibrosis.

### EXAMPLE

Provided herein is an example of specific embodiments for carrying out the present invention. The example is offered for illustrative purposes only, and is not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B (1992).

### Example 1: Clinical Study

A clinical study is conducted to assess the safety, tolerability, pharmacokinetics, pharmacodynamics, and immunogenicity of an isolated antibody, or antigen binding fragment thereof, that binds TSLP and an isolated antibody, or antigen binding fragment thereof, that binds OX40L administered in combination with hyaluronidase or a variant thereof.

In one embodiment, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antibody, or antigen binding fragment thereof, that binds TSLP comprises a VH sequence set forth in SEQ ID NO: 34 and a VL sequence set forth in SEQ ID NO: 41. In one embodiment, the antibody, or antigen binding fragment thereof, that binds TSLP comprises a heavy chain comprising a constant heavy chain sequence set forth in SEQ ID NO: 61 or 173. In one embodiment, the antibody, or antigen binding fragment thereof, that binds TSLP comprises light chain comprising a constant light chain sequence set forth in SEQ ID NO: 520. A C-terminal lysine is present in SEQ ID NO: 173, which may be cleaved off during manufacture or after administration, resulting in the sequence of SEQ ID NO: 61. Accordingly, a composition comprising an antibody comprising the constant heavy chain of SEQ ID NO: 173 that is administered to a subject may comprise antibodies having the constant heavy chain sequence set forth in SEQ ID NO: 173 or SEQ ID NO: 61, or a mixture thereof (e.g., a composition comprising anti-TSLP antibodies may contain a mixture of antibodies having either a constant heavy chain sequence of SEQ ID NO: 173 or a constant heavy chain sequence of SEQ ID NO: 61 and/or antibodies containing both constant heavy chain sequences (e.g., in a single antibody containing two constant heavy chain sequences)). In one embodiment, the antibody that binds TSLP comprises the heavy chain sequence set forth in SEQ ID NO: 524 and the light chain sequence set forth in SEQ ID NO: 525. The C-terminal lysine in SEQ ID NO: 525 may not be present if cleavage occurs during manufacture or after administration (which would result in the heavy chain sequence of SEQ ID NO: 526).

In one embodiment, the antibody, or antigen binding fragment thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antibody, or antigen binding fragment thereof, that binds OX40L comprises a VH sequence set forth in SEQ ID NO: 335 and a VL sequence set forth in SEQ ID NO: 339. In one embodiment, the antibody, or antigen binding fragment thereof, that binds OX40L comprises a heavy chain comprising a constant heavy chain sequence set forth in SEQ ID NO: 61 or 173. In one embodiment, the antibody, or antigen binding fragment thereof, that binds OX40L comprises a light chain comprising a constant light chain sequence set forth in SEQ ID NO: 46. A C-terminal lysine is present in SEQ ID NO: 173, which may be cleaved off during manufacture or after administration, resulting in the sequence of SEQ ID NO: 61. Accordingly, a composition comprising an antibody comprising the constant heavy chain of SEQ ID NO: 173 that is administered to a subject may comprise antibodies having the constant heavy chain sequence set forth in SEQ ID NO: 173 or SEQ ID NO: 61, or a mixture thereof (e.g., a composition comprising anti-OX40L antibodies may contain a mixture of antibodies having either a constant heavy chain sequence of SEQ ID NO: 173 or a constant heavy chain sequence of SEQ ID NO: 61 and/or antibodies containing both constant heavy chain sequences (e.g., in a single antibody containing two constant heavy chain sequences)). In one embodiment, the antibody that binds OX40L comprises the heavy chain sequence set forth in SEQ ID NO: 543 and the light chain sequence set forth in SEQ ID NO: 545. The C-terminal lysine in SEQ ID NO: 543 may not be present if cleavage occurs during manufacture or after administration (which would result in the heavy chain sequence of SEQ ID NO: 544).

Alternatively, a multi-specific antibody comprising a first antigen binding region that binds TSLP and a second antigen binding region that binds OX40L is administered in combination with hyaluronidase or a variant thereof.

In one embodiment, the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In one embodiment, the antigen binding region that binds TSLP comprises a VH sequence set forth in SEQ ID NO: 34 and a VL sequence set forth in SEQ ID NO: 41.

In one embodiment, the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327. In one embodiment, the antigen binding region that binds OX40L comprises a VH sequence set forth in SEQ ID NO: 335 and a VL sequence set forth in SEQ ID NO: 339.

In one embodiment, the hyaluronidase is a recombinant human hyaluronidase, such as rHuPH20 (i.e., a composition comprising one or more of SEQ ID NOs: 455-460). In certain embodiments, the rHuPH20 formulation is ENHANZE®. In certain embodiments, the recombinant human hyaluronidase comprises the amino acid sequence set forth in any one of SEQ ID NOs: 453-462 and 464-519, or a mixture thereof.

In certain embodiments, the hyaluronidase or variant thereof and the antibodies or multi-specific antibody are administered in separate formulations. In certain embodiments, the hyaluronidase or variant thereof and the antibodies or multi-specific antibody are administered simultaneously in separate formulations (e.g., one formulation containing the hyaluronidase or a variant thereof and a second formulation containing the antibodies or multi-specific antibody; or one formulation containing the hyaluronidase or a variant thereof, another formulation containing the TSLP antibody, or antigen binding fragment thereof, and another formulation containing the OX40L antibody, or antigen binding fragment thereof). In certain embodiments, the hyaluronidase or variant thereof is administered to the patient prior to administration of the antibodies or multi-specific antibody. In certain embodiments, the hyaluronidase or variant thereof is administered to the patient after administration of one of the antibodies (either the TSLP antibody, or antigen binding fragment thereof, or the OX40L antibody, or antigen binding fragment thereof) but is administered before the administration of the second of the antibodies (either the TSLP antibody, or antigen binding fragment thereof, or the OX40L antibody, or antigen binding fragment thereof). In certain embodiments, the hyaluronidase or variant thereof is administered to the patient after administration of the antibodies or multi-specific antibody. In certain embodiments, the hyaluronidase or variant thereof and the antibodies or multi-specific antibody are mixed and administered in a single formulation. In certain embodiments, the hyaluronidase or variant thereof and the antibodies or multi-specific antibody are administered by subcutaneous injection. In certain embodiments, the hyaluronidase or variant thereof and the antibodies or multi-specific antibody are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

Exemplary embodiments of the invention are described in the enumerated paragraphs below.

E1 A composition comprising an isolated antibody, or an antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E2. The composition of E1, wherein the isolated antibody, or antigen binding fragment thereof, that binds OX40L comprises a CDR-H1, CDR-H2, CDR-H3, CDR-L1, CDR-L2, and CDR-L3 of an antibody selected from amlitelimab and oxelumab.

E3. The composition of E1, wherein the isolated antibody, or antigen binding fragment thereof, that binds OX40L comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E4. A composition comprising an isolated antibody, or an antigen binding fragment thereof, that binds TSLP, and an isolated antibody, or an antigen binding fragment thereof, that binds OX40L, and a hyaluronidase or variant thereof, wherein the antibody, or antigen binding fragment that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E5. The composition of E4, wherein the isolated antibody, or antigen binding fragment thereof, that binds TSLP comprises a CDR-H1, CDR-H2, CDR-H3, CDR-L1, CDR-L2, and CDR-L3 of an antibody selected from tezepelumab, GR2002, TQC2731, solrikitug, hu23b12, 43B1-H2L2, and 43B1-H6L1.

E6 The composition of E4, wherein the isolated antibody, or antigen binding fragment thereof, that binds TSLP comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E7. The composition of E3 or E6, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E8 The composition of E3 or E6, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E9 The composition of E3 or E6, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E10. The composition of E3 or E6, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E11. The composition of E3 or E6, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E12. The composition of E3 or E6, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E13. The composition of E3 or E6, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E14. The composition of E3 or E6, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E15. The composition of E3 or E6, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E16. The composition of E3 or E6, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E17. The composition of E3 or E6, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E18. The composition of E3 or E6, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E19. The composition of E3 or E6, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E20. The composition of E3 or E6, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E21. The composition of E3 or E6, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E22. The composition of E3 or E6, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E23. The composition of any one of E1-E4 and E6-E22, wherein the antibody, or antigen binding fragment thereof, that binds TSLP comprises a heavy chain variable domain (VH) sequence comprising the amino acid sequence set forth in SEQ ID NO: 34.

E24. The composition of any one of E1 and E3-E23, wherein the antibody, or antigen binding fragment thereof, that binds OX40L comprises a VH sequence comprising the amino acid sequence set forth in any one of SEQ ID NOs: 335-338.

E25. The composition of any one of E1-E4 and E6-E24, wherein the antibody, or antigen binding fragment thereof, that binds TSLP comprises a light chain variable domain (VL) sequence comprising the amino acid sequence set forth in any one of SEQ ID NOs: 41, 43, 415, and 421.

E26. The composition of any one of E1 and E3-E25, wherein the antibody, or antigen binding fragment thereof, that binds OX40L comprises a VL sequence comprising the amino acid sequence set forth in any one of SEQ ID NOs: 339-342.

E27. The composition of any one of E23-E26, wherein:
(a) the antibody, or antigen binding fragment thereof, that binds TSLP comprises:
   (i) a VH sequence comprising the amino acid sequence set forth in SEQ ID NO: 34; and/or
   (ii) a VL sequence comprising the amino acid sequence set forth in SEQ ID NO: 41;
   (iii) a VL sequence comprising the amino acid sequence set forth in SEQ ID NO: 43.
   (iv) a VL sequence comprising the amino acid sequence set forth in SEQ ID NO: 415; or
   (v) a VL sequence comprising the amino acid sequence set forth in SEQ ID NO: 421; and/or
(b) the antibody, or antigen binding fragment thereof, that binds OX40L comprises:
   (i) a VH sequence comprising the amino acid sequence set forth in SEQ ID NO: 335;
   (ii) a VH sequence comprising the amino acid sequence set forth in SEQ ID NO: 336;
   (iii) a VH sequence comprising the amino acid sequence set forth in SEQ ID NO: 337; or
   (iv) a VH sequence comprising the amino acid sequence set forth in SEQ ID NO: 338; and/or
   (v) a VL sequence comprising the amino acid sequence set forth in SEQ ID NO: 339;
   (vi) a VL sequence comprising the amino acid sequence set forth in SEQ ID NO: 400;
   (vii) a VL sequence comprising the amino acid sequence set forth in SEQ ID NO: 401; or
   (viii) a VL sequence comprising the amino acid sequence set forth in SEQ ID NO: 402.

E28. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41.

E29. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43.

E30. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415.

E31. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421.

E32. The composition of E27, wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E33. The composition of E27, wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E34. The composition of E27, wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E35. The composition of E27, wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E36. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E37. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E38. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E39. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E40. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E41. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E42. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E43. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E44. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E45. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E46. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E47. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E48. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E49. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E50. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E51. The composition of E27, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E52. The composition of any one of E1-E51, wherein the antibody, or antigen binding fragment thereof, that binds TSLP and/or the antibody, or antigen binding fragment thereof, that binds OX40L is a humanized, human, or chimeric antibody.

E53. The composition of any one of E1-E52, wherein the antibody, or antigen binding fragment thereof, that binds TSLP and/or the antibody, or antigen binding fragment thereof, that binds OX40L is a humanized antibody.

E54. The composition of any one of E1-E53, wherein the antibody, or antigen binding fragment thereof, that binds TSLP and/or the antibody, or antigen binding fragment thereof, that binds OX40L comprises a heavy chain human constant region of a class selected from IgG, IgA, IgD, IgE, and IgM.

E55. The composition of any one of E1-E54, wherein the antibody, or antigen binding fragment thereof, that binds TSLP and/or the antibody, or antigen binding fragment thereof, that binds OX40L comprises a human Fc region, and wherein the human Fc region comprises a human heavy chain constant region of the class IgG and a subclass selected from IgG1, IgG2, IgG3, and IgG4.

E56. The composition of E55, wherein the human Fc region of the antibody, or antigen binding fragment thereof, that binds TSLP and/or the antibody, or antigen binding fragment thereof, that binds OX40L comprises a human IgG1 Fc region

E57. The composition of E55, wherein the human Fc region of the antibody, or antigen binding fragment thereof, that binds TSLP and/or the antibody, or antigen binding fragment thereof, that binds OX40L comprises a human IgG4 Fc region.

E58. The composition of E55, wherein the human Fc region of the antibody, or antigen binding fragment thereof, that binds TSLP and/or the antibody, or antigen binding fragment thereof, that binds OX40L comprises a human IgG2 Fc region.

E59. The composition of any one of E1-E58, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 47-270.

E60. The composition of E59, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 61 and 173.

E61. The composition of any one of E1-E60, wherein the antibody, or antigen binding fragment thereof, that binds TSLP and/or the antibody, or antigen binding fragment thereof, that binds OX40L comprises a light chain comprising a constant light chain sequence set forth in SEQ ID NO: 46 or SEQ ID NO: 520.

E62. The composition of any one of E1-E60, wherein the antibody, or antigen binding fragment thereof, that binds TSLP comprises a light chain comprising a human lambda light chain constant region and/or the antibody, or antigen binding fragment thereof, that binds OX40L comprises a light chain comprising a human kappa light chain constant region.

E63. The composition of E61 or E62, wherein the antibody, or antigen binding fragment thereof, that binds TSLP comprises a constant light chain sequence set forth in SEQ ID NO: 520.

E64. The composition of E61 or E62, wherein the antibody, or antigen binding fragment thereof, that binds OX40L comprises a constant light chain sequence set forth in SEQ ID NO: 46.

E65. The composition of any one of E1-E64, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in an increase in one or more of antibody half-life, ADCC activity, ADCP activity, or CDC activity compared with the Fc without the one or more amino acid substitutions.

E66. The composition of any one of E1-E64, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in a decrease in one or more of, ADCC activity, ADCP activity or CDC activity compared to an antibody comprising a wild-type Fc region.

E67. The composition of E65 or E66, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W (or e.g., L235A, numbering); optionally, wherein the one or more amino acid substitutions comprises a plurality of amino acid substitutions selected from the group consisting of M428L/N434S (LS), M252Y/S254T/T256E (YTE) using EU numbering or M257Y/S259T/T261E (YTE) using direct numbering or M253Y/S255T/T257E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) or L239A/L240A (LALA) using direct numbering or L235A/L236A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

E68. The composition of E67, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

E69. The composition of any one of E1-E4 and E6-E68, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.

E70. The composition of any one of E1-E4 and E6-E69, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61 and/or a constant heavy chain sequence set forth in SEQ ID NO: 173, and a constant light chain sequence set forth in SEQ ID NO: 520.

E71. The composition of E70, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a light chain sequence set forth in SEQ ID NO: 520 (e.g., a heavy chain set forth in SEQ ID NO: 524 and a light chain set forth in SEQ ID NO: 525).

E72. The composition of E70, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520 (e.g., a heavy chain set forth in SEQ ID NO: 526 and a light chain set forth in SEQ ID NO: 525).

E73. The composition of any one of E1 and E3-E72, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, and a human IgG1 Fc region comprising LALA and YTE substitutions.

E74. The composition of any one of E1 and E3-E73, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61 and/or a constant heavy chain sequence set forth in SEQ ID NO: 173, and a constant light chain sequence set forth in SEQ ID NO: 46.

E75. The composition of E74, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173, and a constant light chain sequence set forth in SEQ ID NO: 46 (e.g., a heavy chain set forth in SEQ ID NO: 543 and a light chain set forth in SEQ ID NO: 545).

E76. The composition of E74, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61, and a constant light chain sequence set forth in SEQ ID NO: 46 (e.g., a heavy chain set forth in SEQ ID NO: 544 and a light chain set forth in SEQ ID NO: 545).

E77. The composition of any one of E1-E76, wherein the Fc region of the antibody, or antigen binding fragment thereof, that binds TSLP and/or the antibody, or antigen binding fragment thereof, that binds OX40L binds to Neonatal Fc receptor (FcRn).

E78. The composition of E77, wherein the Fc region of the antibody, or antigen binding fragment thereof, that binds TSLP and/or the antibody, or antigen binding fragment thereof, that binds OX40L binds an FcRn with higher affinity at pH 6.0 compared to an antibody comprising a wild-type Fc region.

E79. The composition of E77 or E78, wherein the Fc region of the antibody, or antigen binding fragment thereof, that binds TSLP and/or the antibody, or antigen binding fragment thereof, that binds OX40L binds to FcRn with a K_{D} of <1×10⁻⁷ M at pH 6.0.

E80. The composition of any one of E1-E79, wherein the antibody, or antigen binding fragment thereof, that binds TSLP and/or the antibody, or antigen binding fragment thereof, that binds OX40L is a monoclonal antibody.

E81. The composition of any one of E1-E80, wherein the antibody, or antigen binding fragment thereof, that binds TSLP binds a TSLP sequence set forth in the amino acid sequence of SEQ ID NO: 271 or SEQ ID NO: 272.

E82. The composition of any one of E1-E81, wherein the antibody, or antigen binding fragment thereof, that binds OX40L binds an OX40L sequence set forth in the amino acid sequence of SEQ ID NO: 439.

E83. The composition of any one of E1-E82, wherein the hyaluronidase is a recombinant human hyaluronidase.

E84. The composition of E83, wherein the recombinant human hyaluronidase comprises an amino acid sequence set forth in any one of SEQ ID NOs: 453-462 and 464-519, or a mixture thereof.

E85. The composition of E83, wherein the recombinant human hyaluronidase is a soluble hyaluronidase provided as a composition designated rHuPH20.

E86. The composition of E85, wherein the rHuPH20 formulation is ENHANZE®.

E87. The composition of any one of E1-E86, wherein the composition is formulated for subcutaneous injection.

E88. The composition of any one of E1-E86, wherein the composition is formulated for intravenous injection.

E89. The composition of any one of E1-E88, wherein the hyaluronidase is rHuPH20 for administration at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

E90. The composition of any one of E1-E89 for use in the treatment of an inflammatory disorder or disease.

E91. The composition for use according to E90, wherein the composition is for use in the treatment of atopic dermatitis (AD).

E92. The composition for use according to E90, wherein the composition is for use in the treatment of asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; systemic sclerosis; idiopathic pulmonary fibrosis; alopecia areata; allergic rhinitis; eosinophilic fasciitis; scleromyxedema; scleredema; or nephrogenic systemic fibrosis.

E93. The composition for use according to E92, wherein the composition is for use in the treatment of asthma.

E94. The composition for use according to E92, wherein the composition is for use in the treatment of COPD.

E95. A combination comprising an isolated antibody, or an antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E96. The combination of E95, wherein the isolated antibody, or antigen binding fragment thereof, that binds OX40L comprises a CDR-H1, CDR-H2, CDR-H3, CDR-L1, CDR-L2, and CDR-L3 of an antibody selected from amlitelimab and oxelumab.

E97. The combination of E95, wherein the isolated antibody, or antigen binding fragment thereof, that binds OX40L comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E98. A combination comprising an isolated antibody, or an antigen binding fragment thereof, that binds TSLP, and an isolated antibody, or an antigen binding fragment thereof, that binds OX40L, and a hyaluronidase or variant thereof, wherein the antibody, or antigen binding fragment that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E99. The combination of E98, wherein the isolated antibody, or antigen binding fragment thereof, that binds TSLP comprises a CDR-H1, CDR-H2, CDR-H3, CDR-L1, CDR-L2, and CDR-L3 of an antibody selected from tezepelumab, GR2002, TQC2731, solrikitug, hu23b12, 43B1-H2L2, and 43B1-H6L1.

E100. The combination of E98, wherein the isolated antibody, or antigen binding fragment thereof, that binds TSLP comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E101. The combination of E97 or E100, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E102. The combination of E97 or E100, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E103. The combination of E97 or E100, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E104. The combination of E97 or E100, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E105. The combination of E97 or E100, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E106. The combination of E97 or E100, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E107. The combination of E97 or E100, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E108. The combination of E97 or E100, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E109. The combination of E97 or E100, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E110. The combination of E97 or E100, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E111. The combination of E97 or E100, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E112. The combination of E97 or E100, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E113. The combination of E97 or E100, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E114. The combination of E97 or E100, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E115. The combination of E97 or E100, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E116. The combination of E97 or E100, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E117. The combination of any one of E95-E98 and E100-E116, wherein the antibody, or antigen binding fragment thereof, that binds TSLP comprises a heavy chain variable domain (VH) sequence comprising the amino acid sequence set forth in SEQ ID NO: 34.

E118. The combination of any one of E95 and E97-E117, wherein the antibody, or antigen binding fragment thereof, that binds OX40L comprises a VH sequence comprising the amino acid sequence set forth in any one of SEQ ID NOs: 335-338.

E119. The combination of any one of E95-E98 and E100-E118, wherein the antibody, or antigen binding fragment thereof, that binds TSLP comprises a light chain variable domain (VL) sequence comprising the amino acid sequence set forth in any one of SEQ ID NOs: 41, 43, 415, and 421.

E120. The combination of any one of E95 and E97-E119, wherein the antibody, or antigen binding fragment thereof, that binds OX40L comprises a VL sequence comprising the amino acid sequence set forth in any one of SEQ ID NOs: 339-342.

E121. The combination of any one of E117-E120, wherein:
(a) the antibody, or antigen binding fragment thereof, that binds TSLP comprises:
   (i) a VH sequence comprising the amino acid sequence set forth in SEQ ID NO: 34; and/or
   (ii) a VL sequence comprising the amino acid sequence set forth in SEQ ID NO: 41;
   (iii) a VL sequence comprising the amino acid sequence set forth in SEQ ID NO: 43;
   (iv) a VL sequence comprising the amino acid sequence set forth in SEQ ID NO: 415; or
   (v) a VL sequence comprising the amino acid sequence set forth in SEQ ID NO: 421; and/or
(b) the antibody, or antigen binding fragment thereof, that binds OX40L comprises:
   (i) a VH sequence comprising the amino acid sequence set forth in SEQ ID NO: 335;
   (ii) a VH sequence comprising the amino acid sequence set forth in SEQ ID NO: 336;
   (iii) a VH sequence comprising the amino acid sequence set forth in SEQ ID NO: 337; or
   (iv) a VH sequence comprising the amino acid sequence set forth in SEQ ID NO: 338; and/or
   (v) a VL sequence comprising the amino acid sequence set forth in SEQ ID NO: 339;
   (vi) a VL sequence comprising the amino acid sequence set forth in SEQ ID NO: 400;
   (vii) a VL sequence comprising the amino acid sequence set forth in SEQ ID NO: 401; or
   (viii) a VL sequence comprising the amino acid sequence set forth in SEQ ID NO: 402.

E122. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41.

E123. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43.

E124. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415.

E125. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421.

E126. The combination of E121, wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E127. The combination of E121, wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E128. The combination of E121, wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E129. The combination of E121, wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E130. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E131. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E132. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E133. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E134. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E135. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E136. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E137. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E138. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E139. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E140. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E141. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E142. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E143. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E144. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E145. The combination of E121, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E146. The combination of any one of E95-E145, wherein the antibody, or antigen binding fragment thereof, that binds TSLP and/or the antibody, or antigen binding fragment thereof, that binds OX40L is a humanized, human, or chimeric antibody.

E147. The combination of any one of E95-E146, wherein the antibody, or antigen binding fragment thereof, that binds TSLP and/or the antibody, or antigen binding fragment thereof, that binds OX40L is a humanized antibody.

E148. The combination of any one of E95-E147, wherein the antibody, or antigen binding fragment thereof, that binds TSLP and/or the antibody, or antigen binding fragment thereof, that binds OX40L comprises a heavy chain human constant region of a class selected from IgG, IgA, IgD, IgE, and IgM.

E149. The combination of any one of E95-E148, wherein the antibody, or antigen binding fragment thereof, that binds TSLP and/or the antibody, or antigen binding fragment thereof, that binds OX40L comprises a human Fc region, and wherein the human Fc region comprises a human heavy chain constant region of the class IgG and a subclass selected from IgG1, IgG2, IgG3, and IgG4.

E150. The combination of E149, wherein the human Fc region of the antibody, or antigen binding fragment thereof, that binds TSLP and/or the antibody, or antigen binding fragment thereof, that binds OX40L comprises a human IgG1 Fc region

E151. The combination of E149, wherein the human Fc region of the antibody, or antigen binding fragment thereof, that binds TSLP and/or the antibody, or antigen binding fragment thereof, that binds OX40L comprises a human IgG4 Fc region.

E152. The combination of E149, wherein the human Fc region of the antibody, or antigen binding fragment thereof, that binds TSLP and/or the antibody, or antigen binding fragment thereof, that binds OX40L comprises a human IgG2 Fc region.

E153. The combination of any one of E95-E152, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 47-270.

E154. The combination of E153, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 61 and 173.

E155. The combination of any one of E95-E154, wherein the antibody, or antigen binding fragment thereof, that binds TSLP and/or the antibody, or antigen binding fragment thereof, that binds OX40L comprises a light chain comprising a constant light chain sequence set forth in SEQ ID NO: 46 or SEQ ID NO: 520.

E156. The combination of any one of E95-E154, wherein the antibody, or antigen binding fragment thereof, that binds TSLP comprises a light chain comprising a human lambda light chain constant region and/or the antibody, or antigen binding fragment thereof, that binds OX40L comprises a light chain comprising a human kappa light chain constant region.

E157. The combination of E155 or E156, wherein the antibody, or antigen binding fragment thereof, that binds TSLP comprises a constant light chain sequence set forth in SEQ ID NO: 520.

E158. The combination of E155 or E156, wherein the antibody, or antigen binding fragment thereof, that binds OX40L comprises a constant light chain sequence set forth in SEQ ID NO: 46.

E159. The combination of any one of E95-E158, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in an increase in one or more of antibody half-life, ADCC activity, ADCP activity, or CDC activity compared with the Fc without the one or more amino acid substitutions.

E160. The combination of any one of E95-E158, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in a decrease in one or more of, ADCC activity, ADCP activity or CDC activity compared to an antibody comprising a wild-type Fc region.

E161. The combination of E159 or E160, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W (or e.g., L235A, numbering); optionally, wherein the one or more amino acid substitutions comprises a plurality of amino acid substitutions selected from the group consisting of M428L/N434S (LS), M252Y/S254T/T256E (YTE) using EU numbering or M257Y/S259T/T261E (YTE) using direct numbering or M253Y/S255T/T257E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) or L239A/L240A (LALA) using direct numbering or L235A/L236A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

E162. The combination of E161, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

E163. The combination of any one of E95-E98 and E100-E162, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.

E164. The combination of any one of E95-E98 and E100-E163, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61 and/or a constant heavy chain sequence set forth in SEQ ID NO: 173, and a constant light chain sequence set forth in SEQ ID NO: 520.

E165. The combination of E164, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a light chain sequence set forth in SEQ ID NO: 520 (e.g., a heavy chain set forth in SEQ ID NO: 524 and a light chain set forth in SEQ ID NO: 525).

E166. The combination of E164, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520 (e.g., a heavy chain set forth in SEQ ID NO: 526 and a light chain set forth in SEQ ID NO: 525).

E167. The combination of any one of E95 and E97-E166, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, and a human IgG1 Fc region comprising LALA and YTE substitutions.

E168. The combination of any one of E95 and E97-E167, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61 and/or a constant heavy chain sequence set forth in SEQ ID NO: 173, and a constant light chain sequence set forth in SEQ ID NO: 46.

E169. The combination of E168, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173, and a constant light chain sequence set forth in SEQ ID NO: 46 (e.g., a heavy chain set forth in SEQ ID NO: 543 and a light chain set forth in SEQ ID NO: 545).

E170. The combination of E168, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61, and a constant light chain sequence set forth in SEQ ID NO: 46 (e.g., a heavy chain set forth in SEQ ID NO: 544 and a light chain set forth in SEQ ID NO: 545).

E171. The combination of any one of E95-E170, wherein the Fc region of the antibody, or antigen binding fragment thereof, that binds TSLP and/or the antibody, or antigen binding fragment thereof, that binds OX40L binds to Neonatal Fc receptor (FcRn).

E172. The combination of E171, wherein the Fc region of the antibody, or antigen binding fragment thereof, that binds TSLP and/or the antibody, or antigen binding fragment thereof, that binds OX40L binds an FcRn with higher affinity at pH 6.0 compared to an antibody comprising a wild-type Fc region.

E173. The combination of E171 or E172, wherein the Fc region of the antibody, or antigen binding fragment thereof, that binds TSLP and/or the antibody, or antigen binding fragment thereof, that binds OX40L binds to FcRn with a K_{D} of <1×10⁻⁷ M at pH 6.0.

E174. The combination of any one of E95-E173, wherein the antibody, or antigen binding fragment thereof, that binds TSLP and/or the antibody, or antigen binding fragment thereof, that binds OX40L is a monoclonal antibody.

E175. The combination of any one of E95-E174, wherein the antibody, or antigen binding fragment thereof, that binds TSLP binds a TSLP sequence set forth in the amino acid sequence of SEQ ID NO: 271 or SEQ ID NO: 272.

E176. The combination of any one of E95-E175, wherein the antibody, or antigen binding fragment thereof, that binds OX40L binds an OX40L sequence set forth in the amino acid sequence of SEQ ID NO: 439.

E177. The combination of any one of E95-E176, wherein the hyaluronidase is a recombinant human hyaluronidase.

E178. The combination of E177, wherein the recombinant human hyaluronidase comprises an amino acid sequence set forth in any one of SEQ ID NOs: 453-462 and 464-519, or a mixture thereof.

E179. The combination of E177, wherein the recombinant human hyaluronidase is a soluble hyaluronidase provided as a composition designated rHuPH20.

E180. The combination of E179, wherein the rHuPH20 formulation is ENHANZE®.

E181. The combination of any one of E95-E180, wherein the hyaluronidase and antibodies are administered in separate formulations.

E182. The combination of E181, wherein the hyaluronidase and antibodies are administered simultaneously in separate formulations.

E183. The combination of E181, wherein the hyaluronidase is administered to the patient prior to administration of the antibodies.

E184. The combination of E181, wherein the hyaluronidase is administered to the patient after administration of the antibodies.

E185. The combination of E181, wherein the hyaluronidase is administered to the patient after administration of one of the antibodies, but before administration of the other antibody.

E186. The combination of any one of E95-E180, wherein the hyaluronidase and antibodies are administered in a single formulation.

E187. The combination of any one of E95-E186, wherein the hyaluronidase and antibodies are administered by subcutaneous injection.

E188. The combination of any one of E95-E186, wherein the hyaluronidase and antibodies are administered by intravenous injection.

E189. The combination of any one of E95-E188, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

E190. The combination of any one of E95-E189 for use in the treatment of an inflammatory disorder or disease.

E191. The combination for use according to E190, wherein the inflammatory disorder or disease is atopic dermatitis (AD).

E192. The combination for use according to E190, wherein the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; systemic sclerosis; idiopathic pulmonary fibrosis; alopecia areata; allergic rhinitis; eosinophilic fasciitis; scleromyxedema; scleredema; or nephrogenic systemic fibrosis.

E193. The combination for use according to E192, wherein the inflammatory disorder or disease is asthma.

E194. The combination for use according to E192, wherein the inflammatory disorder or disease is COPD.

E195. A method of treating an inflammatory disorder or disease in a mammalian subject in need thereof, the method comprising administering to the mammalian subject an isolated antibody, or an antigen binding fragment thereof, that binds TSLP, an isolated antibody, or antigen binding fragment thereof, that binds OX40L, and a hyaluronidase or variant thereof, wherein the antibody, or antigen binding fragment thereof, that binds TSLP comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E196. The method of E195, wherein the isolated antibody, or antigen binding fragment thereof, that binds OX40L comprises a CDR-H1, CDR-H2, CDR-H3, CDR-L1, CDR-L2, and CDR-L3 of an antibody selected from amlitelimab and oxelumab.

E197. The method of E195, wherein the isolated antibody, or antigen binding fragment thereof, that binds OX40L comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E198. A method of treating an inflammatory disorder or disease in a mammalian subject in need thereof, the method comprising administering to the mammalian subject an isolated antibody, or an antigen binding fragment thereof, that binds TSLP, and an isolated antibody, or an antigen binding fragment thereof, that binds OX40L, and a hyaluronidase or variant thereof, wherein the antibody, or antigen binding fragment that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3;
wherein the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328;
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330;
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332; or
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E199. The method of E198, wherein the isolated antibody, or antigen binding fragment thereof, that binds TSLP comprises a CDR-H1, CDR-H2, CDR-H3, CDR-L1, CDR-L2, and CDR-L3 of an antibody selected from tezepelumab, GR2002, TQC2731, solrikitug, hu23b12, 43B1-H2L2, and 43B1-H6L1.

E200. The method of E198, wherein the isolated antibody, or antigen binding fragment thereof, that binds TSLP comprises: a) a VH chain sequence having three heavy chain CDR sequences, CDR-H1, CDR-H2, and CDR-H3; and b) a VL chain sequence having three light chain CDR sequences, CDR-L1, CDR-L2, and CDR-L3,
wherein the isolated antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27;
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30;
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; or
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396.

E201. The method of E197 or E200, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E202. The method of E197 or E200, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E203. The method of E197 or E200, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E204. The method of E197 or E200, wherein:
(a) the antibody, or antigen binding fragment thereof, that binds TSLP comprises:
   (i) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 21 and 24; a CDR-L2 comprising the amino acid sequence set forth in SEQ ID NO: 26 or the amino acid sequence DDS; and a CDR-L3 comprising the amino acid sequence set forth in SEQ ID NO: 27; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E205. The method of E197 or E200, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E206. The method of E197 or E200, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E207. The method of E197 or E200, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E208. The method of E197 or E200, wherein:
(a) the antibody, or antigen binding fragment thereof, that binds TSLP comprises:
   (ii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 19 and 22; a CDR-L2 comprising the amino acid sequence set forth in SEQ ID NO: 25 or the amino acid sequence DVS; and a CDR-L3 comprising the amino acid sequence set forth in SEQ ID NO: 30; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E209. The method of E197 or E200, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E210. The method of E197 or E200, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E211. The method of E197 or E200, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E212. The method of E197 or E200, wherein:
(a) the antibody, or antigen binding fragment thereof, that binds TSLP comprises:
   (iii) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 351 and 365; a CDR-L2 comprising the amino acid sequence set forth in SEQ ID NO: 26 or the amino acid sequence DDS; and a CDR-L3 comprising the amino acid sequence set forth in SEQ ID NO: 392; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E213. The method of E197 or E200, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (i) a CDR-H1 comprising the amino acid sequence set forth in any one of SEQ ID NOs: 273, 274, and 275; a CDR-H2 comprising the amino acid sequence set forth in any one of SEQ ID NOs: 285, 286, and 287; a CDR-H3 comprising the amino acid sequence set forth in any one of SEQ ID NOs: 297, 298, and 299; a CDR-L1 comprising the amino acid sequence set forth in any one of SEQ ID NOs: 309, 310, and 311; a CDR-L2 comprising the amino acid sequence set forth in SEQ ID NO: 319 or the amino acid sequence ASS; and a CDR-L3 comprising the amino acid sequence set forth in any one of SEQ ID NOs: 327 and 328.

E214. The method of E197 or E200, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (ii) a CDR-H1 comprising the amino acid sequence set forth in any one of SEQ ID NOs: 276, 277, and 278; a CDR-H2 comprising the amino acid sequence set forth in any one of SEQ ID NOs: 288, 289, and 290; a CDR-H3 comprising the amino acid sequence set forth in any one of SEQ ID NOs: 300, 301, and 302; a CDR-L1 comprising the amino acid sequence set forth in any one of SEQ ID NOs: 312, 313, and 343; a CDR-L2 comprising the amino acid sequence set forth in SEQ ID NO: 321 or the amino acid sequence GAT; and a CDR-L3 comprising the amino acid sequence set forth in any one of SEQ ID NOs: 329 and 330.

E215. The method of E197 or E200, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iii) a CDR-H1 comprising the amino acid sequence set forth in any one of SEQ ID NOs: 279, 280, and 281; a CDR-H2 comprising the amino acid sequence set forth in any one of SEQ ID NOs: 291, 292, and 293; a CDR-H3 comprising the amino acid sequence set forth in any one of SEQ ID NOs: 303, 304, and 305; a CDR-L1 comprising the amino acid sequence set forth in any one of SEQ ID NOs: 344, 314, and 315; a CDR-L2 comprising the amino acid sequence set forth in SEQ ID NO: 323 or the amino acid sequence DAS; and a CDR-L3 comprising the amino acid sequence set forth in any one of SEQ ID NOs: 331 and 332.

E216. The method of E197 or E200, wherein:
(a) the antibody, or antigen binding fragment thereof, that binds TSLP comprises:
   (iv) a CDR-H1 comprising the amino acid sequence set forth in any one of SEQ ID NOs: 3, 6, and 9; a CDR-H2 comprising the amino acid sequence set forth in any one of SEQ ID NOs: 10, 11, and 12; a CDR-H3 comprising the amino acid sequence set forth in any one of SEQ ID NOs: 13 and 16; a CDR-L1 comprising the amino acid sequence set forth in any one of SEQ ID NOs: 356 and 372; a CDR-L2 comprising the amino acid sequence set forth in SEQ ID NO: 26 or the amino acid sequence DDS; and a CDR-L3 comprising the amino acid sequence set forth in SEQ ID NO: 396; and
(b) the antibody, or antigen binding fragment thereof, that binds OX40L comprises:
   (iv) a CDR-H1 comprising the amino acid sequence set forth in any one of SEQ ID NOs: 282, 283, and 284; a CDR-H2 comprising the amino acid sequence set forth in any one of SEQ ID NOs: 294, 295, and 296; a CDR-H3 comprising the amino acid sequence set forth in any one of SEQ ID NOs: 306, 307, and 308; a CDR-L1 comprising the amino acid sequence set forth in any one of SEQ ID NOs: 316, 317, and 318; a CDR-L2 comprising the amino acid sequence set forth in SEQ ID NO: 325 or the amino acid sequence ATS; and a CDR-L3 comprising the amino acid sequence set forth in any one of SEQ ID NOs: 333 and 334.

E217. The method of any one of E195-E198 and E200-E216, wherein the antibody, or antigen binding fragment thereof, that binds TSLP comprises a heavy chain variable domain (VH) sequence comprising the amino acid sequence set forth in SEQ ID NO: 34.

E218. The method of any one of E195 and E197-E217, wherein the antibody, or antigen binding fragment thereof, that binds OX40L comprises a VH sequence comprising the amino acid sequence set forth in any one of SEQ ID NOs: 335-338.

E219. The method of any one of E195-E198 and E200-E218, wherein the antibody, or antigen binding fragment thereof, that binds TSLP comprises a light chain variable domain (VL) sequence comprising the amino acid sequence set forth in any one of SEQ ID NOs: 41, 43, 415, and 421.

E220. The method of any one of E195 and E197-E219, wherein the antibody, or antigen binding fragment thereof, that binds OX40L comprises a VL sequence comprising the amino acid sequence set forth in any one of SEQ ID NOs: 339-342.

E221. The method of any one of E217-E220, wherein:
(a) the antibody, or antigen binding fragment thereof, that binds TSLP comprises:
   (i) a VH sequence comprising the amino acid sequence set forth in SEQ ID NO: 34; and/or
   (ii) a VL sequence comprising the amino acid sequence set forth in SEQ ID NO: 41;
   (iii) a VL sequence comprising the amino acid sequence set forth in SEQ ID NO: 43
   (iv) a VL sequence comprising the amino acid sequence set forth in SEQ ID NO: 415; or
   (v) a VL sequence comprising the amino acid sequence set forth in SEQ ID NO: 421; and/or
(b) the antibody, or antigen binding fragment thereof, that binds OX40L comprises:
   (i) a VH sequence comprising the amino acid sequence set forth in SEQ ID NO: 335;
   (ii) a VH sequence comprising the amino acid sequence set forth in SEQ ID NO: 336;
   (iii) a VH sequence comprising the amino acid sequence set forth in SEQ ID NO: 337; or
   (iv) a VH sequence comprising the amino acid sequence set forth in SEQ ID NO: 338; and/or
   (v) a VL sequence comprising the amino acid sequence set forth in SEQ ID NO: 339;
   (vi) a VL sequence comprising the amino acid sequence set forth in SEQ ID NO: 400;
   (vii) a VL sequence comprising the amino acid sequence set forth in SEQ ID NO: 401; or
   (viii) a VL sequence comprising the amino acid sequence set forth in SEQ ID NO: 402.

E222. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41.

E223. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43.

E224. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415.

E225. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421.

E226. The method of E221, wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E227. The method of E221, wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E228. The method of E221, wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E229. The method of E221, wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E230. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E231. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E232. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E233. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 41; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E234. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E235. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E236. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E237. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 43; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E238. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E239. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E240. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E241. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 415; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E242. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 335; and a VL sequence comprising the amino acid sequence set forth in SEQ ID NO: 339.

E243. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 336; and a VL sequence comprising the amino acid sequence set forth in SEQ ID NO: 340.

E244. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 337; and a VL sequence comprising the amino acid sequence set forth in SEQ ID NO: 341.

E245. The method of E221, wherein the antibody, or antigen binding fragment thereof, that binds TSLP comprises a VH sequence comprising the amino acid sequence set forth in SEQ ID NO: 34; and a VL sequence comprising the amino acid sequence set forth in SEQ ID NO: 421; and wherein the antibody, or antigen binding fragment thereof, that binds OX40L comprises a VH sequence comprising the amino acid sequence set forth in SEQ ID NO: 338; and a VL sequence comprising the amino acid sequence set forth in SEQ ID NO: 342.

E246. The method of any one of E195-E245, wherein the antibody, or antigen binding fragment thereof, that binds TSLP and/or the antibody, or antigen binding fragment thereof, that binds OX40L is a humanized, human, or chimeric antibody.

E247. The method of any one of E195-E246, wherein the antibody, or antigen binding fragment thereof, that binds TSLP and/or the antibody, or antigen binding fragment thereof, that binds OX40L is a humanized antibody.

E248. The method of any one of E195-E247, wherein the antibody, or antigen binding fragment thereof, that binds TSLP and/or the antibody, or antigen binding fragment thereof, that binds OX40L comprises a heavy chain human constant region of a class selected from IgG, IgA, IgD, IgE, and IgM.

E249. The method of any one of E195-E248, wherein the antibody, or antigen binding fragment thereof, that binds TSLP and/or the antibody, or antigen binding fragment thereof, that binds OX40L comprises a human Fc region, and wherein the human Fc region comprises a human heavy chain constant region of the class IgG and a subclass selected from IgG1, IgG2, IgG3, and IgG4.

E250. The method of E249, wherein the human Fc region of the antibody, or antigen binding fragment thereof, that binds TSLP and/or the antibody, or antigen binding fragment thereof, that binds OX40L comprises a human IgG1 Fc region

E251. The method of E249, wherein the human Fc region of the antibody, or antigen binding fragment thereof, that binds TSLP and/or the antibody, or antigen binding fragment thereof, that binds OX40L comprises a human IgG4 Fc region.

E252. The method of E249, wherein the human Fc region of the antibody, or antigen binding fragment thereof, that binds TSLP and/or the antibody, or antigen binding fragment thereof, that binds OX40L comprises a human IgG2 Fc region.

E253. The method of any one of E195-E252, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 47-270.

E254. The method of E253, wherein the antibody, or antigen binding fragment thereof, that binds TSLP and/or the antibody, or antigen binding fragment thereof, that binds OX40L comprises a heavy chain comprising a constant heavy chain sequence selected from an amino acid sequence set forth in any one of SEQ ID NOs: 61 and 173.

E255. The method of any one of E195-E254, wherein the antibody, or antigen binding fragment thereof, that binds TSLP and/or the antibody, or antigen binding fragment thereof, that binds OX40L comprises a light chain comprising a constant light chain sequence set forth in SEQ ID NO: 46 or SEQ ID NO: 520.

E256. The method of any one of E195-E254, wherein the antibody, or antigen binding fragment thereof, that binds TSLP comprises a light chain comprising a human lambda light chain constant region and/or the antibody, or antigen binding fragment thereof, that binds OX40L comprises a light chain comprising a human kappa light chain constant region.

E257. The method of E255 or E256, wherein the antibody, or antigen binding fragment thereof, that binds TSLP comprises a constant light chain sequence set forth in SEQ ID NO: 520.

E258. The method of E255 or E256, wherein the antibody, or antigen binding fragment thereof, that binds OX40L comprises a constant light chain sequence set forth in SEQ ID NO: 46.

E259. The method of any one of E195-E258, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in an increase in one or more of antibody half-life, ADCC activity, ADCP activity, or CDC activity compared with the Fc without the one or more amino acid substitutions.

E260. The method of any one of E195-E258, wherein the antibody, or antigen binding fragment thereof, that binds TSLP and/or the antibody, or antigen binding fragment thereof, that binds OX40L comprises an Fc region comprising one or more amino acid substitutions, wherein the one or more substitutions result in a decrease in one or more of, ADCC activity, ADCP activity or CDC activity compared to an antibody comprising a wild-type Fc region.

E261. The method of E259 or E260, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W (or e.g., L235A, L236A, L239A, L240A, M253Y, S255T, T257E, M257Y, S259T, or T261E using direct numbering); optionally, wherein the one or more amino acid substitutions comprises a plurality of amino acid substitutions selected from the group consisting of M428L/N434S (LS), M252Y/S254T/T256E (YTE) using EU numbering or M257Y/S259T/T261E (YTE) using direct numbering or M253Y/S255T/T257E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) or L239A/L240A (LALA) using direct numbering or L235A/L236A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

E262. The method of E261, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

E263. The method of any one of E195-E198 and E200-E262, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.

E264. The method of any one of E195-E198 and E200-E263, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61 and/or a constant heavy chain sequence set forth in SEQ ID NO: 173, and a constant light chain sequence set forth in SEQ ID NO: 520.

E265. The method of E264, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a light chain sequence set forth in SEQ ID NO: 520 (e.g., a heavy chain set forth in SEQ ID NO: 524 and a light chain set forth in SEQ ID NO: 525).

E266. The method of E264, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520 (e.g., a heavy chain set forth in SEQ ID NO: 526 and a light chain set forth in SEQ ID NO: 525).

E267. The method of any one of E195 and E197-E266, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, and a human IgG1 Fc region comprising LALA and YTE substitutions.

E268. The method of any one of E195 and E197-E267, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61 and/or a constant heavy chain sequence set forth in SEQ ID NO: 173, and a constant light chain sequence set forth in SEQ ID NO: 46.

E269. The method of E268, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173, and a constant light chain sequence set forth in SEQ ID NO: 46 (e.g., a heavy chain set forth in SEQ ID NO: 543 and a light chain set forth in SEQ ID NO: 545).

E270. The method of E268, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61, and a constant light chain sequence set forth in SEQ ID NO: 46 (e.g., a heavy chain set forth in SEQ ID NO: 544 and a light chain set forth in SEQ ID NO: 545).

E271. The method of any one of E195-E270, wherein the Fc region of the antibody, or antigen binding fragment thereof, that binds TSLP and/or the antibody, or antigen binding fragment thereof, that binds OX40L binds to Neonatal Fc receptor (FcRn).

E272. The method of E271, wherein the Fc region of the antibody, or antigen binding fragment thereof, that binds TSLP and/or the antibody, or antigen binding fragment thereof, that binds OX40L binds an FcRn with higher affinity at pH 6.0 compared to an antibody comprising a wild-type Fc region.

E273. The method of E271 or E272, wherein the Fc region of the antibody, or antigen binding fragment thereof, that binds TSLP and/or the antibody, or antigen binding fragment thereof, that binds OX40L binds to FcRn with a K_{D} of <1×10⁻⁷ M at pH 6.0.

E274. The method of any one of E195-E273, wherein the antibody, or antigen binding fragment thereof, that binds TSLP and/or the antibody, or antigen binding fragment thereof, that binds OX40L is a monoclonal antibody.

E275. The method of any one of E195-E274, wherein the antibody, or antigen binding fragment thereof, that binds TSLP binds a TSLP sequence set forth in the amino acid sequence of SEQ ID NO: 271 or SEQ ID NO: 272.

E276. The method of any one of E195-E275, wherein the antibody, or antigen binding fragment thereof, that binds OX40L binds an OX40L sequence set forth in the amino acid sequence of SEQ ID NO: 439.

E277. The method of any one of E195-E276, wherein the hyaluronidase is a recombinant human hyaluronidase.

E278. The method of E277, wherein the recombinant human hyaluronidase comprises an amino acid sequence set forth in any one of SEQ ID NOs: 453-462 and 464-519, or a mixture thereof.

E279. The method of E277, wherein the recombinant human hyaluronidase is a soluble hyaluronidase provided as a composition designated rHuPH20.

E280. The method of E279, wherein the rHuPH20 formulation is ENHANZE®.

E281. The method of any one of E195-E280, wherein the hyaluronidase and antibodies are administered in separate formulations.

E282. The method of E281, wherein the hyaluronidase and antibodies are administered simultaneously in separate formulations.

E283. The method of E281, wherein the hyaluronidase is administered to the patient prior to administration of the antibodies.

E284. The method of E281, wherein the hyaluronidase is administered to the patient after administration of the antibodies.

E285. The method of E281, wherein the hyaluronidase is administered to the patient after administration of one of the antibodies, but before administration of the other antibody.

E286. The method of any one of E195-E280, wherein the hyaluronidase and antibodies are administered in a single formulation.

E287. The method of any one of E195-E286, wherein the hyaluronidase and antibodies are administered by subcutaneous injection.

E288. The method of any one of E195-E286, wherein the hyaluronidase and antibodies are administered by intravenous injection.

E289. The method of any one of E195-E188, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

E290. The method of any one of E195-E289, wherein the inflammatory disorder or disease is atopic dermatitis (AD).

E291. The method of any one of E195-E289, wherein the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; systemic sclerosis; idiopathic pulmonary fibrosis; alopecia areata; allergic rhinitis; eosinophilic fasciitis; scleromyxedema; scleredema; or nephrogenic systemic fibrosis.

E292. The method of E291, wherein the inflammatory disorder or disease is asthma.

E293. The method of E291, wherein the inflammatory disorder or disease is COPD.

E294. A kit comprising:
(a) an antibody, or antigen binding fragment thereof, that binds TSLP in unit dosage form, wherein the antibody comprises a VH sequence set forth in SEQ ID NO: 34 and a VL sequence set forth in SEQ ID NO: 41;
(b) an antibody, or antigen binding fragment thereof, that binds OX40L in unit dosage form, wherein the antibody comprises a VH sequence set forth in SEQ ID NO: 335 and a VL sequence set forth in SEQ ID NO: 339;
(c) a hyaluronidase or variant thereof in unit dosage form; and
(d) instructions.

E295. The kit of E294, wherein the hyaluronidase is a recombinant human hyaluronidase.

E296. The kit of E295, wherein the recombinant human hyaluronidase comprises the amino acid sequence set forth in any one of SEQ ID NOs: 453-462 and 464-519, or a mixture thereof.

E297. The kit of E295, wherein the recombinant human hyaluronidase is a soluble hyaluronidase provided as a composition designated rHuPH20.

E298. The kit of E297, wherein the rHuPH20 formulation is ENHANZE®.

E299. The kit of any one of E294-E298, for use in treating an inflammatory disorder or disease.

E300. The kit of E299, wherein the inflammatory disorder or disease is atopic dermatitis.

E301. The kit of E299, wherein the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); psoriasis; hidradenitis suppurativa; celiac disease; systemic sclerosis; idiopathic pulmonary fibrosis; alopecia areata; allergic rhinitis; eosinophilic fasciitis; scleromyxedema; scleredema; or nephrogenic systemic fibrosis.

E302. The kit of any one of E294-E301, wherein the antibody, or antigen binding fragment thereof, that binds TSLP and/or the antibody, or antigen binding fragment thereof, that binds OX40L comprises a human Fc region comprising a human IgG sequence selected from a sequence set forth in any one of SEQ ID NOs: 47-270.

E303. The kit of any one of E294-E302, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.

E304. The kit of any one of E294-E303, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61 and/or a constant heavy chain sequence set forth in SEQ ID NO: 173, and a constant light chain sequence set forth in SEQ ID NO: 520.

E305. The kit of E304, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a light chain sequence set forth in SEQ ID NO: 520 (e.g., a heavy chain set forth in SEQ ID NO: 524 and a light chain set forth in SEQ ID NO: 525).

E306. The kit of E304, wherein the antibody, or antigen binding fragment thereof, that binds TSLP comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 520 (e.g., a heavy chain set forth in SEQ ID NO: 526 and a light chain set forth in SEQ ID NO: 525).

E307. The kit of any one of E294-E306, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, and a human IgG1 Fc region comprising LALA and YTE substitutions.

E308. The kit of any one of E294-E307, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61 and/or a constant heavy chain sequence set forth in SEQ ID NO: 173, and a constant light chain sequence set forth in SEQ ID NO: 46.

E309. The kit of E308, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 173, and a constant light chain sequence set forth in SEQ ID NO: 46 (e.g., a heavy chain set forth in SEQ ID NO: 543 and a light chain set forth in SEQ ID NO: 545).

E310. The kit of E308, wherein the antibody, or antigen binding fragment thereof, that binds OX40L comprises a VH domain comprising the amino acid sequence set forth in SEQ ID NO: 335, a VL domain comprising the amino acid sequence set forth in SEQ ID NO: 339, a constant heavy chain sequence set forth in SEQ ID NO: 61, and a constant light chain sequence set forth in SEQ ID NO: 46 (e.g., a heavy chain set forth in SEQ ID NO: 544 and a light chain set forth in SEQ ID NO: 545).

INFORMAL SEQUENCE LISTING

| | | SEQ ID |
|---|---|---|
| Description | Sequence | NO |
| HCDR1 | TYTMH | 1 |
| HCDR1 | TYPMH | 2 |
| HCDR1 | TYGMH | 3 |
| HCDR1 | GFTFRTYTMH | 4 |
| HCDR1 | GFTFRTYPMH | 5 |
| HCDR1 | GFTFRTYGMH | 6 |
| HCDR1 | GFTFRTYT | 7 |
| HCDR1 | GFTFRTYP | 8 |
| HCDR1 | GFTFRTYG | 9 |
| HCDR2 | VIWYDGSNKHYADSVKG | 10 |
| HCDR2 | WYDGSN | 11 |
| HCDR3 | IWYDGSNK | 12 |
| HCDR3 | APQWELVHEAFDI | 13 |
| HCDR3 | ALIWELFHEYFDI | 14 |
| HCDR3 | ALLWELFHEYFDI | 15 |
| HCDR3 | ARAPQWELVHEAFDI | 16 |
| HCDR3 | ARALIWELFHEYFDI | 17 |
| HCDR3 | ARALLWELFHEYFDI | 18 |
| LCDR1 | GGNNLGSKSVH | 19 |
| LCDR1 | GGNNLISKSVH | 20 |
| LCDR1 | GGNNLVSKSVH | 21 |
| LCDR1 | NLGSKS | 22 |
| LCDR1 | NLISKS | 23 |
| LCDR1 | NLVSKS | 24 |
| LCDR2 | DVSDRPS | 25 |
| LCDR2 | DDSDRPS | 26 |
| LCDR3 | QVWDSSSTFVV | 27 |
| LCDR3 | QVWDSSSTYVV | 28 |
| LCDR3 | QVWDSSSDHVV | 29 |
| LCDR3 | QVWDSSSTIVV | 30 |
| LCDR3 | QVWDKSSTYVV | 31 |
| VH | QMQLVESGGGVVQPGRSLRLSCAASGFTFRTYTMHWVRQA | 32 |
| | PGKGLEWVAVIWYDGSNKHYADSVKGRFTITRDNSKNTLN | |
| | LQMNSLRAEDTAVYYCARAPQWELVHEAFDIWGQGTMVT | |
| | VSS | |
| VH | QMQLVESGGGVVQPGRSLRLSCAASGFTFRTYPMHWVRQA | 33 |
| | PGKGLEWVAVIWYDGSNKHYADSVKGRFTITRDNSKNTLN | |
| | LQMNSLRAEDTAVYYCARAPQWELVHEAFDIWGQGTMVT | |
| | VSS | |
| VH | QMQLVESGGGVVQPGRSLRLSCAASGFTFRTYGMHWVRQ | 34 |
| | APGKGLEWVAVIWYDGSNKHYADSVKGRFTITRDNSKNTL | |
| | NLQMNSLRAEDTAVYYCARAPQWELVHEAFDIWGQGTMV | |
| | TVSS | |
| VH | QMQLVESGGGVVQPGRSLRLSCAASGFTFRTYGMHWVRQ | 35 |
| | APGKGLEWVAVIWYDGSNKHYADSVKGRFTITRDNSKNTL | |
| | NLQMNSLRAEDTAVYYCARALLWELFHEYFDIWGQGTMV | |
| | TVSS | |
| VH | QMQLVESGGGVVQPGRSLRLSCAASGFTFRTYPMHWVRQA | 36 |
| | PGKGLEWVAVIWYDGSNKHYADSVKGRFTITRDNSKNTLN | |
| | LQMNSLRAEDTAVYYCARALLWELFHEYFDIWGQGTMVT | |
| | VSS | |
| VL | SYVLTQPPSVSVAPGQTARITCGGNNLGSKSVHWYQQKPG | 37 |
| | QAPVLVVYDVSDRPSWIPERFSGSNSGNTATLTISRGEAGDE | |
| | ADYYCQVWDSSSTFVVFGGGTKLTVL | |
| VL | SYVLTQPPSVSVAPGQTARITCGGNNLGSKSVHWYQQKPG | 38 |
| | QAPVLVVYDVSDRPSWIPERFSGSNSGNTATLTISRGEAGDE | |
| | ADYYCQVWDSSSTYVVFGGGTKLTVL | |
| VL | SYVLTQPPSVSVAPGQTARITCGGNNLISKSVHWYQQKPGQ | 39 |
| | APVLVVYDDSDRPSWIPERFSGSNSGNTATLTISRGEAGDEA | |
| | DYYCQVWDSSSTYVVFGGGTKLTVL | |
| VL | SYVLTQPPSVSVAPGQTARITCGGNNLVSKSVHWYQQKPG | 40 |
| | QAPVLVVYDVSDRPSWIPERFSGSNSGNTATLTISRGEAGDE | |
| | ADYYCQVWDSSSTYVVFGGGTKLTVL | |
| VL | SYVLTQPPSVSVAPGQTARITCGGNNLVSKSVHWYQQKPG | 41 |
| | QAPVLVVYDDSDRPSWIPERFSGSNSGNTATLTISRGEAGDE | |
| | ADYYCQVWDSSSTFVVFGGGTKLTVL | |
| VL | SYVLTQPPSVSVAPGQTARITCGGNNLVSKSVHWYQQKPG | 42 |
| | QAPVLVVYDVSDRPSWIPERFSGSNSGNTATLTISRGEAGDE | |
| | ADYYCQVWDSSSDHVVFGGGTKLTVL | |
| VL | SYVLTQPPSVSVAPGQTARITCGGNNLGSKSVHWYQQKPG | 43 |
| | QAPVLVVYDVSDRPSWIPERFSGSNSGNTATLTISRGEAGDE | |
| | ADYYCQVWDSSSTIVVFGGGTKLTVL | |
| VL | SYVLTQPPSVSVAPGQTARITCGGNNLGSKSVHWYQQKPG | 44 |
| | QAPVLVVYDDSDRPSWIPERFSGSNSGNTATLTISRGEAGDE | |
| | ADYYCQVWDKSSTYVVFGGGTKLTVL | |
| VL | SYVLTQPPSVSVAPGQTARITCGGNNLVSKSVHWYQQKPG | 45 |
| | QAPVLVVYDDSDRPSWIPERFSGSNSGNTATLTISRGEAGDE | |
| | ADYYCQVWDSSSTYVVFGGGTKLTVL | |
| Human kappa LC | RTVAAPSVFIFPPSDEQLKSGTASVVCLLNNFYPREAKVQW | 46 |
| | KVDNALQSGNSQESVTEQDSKDSTYSLSSTLTLSKADYEKH | |
| | KVYACEVTHQGLSSPVTKSFNRGEC | |
| Heavy Chain | ASTKGPSVFPLAPSSKSTSGGTAALGCLVKDYFPEPVTVSW | 47 |
| Constant Region | NSGALTSGVHTFPAVLQSSGLYSLSSVVTVPSSSLGTQTYIC | |
| hIgG1* | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPCSRSTSESTAALGCLVKDYFPEPVTVSWN | 48 |
| Constant Region | SGALTSGVHTFPAVLQSSGLYSLSSVVTVPSSSLGTKTYTCN | |
| hIgG4 | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 49 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 50 |
| Constant Region | SGALTSGVHTFPAVLQSSGLYSLSSVVTVPSSSLGTKTYTCN | |
| IgG4-SPLE | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 51 |
| Constant Region | SGALTSGVHTFPAVLQSSGLYSLSSVVTVPSSNFGTQTYTCN | |
| IgG2 | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSW | 52 |
| Constant Region | NSGALTSGVHTFPAVLQSSGLYSLSSVVTVPSSSLGTQTYIC | |
| hIgG1-N297A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 53 |
| Constant Region | NSGALTSGVHTFPAVLQSSGLYSLSSVVTVPSSSLGTQTYIC | |
| hIgG1-D265A | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 54 |
| Constant Region | NSGALTSGVHTFPAVLQSSGLYSLSSVVTVPSSSLGTQTYIC | |
| hIgG1-LALA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 55 |
| Constant Region | NSGALTSGVHTFPAVLQSSGLYSLSSVVTVPSSSLGTQTYIC | |
| hIgG1-LAGA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 56 |
| Constant Region | NSGALTSGVHTFPAVLQSSGLYSLSSVVTVPSSSLGTQTYIC | |
| hIgG1-LALAGA | NVNHKPSNTKVDKKVEPKSCDKTHTCPPCPAPEAAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 57 |
| Constant Region | NSGALTSGVHTFPAVLQSSGLYSLSSVVTVPSSSLGTQTYIC | |
| hIgG1-LALAPG | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 58 |
| Constant Region | NSGALTSGVHTFPAVLQSSGLYSLSSVVTVPSSSLGTQTYIC | |
| hIgG1-YTE | NVNHKPSNTKVDKKVEPKSCDKTHTCPPCPAPELLGGPSVF | |
| | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 59 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/YTE | LFPPKPKDTLYITREPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 60 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/YTE | LFPPKPKDTLYITREPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 61 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 62 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 63 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 64 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 65 |
| Constant Region | NSGALTSGVHTFPAVLQSSGLYSLSSVVTVPSSSLGTQTYIC | |
| hIgG1-LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 66 |
| Constant Region | NSGALTSGVHTFPAVLQSSGLYSLSSVVTVPSSSLGTQTYIC | |
| hIgG1-N297A/LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 67 |
| Constant Region | NSGALTSGVHTFPAVLQSSGLYSLSSVVTVPSSSLGTQTYIC | |
| hIgG1-D265A/LS | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 68 |
| Constant Region | NSGALTSGVHTFPAVLQSSGLYSLSSVVTVPSSSLGTQTYIC | |
| hIgG1-LALA/LS | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 69 |
| Constant Region | NSGALTSGVHTFPAVLQSSGLYSLSSVVTVPSSSLGTQTYIC | |
| hIgG1-LAGA/LS | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 70 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 71 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 72 |
| Constant Region | NSGALTSGVHTFPAVLQSSGLYSLSSVVTVPSSSLGTQTYIC | |
| hIgG1-DHS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 73 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DHS | LFPPKPKDTLMISRTPEVTCVVVDVSHEDPEVKFNWYVDGV | |
| | EVHNAKTKPREEQYASTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 74 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DHS | LFPPKPKDTLMISRTPEVTCVVVAVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 75 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 76 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 77 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 78 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPCSRSTSESTAALGCLVKDYFPEPVTVSWN | 79 |
| Constant Region | SGALTSGVHTFPAVLQSSGLYSLSSVVTVPSSSLGTKTYTCN | |
| hIgG4-YTE | VDHKPSNTKVDKRVESKYGPPCPSCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 80 |
| Constant Region | SGALTSGVHTFPAVLQSSGLYSLSSVVTVPSSSLGTKTYTCN | |
| hIgG4-SP/YTE | VDHKPSNTKVDKRVESKYGPPCPPCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 81 |
| Constant Region | SGALTSGVHTFPAVLQSSGLYSLSSVVTVPSSSLGTKTYTCN | |
| hIgG4-SPLE/YTE | VDHKPSNTKVDKRVESKYGPPCPPCPAPEELGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 82 |
| Constant Region | SGALTSGVHTFPAVLQSSGLYSLSSVVTVPSSSLGTKTYTCN | |
| hIgG4-LS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 83 |
| Constant Region | SGALTSGVHTFPAVLQSSGLYSLSSVVTVPSSSLGTKTYTCN | |
| hIgG4-SP/LS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 84 |
| Constant Region | SGALTSGVHTFPAVLQSSGLYSLSSVVTVPSSSLGTKTYTCN | |
| hIgG4-SPLE/LS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 85 |
| Constant Region | SGALTSGVHTFPAVLQSSGLYSLSSVVTVPSSSLGTKTYTCN | |
| hIgG4-DHS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 86 |
| Constant Region | SGALTSGVHTFPAVLQSSGLYSLSSVVTVPSSSLGTKTYTCN | |
| hIgG4-SP/DHS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 87 |
| Constant Region | SGALTSGVHTFPAVLQSSGLYSLSSVVTVPSSSLGTKTYTCN | |
| hIgG4-SPLE/DHS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 88 |
| Constant Region | SGALTSGVHTFPAVLQSSGLYSLSSVVTVPSSNFGTQTYTCN | |
| hIgG2-YTE | VDHKPSNTKVDKTVERKCCVECPPCPAPPVAGPSVFLFPPKP | |
| | KDTLYITREPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPG | |
| Heavy Chain | ASTKGPSVFPLAPCSRSTSESTAALGCLVKDYFPEPVTVSWN | 89 |
| Constant Region | SGALTSGVHTFPAVLQSSGLYSLSSVVTVPSSNFGTQTYTCN | |
| hIgG2-LS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHSHYTQKSLSLSPG | |
| Heavy Chain | ASTKGPSVFPLAPCSRSTSESTAALGCLVKDYFPEPVTVSWN | 90 |
| Constant Region | SGALTSGVHTFPAVLQSSGLYSLSSVVTVPSSNFGTQTYTCN | |
| hIgG2-DHS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVDHHDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHSHYTQKSLSLSPG | |
| Heavy Chain | ASTKGPSVFPLAPCSRSTSESTAALGCLVKDYFPEPVTVSWN | 91 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPSSKSTSGGTAALGCLVKDYFPEPVTVSW | 92 |
| Constant Region | NSGALTSGVHTFPAVLQSSGLYSLSSVVTVPSSSLGTQTYIC | |
| hIgG1-LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 93 |
| Constant Region | NSGALTSGVHTFPAVLQSSGLYSLSSVVTVPSSSLGTQTYIC | |
| hIgG1-N297A/LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 94 |
| Constant Region | NSGALTSGVHTFPAVLQSSGLYSLSSVVTVPSSSLGTQTYIC | |
| hIgG1-D265A/LA | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 95 |
| Constant Region | NSGALTSGVHTFPAVLQSSGLYSLSSVVTVPSSSLGTQTYIC | |
| hIgG1-LALA/LA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 96 |
| Constant Region | NSGALTSGVHTFPAVLQSSGLYSLSSVVTVPSSSLGTQTYIC | |
| hIgG1-LAGA/LA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 97 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 98 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 99 |
| Constant Region | NSGALTSGVHTFPAVLQSSGLYSLSSVVTVPSSSLGTQTYIC | |
| hIgG1-N434A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 100 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434A | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 101 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434A | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 102 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 103 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 104 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 105 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 106 |
| Constant Region | NSGALTSGVHTFPAVLQSSGLYSLSSVVTVPSSSLGTQTYIC | |
| hIgG1-N434W | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 107 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434W | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 108 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434W | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 109 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 110 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 111 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 112 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 113 |
| Constant Region | NSGALTSGVHTFPAVLQSSGLYSLSSVVTVPSSSLGTQTYIC | |
| hIgG1-DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 114 |
| Constant Region | NSGALTSGVHTFPAVLQSSGLYSLSSVVTVPSSSLGTQTYIC | |
| hIgG1-N297A/DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYASTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 115 |
| Constant Region | NSGALTSGVHTFPAVLQSSGLYSLSSVVTVPSSSLGTQTYIC | |
| hIgG1-D265A/DQ | NVNHKPSNTKVDKKVEPKSCDKTHTCPPCPAPELLGGPSVF | |
| | LFPPKPKDTLMISRDPEVTCVVVAVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 116 |
| Constant Region | NSGALTSGVHTFPAVLQSSGLYSLSSVVTVPSSSLGTQTYIC | |
| hIgG1-LALA/DQ | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 117 |
| Constant Region | NSGALTSGVHTFPAVLQSSGLYSLSSVVTVPSSSLGTQTYIC | |
| hIgG1-LAGA/DQ | NVNHKPSNTKVDKKVEPKSCDKTHTCPPCPAPELAGAPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 118 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 119 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 120 |
| Constant Region | NSGALTSGVHTFPAVLQSSGLYSLSSVVTVPSSSLGTQTYIC | |
| hIgG1-DW | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 121 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DW | LFPPKPKDTLMISRDPEVTCVVVDVSHEDPEVKFNWYVDGV | |
| | EVHNAKTKPREEQYASTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 122 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DW | LFPPKPKDTLMISRDPEVTCVVVAVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 123 |
| Constant Region | NSGALTSGVHTFPAVLQSSGLYSLSSVVTVPSSSLGTQTYIC | |
| hIgG1-LALA/DW | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 124 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 125 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 126 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 127 |
| Constant Region | NSGALTSGVHTFPAVLQSSGLYSLSSVVTVPSSSLGTQTYIC | |
| hIgG1-YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 128 |
| Constant Region | NSGALTSGVHTFPAVLQSSGLYSLSSVVTVPSSSLGTQTYIC | |
| hIgG1-N297A/YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 129 |
| Constant Region | NSGALTSGVHTFPAVLQSSGLYSLSSVVTVPSSSLGTQTYIC | |
| hIgG1-D265A/YD | NVNHKPSNTKVDKKVEPKSCDKTHTCPPCPAPELLGGPSVF | |
| | LFPPKPKDTLYISRDPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 130 |
| Constant Region | NSGALTSGVHTFPAVLQSSGLYSLSSVVTVPSSSLGTQTYIC | |
| hIgG1-LALA/YD | NVNHKPSNTKVDKKVEPKSCDKTHTCPPCPAPEAAGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 131 |
| Constant Region | NSGALTSGVHTFPAVLQSSGLYSLSSVVTVPSSSLGTQTYIC | |
| hIgG1-LAGA/YD | NVNHKPSNTKVDKKVEPKSCDKTHTCPPCPAPELAGAPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 132 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 133 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 134 |
| Constant Region | NSGALTSGVHTFPAVLQSSGLYSLSSVVTVPSSSLGTQTYIC | |
| hIgG1-QVV | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 135 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/QVV | LFPPKPKDTLMISRTPEVTCVVVDVSHEDPEVKFNWYVDGV | |
| | EVHNAKTKPREEQYASTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 136 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/QVV | LFPPKPKDTLMISRTPEVTCVVVAVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 137 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 138 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 139 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 140 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 141 |
| Constant Region | NSGALTSGVHTFPAVLQSSGLYSLSSVVTVPSSSLGTQTYIC | |
| hIgG1-DDRVV | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 142 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DDRVV | LFPPKPKDTLMISRDPEVTCVVVDVSHEDPEVKFNWYVDGV | |
| | EVDNAKTKPREEQYASTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 143 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DDRVV | LFPPKPKDTLMISRDPEVTCVVVAVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 144 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 145 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 146 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 147 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 148 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| Q311R/M428L | LFPPKPKDTLMISRTPEVTCVVVDVSHEDPEVKFNWYVDGV | |
| | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPCSRSTSESTAALGCLVKDYFPEPVTVSWN | 149 |
| Constant Region | SGALTSGVHTFPAVLQSSGLYSLSSVVTVPSSSLGTKTYTCN | |
| hIgG4- | VDHKPSNTKVDKRVESKYGPPCPSCPAPEFLGGPSVFLFPPK | |
| Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 150 |
| Constant Region | SGALTSGVHTFPAVLQSSGLYSLSSVVTVPSSSLGTKTYTCN | |
| IgG4- | VDHKPSNTKVDKRVESKYGPPCPPCPAPEFLGGPSVFLFPPK | |
| SP/Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 151 |
| Constant Region | SGALTSGVHTFPAVLQSSGLYSLSSVVTVPSSSLGTKTYTCN | |
| IgG4- | VDHKPSNTKVDKRVESKYGPPCPPCPAPEELGGPSVFLFPPK | |
| SPLE/Q311R/ | PKDTLMISRTPEVTCVVVDVSQEDPEVQFNWYVDGVEVHN | |
| M428L | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 152 |
| Constant Region | SGALTSGVHTFPAVLQSSGLYSLSSVVTVPSSNFGTQTYTCN | |
| IgG2- | VDHKPSNTKVDKTVERKCCVECPPCPAPPVAGPSVFLFPPKP | |
| Q311R/M428L | KDTLMISRTPEVTCVVVDVSHEDPEVQFNWYVDGVEVHNA | |
| | KTKPREEQFNSTFRVVSVLTVVHRDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHNHYTQKSLSLSPG | |
| Heavy Chain | ASTKGPSVFPLAPSSKSTSGGTAALGCLVKDYFPEPVTVSW | 153 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/Q311R/ | LFPPKPKDTLMISRTPEVTCVVVDVSHEDPEVKFNWYVDGV | |
| M428L | EVHNAKTKPREEQYASTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 154 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/Q311R/ | LFPPKPKDTLMISRTPEVTCVVVAVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 155 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 156 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 157 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 158 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 159 |
| Constant Region | NSGALTSGVHTFPAVLQSSGLYSLSSVVTVPSSSLGTQTYIC | |
| hIgG1* | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPCSRSTSESTAALGCLVKDYFPEPVTVSWN | 160 |
| Constant Region | SGALTSGVHTFPAVLQSSGLYSLSSVVTVPSSSLGTKTYTCN | |
| hIgG4 | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 161 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 162 |
| Constant Region | SGALTSGVHTFPAVLQSSGLYSLSSVVTVPSSSLGTKTYTCN | |
| IgG4-SPLE | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 163 |
| Constant Region | SGALTSGVHTFPAVLQSSGLYSLSSVVTVPSSNFGTQTYTCN | |
| IgG2 | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSW | 164 |
| Constant Region | NSGALTSGVHTFPAVLQSSGLYSLSSVVTVPSSSLGTQTYIC | |
| hIgG1-N297A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 165 |
| Constant Region | NSGALTSGVHTFPAVLQSSGLYSLSSVVTVPSSSLGTQTYIC | |
| hIgG1-D265A | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 166 |
| Constant Region | NSGALTSGVHTFPAVLQSSGLYSLSSVVTVPSSSLGTQTYIC | |
| hIgG1-LALA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 167 |
| Constant Region | NSGALTSGVHTFPAVLQSSGLYSLSSVVTVPSSSLGTQTYIC | |
| hIgG1-LAGA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 168 |
| Constant Region | NSGALTSGVHTFPAVLQSSGLYSLSSVVTVPSSSLGTQTYIC | |
| hIgG1-LALAGA | NVNHKPSNTKVDKKVEPKSCDKTHTCPPCPAPEAAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 169 |
| Constant Region | NSGALTSGVHTFPAVLQSSGLYSLSSVVTVPSSSLGTQTYIC | |
| hIgG1-LALAPG | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 170 |
| Constant Region | NSGALTSGVHTFPAVLQSSGLYSLSSVVTVPSSSLGTQTYIC | |
| hIgG1-YTE | NVNHKPSNTKVDKKVEPKSCDKTHTCPPCPAPELLGGPSVF | |
| | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 171 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/YTE | LFPPKPKDTLYITREPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 172 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/YTE | LFPPKPKDTLYITREPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 173 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 174 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 175 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 176 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 177 |
| Constant Region | NSGALTSGVHTFPAVLQSSGLYSLSSVVTVPSSSLGTQTYIC | |
| hIgG1-LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 178 |
| Constant Region | NSGALTSGVHTFPAVLQSSGLYSLSSVVTVPSSSLGTQTYIC | |
| hIgG1-N297A/LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 179 |
| Constant Region | NSGALTSGVHTFPAVLQSSGLYSLSSVVTVPSSSLGTQTYIC | |
| hIgG1-D265A/LS | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 180 |
| Constant Region | NSGALTSGVHTFPAVLQSSGLYSLSSVVTVPSSSLGTQTYIC | |
| hIgG1-LALA/LS | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 181 |
| Constant Region | NSGALTSGVHTFPAVLQSSGLYSLSSVVTVPSSSLGTQTYIC | |
| hIgG1-LAGA/LS | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 182 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 183 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 184 |
| Constant Region | NSGALTSGVHTFPAVLQSSGLYSLSSVVTVPSSSLGTQTYIC | |
| hIgG1-DHS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 185 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DHS | LFPPKPKDTLMISRTPEVTCVVVDVSHEDPEVKFNWYVDGV | |
| | EVHNAKTKPREEQYASTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 186 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DHS | LFPPKPKDTLMISRTPEVTCVVVAVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 187 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 188 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 189 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 190 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPCSRSTSESTAALGCLVKDYFPEPVTVSWN | 191 |
| Constant Region | SGALTSGVHTFPAVLQSSGLYSLSSVVTVPSSSLGTKTYTCN | |
| hIgG4-YTE | VDHKPSNTKVDKRVESKYGPPCPSCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 192 |
| Constant Region | SGALTSGVHTFPAVLQSSGLYSLSSVVTVPSSSLGTKTYTCN | |
| hIgG4-SP/YTE | VDHKPSNTKVDKRVESKYGPPCPPCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 193 |
| Constant Region | SGALTSGVHTFPAVLQSSGLYSLSSVVTVPSSSLGTKTYTCN | |
| hIgG4-SPLE/YTE | VDHKPSNTKVDKRVESKYGPPCPPCPAPEELGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 194 |
| Constant Region | SGALTSGVHTFPAVLQSSGLYSLSSVVTVPSSSLGTKTYTCN | |
| hIgG4-LS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 195 |
| Constant Region | SGALTSGVHTFPAVLQSSGLYSLSSVVTVPSSSLGTKTYTCN | |
| hIgG4-SP/LS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 196 |
| Constant Region | SGALTSGVHTFPAVLQSSGLYSLSSVVTVPSSSLGTKTYTCN | |
| hIgG4-SPLE/LS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 197 |
| Constant Region | SGALTSGVHTFPAVLQSSGLYSLSSVVTVPSSSLGTKTYTCN | |
| hIgG4-DHS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 198 |
| Constant Region | SGALTSGVHTFPAVLQSSGLYSLSSVVTVPSSSLGTKTYTCN | |
| hIgG4-SP/DHS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 199 |
| Constant Region | SGALTSGVHTFPAVLQSSGLYSLSSVVTVPSSSLGTKTYTCN | |
| hIgG4-SPLE/DHS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 200 |
| Constant Region | SGALTSGVHTFPAVLQSSGLYSLSSVVTVPSSNFGTQTYTCN | |
| hIgG2-YTE | VDHKPSNTKVDKTVERKCCVECPPCPAPPVAGPSVFLFPPKP | |
| | KDTLYITREPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPGK | |
| Heavy Chain | ASTKGPSVFPLAPCSRSTSESTAALGCLVKDYFPEPVTVSWN | 201 |
| Constant Region | SGALTSGVHTFPAVLQSSGLYSLSSVVTVPSSNFGTQTYTCN | |
| hIgG2-LS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHSHYTQKSLSLSPGK | |
| Heavy Chain | ASTKGPSVFPLAPCSRSTSESTAALGCLVKDYFPEPVTVSWN | 202 |
| Constant Region | SGALTSGVHTFPAVLQSSGLYSLSSVVTVPSSNFGTQTYTCN | |
| hIgG2-DHS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVDHHDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHSHYTQKSLSLSPGK | |
| Heavy Chain | ASTKGPSVFPLAPCSRSTSESTAALGCLVKDYFPEPVTVSWN | 203 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPSSKSTSGGTAALGCLVKDYFPEPVTVSW | 204 |
| Constant Region | NSGALTSGVHTFPAVLQSSGLYSLSSVVTVPSSSLGTQTYIC | |
| hIgG1-LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 205 |
| Constant Region | NSGALTSGVHTFPAVLQSSGLYSLSSVVTVPSSSLGTQTYIC | |
| hIgG1-N297A/LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 206 |
| Constant Region | NSGALTSGVHTFPAVLQSSGLYSLSSVVTVPSSSLGTQTYIC | |
| hIgG1-D265A/LA | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 207 |
| Constant Region | NSGALTSGVHTFPAVLQSSGLYSLSSVVTVPSSSLGTQTYIC | |
| hIgG1-LALA/LA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 208 |
| Constant Region | NSGALTSGVHTFPAVLQSSGLYSLSSVVTVPSSSLGTQTYIC | |
| hIgG1-LAGA/LA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 209 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 210 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 211 |
| Constant Region | NSGALTSGVHTFPAVLQSSGLYSLSSVVTVPSSSLGTQTYIC | |
| hIgG1-N434A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 212 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434A | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 213 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434A | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 214 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 215 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 216 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 217 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 218 |
| Constant Region | NSGALTSGVHTFPAVLQSSGLYSLSSVVTVPSSSLGTQTYIC | |
| hIgG1-N434W | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 219 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434W | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 220 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434W | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 221 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 222 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 223 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 224 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 225 |
| Constant Region | NSGALTSGVHTFPAVLQSSGLYSLSSVVTVPSSSLGTQTYIC | |
| hIgG1-DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 226 |
| Constant Region | NSGALTSGVHTFPAVLQSSGLYSLSSVVTVPSSSLGTQTYIC | |
| hIgG1-N297A/DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYASTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 227 |
| Constant Region | NSGALTSGVHTFPAVLQSSGLYSLSSVVTVPSSSLGTQTYIC | |
| hIgG1-D265A/DQ | NVNHKPSNTKVDKKVEPKSCDKTHTCPPCPAPELLGGPSVF | |
| | LFPPKPKDTLMISRDPEVTCVVVAVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 228 |
| Constant Region | NSGALTSGVHTFPAVLQSSGLYSLSSVVTVPSSSLGTQTYIC | |
| hIgG1-LALA/DQ | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 229 |
| Constant Region | NSGALTSGVHTFPAVLQSSGLYSLSSVVTVPSSSLGTQTYIC | |
| hIgG1-LAGA/DQ | NVNHKPSNTKVDKKVEPKSCDKTHTCPPCPAPELAGAPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 230 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 231 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 232 |
| Constant Region | NSGALTSGVHTFPAVLQSSGLYSLSSVVTVPSSSLGTQTYIC | |
| hIgG1-DW | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 233 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DW | LFPPKPKDTLMISRDPEVTCVVVDVSHEDPEVKFNWYVDGV | |
| | EVHNAKTKPREEQYASTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 234 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DW | LFPPKPKDTLMISRDPEVTCVVVAVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 235 |
| Constant Region | NSGALTSGVHTFPAVLQSSGLYSLSSVVTVPSSSLGTQTYIC | |
| hIgG1-LALA/DW | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 236 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 237 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 238 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 239 |
| Constant Region | NSGALTSGVHTFPAVLQSSGLYSLSSVVTVPSSSLGTQTYIC | |
| hIgG1-YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 240 |
| Constant Region | NSGALTSGVHTFPAVLQSSGLYSLSSVVTVPSSSLGTQTYIC | |
| hIgG1-N297A/YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 241 |
| Constant Region | NSGALTSGVHTFPAVLQSSGLYSLSSVVTVPSSSLGTQTYIC | |
| hIgG1-D265A/YD | NVNHKPSNTKVDKKVEPKSCDKTHTCPPCPAPELLGGPSVF | |
| | LFPPKPKDTLYISRDPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 242 |
| Constant Region | NSGALTSGVHTFPAVLQSSGLYSLSSVVTVPSSSLGTQTYIC | |
| hIgG1-LALA/YD | NVNHKPSNTKVDKKVEPKSCDKTHTCPPCPAPEAAGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 243 |
| Constant Region | NSGALTSGVHTFPAVLQSSGLYSLSSVVTVPSSSLGTQTYIC | |
| hIgG1-LAGA/YD | NVNHKPSNTKVDKKVEPKSCDKTHTCPPCPAPELAGAPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 244 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 245 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 246 |
| Constant Region | NSGALTSGVHTFPAVLQSSGLYSLSSVVTVPSSSLGTQTYIC | |
| hIgG1-QVV | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 247 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/QVV | LFPPKPKDTLMISRTPEVTCVVVDVSHEDPEVKFNWYVDGV | |
| | EVHNAKTKPREEQYASTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 248 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/QVV | LFPPKPKDTLMISRTPEVTCVVVAVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 249 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 250 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 251 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 252 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 253 |
| Constant Region | NSGALTSGVHTFPAVLQSSGLYSLSSVVTVPSSSLGTQTYIC | |
| hIgG1-DDRVV | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 254 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DDRVV | LFPPKPKDTLMISRDPEVTCVVVDVSHEDPEVKFNWYVDGV | |
| | EVDNAKTKPREEQYASTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 255 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DDRVV | LFPPKPKDTLMISRDPEVTCVVVAVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 256 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 257 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 258 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 259 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 260 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| Q311R/M428L | LFPPKPKDTLMISRTPEVTCVVVDVSHEDPEVKFNWYVDGV | |
| | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPCSRSTSESTAALGCLVKDYFPEPVTVSWN | 261 |
| Constant Region | SGALTSGVHTFPAVLQSSGLYSLSSVVTVPSSSLGTKTYTCN | |
| hIgG4- | VDHKPSNTKVDKRVESKYGPPCPSCPAPEFLGGPSVFLFPPK | |
| Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 262 |
| Constant Region | SGALTSGVHTFPAVLQSSGLYSLSSVVTVPSSSLGTKTYTCN | |
| IgG4- | VDHKPSNTKVDKRVESKYGPPCPPCPAPEFLGGPSVFLFPPK | |
| SP/Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 263 |
| Constant Region | SGALTSGVHTFPAVLQSSGLYSLSSVVTVPSSSLGTKTYTCN | |
| IgG4- | VDHKPSNTKVDKRVESKYGPPCPPCPAPEELGGPSVFLFPPK | |
| SPLE/Q311R/ | PKDTLMISRTPEVTCVVVDVSQEDPEVQFNWYVDGVEVHN | |
| M428L | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 264 |
| Constant Region | SGALTSGVHTFPAVLQSSGLYSLSSVVTVPSSNFGTQTYTCN | |
| IgG2- | VDHKPSNTKVDKTVERKCCVECPPCPAPPVAGPSVFLFPPKP | |
| Q311R/M428L | KDTLMISRTPEVTCVVVDVSHEDPEVQFNWYVDGVEVHNA | |
| | KTKPREEQFNSTFRVVSVLTVVHRDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHNHYTQKSLSLSPGK | |
| Heavy Chain | ASTKGPSVFPLAPSSKSTSGGTAALGCLVKDYFPEPVTVSW | 265 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/Q311R/ | LFPPKPKDTLMISRTPEVTCVVVDVSHEDPEVKFNWYVDGV | |
| M428L | EVHNAKTKPREEQYASTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 266 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/Q311R/ | LFPPKPKDTLMISRTPEVTCVVVAVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 267 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 268 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 269 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 270 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| TSLP (human) | MFPFALLYVLSVSFRKIFILQLVGLVLTYDFTNCDFEKIKAA | 271 |
| UniProt Accession | YLSTISKDLITYMSGTKSTEFNNTVSCSNRPHCLTEIQSLTFN | |
| No. Q969D9 | PTAGCASLAKEMFAMKTKAALAIWCPGYSETQINATQAMK | |
| | KRRKRKVTTNKCLEQVSQLQGLWRRFNRPLLKQQ | |
| TSLP | MKSLGQSKKEEVSFRKFFIFQLVGLVLTYDFTNCDFQKIEAD | 272 |
| (Cynomolgus | YLRTISKDLITYMSGTKSTDFNNTVSCSNRPHCLTEIQSLTFN | |
| monkey) | PTPRCASLAKEMFARKTKATLALWCPGYSETQINATQAMK | |
| UniProt Accession | KRRKRKVTTNKCLEQVSQLLGLWRRFIRTLLKKQ | |
| No. G7P819 | | |
| OX40L Construct | NYAMT | 273 |
| 114 Kabat | | |
| HCDR1 | | |
| OX40L Construct | GFTFSNY | 274 |
| 114 Chothia | | |
| HCDR1 | | |
| OX40L Construct | GFTFSNYA | 275 |
| 114 IMGT | | |
| HCDR1 | | |
| OX40L Construct | NYDIN | 276 |
| 231 Kabat | | |
| HCDR1 | | |
| OX40L Construct | GYTFTNY | 277 |
| 231 Chothia | | |
| HCDR1 | | |
| OX40L Construct | GYTFTNYD | 278 |
| 231 IMGT | | |
| HCDR1 | | |
| OX40L Construct | SGGYYWS | 279 |
| 328 Kabat | | |
| HCDR1 | | |
| OX40L Construct | GGSISSGGY | 280 |
| 328 Chothia | | |
| HCDR1 | | |
| OX40L Construct | GGSISSGGYY | 281 |
| 328 IMGT | | |
| HCDR1 | | |
| OX40L Construct | SYAMT | 282 |
| 105 Kabat | | |
| HCDR1 | | |
| OX40L Construct | GFTFSSY | 283 |
| 105 Chothia | | |
| HCDR1 | | |
| OX40L Construct | GFTFSSYA | 284 |
| 105 IMGT | | |
| HCDR1 | | |
| OX40L Construct | LISGSGGLTKYADSVKG | 285 |
| 114 Kabat | | |
| HCDR2 | | |
| OX40L Construct | SGSGGL | 286 |
| 114 Chothia | | |
| HCDR2 | | |
| OX40L Construct | ISGSGGLT | 287 |
| 114 IMGT | | |
| HCDR2 | | |
| OX40L Construct | WIHPNTGKTGYAQKFQG | 288 |
| 231 Kabat | | |
| HCDR2 | | |
| OX40L Construct | HPNTGK | 289 |
| 231 Chothia | | |
| HCDR2 | | |
| OX40L Construct | IHPNTGKT | 290 |
| 231 IMGT | | |
| HCDR2 | | |
| OX40L Construct | HIYYSGSTKYNPSLKS | 291 |
| 328 Kabat | | |
| HCDR2 | | |
| OX40L Construct | YYSGS | 292 |
| 328 Chothia | | |
| HCDR2 | | |
| OX40L Construct | IYYSGST | 293 |
| 328 IMGT | | |
| HCDR2 | | |
| OX40L Construct | LISGSGGLTKYADSVKG | 294 |
| 105 Kabat | | |
| HCDR2 | | |
| OX40L Construct | SGSGGL | 295 |
| 105 Chothia | | |
| HCDR2 | | |
| OX40L Construct | ISGSGGLT | 296 |
| 105 IMGT | | |
| HCDR2 | | |
| OX40L Construct | DEGLTTGEY | 297 |
| 114 Kabat | | |
| HCDR3 | | |
| OX40L Construct | EGLTTGE | 298 |
| 114 Chothia | | |
| HCDR3 | | |
| OX40L Construct | VKDEGLTTGEY | 299 |
| 114 IMGT | | |
| HCDR3 | | |
| OX40L Construct | ESYDLWSGHSFNWFDP | 300 |
| 231 Kabat | | |
| HCDR3 | | |
| OX40L Construct | SYDLWSGHSFNWFD | 301 |
| 231 Chothia | | |
| HCDR3 | | |
| OX40L Construct | ARESYDLWSGHSFNWFDP | 302 |
| 231 IMGT | | |
| HCDR3 | | |
| OX40L Construct | DNWGSGWYYMDV | 303 |
| 328 Kabat | | |
| HCDR3 | | |
| OX40L Construct | NWGSGWYYMD | 304 |
| 328 Chothia | | |
| HCDR3 | | |
| OX40L Construct | ARDNWGSGWYYMDV | 305 |
| 328 IMGT | | |
| HCDR3 | | |
| OX40L Construct | DEGLTTGEY | 306 |
| 105 Kabat | | |
| HCDR3 | | |
| OX40L Construct | EGLTTGE | 307 |
| 105 Chothia | | |
| HCDR3 | | |
| OX40L Construct | AKDEGLTTGEY | 308 |
| 105 IMGT | | |
| HCDR3 | | |
| OX40L Construct | RASQDIRNDLA | 309 |
| 114 Kabat LCDR1 | | |
| OX40L Construct | SQDIRND | 310 |
| 114 Chothia | | |
| LCDR1 | | |
| OX40L Construct | QDIRND | 311 |
| 114 IMGT | | |
| LCDR1 | | |
| OX40L Construct | RASQGITSYLA | 312 |
| 231 Kabat LCDR1 | | |
| OX40L Construct | SQGITSY | 313 |
| 231 Chothia | | |
| LCDR1 | | |
| OX40L Construct | QGITSY | 343 |
| 231 IMGT | | |
| LCDR1 | | |
| OX40L Construct | RASQSVSNYFA | 344 |
| 328 Kabat LCDR1 | | |
| OX40L Construct | SQSVSNY | 314 |
| 328 Chothia | | |
| LCDR1 | | |
| OX40L Construct | QSVSNY | 315 |
| 328 IMGT | | |
| LCDR1 | | |
| OX40L Construct | RASQGIRNDLG | 316 |
| 105 Kabat LCDR1 | | |
| OX40L Construct | SQGIRND | 317 |
| 105 Chothia | | |
| LCDR1 | | |
| OX40L Construct | QGIRND | 318 |
| 105 IMGT | | |
| LCDR1 | | |
| OX40L Construct | ASSSLQS | 319 |
| 114 Kabat LCDR2 | | |
| OX40L Construct | ASS | — |
| 114 Chothia and | | |
| IMGT LCDR2 | | |
| OX40L Construct | GATTLQS | 321 |
| 231 Kabat LCDR2 | | |
| OX40L Construct | GAT | — |
| 231 Chothia and | | |
| IMGT LCDR2 | | |
| OX40L Construct | DASNRAT | 323 |
| 328 Kabat LCDR2 | | |
| OX40L Construct | DAS | — |
| 328 Chothia and | | |
| IMGT LCDR2 | | |
| OX40L Construct | ATSSFQS | 325 |
| 105 Kabat LCDR2 | | |
| OX40L Construct | ATS | — |
| 105 Chothia | | |
| LCDR2 | | |
| OX40L Construct | LQHNNYPFT | 327 |
| 114 Kabat and | | |
| IMGT LCDR3 | | |
| OX40L Construct | HNNYPF | 328 |
| 114 Chothia | | |
| LCDR3 | | |
| OX40L Construct | QQLQSYPYT | 329 |
| 231 Kabat and | | |
| IMGT LCDR3 | | |
| OX40L Construct | LQSYPY | 330 |
| 231 Chothia | | |
| LCDR3 | | |
| OX40L Construct | QHRSNWPLT | 331 |
| 328 Kabat and | | |
| IMGT LCDR3 | | |
| OX40L Construct | RSNWPL | 332 |
| 328 Chothia | | |
| LCDR3 | | |
| OX40L Construct | LQHNSYPFT | 333 |
| 105 Kabat and | | |
| IMGT LCDR3 | | |
| OX40L Construct | HNSYPF | 334 |
| 105 Chothia | | |
| LCDR3 | | |
| OX40L Construct | EVQVVESGGGLVQPGGSLRLSCAASGFTFSNYAMTWVRQS | 335 |
| 114 Heavy Chain | PGKGLEWVSLISGSGGLTKYADSVKGRFTISRDNSKKTLFLQ | |
| Variable Domain | MNSLRAEDTAAYYCVKDEGLTTGEYWGQGTQVTVSS | |
| OX40L Construct | QVQLVQSGTEVKKPGASVKVSCKASGYTFTNYDINWVRQA | 336 |
| 231 Heavy Chain | TGQGLEWVGWIHPNTGKTGYAQKFQGRVTMTRDISIGTAY | |
| Variable Domain | MELNSLRSEDTAVYYCARESYDLWSGHSFNWFDPWGQGTL | |
| | VTVSS | |
| OX40L Construct | QVQLQESGPGLVKPSQTLSLTCTVSGGSISSGGYYWSWIRQ | 337 |
| 328 Heavy Chain | YPGKGLEWIGHIYYSGSTKYNPSLKSRLTISVDTSKNQFSLK | |
| Variable Domain | LSSVTAADTAVYYCARDNWGSGWYYMDVWGKGTTVTVS | |
| | S | |
| OX40L Construct | EVRLVESGGGLVQPGGSLRLSCAASGFTFSSYAMTWVRQAP | 338 |
| 105 Heavy Chain | GKGLEWVSLISGSGGLTKYADSVKGRFTISRDNSKKTLYLQ | |
| Variable Domain | MNSLRAEDTAVYYCAKDEGLTTGEYWGQGTLVTVSS | |
| OX40L Construct | DIQMTQSPSSLSASVGDRVTITCRASQDIRNDLAWYQQKPG | 339 |
| 114 Light Chain | KAPKRLIYASSSLQSGVPSRFSGSGSGTEFTLTISSLQPEDFAT | |
| Variable Domain | YYCLQHNNYPFTFGPGTKVDIK | |
| OX40L Construct | DIQLTQSPSFLSASVGDRVIITCRASQGITSYLAWYQRKPGK | 340 |
| 231 Light Chain | APKLLIYGATTLQSGVPSRFSGSGSGTEFTLTISSLQPEDFAT | |
| Variable Domain | YYCQQLQSYPYTFGQGTKLEIK | |
| OX40L Construct | EIVLTQSPATLSLSPGERATLSCRASQSVSNYFAWYRQKPGQ | 341 |
| 328 Light Chain | APRLLIYDASNRATGIPARFSGSGSGTDFTLTISSLEPEDFAV | |
| Variable Domain | YYCQHRSNWPLTFGGGTKVEIK | |
| OX40L Construct | DIQMTQSPSSLSASVGDRVTITCRASQGIRNDLGWYQQKPG | 342 |
| 105 Light Chain | KAPKRLIYATSSFQSGVPSRFSGSGSGTEFTLTISSLQPEDFAT | |
| Variable Domain | YYCLQHNSYPFTFGPGTKVDVK | |
| TSLP LCDR1 | RTSQNIRYYLN | 345 |
| TSLP LCDR1 | GGDNIGSKSVH | 346 |
| TSLP LCDR1 | GGQNIGTKSVH | 347 |
| TSLP LCDR1 | GGNKIGRKSVH | 348 |
| TSLP LCDR1 | GGNNIGGKSVH | 349 |
| TSLP LCDR1 | GGNNIGSKSVH | 350 |
| TSLP LCDR1 | GGHNIGTKSVH | 351 |
| TSLP LCDR1 | GGDSIGSKYVH | 352 |
| TSLP LCDR1 | GGQNIGTRSVH | 353 |
| TSLP LCDR1 | GGNNIGSETVH | 354 |
| TSLP LCDR1 | RGDNIETYSVH | 355 |
| TSLP LCDR1 | GGNNIGRKSAH | 356 |
| TSLP LCDR1 | EGTYLGAKSVH | 357 |
| TSLP LCDR1 | GGNNIGTKSVH | 358 |
| TSLP LCDR1 | GGQSIGTKSVH | 359 |
| TSLP LCDR1 | GANSIEDKNVH | 360 |
| TSLP LCDR1 | GGDNIGSYSVH | 361 |
| TSLP LCDR1 | GGNNVEDYSVH | 362 |
| TSLP LCDR1 | QNIRYY | 363 |
| TSLP LCDR1 | NIGSKS | 364 |
| TSLP LCDR1 | NIGTKS | 365 |
| TSLP LCDR1 | KIGRKS | 366 |
| TSLP LCDR1 | NIGGKS | 367 |
| TSLP LCDR1 | SIGSKY | 368 |
| TSLP LCDR1 | NIGTRS | 369 |
| TSLP LCDR1 | NIGSET | 370 |
| TSLP LCDR1 | NIETYS | 371 |
| TSLP LCDR1 | NIGRKS | 372 |
| TSLP LCDR1 | SIGTKS | 373 |
| TSLP LCDR1 | SIEDKN | 374 |
| TSLP LCDR1 | NIGSYS | 375 |
| TSLP LCDR1 | NVEDYS | 376 |
| TSLP LCDR2 | DVSSLQS | 377 |
| TSLP LCDR2 | EDSDRPS | 378 |
| TSLP LCDR2 | DDTDRPS | 379 |
| TSLP LCDR2 | DDSDRPP | 380 |
| TSLP LCDR2 | DDKERPS | 381 |
| TSLP LCDR2 | DDSERPS | 382 |
| TSLP LCDR2 | DDLKRPS | 383 |
| TSLP LCDR2 | DDNDRPS | 384 |
| TSLP LCDR3 | QQGYSPPLT | 385 |
| TSLP LCDR3 | QVWDSDSDHVV | 386 |
| TSLP LCDR3 | QVWDSSSDQSV | 387 |
| TSLP LCDR3 | QVWDSSSDEGV | 388 |
| TSLP LCDR3 | QVWDGSSDQSV | 389 |
| TSLP LCDR3 | QVWDTTTDHVV | 390 |
| TSLP LCDR3 | QVWDSSSDPVV | 391 |
| TSLP LCDR3 | QVWDRSSDQSV | 392 |
| TSLP LCDR3 | QVWDSGSGQTV | 393 |
| TSLP LCDR3 | HVWDSSSDHVM | 394 |
| TSLP LCDR3 | QVWDSDSDHLE | 395 |
| TSLP LCDR3 | QVWDSYSNHVV | 396 |
| TSLP LCDR3 | QVWDSGGDHVV | 397 |
| TSLP LCDR3 | QVWDDISDHIV | 398 |
| TSLP LCDR3 | QVWDMASDRVV | 399 |
| TSLP LCDR3 | QVWDTSSDQGV | 400 |
| TSLP LCDR3 | QVWDSTSGVVF | 401 |
| TSLP VL | DIQLTQSPSSLSASVGDRVAITCRTSQNIRYYLNWYQRKPGK | 402 |
| | APRLLIYDVSSLQSGVPSRFSGRGSGTDFTLTISSLQAEDFAT | |
| | YYCQQGYSPPLTFGGGTKVEIK | |
| TSLP VL | NFMLTQPPSVSVAPGKTAKMTCGGDNIGSKSVHWYQQMPG | 403 |
| | QAPVLVIYEDSDRPSGTPERFSGSNSGHTATLTISRVEAGDE | |
| | ADYYCQVWDSDSDHVVFGGGTTLTVL | |
| TSLP VL | QSVLTQPPSVSVAPGQTARITCGGQNIGTKSVHWYQQKSGQ | 404 |
| | APILVVYDDSDRPSWIPERFSGSNFGTTATLTISRVEAGDEA | |
| | DYFCQVWDSSSDQSVFGPGTRVTVL | |
| TSLP VL | SYELTQPPSVSVAPGQTARITCGGQNIGTKSVHWYQQKSGQ | 405 |
| | APILVVYDDSDRPSWIPERFSGSNFGTTATLTISRVEAGDEA | |
| | DYFCQVWDSSSDQSVFGPGTRVTVL | |
| TSLP VL | SYVLTQPPSVSVAPGQTARITCGGQNIGTKSVHWYQQKSGQ | 406 |
| | APILVVYDDSDRPSWIPERFSGSNFGTTATLTISRVEAGDEA | |
| | DYFCQVWDSSSDQSVFGPGTRVTVL | |
| TSLP VL | SYVLTQPPSVSVAPGQTARITCGGNKIGRKSVHWYQQRPGQ | 407 |
| | APVLVVFDDSDRPSWISERFSGSNSGNTATLTISRVEAGDEA | |
| | DYYCQVWDSSSDEGVFGGGTKLTVL | |
| TSLP VL | SYELTQPPSVSVAPGQAARITCGGNKIGRKSVHWYQQRPGQ | 408 |
| | APVLVVFDDSDRPSWISERFSGSNSGNTATLTISRVEAGDEA | |
| | DYYCQVWDSSSDEGVFGGGTKLTVL | |
| TSLP VL | QSVLTQPPSVSVAPGQTARITCGGQNIGTKSVHWYQQKSGQ | 409 |
| | APILVVYDDSDRPSWIPERFSGSNFGTTATLTISRVEAGDEA | |
| | DYFCQVWDGSSDQSVFGPGTRVTVL | |
| TSLP VL | SYELTQPPSVSVAPGQTASITCGGNNIGGKSVHWYQLKPGQ | 410 |
| | APVLVVYDDTDRPSWIPERFSGSNSANTATLTISGVEAGDEA | |
| | DYQCQVWDTTTDHVVFGGGTKLTVL | |
| TSLP VL | SYVLTQPPSVSVAPGQTARITCGGNNIGSKSVHWYQQKPGQ | 411 |
| | APLLVVYDDSDRPSWIPERFSGSNSGNAATLTVSRVEAGDE | |
| | ADYYCQVWDSSSDPVVFGGGTKLTVL | |
| TSLP VL | FYVLTQPPSVSVAPGQTARITCGGQNIGTKSVHWYQQKSGQ | 412 |
| | APILVVYDDSDRPSWIPERFSGSNFGTTATLTISRVEAGDEA | |
| | DYFCQVWDSSSDQSVFGPGTRVTVL | |
| TSLP VL | QSVLTQPPSVSVAPGQTARITCGGQNIGTKSVHWYQQKSGQ | 413 |
| | APILVVYDDSDRPPWIPERFSGSNFGTTATLTISRVEAGDEA | |
| | DYFCQVWDSSSDQSVFGPGTRVTVL | |
| TSLP VL | QAVLTQPPSVSVAPGQTARITCGGNKIGRKSVHWYQQRPGQ | 414 |
| | APVLVVFDDSDRPSWISERFSGSNSGNTATLTISRVEAGDEA | |
| | DYYCQVWDSSSDEGVFGGGTKLTVL | |
| TSLP VL | SYELTQPPSVSVAPGQTARITCGGHNIGTKSVHWYQQKSGQ | 415 |
| | APILVVYDDSDRPSWIPERFSGSNFGTTATLTISRVEAGDEA | |
| | DYFCQVWDRSSDQSVFGPGTRVTVL | |
| TSLP VL | SYVLTQPPSVSVAPGQTARVTCGGDSIGSKYVHWYQQKPG | 416 |
| | QAPVLVVYDDSDRPSWIPERFSGSNFGTTATLTISRVEAGDE | |
| | ADYFCQVWDRSSDQSVFGPGTRVTVL | |
| TSLP VL | SYELTQPPSVSVAPGQTARITCGGQNIGTKSVHWYQQKSGQ | 417 |
| | APILVVFDDSDRPSWVTERFSGSNFGTTATLTINGVEAGDEA | |
| | DYFCQVWDSGSGQTVFGPGTRVTVS | |
| TSLP VL | SYELTQPPSVSVAPGQTARITCGGQNIGTRSVHWYQQKSGQ | 418 |
| | APILVVYDDSDRPSWIPERFSGSNFGTTATLTISRVEAGDEA | |
| | DYFCQVWDSSSDQSVFGPGTRVTVL | |
| TSLP VL | SYELTQPPSVSVAPGQTARITCGGNNIGSETVHWYRQKAGQ | 419 |
| | APVLVVYDDSDRPSGIPERFSGSNSGNTAALTISRVEVGDEA | |
| | DYYCHVWDSSSDHVMFGGGTKLTVL | |
| TSLP VL | SYELTQPPSVSVGPGKTATITCRGDNIETYSVHWYQQKPGQ | 420 |
| | APVLVVYDDSDRPSGIPERFSGSNSGNTATLTINRVGAGDEA | |
| | DYYCQVWDSDSDHLEFGGGTKLTVL | |
| TSLP VL | SYELTQPPSVSVAPGQTARITCGGNNIGRKSAHWYQQRPGQ | 421 |
| | APVLVVYDDSDRPSWIPERFSGSNSGNTATLTISRVEAGDEA | |
| | DYYCQVWDSYSNHVVFGGGTKLTVL | |
| TSLP VL | LPVLTQPPSVSVAPGQTARITCGGNKIGRKSVHWYQQRPGQ | 422 |
| | APVLVVFDDSDRPSWISERFSGSNSGNTATLTISRVEAGDEA | |
| | DYYCQVWDSSSDEGVFGGGTKLTVL | |
| TSLP VL | SYVLTQPPSVSVAPGQTARITCGGHNIGTKSVHWYQQKSGQ | 423 |
| | APILAVYDDSDRPSWIPERFSGSNFGTTATLTISRVEAGDEA | |
| | DYFCQVWDRSSDQSVFGPGTRVTVL | |
| TSLP VL | SYELTQPPSVSVGPGKTATITCRGDNIETYSVHWYQQKPGQ | 424 |
| | APVLVVYDDSDRPSGIPERFSGSNSGNTATLTINRVEARDEA | |
| | DYFCQVWDSGGDHVVFGGGTKLTVL | |
| TSLP VL | SYVLTQPPSVSVAPGQTARITCGGNNIGSKSVHWYQQKPGQ | 425 |
| | APVLVVYDDSDRPSWIPERFSGTNSGNTATLTISRVEAGDEA | |
| | DYYCQVWDSSSDHVVFGGGTKLTVL | |
| TSLP VL | SYELTQPPSVSVAPGKRARITCEGTYLGAKSVHWYQQKPGQ | 426 |
| | APVLVIYDDKERPSGIPERFSASNSGNTATLTITRVEAGDEA | |
| | DYHCQVWDDISDHIVFGGGTKLSVL | |
| TSLP VL | SYVLTQPPSVSVAPGQTARITCGGNNIGTKSVHWYQQKSGQ | 427 |
| | APILVVYDDSDRPSWIPERFSGSNFGTTATLTISRVEAGDEA | |
| | DYFCQVWDSSSDQSVFGPGTRVTVL | |
| TSLP VL | SYVLTQPPSVSVAPGQTARITCGGQSIGTKSVHWYQQKSGQ | 428 |
| | APILVVYDDSDRPSWIPERFSGSNFGTTATLTISRVEAGDEA | |
| | DYFCQVWDSSSDQSVFGPGTRVTVL | |
| TSLP VL | SYELTQPPSVSVAPGQAASLTCGANSIEDKNVHWYQQRPGQ | 429 |
| | APLLVIYDDSERPSGIPERFSGSNSGNTATLTITRVAAGDEAD | |
| | YYCQVWDMASDRVVFGPGTTVSVL | |
| TSLP VL | SYELTQPPSVSVAPGQTARITCGGNKIGRKSVHWYQQRPGQ | 430 |
| | APVLVVFDDSDRPSWISERFSGSNSGNTATLTIGRVEAGDEA | |
| | DYYCQVWDSSSDEGVFGGGTKLAVL | |
| TSLP VL | SYELTQPPSVSVAPGRTARITCGGQNIGTKSVHWYQQKSGQ | 431 |
| | APILVVYDDSDRPSWIPERFSGSNFGTTATLTISRVEAGDEA | |
| | DYFCQVWDSSSDQSVFGPGTRVTVL | |
| TSLP VL | QAGLTQPPSVSVAPGQTARITCGGNKIGRKSVHWYQQRPGQ | 432 |
| | APVLVVFDDSDRPSWISERFSGSNSGNTATLTISRVEAGDEA | |
| | DYYCQVWDSSSDEGVFGGGTKLTVL | |
| TSLP VL | QSVLTQPPSVSVAPGQTARITCGGQNIGTKSVHWYQQKSGQ | 433 |
| | APILVVYDDSDRPSWIPERFSGSNFGTTATLTISRVEAGDEA | |
| | DYFCQVWDSSSDQSVFGTGTKVTVL | |
| TSLP VL | SYVLTQPPSVSVGPGKTATITCRGDNIETYSVHWYQRKPGQ | 434 |
| | APVLVVYDDSDRPSGIPERFSGSNSGNTATLTISRVEAGDEA | |
| | DYYCQVWDSSSDHVVFGGGTKLTVL | |
| TSLP VL | SYELTQPPSVSVAPGQTARITCGGNKIGRKSVHWYQQRPGQ | 435 |
| | APVLVVFDDSDRPSWISERFSGSNSGNTATLTISRVDAGDEA | |
| | DYYCQVWDSSSDEGVFGGGTKLTVL | |
| TSLP VL | SYELTQPPSVSVAPGQTARITCGGNKIGRKSVHWYQQKPGQ | 436 |
| | APVLVVFDDSDRPSWISERFSGSNSGNTATLTISRVEAGDEA | |
| | DYHCQVWDTSSDQGVFGGGTKLTVL | |
| TSLP VL | SYVLTQPPSVAVAPGQTAKITCGGDNIGSYSVHWYQQKPGQ | 437 |
| | APVLVVYDDLKRPSWIPERFFGSNSGNTATLTISRVEAGDEA | |
| | VYYCQVWDSTSGVVFGGGTTVTVL | |
| TSLP VL | SYELTQPPSVSVAPGKTARITCGGNNVEDYSVHWYHQKAG | 438 |
| | QAPVVVMYDDNDRPSGVPERFSGSNSGNTATLTIGRVEAGD | |
| | EADYYCQVWDSSSDHVVFGGGTKLTVL | |
| OX40L protein | MERVQPLEENVGNAARPRFERNKLLLVASVIQGLGLLLCFT | 439 |
| | YICLHFSALQVSHRYPRIQSIKVQFTEYKKEKGFILTSQKEDE | |
| | IMKVQNNSVIINCDGFYLISLKGYFSQEVNISLHYQKDEEPLF | |
| | QLKKVRSVNSLMVASLTYKDKVYLNVTTDNTSLDDFHVNG | |
| | GELILIHQNPGEFCVL | |
| lambda-1 light | QSALTQPPSASGSLGQSVTISCTGTSSDVGGYNYVSWYQQH | 440 |
| chain | AGKAPKVIIYEVNKRPSGVPDRFSGSKSGNTASLTVSGLQAE | |
| | DEADYYCSSYEGSDNFVFGTGTKVTVLGQPKANPTVTLFPP | |
| | SSEELQANKATLVCLISDFYPGAVTVAWKADGSPVKAGVET | |
| | TKPSKQSNNKYAASSYLSLTPEQWKSHRSYSCQVTHEGSTV | |
| | EKTVAPTECS | |
| Tezepelumab light | SYVLTQPPSVSVAPGQTARITCGGNNLGSKSVHWYQQKPG | 441 |
| chain | QAPVLVVYDDSDRPSWIPERFSGSNSGNTATLTISRGEAGDE | |
| | ADYYCQVWDSSSDHVVFGGGTKLTVL | |
| hu23b12 VH | QVQLVQSGAEVKKPGASVKVSCKASGYIFTDYAMHWVRQ | 442 |
| | APGQGLEWMGTFIPLLDTSDYNQNFKGRVTMTTDTSTSTAY | |
| | MELRSLRSDDTAVYYCARMGVTHSYVMDAWGQGTLVTVS | |
| | S | |
| hu23b12 VL | EIVLTQSPGTLSLSPGERATLSCRASQPISISVHWYQQKPGQA | 443 |
| | PRLLIYFASQSISGIPDRFSGSGSGTDFTLTISRLEPEDFAVYY | |
| | CQQTFSLPYTFGQGTKVEIKR | |
| 43B1-H2L2 VH | QMQLVESGGGAVQPGGSLRLSCAASGFTFRSYDMHWVRQ | 444 |
| | APGKGLEWVAVIWYDGSNENYADSVKGRFTTSRDNSKNTL | |
| | FLQMNSLRAEDTAVYYCARSPLWYGEPDDAFDIWGQGTLV | |
| | TVSS | |
| 43B1-H2L2 VL | EIVMTQSPATLSVSPGERATLSCRASQSVSSNLAWYQQKPG | 445 |
| | QAPRLLISGASTRATGIPARFSGSGSGTDFTLTISSLQSEDFAV | |
| | YYCQHYNNWPLTFGGGTKVEIK | |
| 43B1-H6L1 VH | QMQLVESGGGAVQPGRSLRLSCAASGFTFRSYDMHWVRQ | 446 |
| | APGKGLEWVAVIWYDGSNENYADSVKGRFTTSRDNSKNTL | |
| | FLQMNSLRAEDTAVYYCARSPLWYGEPDDAFDIWGQGTLV | |
| | TVSS | |
| 43B1-H6L1 VL | EIVMTQSPATLSVSPGERATLSCRASQSVSSNLAWYQQKPG | 447 |
| | QAPRLLISGASTRATGIPARFSGSGSGTDFTLTISSLRSEDFAV | |
| | YYCQHYNNWPLTFGGGTKVEIK | |
| Amlitelimab VH | EVQLVESGGGLVQPGGSLRLSCAASGFTFSNYAMNWVRQA | 448 |
| | PGKGLEWVSTISGSGGATRYADSVKGRFTISRDNSRNTVYL | |
| | QMNSLRVEDTAVFYCTKDRLIMATVRGPYYYGMDVWGQG | |
| | TTVTVSS | |
| Amlitelimab VL | DIQMTQSPSSLSASVGDRVTITCRASQSISSYLNWYQQKPGK | 449 |
| | APNLLIYAASSLQSGVPSRFSGSGSETDFTLTISSLQPEDFATY | |
| | YCQQSHSVSFTFGPGTKVDIK | |
| Oxelumab heavy | EVQLLESGGGLVQPGGSLRLSCAASGFTFNSYAMSWVRQA | 450 |
| chain | PGKGLEWVSIISGSGGFTYYADSVKGRFTISRDNSRTTLYLQ | |
| | MNSLRAEDTAVYYCAKDRLVAPGTFDYWGQGALVTVSSA | |
| | STKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKV | |
| | SNKALPAPIEKTISKAKGQPREPQVYTLPPSRDELTKNQVSL | |
| | TCLVKGFYPSDIAVEWESNGQPENNYKTTPPVLDSDGSFFL | |
| | YSKLTVDKSRWQQGNVFSCSVMHEALHNHYTQKSLSLSPG | |
| Oxelumab light | DIQMTQSPSSLSASVGDRVTITCRASQGISSWLAWYQQKPE | 451 |
| chain | KAPKSLIYAASSLQSGVPSRFSGSGSGTDFTLTISSLQPEDFA | |
| | TYYCQQYNSYPYTFGQGTKLEIKRTVAAPSVFIFPPSDEQLK | |
| | SGTASVVCLLNNFYPREAKVQWKVDNALQSGNSQESVTEQ | |
| | DSKDSTYSLSSTLTLSKADYEKHKVYACEVTHQGLSSPVTK | |
| | SFNRGEC | |
| TSLP Antibody 3 | QMQLVESGGGVVQPGRSLRLSCAASGFTFRTYPMHWVRQA | 452 |
| VH | PGKGLEWVAVIWYDGSNKHYADSVKGRFTITRDNSKNTLN | |
| | LQMNSLRAEDTAVYYCARALIWELFHEYFDIWGQGTMVTV | |
| | SS | |

*SEQ ID NOs: 47 and 159 are hIgG1 sequences having a SRDEL (SEQ ID NO: 588) allotype. The disclosure also encompasses SREEM (SEQ ID NO: 589) allotype variants of any of the SRDEL (SEQ ID NO: 588) allotype sequences set forth in the table above, and either a SRDEL (SEQ ID NO: 588) allotype heavy chain constant region sequence or a SREEM (SEQ ID NO: 589) allotype heavy chain constant region sequence may be included in an anti-OX40L antibody or an anti-TSLP antibody described herein.

SEQ ID NO: 453
MGVLKFKHIFFRSFVKSSGVSQIVFTFLLIPCCLTLNFRAPPVIPNVPELWAWNAPSEFCLGKED
EPLDMSLESFIGSPRINATGQGVTIFYVDRLGYYPYIDSITGVTVNGGIPQKISLQDHLDKAKKD
ITFYMPVDNLGMAVIDWEEWRPTWARNWKPKDVYKNRSIELVQQQNVQLSLTEATEKAKQEFEKA
GKDFLVETIKLGKLLRPNHLWGYYLFPDCYNHHYKKPGYNGSCENVEIKRNDDLSWLWNESTALY
PSIYLNTQQSPVAATLYVRNRVREAIRVSKIPDAKSPLPVFAYTRIVFTDQVLKELSQDELVYTE
GETVALGASGIVIWGTLSIMRSMKSCLLLDNYMETILNPYIINVTLAAKMCSQVLCQEQGVCIRK
NWNSSDYLHLNPDNFAIQLEKGGKFTVRGKPTLEDLEQFSEKFYCSCYSTLSCKEKADVKDTDAV
DVCIADGVCIDAFLKPPMETEEPQIFYNASPSTLSATMFIVSILFLIISSVASL
SEQ ID NO: 454
LNFRAPPVIPNVPELWAWNAPSEFCLGKFDEPLDMSLESFIGSPRINATGQGVTIFYVDRLGYYP
YIDSITGVTVNGGIPQKISLQDHLDKAKKDITFYMPVDNLGMAVIDWEEWRPTWARNWKPKDVYK
NRSIELVQQQNVQLSLTEATEKAKQEFEKAGKDELVETIKLGKLLRPNHLWGYYLFPDCYNHHYK
KPGYNGSCENVEIKRNDDLSWLWNESTALYPSIYLNTQQSPVAATLYVRNRVREAIRVSKIPDAK
SPLPVFAYTRIVFTDQVLKELSQDELVYTFGETVALGASGIVIWGTLSIMRSMKSCLLLDNYMET
ILNPYIINVTLAAKMCSQVLCQEQGVCIRKNWNSSDYLHLNPDNFAIQLEKGGKFTVRGKPTLED
LEQFSEKFYCSCYSTLSCKEKADVKDTDAVDVCIADGVCIDAFLKPPMETEEPQIFYNASPSTLS
ATMFIVSILFLIISSVASL
SEQ ID NO: 455
LNFRAPPVIPNVPELWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFY
SEQ ID NO: 456
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKELSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIF
SEQ ID NO: 457
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQI
SEQ ID NO: 458
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKELSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQ
SEQ ID NO: 459
LNFRAPPVIPNVPELWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEP
SEQ ID NO: 460
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKELSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEE
SEQ ID NO: 461
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKELSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDA
SEQ ID NO: 462
LNFRAPPVIPNVPFLWAWNAPSEFCLGKFDEPLDMSLESFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFYN
SEQ ID NO: 463
atgggagtgc taaaattcaa gcacatcttt ttcagaagct ttgttaaatc aagtggagta
tcccagatag ttttcacctt ccttctgatt ccatgttgct tgactctgaa tttcagagca
cctcctgtta ttccaaatgt gcctttcctc tgggcctgga atgccccaag tgaattttgt
cttggaaaat ttgatgagcc actagatatg agcctcttct ctttcatagg aagcccccga
ataaacgcca ccgggcaagg tgttacaata ttttatgttg atagacttgg ctactatcct
tacatagatt caatcacagg agtaactgtg aatggaggaa tcccccagaa gatttcctta
caagaccato tggacaaagc taagaaagac attacatttt atatgccagt agacaatttg
ggaatggctg ttattgactg ggaagaatgg agacccactt gggcaagaaa ctggaaacct
aaagatgttt acaagaatag gtctattgaa ttggttcagc aacaaaatgt acaacttagt
ctcacagagg ccactgagaa agcaaaacaa gaatttgaaa aggcagggaa ggatttcctg
gtagagacta taaaattggg aaaattactt cggccaaatc acttgtgggg ttattatctt
tttccggatt gttacaacca tcactataag aaacccggtt acaatggaag ttgcttcaat
gtagaaataa aaagaaatga tgatctcagc tggttgtgga atgaaagcac tgctctttac
ccatccattt atttgaacac tcagcagtct cctgtagctg ctacactcta tgtgcgcaat
cgagtteggg aagccatcag agtttccaaa atacctgatg caaaaagtcc acttccggtt
tttgcatata cccgcatagt ttttactgat caagttttga aattcctttc tcaagatgaa
cttgtgtata catttggcga aactgttgct ctgggtgctt ctggaattgt aatatgggga
accctcagta taatgcgaag tatgaaatct tgcttgctcc tagacaatta catggagact
atactgaatc cttacataat caacgtcaca ctagcagcca aaatgtgtag ccaagtgctt
tgccaggagc aaggagtgtg tataaggaaa aactggaatt caagtgacta tettcacctc
aacccagata attttgctat tcaacttgag aaaggtggaa agttcacagt acgtggaaaa
ccgacacttg aagacctgga gcaattttct gaaaaatttt attgcagctg ttatagcacc
ttgagttgta aggagaaagc tgatgtaaaa gacactgatg ctgttgatgt gtgtattgct
gatggtgtct gtatagatgc ttttctaaaa cctcccatgg agacagaaga acctcaaatt
ttctac
SEQ ID NO: 464
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Gly Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 465
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Phe Ile Leu Asn Val Thr Ser
Gly Ala Leu Leu Cys Ser Gln Ala Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 466
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Gly Pro Phe Ile Leu Asn Val Thr Ser
Gly Ala Leu Leu Cys Ser Gln Ala Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 467
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Val Ser Trp Glu Asn Thr Arg Thr Lys Glu Ser Cys Gln Ala Ile Lys
Glu Tyr Met Asp Thr Thr Leu Gly Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 468
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 469
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Val Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 470
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 471
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 472
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 473
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Phe Ile Leu Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 474
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Phe Ile Leu Asn Val Thr Ser
Gly Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 475
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Leu Ser Ile Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile Phe Tyr Asn Ala Ser
Pro Ser Thr Leu Ser
SEQ ID NO: 476
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile
SEQ ID NO: 477
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe
SEQ ID NO: 478
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro
SEQ ID NO: 479
Phe Arg Gly Pro Leu Leu Pro Asn Arg Pro Phe Leu Trp Ala Trp Asn
Ala Pro Ser Glu Phe Cys Leu Gly Lys Phe Asp Glu Pro Leu Asp Met
Ser Leu Phe Ser Phe Ile Gly Ser Pro Arg Ile Asn Ala Thr Gly Gln
Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile
Ser Leu Gln Asp His Leu Asp Lys Ala Lys Lys Asp Ile Thr Phe Tyr
Met Pro Val Asp Asn Leu Gly Met Ala Val Ile Asp Trp Glu Glu Trp
Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp
Phe Leu Val Glu Thr Ile Lys Leu Gly Lys Leu Leu Arg Pro Asn His
Leu Trp Gly Tyr Tyr Leu Phe Pro Asp Cys Tyr Asn His His Tyr Lys
Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser
Ile Tyr Leu Asn Thr Gln Gln Ser Pro Val Ala Ala Thr Leu Tyr Val
Arg Asn Arg Val Arg Glu Ala Ile Arg Val Ser Lys Ile Pro Asp Ala
Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly
Glu Thr Val Ala Leu Gly Ala Ser Gly Ile Val Ile Trp Gly Thr Leu
Ser Ile Thr Arg Thr Lys Glu Ser Cys Gln Ala Ile Lys Glu Tyr Met
Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys
Asn Trp Asn Ser Ser Asp Tyr Leu His Leu Asn Pro Asp Asn Phe Ala
Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala
Val Asp Val Cys Ile Ala Asp Gly Val Cys Ile Asp Ala Phe Leu Lys
Pro Pro Met Glu Thr Glu Glu Pro Gln Ile Phe Tyr Asn Ala Ser Pro
Ser Thr Leu Ser
SEQ ID NO: 480
Phe Arg Gly Pro Leu Leu Pro Asn Arg Pro Phe Thr Thr Val Trp Asn
Ala Pro Ser Glu Phe Cys Leu Gly Lys Phe Asp Glu Pro Leu Asp Met
Ser Leu Phe Ser Phe Ile Gly Ser Pro Arg Ile Asn Ala Thr Gly Gln
Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile
Ser Leu Gln Asp His Leu Asp Lys Ala Lys Lys Asp Ile Thr Phe Tyr
Met Pro Val Asp Asn Leu Gly Met Ala Val Ile Asp Trp Glu Glu Trp
Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp
Phe Leu Val Glu Thr Ile Lys Leu Gly Lys Leu Leu Arg Pro Asn His
Leu Trp Gly Tyr Tyr Leu Phe Pro Asp Cys Tyr Asn His His Tyr Lys
Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser
Ile Tyr Leu Asn Thr Gln Gln Ser Pro Val Ala Ala Thr Leu Tyr Val
Arg Asn Arg Val Arg Glu Ala Ile Arg Val Ser Lys Ile Pro Asp Ala
Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly
Glu Thr Val Ala Leu Gly Ala Ser Gly Ile Val Ile Trp Gly Thr Leu
Ser Ile Thr Arg Thr Lys Glu Ser Cys Gln Ala Ile Lys Glu Tyr Met
Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys
Asn Trp Asn Ser Ser Asp Tyr Leu His Leu Asn Pro Asp Asn Phe Ala
Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala
Val Asp Val Cys Ile Ala Asp Gly Val Cys Ile Asp Ala Phe Leu Lys
Pro Pro Met Glu Thr Glu Glu Pro Gln Ile Phe Tyr Asn Ala Ser Pro
Ser Thr Leu Ser
SEQ ID NO: 481
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys
SEQ ID NO: 482
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 483
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 484
Ala Pro Pro Val Ile Pro Asn Val Pro Phe Leu Trp Ala Trp Asn Ala
Pro Ser Glu Phe Cys Leu Gly Lys Phe Asp Glu Pro Leu Asp Met Ser
Leu Phe Ser Phe Ile Gly Ser Pro Arg Ile Asn Ala Thr Gly Gln Gly
Val Thr Ile Phe Tyr Val Asp Arg Leu Gly Tyr Tyr Pro Tyr Ile Asp
Ser Ile Thr Gly Val Thr Val Asn Gly Gly Ile Pro Gln Lys Ile Ser
Leu Gln Asp His Leu Asp Lys Ala Lys Lys Asp Ile Thr Phe Tyr Met
Pro Val Asp Asn Leu Gly Met Ala Val Ile Asp Trp Glu Glu Trp Arg
Pro Thr Trp Ala Arg Asn Trp Lys Pro Lys Asp Val Tyr Lys Asn Arg
Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe
Leu Val Glu Thr Ile Lys Leu Gly Lys Leu Leu Arg Pro Asn His Leu
Trp Gly Tyr Tyr Leu Phe Pro Asp Cys Tyr Asn His His Tyr Lys Lys
Pro Gly Tyr Asn Gly Ser Cys Phe Asn Val Glu Ile Lys Arg Asn Asp
Asp Leu Ser Trp Leu Trp Asn Glu Ser Thr Ala Leu Tyr Pro Ser Ile
Tyr Leu Asn Thr Gln Gln Ser Pro Val Ala Ala Thr Leu Tyr Val Arg
Asn Arg Val Arg Glu Ala Ile Arg Val Ser Lys Ile Pro Asp Ala Lys
Ser Pro Leu Pro Val Phe Ala Tyr Thr Arg Ile Val Phe Thr Asp Gln
Val Leu Lys Phe Leu Ser Gln Asp Glu Leu Val Tyr Thr Phe Gly Glu
Thr Val Ala Leu Gly Ala Ser Gly Ile Val Ile Trp Gly Thr Leu Ser
Ile Thr Arg Thr Lys Glu Ser Cys Gln Ala Ile Lys Glu Tyr Met Asp
Thr Thr Leu Asn Pro Tyr Ile Ile Asn Val Thr Leu Ala Ala Lys Met
Cys Ser Gln Val Leu Cys Gln Glu Gln Gly Val Cys Ile Arg Lys Asn
Trp Asn Ser Ser Asp Tyr Leu His Leu Asn Pro Asp Asn Phe Ala Ile
Gln Leu Glu Lys Gly Gly Lys Phe Thr Val Arg Gly Lys Pro Thr Leu
Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val
Asp Val Cys Ile Ala Asp Gly Val Cys Ile Asp Ala Phe Leu Lys Pro
Pro Met Glu Thr Glu Glu Pro Gln Ile Phe Tyr Asn Ala Ser Pro Ser
Thr Leu Ser
SEQ ID NO: 485
Pro Val Ile Pro Asn Val Pro Phe Leu Trp Ala Trp Asn Ala Pro Ser
Glu Phe Cys Leu Gly Lys Phe Asp Glu Pro Leu Asp Met Ser Leu Phe
Ser Phe Ile Gly Ser Pro Arg Ile Asn Ala Thr Gly Gln Gly Val Thr
Ile Phe Tyr Val Asp Arg Leu Gly Tyr Tyr Pro Tyr Ile Asp Ser Ile
Thr Gly Val Thr Val Asn Gly Gly Ile Pro Gln Lys Ile Ser Leu Gln
Asp His Leu Asp Lys Ala Lys Lys Asp Ile Thr Phe Tyr Met Pro Val
Asp Asn Leu Gly Met Ala Val Ile Asp Trp Glu Glu Trp Arg Pro Thr
Trp Ala Arg Asn Trp Lys Pro Lys Asp Val Tyr Lys Asn Arg Ser Ile
Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val
Glu Thr Ile Lys Leu Gly Lys Leu Leu Arg Pro Asn His Leu Trp Gly
Tyr Tyr Leu Phe Pro Asp Cys Tyr Asn His His Tyr Lys Lys Pro Gly
Tyr Asn Gly Ser Cys Phe Asn Val Glu Ile Lys Arg Asn Asp Asp Leu
Ser Trp Leu Trp Asn Glu Ser Thr Ala Leu Tyr Pro Ser Ile Tyr Leu
Asn Thr Gln Gln Ser Pro Val Ala Ala Thr Leu Tyr Val Arg Asn Arg
Val Arg Glu Ala Ile Arg Val Ser Lys Ile Pro Asp Ala Lys Ser Pro
Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val
Ala Leu Gly Ala Ser Gly Ile Val Ile Trp Gly Thr Leu Ser Ile Thr
Arg Thr Lys Glu Ser Cys Gln Ala Ile Lys Glu Tyr Met Asp Thr Thr
Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn
Ser Ser Asp Tyr Leu His Leu Asn Pro Asp Asn Phe Ala Ile Gln Leu
Glu Lys Gly Gly Lys Phe Thr Val Arg Gly Lys Pro Thr Leu Glu Asp
Leu Glu Gln Phe Ser Glu Lys Phe Tyr Cys Ser Cys Tyr Ser Thr Leu
Ser Cys Lys Glu Lys Ala Asp Val Lys Asp Thr Asp Ala Val Asp Val
Cys Ile Ala Asp Gly Val Cys Ile Asp Ala Phe Leu Lys Pro Pro Met
Glu Thr Glu Glu Pro Gln Ile Phe Tyr Asn Ala Ser Pro Ser Thr Leu
Ser
SEQ ID NO: 486
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Ser Thr Leu Ser Cys Lys Glu Lys Ala Asp Val Lys Asp Thr Asp Ala
Val Asp Val Cys Ile Ala Asp
SEQ ID NO: 489
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys
SEQ ID NO: 490
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val
SEQ ID NO: 491
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys
SEQ ID NO: 492
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro
SEQ ID NO: 493
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met
SEQ ID NO: 494
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu
SEQ ID NO: 495
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr
SEQ ID NO: 496
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu
SEQ ID NO: 497
Asn Phe Arg Ala Pro Pro Val Ile Pro Asn Val Pro Phe Leu Trp Ala
Trp Asn Ala Pro Ser Glu Phe Cys Leu Gly Lys Phe Asp Glu Pro Leu
Asp Met Ser Leu Phe Ser Phe Ile Gly Ser Pro Arg Ile Asn Ala Thr
Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln
Lys Ile Ser Leu Gln Asp His Leu Asp Lys Ala Lys Lys Asp Ile Thr
Phe Tyr Met Pro Val Asp Asn Leu Gly Met Ala Val Ile Asp Trp Glu
Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser
Leu Thr Glu Ala Thr Glu Lys Ala Lys Gln Glu Phe Glu Lys Ala Gly
Lys Asp Phe Leu Val Glu Thr Ile Lys Leu Gly Lys Leu Leu Arg Pro
Asn His Leu Trp Gly Tyr Tyr Leu Phe Pro Asp Cys Tyr Asn His His
Tyr Lys Lys Pro Gly Tyr Asn Gly Ser Cys Phe Asn Val Glu Ile Lys
Arg Asn Asp Asp Leu Ser Trp Leu Trp Asn Glu Ser Thr Ala Leu Tyr
Pro Ser Ile Tyr Leu Asn Thr Gln Gln Ser Pro Val Ala Ala Thr Leu
Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe
Thr Asp Gln Val Leu Lys Phe Leu Ser Gln Asp Glu Leu Val Tyr Thr
Phe Gly Glu Thr Val Ala Leu Gly Ala Ser Gly Ile Val Ile Trp Gly
Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala
Ala Lys Met Cys Ser Gln Val Leu Cys Gln Glu Gln Gly Val Cys Ile
Arg Lys Asn Trp Asn Ser Ser Asp Tyr Leu His Leu Asn Pro Asp Asn
Phe Ala Ile Gln Leu Glu Lys Gly Gly Lys Phe Thr Val Arg Gly Lys
Pro Thr Leu Glu Asp Leu Glu Gln Phe Ser Glu Lys Phe Tyr Cys Ser
Cys Tyr Ser Thr Leu Ser Cys Lys Glu Lys Ala Asp Val Lys Asp Thr
Asp Ala Val Asp Val Cys Ile Ala Asp Gly Val Cys Ile Asp Ala Phe
Leu Lys Pro Pro Met Glu Thr Glu Glu Pro Gln Ile Phe Tyr Asn Ala
Ser Pro Ser Thr Leu Ser
SEQ ID NO: 498
Arg Ala Pro Pro Val Ile Pro Asn Val Pro Phe Leu Trp Ala Trp Asn
Ala Pro Ser Glu Phe Cys Leu Gly Lys Phe Asp Glu Pro Leu Asp Met
Ser Leu Phe Ser Phe Ile Gly Ser Pro Arg Ile Asn Ala Thr Gly Gln
Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile
Ser Leu Gln Asp His Leu Asp Lys Ala Lys Lys Asp Ile Thr Phe Tyr
Met Pro Val Asp Asn Leu Gly Met Ala Val Ile Asp Trp Glu Glu Trp
Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp
Phe Leu Val Glu Thr Ile Lys Leu Gly Lys Leu Leu Arg Pro Asn His
Leu Trp Gly Tyr Tyr Leu Phe Pro Asp Cys Tyr Asn His His Tyr Lys
Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser
Ile Tyr Leu Asn Thr Gln Gln Ser Pro Val Ala Ala Thr Leu Tyr Val
Arg Asn Arg Val Arg Glu Ala Ile Arg Val Ser Lys Ile Pro Asp Ala
Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly
Glu Thr Val Ala Leu Gly Ala Ser Gly Ile Val Ile Trp Gly Thr Leu
Ser Ile Thr Arg Thr Lys Glu Ser Cys Gln Ala Ile Lys Glu Tyr Met
Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys
Asn Trp Asn Ser Ser Asp Tyr Leu His Leu Asn Pro Asp Asn Phe Ala
Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala
Val Asp Val Cys Ile Ala Asp Gly Val Cys Ile Asp Ala Phe Leu Lys
Pro Pro Met Glu Thr Glu Glu Pro Gln Ile Phe Tyr Asn Ala Ser Pro
Ser Thr Leu Ser
SEQ ID NO: 499
Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu
Phe Ser Phe Ile Gly Ser Pro Arg Ile Asn Ala Thr Gly Gln Gly Val
Thr Ile Phe Tyr Val Asp Arg Leu Gly Tyr Tyr Pro Tyr Ile Asp Ser
Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro
Val Asp Asn Leu Gly Met Ala Val Ile Asp Trp Glu Glu Trp Arg Pro
Thr Trp Ala Arg Asn Trp Lys Pro Lys Asp Val Tyr Lys Asn Arg Ser
Ile Glu Leu Val Gln Gln Gln Asn Val Gln Leu Ser Leu Thr Glu Ala
Thr Glu Lys Ala Lys Gln Glu Phe Glu Lys Ala Gly Lys Asp Phe Leu
Val Glu Thr Ile Lys Leu Gly Lys Leu Leu Arg Pro Asn His Leu Trp
Gly Tyr Tyr Leu Phe Pro Asp Cys Tyr Asn His His Tyr Lys Lys Pro
Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr
Leu Asn Thr Gln Gln Ser Pro Val Ala Ala Thr Leu Tyr Val Arg Asn
Arg Val Arg Glu Ala Ile Arg Val Ser Lys Ile Pro Asp Ala Lys Ser
Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr
Val Ala Leu Gly Ala Ser Gly Ile Val Ile Trp Gly Thr Leu Ser Ile
Thr Arg Thr Lys Glu Ser Cys Gln Ala Ile Lys Glu Tyr Met Asp Thr
Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp
Asn Ser Ser Asp Tyr Leu His Leu Asn Pro Asp Asn Phe Ala Ile Gln
Leu Glu Lys Gly Gly Lys Phe Thr Val Arg Gly Lys Pro Thr Leu Glu
Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp
Val Cys Ile Ala Asp Gly Val Cys Ile Asp Ala Phe Leu Lys Pro Pro
Met Glu Thr Glu Glu Pro Gln Ile Phe Tyr Asn Ala Ser Pro Ser Thr
Leu Ser
SEQ ID NO: 500
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile
SEQ ID NO: 501
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp
SEQ ID NO: 502
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala
SEQ ID NO: 503
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 504
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro
SEQ ID NO: 505
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile
SEQ ID NO: 506
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile Phe Tyr
SEQ ID NO: 507
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile Phe Tyr Asn Ala
SEQ ID NO: 508
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile Phe Tyr Asn Ala Ser
Pro
SEQ ID NO: 509
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu
Lys Pro Pro Met Glu Thr Glu Glu Pro Gln Ile Phe Tyr Asn Ala Ser
Pro Ser Thr
SEQ ID NO: 510
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Gly Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 511
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Ala Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 512
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Cys Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 513
Leu Asn Phe Arg Ala Pro Pro Val Ile Pro Asn Val Pro Phe Leu Trp
Ala Trp Asn Ala Pro Ser Glu Phe Cys Leu Gly Lys Phe Asp Glu Pro
Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro
Gln Lys Ile Ser Leu Gln Asp His Leu Asp Lys Ala Lys Lys Asp Ile
Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu
Ser Leu Thr Glu Ala Thr Glu Lys Ala Lys Gln Glu Phe Glu Lys Ala
Gly Lys Asp Phe Leu Val Glu Thr Ile Lys Leu Gly Lys Leu Leu Arg
Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile
Lys Arg Asn Asp Asp Leu Ser Trp Leu Trp Asn Glu Ser Thr Ala Leu
Tyr Pro Ser Ile Tyr Leu Asn Thr Gln Gln Ser Pro Val Ala Ala Thr
Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr
Thr Phe Gly Glu Thr Val Ala Leu Gly Ala Ser Gly Ile Val Ile Trp
Gly Asp Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys
Ile Arg Lys Asn Trp Asn Ser Ser Asp Tyr Leu His Leu Asn Pro Asp
Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys
Ser Cys Tyr Ser Thr Leu Ser Cys Lys Glu Lys Ala Asp Val Lys Asp
Thr Asp Ala Val Asp Val Cys Ile Ala Asp Gly Val Cys Ile Asp Ala
Phe Leu Lys Pro Pro Met Glu Thr Glu Glu Pro Gln Ile Phe Tyr Asn
Ala Ser Pro Ser Thr Leu Ser
SEQ ID NO: 514
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Leu Ser Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 515
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Thr
Leu Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 516
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ala
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 517
Phe Arg Ala Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Gly
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 518
Phe Arg Gly Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe
SEQ ID NO: 519
Phe Arg Gly Pro Pro Val Ile Pro Asn Val Pro Phe Leu Trp Ala Trp
Asn Ala Pro Ser Glu Phe Cys Leu Gly Lys Phe Asp Glu Pro Leu Asp
Met Ser Leu Phe Ser Phe Ile Gly Ser Pro Arg Ile Asn Ala Thr Gly
Gln Gly Val Thr Ile Phe Tyr Val Asp Arg Leu Gly Tyr Tyr Pro Tyr
Ile Asp Ser Ile Thr Gly Val Thr Val Asn Gly Gly Ile Pro Gln Lys
Ile Ser Leu Gln Asp His Leu Asp Lys Ala Lys Lys Asp Ile Thr Phe
Tyr Met Pro Val Asp Asn Leu Gly Met Ala Val Ile Asp Trp Glu Glu
Trp Arg Pro Thr Trp Ala Arg Asn Trp Lys Pro Lys Asp Val Tyr Lys
Asn Arg Ser Ile Glu Leu Val Gln Gln Gln Asn Val Gln Leu Ser Leu
Thr Glu Ala Thr Glu Lys Ala Lys Gln Glu Phe Glu Lys Ala Gly Lys
Asp Phe Leu Val Glu Thr Ile Lys Leu Gly Lys Leu Leu Arg Pro Asn
His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg
Asn Asp Asp Leu Ser Trp Leu Trp Asn Glu Ser Thr Ala Leu Tyr Pro
Ser Ile Tyr Leu Asn Thr Gln Gln Ser Pro Val Ala Ala Thr Leu Tyr
Val Arg Asn Arg Val Arg Glu Ala Ile Arg Val Ser Lys Ile Pro Asp
Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe
Gly Glu Thr Val Ala Leu Gly Ala Ser Gly Ile Val Ile Trp Gly Ser
Trp Glu Asn Thr Arg Thr Lys Glu Ser Cys Gln Ala Ile Lys Glu Tyr
Met Asp Thr Thr Leu Asn Pro Tyr Ile Ile Asn Val Thr Leu Ala Ala
Lys Met Cys Ser Gln Val Leu Cys Gln Glu Gln Gly Val Cys Ile Arg
Lys Asn Trp Asn Ser Ser Asp Tyr Leu His Leu Asn Pro Asp Asn Phe
Ala Ile Gln Leu Glu Lys Gly Gly Lys Phe Thr Val Arg Gly Lys Pro
Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe

| | | SEQ ID |
|---|---|---|
| Description | Sequence | NO |
| Human lambda-2 | GQPKAAPSVTLFPPSSEELQANKATLVCLISDFYPGAVTVA | 520 |
| light chain | WKADSSPVKAGVETTTPSKQSNNKYAASSYLSLTPEQWKS | |
| constant region | HRSYSCQVTHEGSTVEKTVAPTECS | |
| (lambda light | | |
| chain constant | | |
| region, subtype 2) | | |
| UniProt P0DOY2 | | |
| Human lambda-1 | GQPKANPTVTLFPPSSEELQANKATLVCLISDFYPGAVTVA | 521 |
| light chain | WKADGSPVKAGVETTKPSKQSNNKYAASSYLSLTPEQWKS | |
| constant region | HRSYSCQVTHEGSTVEKTVAPTECS | |
| (lambda light | | |
| chain constant | | |
| region, subtype 1) | | |
| UniProt P0CG04 | | |
| Human lambda-3 | GQPKAAPSVTLFPPSSEELQANKATLVCLISDFYPGAVTVA | 522 |
| light chain | WKADSSPVKAGVETTTPSKQSNNKYAASSYLSLTPEQWKS | |
| constant region | HKSYSCQVTHEGSTVEKTVAPTECS | |
| (lambda light | | |
| chain constant | | |
| region, subtype 3) | | |
| UniProt PODOY3 | | |
| Human lambda-7 | GQPKAAPSVTLFPPSSEELQANKATLVCLVSDFNPGAVTVA | 523 |
| light chain | WKADGSPVKVGVETTKPSKQSNNKYAASSYLSLTPEQWKS | |
| constant region | HRSYSCRVTHEGSTVEKTVAPAECS | |
| (lambda light | | |
| chain constant | | |
| region, subtype 7) | | |
| UniProt A0M8Q6 | | |
| Antibody 5 Heavy | QMQLVESGGGVVQPGRSLRLSCAASGFTFRTYGMHWVRQ | 524 |
| Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 5 Light | SYVLTQPPSVSVAPGQTARITCGGNNLVSKSVHWYQQKPG | 525 |
| Chain | QAPVLVVYDDSDRPSWIPERFSGSNSGNTATLTISRGEAGDE | |
| | ADYYCQVWDSSSTFVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 5 Heavy | QMQLVESGGGVVQPGRSLRLSCAASGFTFRTYGMHWVRQ | 526 |
| Chain without C- | APGKGLEWVAVIWYDGSNKHYADSVKGRFTITRDNSKNTL | |
| terminal lysine | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPG | |
| Antibody 7 Heavy | QMQLVESGGGVVQPGRSLRLSCAASGFTFRTYGMHWVRQ | 527 |
| Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 7 Light | SYVLTQPPSVSVAPGQTARITCGGNNLGSKSVHWYQQKPG | 528 |
| Chain | QAPVLVVYDVSDRPSWIPERFSGSNSGNTATLTISRGEAGDE | |
| | ADYYCQVWDSSSTIVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 23 | QMQLVESGGGVVQPGRSLRLSCAASGFTFRTYGMHWVRQ | 529 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 23 Light | SYELTQPPSVSVAPGQTARITCGGHNIGTKSVHWYQQKSGQ | 530 |
| Chain | APILVVYDDSDRPSWIPERFSGSNFGTTATLTISRVEAGDEA | |
| | DYFCQVWDRSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 29 | QMQLVESGGGVVQPGRSLRLSCAASGFTFRTYGMHWVRQ | 531 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 29 Light | SYELTQPPSVSVAPGQTARITCGGNNIGRKSAHWYQQRPGQ | 532 |
| Chain | APVLVVYDDSDRPSWIPERFSGSNSGNTATLTISRVEAGDEA | |
| | DYYCQVWDSYSNHVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 31 | QMQLVESGGGVVQPGRSLRLSCAASGFTFRTYGMHWVRQ | 533 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 31 Light | SYVLTQPPSVSVAPGQTARITCGGHNIGTKSVHWYQQKSGQ | 534 |
| Chain | APILAVYDDSDRPSWIPERFSGSNFGTTATLTISRVEAGDEA | |
| | DYFCQVWDRSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 36 | QMQLVESGGGVVQPGRSLRLSCAASGFTFRTYGMHWVRQ | 535 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 36 Light | SYVLTQPPSVSVAPGQTARITCGGQSIGTKSVHWYQQKSGQ | 536 |
| Chain | APILVVYDDSDRPSWIPERFSGSNFGTTATLTISRVEAGDEA | |
| | DYFCQVWDSSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 38 | QMQLVESGGGVVQPGRSLRLSCAASGFTFRTYGMHWVRQ | 537 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 38 Light | SYELTQPPSVSVAPGQTARITCGGNKIGRKSVHWYQQRPGQ | 538 |
| Chain | APVLVVFDDSDRPSWISERFSGSNSGNTATLTIGRVEAGDEA | |
| | DYYCQVWDSSSDEGVFGGGTKLAVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 39 | QMQLVESGGGVVQPGRSLRLSCAASGFTFRTYGMHWVRQ | 539 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 39 Light | SYELTQPPSVSVAPGRTARITCGGQNIGTKSVHWYQQKSGQ | 540 |
| Chain | APILVVYDDSDRPSWIPERFSGSNFGTTATLTISRVEAGDEA | |
| | DYFCQVWDSSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 44 | QMQLVESGGGVVQPGRSLRLSCAASGFTFRTYGMHWVRQ | 541 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 44 Light | SYELTQPPSVSVAPGQTARITCGGNKIGRKSVHWYQQKPGQ | 542 |
| Chain | APVLVVFDDSDRPSWISERFSGSNSGNTATLTISRVEAGDEA | |
| | DYHCQVWDTSSDQGVFGGGTKLTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Construct 114 | EVQVVESGGGLVQPGGSLRLSCAASGFTFSNYAMTWVRQS | 543 |
| Heavy Chain | PGKGLEWVSLISGSGGLTKYADSVKGRFTISRDNSKKTLFLQ | |
| | MNSLRAEDTAAYYCVKDEGLTTGEYWGQGTQVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKKVEPKSCDKTHTCPPCPAPEAAGGPSVFLFPP | |
| | KPKDTLYITREPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSRDELTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| Construct 114 | EVQVVESGGGLVQPGGSLRLSCAASGFTFSNYAMTWVRQS | 544 |
| Heavy Chain | PGKGLEWVSLISGSGGLTKYADSVKGRFTISRDNSKKTLFLQ | |
| without C- | MNSLRAEDTAAYYCVKDEGLTTGEYWGQGTQVTVSSAST | |
| terminal lysine | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKKVEPKSCDKTHTCPPCPAPEAAGGPSVFLFPP | |
| | KPKDTLYITREPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSRDELTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPG | |
| Construct 114 | DIQMTQSPSSLSASVGDRVTITCRASQDIRNDLAWYQQKPG | 545 |
| light chain | KAPKRLIYASSSLQSGVPSRFSGSGSGTEFTLTISSLQPEDFAT | |
| | YYCLQHNNYPFTFGPGTKVDIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| Oxelumab VH | EVQLLESGGGLVQPGGSLRLSCAASGFTFNSYAMSWVRQA | 546 |
| | PGKGLEWVSIISGSGGFTYYADSVKGRFTISRDNSRTTLYLQ | |
| | MNSLRAEDTAVYYCAKDRLVAPGTFDYWGQGALVTVSS | |
| Oxelumab VL | DIQMTQSPSSLSASVGDRVTITCRASQGISSWLAWYQQKPE | 547 |
| | KAPKSLIYAASSLQSGVPSRFSGSGSGTDFTLTISSLQPEDFA | |
| | TYYCQQYNSYPYTFGQGTKLEIK | |
| Amlitelimab | EVQLVESGGGLVQPGGSLRLSCAASGFTFSNYAMNWVRQA | 548 |
| Heavy Chain | PGKGLEWVSTISGSGGATRYADSVKGRFTISRDNSRNTVYL | |
| | QMNSLRVEDTAVFYCTKDRLIMATVRGPYYYGMDVWGQG | |
| | TTVTVSSASTKGPSVFPLAPCSRSTSESTAALGCLVKDYFPEP | |
| | VTVSWNSGALTSGVHTFPAVLQSSGLYSLSSVVTVPSSSLGT | |
| | KTYTCNVDHKPSNTKVDKRVESKYGPPCPPCPAPEFEGGPS | |
| | VFLFPPKPKDTLMISRTPEVTCVVVDVSQEDPEVQFNWYVD | |
| | GVEVHNAKTKPREEQFNSTYRVVSVLTVLHQDWLNGKEYK | |
| | CKVSNKGLPSSIEKTISKAKGQPREPQVYTLPPSQEEMTKNQ | |
| | VSLTCLVKGFYPSDIAVEWESNGQPENNYKTTPPVLDSDGS | |
| | FFLYSRLTVDKSRWQEGNVFSCSVMHEALHNHYTQKSLSLS | |
| | LGK | |
| Amlitelimab Light | DIQMTQSPSSLSASVGDRVTITCRASQSISSYLNWYQQKPGK | 549 |
| Chain | APNLLIYAASSLQSGVPSRFSGSGSETDFTLTISSLQPEDFATY | |
| | YCQQSHSVSFTFGPGTKVDIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFN | |
| | RGEC | |
| Tezepelumab | QMQLVESGGGVVQPGRSLRLSCAASGFTFRTYGMHWVRQ | 550 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPCSRSTSESTAALGCLVKDYFPEPVT | |
| | VSWNSGALTSGVHTFPAVLQSSGLYSLSSVVTVPSSNFGTQT | |
| | YTCNVDHKPSNTKVDKTVERKCCVECPPCPAPPVAGPSVFL | |
| | FPPKPKDTLMISRTPEVTCVVVDVSHEDPEVQFNWYVDGVE | |
| | VHNAKTKPREEQFNSTFRVVSVLTVVHQDWLNGKEYKCKV | |
| | SNKGLPAPIEKTISKTKGQPREPQVYTLPPSREEMTKNQVSL | |
| | TCLVKGFYPSDIAVEWESNGQPENNYKTTPPMLDSDGSFFL | |
| | YSKLTVDKSRWQQGNVFSCSVMHEALHNHYTQKSLSLSPG | |
| | K | |
| Tezepelumab | SYVLTQPPSVSVAPGQTARITCGGNNLGSKSVHWYQQKPG | 551 |
| Light Chain | QAPVLVVYDDSDRPSWIPERFSGSNSGNTATLTISRGEAGDE | |
| | ADYYCQVWDSSSDHVVFGGGTKLTVLGQPKAAPSVTLFPP | |
| | SSEELQANKATLVCLISDFYPGAVTVAWKADSSPVKAGVET | |
| | TTPSKQSNNKYAASSYLSLTPEQWKSHRSYSCQVTHEGSTV | |
| | EKTVAPTECS | |
| TQC2731 | QVQLVQSGAEVKKPGASVKVSCKASGYTFTTYWMHWVRQ | 552 |
| (Bosakitug) Heavy | APGQGLEWMGVIDPSDSDTTYNQKFKGRVTMTRDTSTSTV | |
| Chain | YMELSSLRSEDTAVYYCTRSLDGYYDYWGQGTLVTVSSAS | |
| | TKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKV | |
| | SNKALPAPIEKTISKAKGQPREPQVYTLPPSRDELTKNQVSL | |
| | TCLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| TQC2731 | DIQMTQSPSSLSASVGDRVTITCRPTENIYSYLAWYQQKPGK | 553 |
| (Bosakitug) Light | APKLLIYFARTLAEGVPSRFSGSGSGTDFTLTISSLQPEDFAT | |
| Chain | YYCQHHYGTPWTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| TQC2731 | QVQLVQSGAEVKKPGASVKVSCKASGYTFTTYWMHWVRQ | 554 |
| (Bosakitug) VH | APGQGLEWMGVIDPSDSDTTYNQKFKGRVTMTRDTSTSTV | |
| | YMELSSLRSEDTAVYYCTRSLDGYYDYWGQGTLVTVSS | |
| TQC2731 | DIQMTQSPSSLSASVGDRVTITCRPTENIYSYLAWYQQKPGK | 555 |
| (Bosakitug) VL | APKLLIYFARTLAEGVPSRFSGSGSGTDFTLTISSLQPEDFAT | |
| | YYCQHHYGTPWTFGGGTKVEIK | |
| hu23b12 heavy | QVQLVQSGAEVKKPGASVKVSCKASGYIFTDYAMHWVRQ | 556 |
| chain | APGQGLEWMGTFIPLLDTSDYNQNFKGRVTMTTDTSTSTAY | |
| | MELRSLRSDDTAVYYCARMGVTHSYVMDAWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| hu23b12 light | EIVLTQSPGTLSLSPGERATLSCRASQPISISVHWYQQKPGQA | 557 |
| chain | PRLLIYFASQSISGIPDRFSGSGSGTDFTLTISRLEPEDFAVYY | |
| | CQQTFSLPYTFGQGTKVEIKRTVAAPSVFIFPPSDEQLKSGTA | |
| | SVVCLLNNFYPREAKVQWKVDNALQSGNSQESVTEQDSKD | |
| | STYSLSSTLTLSKADYEKHKVYACEVTHQGLSSPVTKSFNR | |
| | GEC | |
| 43B1-H2L2 heavy | QMQLVESGGGAVQPGGSLRLSCAASGFTFRSYDMHWVRQ | 558 |
| chain | APGKGLEWVAVIWYDGSNENYADSVKGRFTTSRDNSKNTL | |
| | FLQMNSLRAEDTAVYYCARSPLWYGEPDDAFDIWGQGTLV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHAHYTQKSL | |
| | SLSPGK | |
| 43B1-H2L2 light | EIVMTQSPATLSVSPGERATLSCRASQSVSSNLAWYQQKPG | 559 |
| chain | QAPRLLISGASTRATGIPARFSGSGSGTDFTLTISSLQSEDFAV | |
| | YYCQHYNNWPLTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| 43B1-H6L1 heavy | QMQLVESGGGAVQPGRSLRLSCAASGFTFRSYDMHWVRQ | 560 |
| chain | APGKGLEWVAVIWYDGSNENYADSVKGRFTTSRDNSKNTL | |
| | FLQMNSLRAEDTAVYYCARSPLWYGEPDDAFDIWGQGTLV | |
| | TVSSASTKGPSVFPLAPCSRSTSESTAALGCLVKDYFPEPVT | |
| | VSWNSGALTSGVHTFPAVLQSSGLYSLSSVVTVPSSSLGTKT | |
| | YTCNVDHKPSNTKVDKRVESKYGPPCPPCPAPEFLGGPSVF | |
| | LFPPKPKDTLMISRTPEVTCVVVDVSQEDPEVQFNWYVDGV | |
| | EVHNAKTKPREEQFNSTYRVVSVLTVLHQDWLNGKEYKCK | |
| | VSNKGLPSSIEKTISKAKGQPREPQVYTLPPSQEE | |
| | MTKNQVSLTCLVKGFYPSDIAVEWESNGQPENNYKTTPPVL | |
| | DSDGSFFLYSRLTVDKSRWQEGNVFSCSVMHEALHAHYTQ | |
| | KSLSLSLGK | |
| 43B1-H6L1 light | EIVMTQSPATLSVSPGERATLSCRASQSVSSNLAWYQQKPG | 561 |
| chain | QAPRLLISGASTRATGIPARFSGSGSGTDFTLTISSLRSEDFAV | |
| | YYCQHYNNWPLTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| Solrikitug VH | QVQLVQSGAEVKKPGASVKVSCKASGYIFTDYAMHWVRQ | 562 |
| | APGQGLEWMGTFIPLLDTSDYAQKFQGRVTMTADTSTSTA | |
| | YMELRSLRSDDTAVYYCARMGVTHSYVMDAWGQGTLVT | |
| | VSS | |
| Solrikitug VL | EIVLTQSPGTLSLSPGERATLSCRASQPISISVHWYQQKPGQA | 563 |
| | PRLLIYFASQSISGIPDRFSGSGSGTDFTLTISRLEPEDFAVYY | |
| | CQQTFSLPYTFGQGTKVEIK | |
| Solrikitug heavy | QVQLVQSGAEVKKPGASVKVSCKASGYIFTDYAMHWVRQ | 564 |
| chain | APGQGLEWMGTFIPLLDTSDYAQKFQGRVTMTADTSTSTA | |
| | YMELRSLRSDDTAVYYCARMGVTHSYVMDAWGQGTLVT | |
| | VSSASTKGPSVFPLAPSSKSTSGGTAALGCLVKDYFPEPVTV | |
| | SWNSGALTSGVHTFPAVLQSSGLYSLSSVVTVPSSSLGTQTY | |
| | ICNVNHKPSNTKVDKKVEPKSCDKTHTCPPCPAPELLGGPS | |
| | VFLFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVYTLPPSRDELTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLS | |
| | LSPGK | |
| Solrikitug light | EIVLTQSPGTLSLSPGERATLSCRASQPISISVHWYQQKPGQA | 565 |
| chain | PRLLIYFASQSISGIPDRFSGSGSGTDFTLTISRLEPEDFAVYY | |
| | CQQTFSLPYTFGQGTKVEIKRTVAAPSVFIFPPSDEQLKSGTA | |
| | SVVCLLNNFYPREAKVQWKVDNALQSGNSQESVTEQDSKD | |
| | STYSLSSTLTLSKADYEKHKVYACEVTHQGLSSPVTKSFNR | |
| | GEC | |
| TSLP LCDR3 | QVWDSSSDFVV | 566 |
| TSLP LCDR3 | QVWDSSSTHVV | 567 |
| TSLP LCDR3 | QVWDSSSTFVV | 568 |
| TSLP LCDR3 | QVWDSSSTIVV | 569 |
| TSLP VL | SYVLTQPPSVSVAPGQTARITCGGNNLGSKSVHWYQQKPG | 570 |
| | QAPVLVVYDVSDRPSWIPERFSGSNSGNTATLTISRGEAGDE | |
| | ADYYCQVWDSSSDHVVFGGGTKLTVL | |
| TSLP VL | SYVLTQPPSVSVAPGQTARITCGGNNLGSKSVHWYQQKPG | 571 |
| | QAPVLVVYDDSDRPSWIPERFSGSNSGNTATLTISRGEAGDE | |
| | ADYYCQVWDSSSDFVVFGGGTKLTVL | |
| TSLP VL | SYVLTQPPSVSVAPGQTARITCGGNNLVSKSVHWYQQKPG | 572 |
| | QAPVLVVYDVSDRPSWIPERFSGSNSGNTATLTISRGEAGDE | |
| | ADYYCQVWDSSSDHVVFGGGTKLTVL | |
| TSLP VL | SYVLTQPPSVSVAPGQTARITCGGNNLVSKSVHWYQQKPG | 573 |
| | QAPVLVVYDDSDRPSWIPERFSGSNSGNTATLTISRGEAGDE | |
| | ADYYCQVWDSSSDFVVFGGGTKLTVL | |
| TSLP VL | SYVLTQPPSVSVAPGQTARITCGGNNLGSKSVHWYQQKPG | 574 |
| | QAPVLVVYDVSDRPSWIPERFSGSNSGNTATLTISRGEAGDE | |
| | ADYYCQVWDSSSTHVVFGGGTKLTVL | |
| TSLP VL | SYVLTQPPSVSVAPGQTARITCGGNNLGSKSVHWYQQKPG | 575 |
| | QAPVLVVYDVSDRPSWIPERFSGSNSGNTATLTISRGEAGDE | |
| | ADYYCQVWDSSSDFVVFGGGTKLTVL | |
| TSLP VL | SYVLTQPPSVSVAPGQTARITCGGNNLGSKSVHWYQQKPG | 576 |
| | QAPVLVVYDDSDRPSWIPERFSGSNSGNTATLTISRGEAGDE | |
| | ADYYCQVWDSSSTFVVFGGGTKLTVL | |
| TSLP VL | SYVLTQPPSVSVAPGQTARITCGGNNLVSKSVHWYQQKPG | 577 |
| | QAPVLVVYDVSDRPSWIPERFSGSNSGNTATLTISRGEAGDE | |
| | ADYYCQVWDSSSTHVVFGGGTKLTVL | |
| TSLP VL | SYVLTQPPSVSVAPGQTARITCGGNNLVSKSVHWYQQKPG | 578 |
| | QAPVLVVYDVSDRPSWIPERFSGSNSGNTATLTISRGEAGDE | |
| | ADYYCQVWDSSSDFVVFGGGTKLTVL | |
| TSLP VL | SYVLTQPPSVSVAPGQTARITCGGNNLVSKSVHWYQQKPG | 579 |
| | QAPVLVVYDDSDRPSWIPERFSGSNSGNTATLTISRGEAGDE | |
| | ADYYCQVWDSSSTIVVFGGGTKLTVL | |
| TSLP VL | SYVLTQPPSVSVAPGQTARITCGGNNLGSKSVHWYQQKPG | 580 |
| | QAPVLVVYDVSDRPSWIPERFSGSNSGNTATLTISRGEAGDE | |
| | ADYYCQVWDSSSTFVVFGGGTKLTVL | |
| TSLP VL | SYVLTQPPSVSVAPGQTARITCGGNNLVSKSVHWYQQKPG | 581 |
| | QAPVLVVYDVSDRPSWIPERFSGSNSGNTATLTISRGEAGDE | |
| | ADYYCQVWDSSSTFVVFGGGTKLTVL | |
| TSLP VL | SYVLTQPPSVSVAPGQTARITCGGNNLVSKSVHWYQQKPG | 582 |
| | QAPVLVVYDVSDRPSWIPERFSGSNSGNTATLTISRGEAGDE | |
| | ADYYCQVWDSSSTIVVFGGGTKLTVL | |
| TSLP VL | SYVLTQPPSVSVAPGQTARITCGGNNLGSKSVHWYQQKPG | 583 |
| | QAPVLVVYDDSDRPSWIPERFSGSNSGNTATLTISRGEAGDE | |
| | ADYYCQVWDSSSDHVVFGGGTKLTVL | |
| Antibody 51 | QMQLVESGGGVVQPGRSLRLSCAASGFTFRTYGMHWVRQ | 584 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 51 Light | SYVLTQPPSVSVAPGQTARITCGGNNLGSKSVHWYQQKPG | 585 |
| Chain | QAPVLVVYDVSDRPSWIPERFSGSNSGNTATLTISRGEAGDE | |
| | ADYYCQVWDSSSTHVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 59 | QMQLVESGGGVVQPGRSLRLSCAASGFTFRTYGMHWVRQ | 586 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 59 Light | SYVLTQPPSVSVAPGQTARITCGGNNLVSKSVHWYQQKPG | 587 |
| Chain | QAPVLVVYDVSDRPSWIPERFSGSNSGNTATLTISRGEAGDE | |
| | ADYYCQVWDSSSTFVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| SRDEL allotype | SRDEL | 588 |
| SREEM allotype | SREEM | 589 |
| TSLP CDR-L1 | YLGAKS | 590 |
| Amlitelimab | NYAMN | 591 |
| CDR-H1 Kabat | | |
| Amlitelimab | TISGSGGATRYADSVKG | 592 |
| CDR- | | |
| H2 Kabat | | |
| Amlitelimab | DRLIMATVRGPYYYGMDV | 593 |
| CDR- | | |
| H3 Kabat and | | |
| Chothia | | |
| Amlitelimab | GFTFSNY | 594 |
| CDR-H1 Chothia | | |
| Amlitelimab | SGSGGA | 595 |
| CDR- | | |
| H2 Chothia | | |
| Amlitelimab | GFTFSNYA | 596 |
| CDR-H1 IMGT | | |
| Amlitelimab | ISGSGGAT | 597 |
| CDR- | | |
| H2 IMGT | | |
| Amlitelimab | TKDRLIMATVRGPYYYGMDV | 598 |
| CDR- | | |
| H3 IMGT | | |
| Amlitelimab | RASQSISSYLN | 599 |
| CDR-L1 Kabat | | |
| and Chothia | | |
| Amlitelimab | AASSLQS | 600 |
| CDR- | | |
| L2 Kabat and | | |
| Chothia | | |
| Amlitelimab | QQSHSVSFT | 601 |
| CDR- | | |
| L3 Kabat, | | |
| Chothia, and | | |
| IMGT | | |
| Amlitelimab | QSISSY | 602 |
| CDR-L1 IMGT | | |
| Amlitelimab | AAS | — |
| CDR- | | |
| L2 IMGT | | |

1. A composition comprising an isolated antibody, or antigen binding region thereof, that binds thymic stromal lymphopoietin (TSLP), an isolated antibody, or antigen binding region thereof, that binds OX40L, and a hyaluronidase or variant thereof.

2. A composition comprising an isolated antibody, or antigen binding region thereof, that binds TSLP, an isolated antibody, or antigen binding region thereof, that binds OX40L, and a hyaluronidase or variant thereof, wherein the antibody, or antigen binding region thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

3. A composition comprising an isolated antibody, or antigen binding region thereof, that binds TSLP, an isolated antibody, or antigen binding region thereof, that binds OX40L, and a hyaluronidase or variant thereof, wherein the antibody, or antigen binding region thereof, that binds TSLP comprises a VH sequence set forth in SEQ ID NO: 34 and a VL sequence set forth in SEQ ID NO: 41.

4. A composition comprising an isolated antibody, or antigen binding region thereof, that binds TSLP, an isolated antibody, or antigen binding region thereof, that binds OX40L, and a hyaluronidase or variant thereof, wherein the antibody, or antigen binding region thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

5. A composition comprising an isolated antibody, or antigen binding region thereof, that binds TSLP, an isolated antibody, or antigen binding region thereof, that binds OX40L, and a hyaluronidase or variant thereof, wherein the antibody, or antigen binding region thereof, that binds OX40L comprises a VH sequence set forth in SEQ ID NO: 335 and a VL sequence set forth in SEQ ID NO: 339.

6. A composition comprising an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antibody, or antigen binding region thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

7. A composition comprising an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a VH sequence set forth in SEQ ID NO: 34 and a VL sequence set forth in SEQ ID NO: 41; and
wherein the antibody, or antigen binding region thereof, that binds OX40L comprises a VH sequence set forth in SEQ ID NO: 335 and a VL sequence set forth in SEQ ID NO: 339.

8. A composition comprising hyaluronidase or variant thereof and a multi-specific antibody comprising a first antigen binding region that binds TSLP and a second antigen binding region that binds OX40L,
wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

9. A composition comprising hyaluronidase or variant thereof and a multi-specific antibody comprising a first antigen binding region that binds TSLP and a second antigen binding region that binds OX40L,
wherein the antigen binding region that binds TSLP comprises a VH sequence set forth in SEQ ID NO: 34 and a VL sequence set forth in SEQ ID NO: 41; and
wherein the antigen binding region that binds OX40L comprises a VH sequence set forth in SEQ ID NO: 335 and a VL sequence set forth in SEQ ID NO: 339.

10. A combination comprising an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antibody, or antigen binding region thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

11. A combination comprising an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a VH sequence set forth in SEQ ID NO: 34 and a VL sequence set forth in SEQ ID NO: 41; and
wherein the antibody, or antigen binding region thereof, that binds OX40L comprises a VH sequence set forth in SEQ ID NO: 335 and a VL sequence set forth in SEQ ID NO: 339.

12. A combination comprising hyaluronidase or variant thereof and a multi-specific antibody comprising a first antigen binding region that binds TSLP and a second antigen binding region that binds OX40L,
wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

13. A combination comprising hyaluronidase or variant thereof and a multi-specific antibody comprising a first antigen binding region that binds TSLP and a second antigen binding region that binds OX40L,
wherein the antigen binding region that binds TSLP comprises a VH sequence set forth in SEQ ID NO: 34 and a VL sequence set forth in SEQ ID NO: 41; and
wherein the antigen binding region that binds OX40L comprises a VH sequence set forth in SEQ ID NO: 335 and a VL sequence set forth in SEQ ID NO: 339.

14. A method of treating an inflammatory disorder or disease in a human patient comprising administering to the patient an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antibody, or antigen binding region thereof, that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

15. A method of treating an inflammatory disorder or disease in a human patient comprising administering to the patient an isolated antibody, or antigen binding region thereof, that binds TSLP, an isolated antibody, or antigen binding region thereof, that binds OX40L, and a hyaluronidase or variant thereof,
wherein the antibody, or antigen binding region thereof, that binds TSLP comprises a VH sequence set forth in SEQ ID NO: 34 and a VL sequence set forth in SEQ ID NO: 41; and
wherein the antibody, or antigen binding region thereof, that binds OX40L comprises a VH sequence set forth in SEQ ID NO: 335 and a VL sequence set forth in SEQ ID NO: 339.

16. A method of treating an inflammatory disorder or disease in a human patient comprising administering to the patient hyaluronidase or variant thereof and a multi-specific antibody comprising a first antigen binding region that binds TSLP and a second antigen binding region that binds OX40L,
wherein the antigen binding region that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and
wherein the antigen binding region that binds OX40L comprises a CDR-H1 comprising the sequence set forth in SEQ ID NO: 273; a CDR-H2 comprising the sequence set forth in SEQ ID NO: 285; a CDR-H3 comprising the sequence set forth in SEQ ID NO: 297; a CDR-L1 comprising the sequence set forth in SEQ ID NO: 309; a CDR-L2 comprising the sequence set forth in SEQ ID NO: 319; and a CDR-L3 comprising the sequence set forth in SEQ ID NO: 327.

17. A method of treating an inflammatory disorder or disease in a human patient comprising administering to the patient hyaluronidase or variant thereof and a multi-specific antibody comprising a first antigen binding region that binds TSLP and a second antigen binding region that binds OX40L,
wherein the antigen binding region that binds TSLP comprises a VH sequence set forth in SEQ ID NO: 34 and a VL sequence set forth in SEQ ID NO: 41; and
wherein the antigen binding region that binds OX40L comprises a VH sequence set forth in SEQ ID NO: 335 and a VL sequence set forth in SEQ ID NO: 339.

18. The composition of any one of claims 1-9, the combination of any one of claims 10-13, or the method of any one of claims 14-17, wherein the antibody, or antigen binding region thereof, that binds TSLP and/or the antibody, or antigen binding region thereof, that binds OX40L is a humanized, human, or chimeric antibody.

19. The composition of any one of claims 1-9, the combination of any one of claims 10-13, or the method of any one of claims 14-17, wherein the antibody, or antigen binding region thereof, that binds TSLP and/or the antibody, or antigen binding region thereof, that binds OX40L is a humanized antibody.

20. The composition of any one of claims 1-9, the combination of any one of claims 10-13, or the method of any one of claims 14-17, wherein the antibody or antigen binding region that binds TSLP and/or the antibody or antigen binding region that binds OX40L comprises a heavy chain human constant region of a class selected from IgG, IgA, IgD, IgE, and IgM.

21. The composition of any one of claims 1-9, the combination of any one of claims 10-13, or the method of any one of claims 14-17, wherein the antibody or antigen binding region that binds TSLP and/or the antibody or antigen binding region that binds OX40L comprises a human Fc region, and wherein the human Fc region comprises a human heavy chain constant region of the class IgG and a subclass selected from IgG1, IgG2, IgG3, and IgG4.

22. The composition of any one of claims 1-9, the combination of any one of claims 10-13, or the method of any one of claims 14-17, wherein the antibody or antigen binding region that binds TSLP and/or the antibody or antigen binding region that binds OX40L comprises a human IgG1 Fc region.

23. The composition of any one of claims 1-9, the combination of any one of claims 10-13, or the method of any one of claims 14-17, wherein the antibody or antigen binding region that binds TSLP and/or the antibody or antigen binding region that binds OX40L comprises a human IgG4 Fc region.

24. The composition of any one of claims 1-9, the combination of any one of claims 10-13, or the method of any one of claims 14-17, wherein the antibody or antigen binding region that binds TSLP and/or the antibody or antigen binding region that binds OX40L comprises a human IgG2 Fc region.

25. The composition of any one of claims 1-9, the combination of any one of claims 10-13, or the method of any one of claims 14-17, wherein the antibody or antigen binding region that binds TSLP and/or the antibody or antigen binding region that binds OX40L comprises a human IgG1 Fc region with LALA and YTE mutations.

26. The composition of any one of claims 1-9, the combination of any one of claims 10-13, or the method of any one of claims 14-17, wherein the antibody or antigen binding region that binds TSLP comprises a human IgG1 Fc region with LALA mutations at positions 239 and 240 (L239A/L240A) by direct numbering and YTE mutations at positions 257, 259, and 261 (M257Y/S259T/T261E) by direct numbering and the antibody or antigen binding region that binds OX40L comprises a human IgG1 Fc with LALA mutations at positions 235 and 236 (L235A/L236A) by direct numbering and YTE mutations at positions 253, 255, and 257 (M253Y/S255T/T257E) by direct numbering.

27. The composition of any one of claims 1-9, the combination of any one of claims 10-13, or the method of any one of claims 14-17, wherein the antibody or antigen binding region that binds TSLP and/or the antibody or antigen binding region that binds OX40L comprises an Fc region that binds to Neonatal Fc receptor (FcRn).

28. The composition of any one of claims 1-9, the combination of any one of claims 10-13, or the method of any one of claims 14-17, wherein the antibody or antigen binding region that binds TSLP and/or the antibody or antigen binding region that binds OX40L comprises an Fc region that binds an FcRn with higher affinity at pH 6.0 compared to an antibody comprising a wild-type Fc region.

29. The composition of any one of claims 1-9, the combination of any one of claims 10-13, or the method of any one of claims 14-17, wherein the antibody or antigen binding region that binds TSLP and/or the antibody or antigen binding region that binds OX40L comprises an Fc region that binds to FcRn with a K_{D} of <1×10⁻⁷ M at pH 6.0.

30. The composition of any one of claims 1-9, the combination of any one of claims 10-13, or the method of any one of claims 14-17, wherein the antibody or antigen binding region that binds TSLP and/or the antibody or antigen binding region that binds OX40L is a monoclonal antibody.

31. The composition of any one of claims 1-9, the combination of any one of claims 10-13, or the method of any one of claims 14-17, wherein the antibody or antigen binding region that binds TSLP binds a TSLP sequence set forth in any one of the amino acid sequences of SEQ ID NOs: 271-272.

32. The composition of any one of claims 1-9, the combination of any one of claims 10-13, or the method of any one of claims 14-17, wherein the antibody or antigen binding region that binds OX40L binds an OX40L sequence set forth in the amino acid sequence of SEQ ID NO: 439.

33. The composition of any one of claims 1-9, the combination of any one of claims 10-13, or the method of any one of claims 14-17, wherein the antibody or antigen binding region that binds TSLP comprises a heavy chain comprising a constant heavy chain sequence set forth in SEQ ID NO: 61 or 173.

34. The composition of any one of claims 1-9, the combination of any one of claims 10-13, or the method of any one of claims 14-17, wherein the antibody or antigen binding region that binds TSLP comprises a light chain comprising a constant light chain sequence set forth in SEQ ID NO: 520.

35. The composition of any one of claims 1-9, the combination of any one of claims 10-13, or the method of any one of claims 14-17, wherein the antibody or antigen binding region that binds TSLP comprises a heavy chain comprising a constant heavy chain sequence set forth in SEQ ID NO: 61 or 173 and a light chain comprising a constant light chain sequence set forth in SEQ ID NO: 520.

36. The composition of any one of claims 1-9, the combination of any one of claims 10-13, or the method of any one of claims 14-17, wherein the antibody or antigen binding region that binds OX40L comprises a heavy chain comprising a constant heavy chain sequence set forth in SEQ ID NO: 61 or 173.

37. The composition of any one of claims 1-9, the combination of any one of claims 10-13, or the method of any one of claims 14-17, wherein the antibody or antigen binding region that binds OX40L comprises a light chain comprising a constant light chain sequence set forth in SEQ ID NO: 46.

38. The composition of any one of claims 1-9, the combination of any one of claims 10-13, or the method of any one of claims 14-17, wherein the antibody or antigen binding region that binds OX40L comprises a heavy chain comprising a constant heavy chain sequence set forth in SEQ ID NO: 61 or 173 and a light chain comprising a constant light chain sequence set forth in SEQ ID NO: 46.

39. The composition of any one of claims 1-9, the combination of any one of claims 10-13, or the method of any one of claims 14-17, wherein the hyaluronidase is a recombinant human hyaluronidase.

40. The composition, combination, or method of claim 39, wherein the recombinant human hyaluronidase comprises the amino acid sequence set forth in any one of SEQ ID NOs: 453-462 and 464-519.

41. The composition, combination, or method of claim 39, wherein the recombinant human hyaluronidase is rHuPH20.

42. The composition, combination, or method of claim 41, wherein the rHuPH20 formulation is ENHANZE®.

43. The combination of any one of claims 10-13 or the method of any one of claims 14-17, wherein the hyaluronidase and antibody or antibodies are administered in separate formulations.

44. The combination or method of claim 43, wherein the hyaluronidase and antibody or antibodies are administered simultaneously in separate formulations.

45. The combination or method of claim 43, wherein the hyaluronidase is administered to the patient prior to administration of the antibody or antibodies.

46. The combination or method of claim 43, wherein the hyaluronidase is administered to the patient after administration of the antibody or antibodies.

47. The combination or method of claim 43, wherein the hyaluronidase is administered to the patient after administration of one of the antibodies, but before administration of the other antibody.

48. The composition of any one of claims 1-9, the combination of any one of claims 10-13, or the method of any one of claims 14-17, wherein the hyaluronidase and antibody or antibodies are administered in a single formulation.

49. The composition of any one of claims 1-9, the combination of any one of claims 10-13, or the method of any one of claims 14-17, wherein the hyaluronidase and antibody or antibodies are administered by subcutaneous injection.

50. The composition of any one of claims 1-9, the combination of any one of claims 10-13, or the method of any one of claims 14-17, wherein the hyaluronidase and antibody or antibodies are administered by intravenous injection.

51. The composition of any one of claims 1-9, the combination of any one of claims 10-13, or the method of any one of claims 14-17, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

52. The method of any one of claims 14-17, wherein the inflammatory disorder or disease is atopic dermatitis.

53. The method of claim 52, wherein the treatment reduces disease severity in the patient and wherein disease severity is assessed by an atopic dermatitis disease severity outcome measure.

54. The method of any one of claims 14-17, wherein the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); Crohn's disease; lupus; rheumatoid arthritis (RA); psoriasis; ulcerative colitis; hidradenitis suppurativa; celiac disease; idiopathic pulmonary fibrosis; alopecia areata; systemic sclerosis; allergic rhinitis; eosinophilic fasciitis; scleromyxedema; scleredema; or nephrogenic systemic fibrosis.

55. A kit for treating an inflammatory disorder or disease in a human patient, the kit comprising:
(e) a dose of an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41;
(f) a dose of an antibody, or antigen binding fragment thereof, that binds OX40L, wherein the antibody, or antigen binding fragment thereof, comprises a VH sequence set forth in SEQ ID NO: 335 and a VL sequence set forth in SEQ ID NO: 339;
(g) a dose of a hyaluronidase or variant thereof; and
(h) instructions.

56. The kit of claim 55, wherein the hyaluronidase is a recombinant human hyaluronidase.

57. The kit of claim 56, wherein the recombinant human hyaluronidase comprises the amino acid sequence set forth in any one of SEQ ID NOs: 453-462 and 464-519.

58. The kit of claim 56, wherein the recombinant human hyaluronidase is rHuPH20.

59. The kit of claim 58, wherein the rHuPH20 formulation is ENHANZE®.

60. The kit of claim 55, for use in treating an inflammatory disorder or disease.

61. The kit of claim 60, wherein the inflammatory disorder or disease is atopic dermatitis.

62. The kit of claim 60, wherein the inflammatory disorder or disease is asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); Crohn's disease; lupus; rheumatoid arthritis (RA); psoriasis; ulcerative colitis; hidradenitis suppurativa; celiac disease; idiopathic pulmonary fibrosis; alopecia areata; systemic sclerosis; allergic rhinitis; eosinophilic fasciitis; scleromyxedema; scleredema; or nephrogenic systemic fibrosis.
